(19) United States
(12) Patent Application Publication
(10) Pub. No.: US 2023/0053649 A1
(43) Pub. Date: Feb. 23, 2023

(54) TEAD INHIBITORS AND USES THEREOF

(71) Applicant: Cedilla Therapeutics, Inc., Cambridge, MA (US)

(72) Inventors: Glen Robert Rennie, Somerville, MA (US); C. Eric Schwartz, Marblehead, MA (US); Dale Porter, Cambridge, MA (US); Ling Song, Cambridge, MA (US)

(21) Appl. No.: 17/808,868

(22) Filed: Jun. 24, 2022

Related U.S. Application Data
(60) Provisional application No. 63/226,967, filed on Jul. 29, 2021.
(60) Provisional application No. 63/214,869, filed on Jun. 25, 2021.

(51) Int. Cl. C07D 401/04; C07D 401/14; C07D 215/20; C07D 213/06; C07D 401/12; C07C 39/205

(52) U.S. Cl. CPC C07D 401/04; C07D 401/14; C07D 215/20; C07D 213/06; C07D 401/12; C07C 39/205

(57) ABSTRACT

The present disclosure provides compounds, pharmaceutically acceptable compositions thereof, and methods of using the same.

### TECHNICAL FIELD OF INVENTION

The present disclosure relates to compounds and methods useful for inhibition of Transcriptional Enhancer Associate Domain (TEAD) transcription factors. The disclosure also provides pharmaceutically acceptable compositions comprising compounds of the present disclosure and methods of using said compositions in the treatment of various diseases, disorders, and conditions as described herein.

### BACKGROUND OF THE INVENTION

Yes-associated protein (YAP) and transcriptional co-activator with PDZ-binding motif (TAZ) are transcriptional co-activators of the Hippo signaling pathway and regulate cell proliferation, migration, and apoptosis. Inhibition of the Hippo signaling pathway promotes YAP/TAZ translocation to the nucleus, where YAP/TAZ interact with TEAD transcription factors to co-activate the expression of target genes and promote cell proliferation. Hyperactivation of YAP and TAZ and/or mutations in one or more members of the Hippo signaling pathway have been implicated in various diseases, disorders, and conditions.

### SUMMARY OF THE INVENTION

In some embodiments, the present disclosure provides the recognition that there remains a need to find inhibitors of the Hippo signaling pathway useful as therapeutic agents. It has now been found that compounds of the present disclosure, and pharmaceutically acceptable salts and compositions thereof, are effective as inhibitors of TEAD transcription factors (e.g., TEAD1, TEAD2, TEAD3, and/or TEAD4). Such compounds have general Formula I:

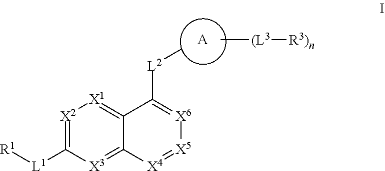

or a pharmaceutically acceptable salt thereof, wherein:
X¹ is C—R^{x1} or N;
X² is C—R^{x2} or N;
X³ is C—R^{x3} or N;
X⁴ is C—R^{x4} or N;
X⁵ is C—R^{x5} or N;
X⁶ is C—R^{x6} or N;
wherein no more than three of X¹, X², X³, X⁴, X⁵, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
   R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
L² is a covalent bond, —OCH₂—^{#}, or —N(R)CH₂—^{#}, wherein ^{#} represents the point of attachment to Ring A;
Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur; and
n is 0-5;
provided that when L² is a covalent bond and Ring A is phenyl, then at least one L³ is —O— or —NR—.

Compounds described herein, and pharmaceutically acceptable compositions thereof, are useful for treating a variety of diseases, disorders, or conditions associated with the Hippo signaling pathway. Such diseases, disorders, or conditions include those described herein.

Compounds provided herein are also useful for the study of the Hippo signaling pathway in, e.g., biological and pathological phenomena, and the comparative evaluation of new TEAD transcription factor inhibitors.

### BRIEF DESCRIPTION OF THE DRAWING

FIG. 1 depicts administration of compound I-1 in combination with Osimertinib to PC-9 cells.

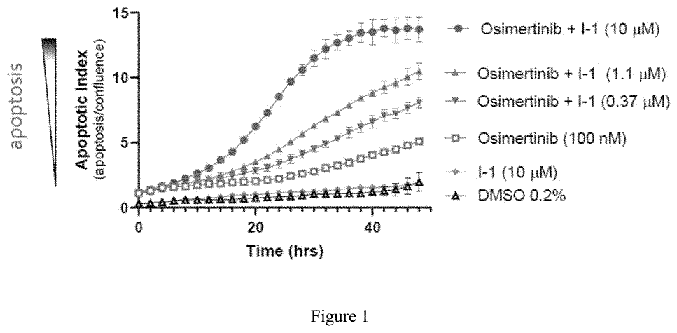

### DETAILED DESCRIPTION OF CERTAIN EMBODIMENTS

### 1. General Description of Certain Embodiments of the Invention:

In certain embodiments, the present disclosure provides inhibitors of TEAD transcription factors. In some embodiments, such compounds include those of the formulae described herein, or a pharmaceutically acceptable salt thereof, wherein each variable is as defined and described herein.

In one aspect, the present disclosure provides compounds of Formula I:

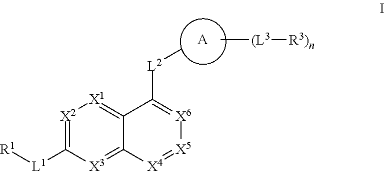

or a pharmaceutically acceptable salt thereof, wherein:
X¹ is C—R^{x1} or N;
X² is C—R^{x2} or N;
X³ is C—R^{x3} or N;
X⁴ is C—R^{x4} or N;
X⁵ is C—R^{x5} or N;
X⁶ is C—R^{x6} or N;
wherein no more than three of X¹, X², X³, X⁴, X⁵, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
   R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
L² is a covalent bond, —OCH₂—^{#}, or —N(R)CH₂—^{#}, wherein ^{#} represents the point of attachment to Ring A;
Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur; and
n is 0-5;
provided that when L² is a covalent bond and Ring A is phenyl, then at least one L³ is —O— or —NR—.

### 2. Compounds and Definitions:

Compounds of the present disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle”, “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “partially unsaturated”, as used herein, refers to a ring moiety that includes at least one double or triple bond. The term “partially unsaturated”, as used herein, is intended to encompass rings having multiple sites of unsaturation, but is not intended to include aryl or heteroaryl moieties, as herein defined.

The term “lower alkyl”, as used herein, refers to a C₁₋₄ straight or branched alkyl group. Exemplary lower alkyl groups are methyl, ethyl, propyl, isopropyl, butyl, isobutyl, and tert-butyl.

The term “halogen” means F, Cl, Br, or I.

The term “aryl”, as used herein, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Also included within the scope of the term “aryl” is a group in which an aromatic ring is fused to one or more non-aromatic rings, such as indanyl, phthalimidyl, naphthimidyl, phenanthridinyl, or tetrahydronaphthyl, and the like.

The term “heteroaryl”, as used herein, does not differ significantly from the common meaning of the term in the art, and refers to a cyclic aromatic radical having from five to twelve ring atoms of which one ring atom is selected from S, O and N; zero, one, two, three, four, or five ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

The term “heteroaryl” as used herein, refers to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” as used herein, refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, tetrahydroquinolinyl, and tetrahydroisoquinolinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples of heteroaryl rings on compounds of Formula I and subgenera thereof include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

Additionally, it will be appreciated that, when two groups cyclize to form an optionally substituted heteroaryl ring having at least one nitrogen atom, the nitrogen atom in the ring can be, as valency permits, N or N-RT, as defined infra.

As used herein, the terms “heterocycle”, “heterocyclyl”, and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, tetrahydroquinolinyl, or tetrahydroisoquinolinyl where the radical or point of attachment is on the heterocyclyl ring. A heterocyclyl group may be mono- or bicyclic.

Additionally, it will be appreciated that, when two groups cyclize to form an optionally substituted heterocyclic ring having at least one nitrogen atom, the nitrogen atom in the ring can be, as valency permits, N or N-RT, as defined infra.

As described herein, compounds may contain “optionally substituted” moieties. In general, the term “substituted”, whether preceded by the term “optionally” or not, means that one or more hydrogens of the designated moiety of compounds are replaced with a suitable substituent. “Substituted” applies to one or more hydrogens that are either explicit or implicit from the structure

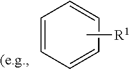

refers to at least

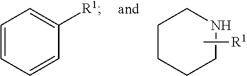

refers to at least

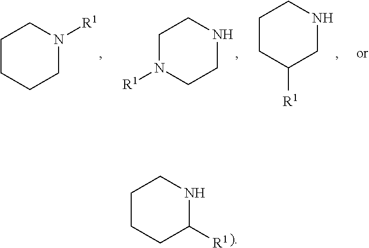

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-to 6 membered heteroaryl ring), or a 5- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, -(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group of a compound of Formula I, and subgenera thereof, include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R* is unsubstituted or where preceded by “halo” is substituted only with one or more halogens, and is independently C₁_₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms are within the scope of the disclosure. Additionally, unless otherwise stated, the present disclosure also includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure. In some embodiments, compounds of this disclosure comprise one or more deuterium atoms.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

As used herein, a “disease or disorder associated with TEAD” or, alternatively, “a TEAD-mediated disease or disorder” means any disease or other deleterious condition in which TEAD, or a mutant thereof, is known or suspected to play a role.

The term “subject”, as used herein, means a mammal and includes human and animal subjects, such as domestic animals (e.g., horses, dogs, cats, etc.). The terms “subject” and “patient” are used interchangeably. In some embodiments, the “patient” or “subject” means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The amount of compounds described herein that may be combined with the carrier materials to produce a composition in a single dosage form will vary depending upon the host treated, the particular mode of administration, etc.

The expression “unit dosage form” as used herein refers to a physically discrete unit of a provided compound and/or compositions thereof appropriate for the subject to be treated. It will be understood, however, that the total daily usage of the active agent (i.e., compounds and compositions described herein) will be decided by the attending physician within the scope of sound medical judgment. The specific effective dose level for any particular subject (i.e., patient) or organism will depend upon a variety of factors including the disorder being treated and the severity of the disorder; activity of specific active agent employed; specific composition employed; age, body weight, general health, sex and diet of the subject; time of administration, route of administration, and rate of excretion of the specific active agent employed; duration of the treatment; and like factors well known in the medical arts.

The term “parenteral” as used herein includes subcutaneous, intravenous, intramuscular, intra-articular, intra-synovial, intrasternal, intrathecal, intrahepatic, intralesional and intracranial injection or infusion techniques.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an TEAD-mediated disease or disorder.

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

### 3. Description of Exemplary Embodiments

In some embodiments, the present disclosure provides a compound of Formula I:

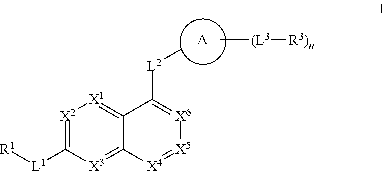

or a pharmaceutically acceptable salt thereof, wherein:
X¹ is C—R^{x1} or N;
X² is C—R^{x2} or N;
X³ is C—R^{x3} or N;
X⁴ is C—R^{x4} or N;
X⁵ is C—R^{x5} or N;
X⁶ is C—R^{x6} or N;
wherein no more than three of X¹, X², X³, X⁴, X⁵, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
   R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
L² is a covalent bond, —OCH₂—^{#}, or —N(R)CH₂—^{#}, wherein ^{#} represents the point of attachment to Ring A;
Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur; and
n is 0-5;
provided that when L² is a covalent bond and Ring A is phenyl, then at least one L³ is —O— or —NR—.

As defined generally above, when L² is a covalent bond and Ring A is phenyl, then at least one L³ is —O— or —NR—. In some embodiments, when L² is a covalent bond and Ring A is phenyl, then at least one L³ is —O—. In some embodiments, when L² is a covalent bond and Ring A is phenyl, then at least one L³ is —NR—.

As defined generally above, X¹ is C—R^{x1} or N. In some embodiments, X¹ is N. In some embodiments, X¹ is C—R^{x1}.

As defined generally above, R^{x1} is selected from hydrogen, —CN, halogen, —OR, —N(R)₂, or an optionally substituted group selected from the group consisting of C₁₋₆ aliphatic, phenyl, a 5-to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x1} is selected from hydrogen, —CN, halogen, —OR, —N(R)₂, or an optionally substituted C₁₋₆ aliphatic. In some embodiments, R^{x1} is hydrogen. In some embodiments, R^{x1} is —OR. In some embodiments, R^{x1} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x1} is —OR, wherein R is an optionally substituted group selected from the group consisting of C₁₋₆ aliphatic and 3- to 7-membered saturated or partially unsaturated carbocyclic ring. In some embodiments, R^{x1} is —OR, wherein R is C₁₋₃ aliphatic optionally substituted with halogen. In some embodiments, R^{x1} is —OR, wherein R is C₁₋₃ aliphatic optionally substituted with fluoro. In some embodiments, R^{x1} is —OR, wherein R is methyl optionally substituted with 1-3 fluoro groups. In some embodiments, R^{x1} is —OR, wherein R is cyclopropyl. In some embodiments, R^{x1} is hydrogen, —OCH₃, —OCF₂H, —OCF₃, or

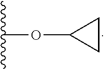 [SMILES: *OC1CC1]

In some embodiments, R^{x1} is —OCH₃, —OCF₂H, —OCF₃, or

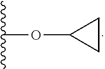 [SMILES: *OC1CC1]

As defined generally above, X² is C—R^{x2} or N. In some embodiments, X² is N. In some embodiments, X² is C—R^{x2}.

As defined generally above, R^{x2} is selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x2} is selected from hydrogen, —CN, halogen, —OR, —N(R)₂, or an optionally substituted C₁₋₆ aliphatic. In some embodiments, R^{x2} is hydrogen. In some embodiments, R^{x2} is —OR. In some embodiments, R^{x2} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x2} is —OR, wherein R is an optionally substituted group selected from the group consisting of C₁₋₆ aliphatic and 3- to 7-membered saturated or partially unsaturated carbocyclic ring. In some embodiments, R^{x2} is —OR, wherein R is C₁₋₃ aliphatic optionally substituted with halogen. In some embodiments, R^{x2} is —OR, wherein R is C₁₋₃ aliphatic optionally substituted with fluoro. In some embodiments, R^{x2} is —OR, wherein R is methyl optionally substituted with 1-3 fluoro groups. In some embodiments, R^{x2} is —OR, wherein R is cyclopropyl. In some embodiments, R^{x2} is hydrogen, —OCH₃, —OCF₂H, —OCF₃, or

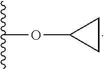 [SMILES: *OC1CC1]

In some embodiments, R^{x2} is —OCH₃, —OCF₂H, —OCF₃, or

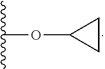 [SMILES: *OC1CC1]

As defined generally above, X³ is C—R^{x3} or N. In some embodiments, X³ is N. In some embodiments, X¹ is C—R^{x3}.

As defined generally above, R^{x3} is selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x3} is selected from hydrogen, —CN, halogen, —OR, —N(R)₂, or an optionally substituted group selected from the group consisting of C₁₋₆ aliphatic. In some embodiments, R^{x3} is hydrogen. In some embodiments, R^{x3} is —OR. In some embodiments, R^{x3} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x3} is —OR, wherein R is an optionally substituted group selected from the group consisting of C₁₋₆ aliphatic and 3- to 7-membered saturated or partially unsaturated carbocyclic ring. In some embodiments, R^{x3} is —OR, wherein R is C₁₋₃ aliphatic optionally substituted with halogen. In some embodiments, R^{x3} is —OR, wherein R is C₁₋₃ aliphatic optionally substituted with fluoro. In some embodiments, R^{x3} is —OR, wherein R is methyl optionally substituted with 1-3 fluoro groups. In some embodiments, R^{x3} is —OR, wherein R is cyclopropyl. In some embodiments, R^{x3} is hydrogen, —OCH₃, —OCF₂H, —OCF₃, or

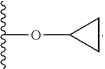 [SMILES: *OC1CC1]

In some embodiments, R^{x3} is —OCH₃, —OCF₂H, —OCF₃, or.

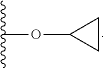 [SMILES: *OC1CC1]

As defined generally above, X⁴ is C—R^{x4} or N. In some embodiments, X⁴ is N. In some embodiments, X⁴ is C—R^{x4}.

As defined generally above, R^{x4} is selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x4} is selected from hydrogen, —CN, halogen, —OR, —N(R)₂, or an optionally substituted C₁₋₆ aliphatic. In some embodiments, R^{x4} is hydrogen. In some embodiments, R^{x4} is —OR. In some embodiments, R^{x4} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x4} is —OR, wherein R is an optionally substituted group selected from the group consisting of C₁₋₆ aliphatic and 3- to 7-membered saturated or partially unsaturated carbocyclic ring. In some embodiments, R^{x4} is —OR, wherein R is C₁₋₃ aliphatic optionally substituted with halogen. In some embodiments, R^{x4} is —OR, wherein R is C₁₋₃ aliphatic optionally substituted with fluoro. In some embodiments, R^{x4} is —OR, wherein R is methyl optionally substituted with 1-3 fluoro groups. In some embodiments, R^{x4} is —OR, wherein R is cyclopropyl. In some embodiments, R^{x4} is hydrogen, —OCH₃, —OCF₂H, —OCF₃, or

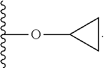 [SMILES: *OC1CC1]

In some embodiments, R^{x4} is —OCH₃, —OCF₂H, —OCF₃, or

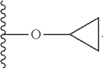 [SMILES: *OC1CC1]

As defined generally above, X⁵ is C—R^{x5} or N. In some embodiments, X⁵ is N. In some embodiments, X⁵ is C—R^{x5}.

As defined generally above, R^{x5} is selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x5} is selected from hydrogen, —CN, halogen, —OR, —N(R)₂, or an optionally substituted C₁₋₆ aliphatic. In some embodiments, R^{x5} is hydrogen. In some embodiments, R^{x5} is —OR. In some embodiments, R^{x5} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x5} is —OR, wherein R is an optionally substituted group selected from the group consisting of C₁₋₆ aliphatic and 3- to 7-membered saturated or partially unsaturated carbocyclic ring. In some embodiments, R^{x5} is —OR, wherein R is C₁₋₃ aliphatic optionally substituted with halogen. In some embodiments, R^{x5} is —OR, wherein R is C₁₋₃ aliphatic optionally substituted with fluoro. In some embodiments, R^{x5} is —OR, wherein R is methyl optionally substituted with 1-3 fluoro groups. In some embodiments, R^{x5} is —OR, wherein R is cyclopropyl. In some embodiments, R^{x5} is hydrogen, —OCH₃, —OCF₂H, —OCF₃, or

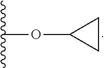 [SMILES: *OC1CC1]

In some embodiments, R^{x5} is —OCH₃, —OCF₂H, —OCF₃, or.

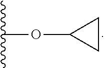 [SMILES: *OC1CC1]

In some embodiments, R^{x5} is optionally substituted C₁₋₆ aliphatic. In some embodiments, R^{x5} is methyl. In some embodiments, R^{x5} is C₁₋₆ aliphatic optionally substituted with halogen. In some embodiments, R^{x5} is methyl, optionally substituted with halogen. In some embodiments, R^{x5} is —CF₃. In some embodiments, R^{x5} is —OH. In some embodiments, R^{x5} is —OCH₂CH₃.

In some embodiments, R^{x5} is hydrogen, —OCH₃, —OCH₂CH₃, —OCF₂H, —OCF₃,

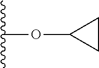 [SMILES: *OC1CC1]

—OH, methyl, or —CF₃. In some embodiments, R^{x5} is —OCH₃, —OCH₂CH₃, —OCF₂H, —OCF₃,

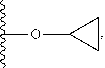 [SMILES: *OC1CC1]

—OH, methyl, or —CF₃. In some embodiments, R^{x5} is —OCH₂CH₃, —OH, methyl, or —CF₃.

As defined generally above, X⁶ is C—R^{x6} or N. In some embodiments, X⁶ is N. In some embodiments, X⁶ is C—R^{x6}.

As defined generally above, R^{x6} is selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x6} is selected from hydrogen, —CN, halogen, —OR, —N(R)₂, or an optionally substituted C₁₋₆ aliphatic. In some embodiments, R^{x6} is hydrogen. In some embodiments, R^{x6} is —OR. In some embodiments, R^{x6} is —OR, wherein R is hydrogen or an optionally substituted group selected from the group consisting of C₁₋₆ aliphatic, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x6} is —OR, wherein R is an optionally substituted group selected from the group consisting of C₁₋₆ aliphatic and 3- to 7-membered saturated or partially unsaturated carbocyclic ring. In some embodiments, R^{x6} is —OR, wherein R is C₁₋₃ aliphatic optionally substituted with halogen. In some embodiments, R^{x6} is —OR, wherein R is C₁₋₃ aliphatic optionally substituted with fluoro. In some embodiments, R^{x6} is —OR, wherein R is methyl optionally substituted with 1-3 fluoro groups. In some embodiments, R^{x6} is —OR, wherein R is cyclopropyl. In some embodiments, R^{x6} is hydrogen, —OCH₃, —OCF₂H, —OCF₃, or

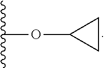 [SMILES: *OC1CC1]

In some embodiments, R^{x6} is —OCH₃, —OCF₂H, —OCF₃, or

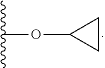 [SMILES: *OC1CC1]

As defined generally above, no more than three of X¹, X², X³, X⁴, X⁵, or X⁶ are N. In some embodiments, no more than two of X¹, X², X³, X⁴, X⁵, or X⁶ are N. In some embodiments, no more than one of X¹, X², X³, X⁴, X⁵, or X⁶ are N. In some embodiments, one or two of X¹, X², X³, X⁴, X⁵, or X⁶ are N. In some embodiments, one of X¹, X², X³, X⁴, X⁵, or X⁶ is N. In some embodiments, two of X¹, X², X³, X⁴, X⁵, or X⁶ are N.

As defined generally above, L¹ is —C(O)N(R²)—*, —S(O)₂—*, —S(O)₂N(R²)—*, or —C(O)O—*, wherein * represents the point of attachment to R¹.

In some embodiments, L¹ is —C(O)N(R²)—*, —S(O)₂N(R²)—*, or —C(O)O—*. In some embodiments, L¹ is —C(O)N(R²)—* or —C(O)O—*. In some embodiments, L¹ is —C(O)N(R²)—*. In some embodiments, L¹ is —C(O)NH—*. In some embodiments, L¹ is —S(O)₂—*. In some embodiments, L¹ is —S(O)₂N(R²)—*. In some embodiments, L¹ is —S(O)₂NH—*. In some embodiments, L¹ is —C(O)O—*.

As defined generally above, R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R¹ is hydrogen. In some embodiments, R¹ is an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R¹ is an optionally substituted group selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur.

In some embodiments, R¹ is an optionally substituted C₁₋₆ aliphatic. In some embodiments, R¹ is an optionally substituted C₃₋₄ aliphatic. In some embodiments, R¹ is C_{30.4} aliphatic optionally substituted with —N(R^{∘})₂ or —OR^{∘}, wherein R^{∘} is selected from hydrogen or C₁₋₆ aliphatic, and wherein R^{∘} may be substituted with halogen. In some embodiments, R¹ is C₃₋₄ aliphatic optionally substituted with —OR^{∘}, wherein R^{∘} is selected from hydrogen or C₁₋₆ aliphatic, and wherein R^{∘} may be substituted with halogen. In some embodiments, R¹ is C₃₋₄ aliphatic optionally substituted with —OR^{∘}, wherein R^{∘} is selected from hydrogen or C₁₋₆ aliphatic, and wherein R^{∘} may be substituted with flourine. In some embodiments, R¹ is selected from the group consisting of:

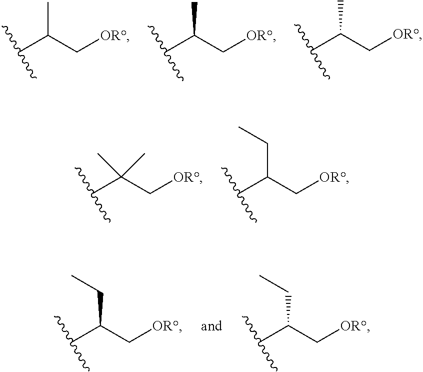

wherein R^{∘} is selected from hydrogen or C₁₋₆ aliphatic, which may be substituted with halogen.

In some such embodiments, R^{∘} is hydrogen, —CH₃, or CF₃.

In some embodiments, R¹ is selected from the group consisting of:

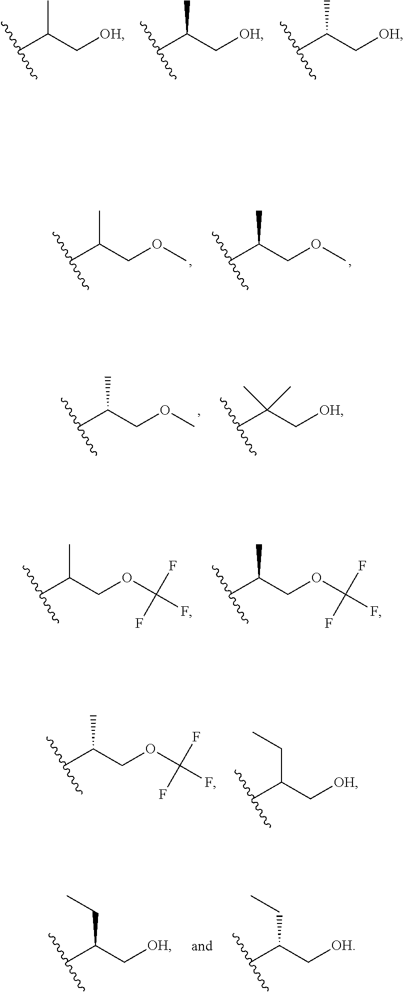

In some embodiments, R¹ is an optionally substituted C₁₋₂ aliphatic. In some embodiments, R¹ is an optionally substituted C₂ aliphatic. In some embodiments, R¹ is C₂ aliphatic optionally substituted with R^{∘}. In some embodiments, R¹ is C₂ aliphatic optionally substituted with R^{∘}, wherein R^{∘} is a 5- to 6-membered saturated, partially unsaturated, or aryl ring having 0-4 heteroatoms independently selected from nitrogen, oxygen, or sulfur. In some embodiments, R¹ is C₂ aliphatic optionally substituted with R^{∘}, wherein R^{∘} is phenyl or pyridyl. In some embodiments, R¹ is C₂ aliphatic optionally substituted with R^{∘}, wherein R^{∘} is phenyl or pyridyl, wherein R^{∘} is substituted with —OH, —OR^{•}, —C(O)OH, or —C(O)OR^{•}. In some embodiments, R¹ is C₂ aliphatic optionally substituted with R^{∘}, wherein R^{∘} is phenyl or pyridyl, wherein R^{∘} is substituted with —OH, —OR^{•}, —C(O)OH, or —C(O)OR^{•}, where R^{•} is C₁₋₄ aliphatic. In some embodiments, R¹ is selected from the group consisting of:

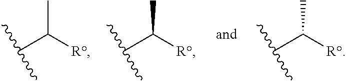

In some embodiments, R¹ is selected from the group consisting of:

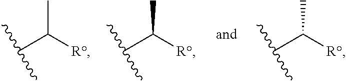

wherein R^{∘} is a 5- to 6-membered saturated, partially unsaturated, or aryl ring having 0-4 heteroatoms independently selected from nitrogen, oxygen, or sulfur. In some such embodiments, R^{∘} is phenyl or pyridyl, wherein R^{∘} may be substituted with —OH, —OR^{•}, —C(O)OH, or —C(O)OR^{•}. In some such embodiments, R^{∘} is phenyl or pyridyl, wherein R^{∘} may be substituted with —OH, —OR^{•}, —C(O)OH, or —C(O)OR^{•}, where R^{•} is C₁₋₄ aliphatic. In some embodiments, R¹ is selected from the group consisting of:

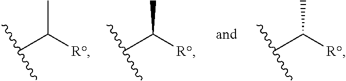

wherein R^{∘} is

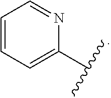 [SMILES: *c1ccccn1]

In some embodiments, R¹ is selected from the group consisting of:

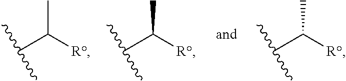

wherein R^{∘} is phenyl. In some embodiments, R¹ is selected from the group consisting of:

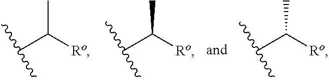

wherein R^{∘} is phenyl substituted with —OCH₃ or —C(O)OH.

In some embodiments, R¹ is C₂ aliphatic optionally substituted with —C(O)OR^{∘}. In some embodiments, R¹ is C₂ aliphatic optionally substituted with —C(O)OR^{∘}, wherein R^{∘} is hydrogen or C₁₋₆ aliphatic. In some embodiments, R¹ is selected from the group consisting of:

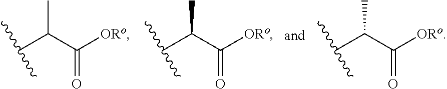

In some embodiments, R¹ is optionally substituted C₃ aliphatic. In some embodiments, R¹ is

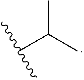 [SMILES: *C(C)C]

In some embodiments, R¹ is optionally substituted C₅ aliphatic. In some embodiments, R¹ is C₅ aliphatic optionally substituted with R^{∘}. In some embodiments, R¹ is selected from the group consisting of:

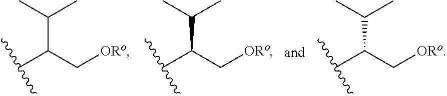

In some embodiments, R¹ is selected from the group consisting of:

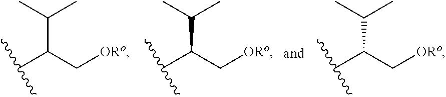

wherein R^{∘} is selected from hydrogen or C₁₋₆ aliphatic. In some embodiments, R¹ is selected from the group consisting of:

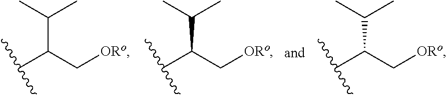

wherein R^{∘} is selected from hydrogen or C₁₋₆ aliphatic, which may be substituted with halogen.

In some embodiments, R¹ is selected from the group consisting of:

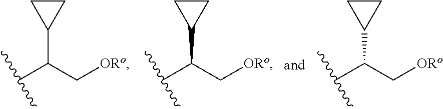

wherein R^{∘} is selected from hydrogen or C₁₋₆ aliphatic. In some embodiments, R¹ is selected from the group consisting of:

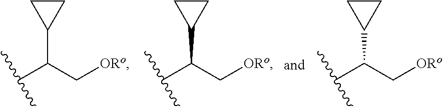

wherein R^{∘} is selected from hydrogen or C₁₋₆ aliphatic, which may be substituted with halogen.

In some embodiments, R¹ is an optionally substituted C₆ aliphatic. In some embodiments, R¹ is C₆ aliphatic optionally substituted with —N(R^{∘})₂ or —OR^{∘}. In some embodiments, R¹ is C₆ aliphatic optionally substituted with —N(R^{∘})₂ or —OR^{∘}, wherein R^{∘} is selected from hydrogen or C₁₋₆ aliphatic. In some embodiments, R¹ is C₆ aliphatic optionally substituted with —OR^{∘}, wherein R^{∘} is selected from hydrogen or C₁₋₆ aliphatic. In some embodiments, R¹ is selected from the group consisting of:

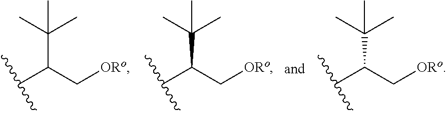

In some embodiments, R¹ is selected from the group consisting of:

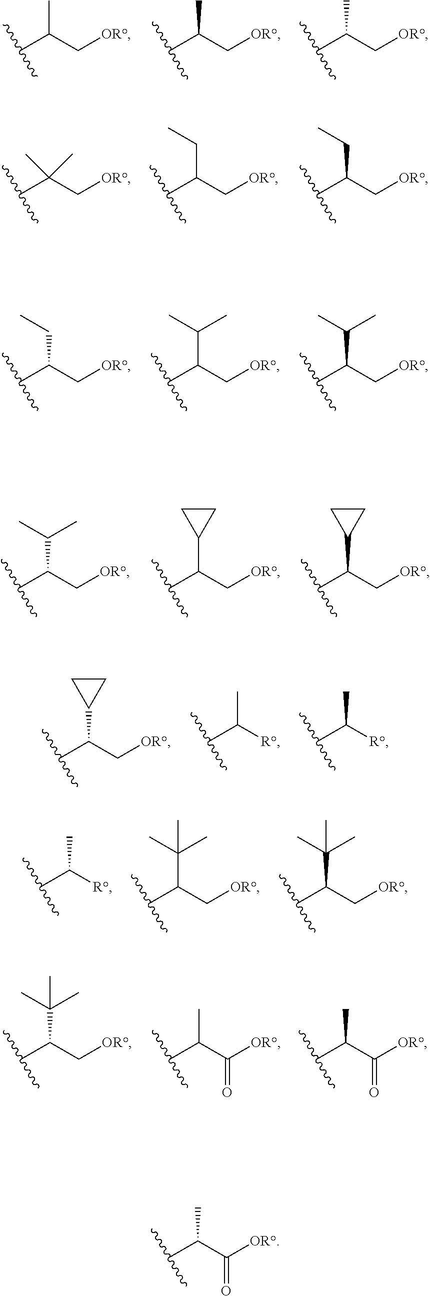

wherein R^{∘} is selected from hydrogen; C₁₋₆ aliphatic, which may be substituted with halogen; or pyridyl or phenyl, which may be substituted with —OH, —OR^{•}, —C(O)OH, or —C(O)OR^{•}, where R^{•} is C₁₋₄ aliphatic.

In some embodiments, R¹ is selected from the group consisting of:

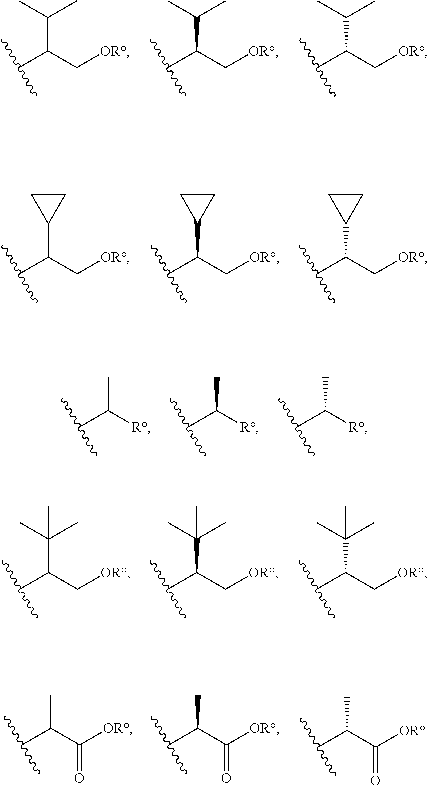

wherein R^{∘} is selected from hydrogen; C₁₋₆ aliphatic, which may be substituted with halogen; or pyridyl or phenyl, which may be substituted with —OH, —OR^{•}, —C(O)OH, or —C(O)OR^{•}, where R^{•} is C₁₋₄ aliphatic. In some such embodiments, R^{∘} is hydrogen, —CH₃, —CF₃, pyridyl, or phenyl substituted with —OMe or —C(O)OH.

In some embodiments, R¹ is selected from the group consisting of:

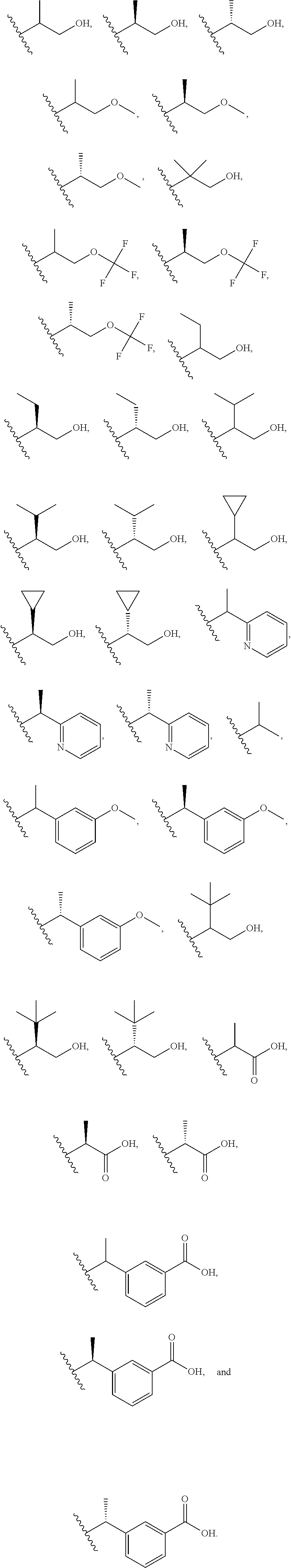

In some embodiments, R¹ is selected from the group consisting of:

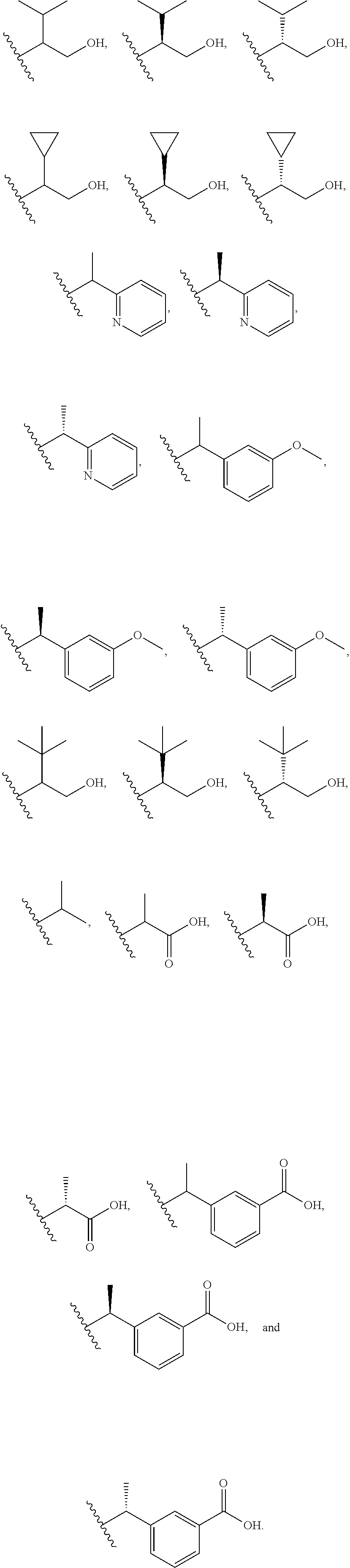

As defined generally above, R² is hydrogen or an optionally substituted C₁₋₆ aliphatic. In some embodiments, R² is hydrogen. In some embodiments, R² is an optionally substituted C₁₋₆ aliphatic. In some embodiments, R² is methyl.

As defined generally above, R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur. In some embodiments, R¹ and R², together with their intervening atoms, form an optionally substituted 3- to 7-membered saturated or partially unsaturated heterocyclic ring having 1-3 heteroatoms atoms independently selected from nitrogen, oxygen, and sulfur. In some embodiments, R¹ and R², together with their intervening atoms, form an optionally substituted 3- to 7-membered saturated or partially unsaturated heterocyclic ring having 1 nitrogen heteroatom. In some embodiments, R¹ and R², together with their intervening atoms, form an optionally substituted 4-membered saturated or partially unsaturated heterocyclic ring having 1 nitrogen heteroatom. In some embodiments, R¹ and R², together with their intervening atoms, form an optionally substituted

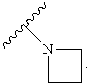 [SMILES: *N1CCC1]

In some embodiments, R¹ and R², together with their intervening atoms, form

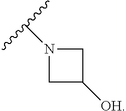 [SMILES: *N1CC(O)C1]

In some embodiments, the moiety L¹-R¹ is OH.

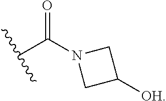 [SMILES: *C(=O)N1CC(O)C1]

As defined generally above, L² is a covalent bond, —OCH₂—^{#}, or —N(R)CH₂—^{#}, wherein ^{#} represents the point of attachment to Ring A. In some embodiments, L² is a covalent bond. In some embodiments, L² is —OCH₂—^{#}or —N(R)CH₂—^{#}. In some embodiments, L² is —OCH₂—^{#}. In some embodiments, L² is —N(R)CH₂—^{#}. In some embodiments, L² is a covalent bond or —OCH₂—^{#}.

As defined generally above, Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur. In some embodiments, Ring A is phenyl. In some embodiments, Ring A is selected from the group consisting of a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur.

In some embodiments, Ring A is a 5- to 6-membered heteroaryl ring having 1-3 heteroatoms independently selected from nitrogen, oxygen, and sulfur. In some embodiments, Ring A is a 5- to 6-membered heteroaryl ring having 1-3 nitrogen heteroatoms.

In some embodiments, Ring A is a 3- to 7-membered saturated or partially unsaturated carbocyclic ring. In some embodiments, Ring A is a 5-membered saturated or partially unsaturated carbocyclic ring. In some embodiments, Ring A is a 6-membered saturated or partially unsaturated carbocyclic ring. In some embodiments, Ring A is cyclohexyl.

In some embodiments, Ring A is a 3- to 7-membered saturated or partially unsaturated heterocyclic ring having 1-3 heteroatoms independently selected from nitrogen, oxygen, and sulfur. In some embodiments, Ring A is a 6-membered saturated or partially unsaturated heterocyclic ring having 1-3 heteroatoms independently selected from nitrogen, oxygen, and sulfur. In some embodiments, Ring A is a 6-membered saturated or partially unsaturated heterocyclic ring having 1-2 nitrogen heteroatoms. In some embodiments, Ring A is piperidinyl or piperazinyl.

In some embodiments, Ring A is a 8- to 11-membered spirofused saturated or partially unsaturated heterocyclic ring having 1-3 heteroatoms independently selected from nitrogen, oxygen, and sulfur. In some embodiments, Ring A is a 8- to 10-membered spirofused saturated or partially unsaturated heterocyclic ring having 1 nitrogen heteroatom. In some embodiments, Ring A is an 8-membered spirofused saturated or partially unsaturated heterocyclic ring having 1 nitrogen heteroatom. In some embodiments, Ring A is a 9-membered spirofused saturated or partially unsaturated heterocyclic ring having 1 nitrogen heteroatom. In some embodiments, Ring A is a 10-membered spirofused saturated or partially unsaturated heterocyclic ring having 1 nitrogen heteroatom. In some embodiments, Ring A is 6-azaspiro[2.5]octanyl, 7-azaspiro[3.5]nonanyl, or 8-azaspiro[4.5]decanyl.

In some embodiments, Ring A is selected from the group consisting of phenyl, cyclohexyl, piperidinyl, piperazinyl, 6-azaspiro[2.5]octanyl, 7-azaspiro[3.5]nonanyl, and 8-azaspiro[4.5]decanyl. In some embodiments, Ring A is selected from the group consisting of cyclohexyl, piperidinyl, piperazinyl, 6-azaspiro[2.5]octanyl, 7-azaspiro[3.5]nonanyl, and 8-azaspiro[4.5]decanyl.

In some embodiments, Ring A is selected from the group consisting of:

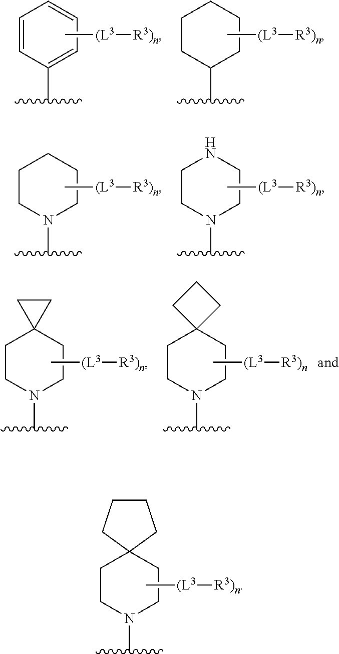

In some embodiments, Ring A is selected from the group consisting of:

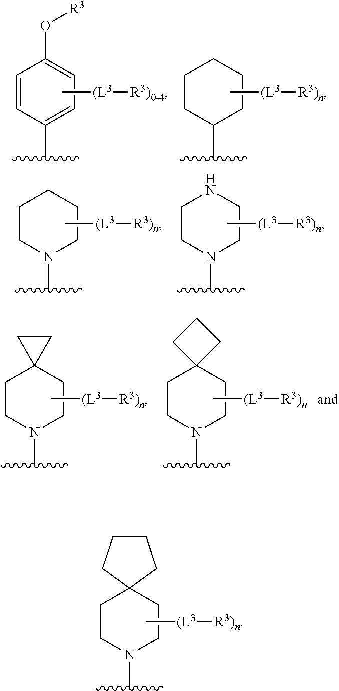

As defined generally above, each L³ is independently a covalent bond, —O—, or —NR—. In some embodiments, L³ is a covalent bond or —O—. In some embodiments, L³ is a covalent bond. In some embodiments, L³ is —O—. In some embodiments, L³ is —NR—. In some embodiments, L³ is —NH—.

As defined generally above, each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur.

In some embodiments, R³ is halogen or an optionally substituted group selected from the group consisting of C₁₋₆ aliphatic, phenyl, or a 3- to 7-membered saturated or partially unsaturated carbocyclic ring.

In some embodiments, R³ is halogen. In some embodiments, R³ is fluoro or chloro. In some embodiments, R³ is fluoro. In some embodiments, R³ is chloro. In some embodiments, R³ is an optionally substituted C₁₋₆ aliphatic. In some embodiments, R³ is an optionally substituted C₁₋₄ aliphatic. In some embodiments, R³ is C₁₋₄ aliphatic optionally substituted with halogen. In some embodiments, R³ is C₁₋₂ aliphatic optionally substituted with halogen. In some embodiments, R³ is t-butyl, —CHF₂, —CF₃, or —CH₂CF₃. In some embodiments, R³ is optionally substituted phenyl. In some embodiments, R³ is phenyl. In some embodiments, R³ is an optionally substituted 3- to 7-membered saturated or partially unsaturated carbocyclic ring. In some embodiments, R³ is an optionally substituted 6-membered saturated or partially unsaturated carbocyclic ring. In some embodiments, R³ is cyclopropyl. In some embodiments, R³ is fluoro, t-butyl, —CHF₂, —CF₃, —CH₂CF₃, phenyl, or cyclopropyl.

In some embodiments, L³ is —O—, and R³ is an optionally substituted group selected from the group consisting of C₁₋₆ aliphatic, phenyl, or a 3- to 7-membered saturated or partially unsaturated carbocyclic ring. In some embodiments, L³ is —O—, and R³ is t-butyl, —CHF₂, —CF₃, —CH₂CF₃, phenyl, or cyclopropyl.

In some embodiments, L³ is a covalent bond, and R³ is halogen or optionally substituted C₁₋₆ aliphatic. In some embodiments, L³ is a covalent bond, and R³ is fluoro, t-butyl, —CHF₂, —CF₃, or —CH₂CF₃.

In some embodiments, -L³-R³ is selected from the group consisting of:

fluoro, t-butyl, —CHF₂, —CF₃, —CH₂CF₃, —OCHF₂, —OCF₃, —OCH₂CF₃, —O-t-butyl, —O-phenyl, and —O-cyclopropyl.

As defined generally above, n is 0-5. In some embodiments, n is 0-4. In some embodiments, n is 0-3. In some embodiments, n is 0-2. In some embodiments, n is 1-2. In some embodiments, n is 1-3. In some embodiments, n is 2-3. In some embodiments, n is 0. In some embodiments, n is 1. In some embodiments, n is 2. In some embodiments, n is 3. In some embodiments, n is 4. In some embodiments, n is 5.

In some embodiments, the present disclosure provides a compound of Formulae II-a, II-a1, II-a2, II-b, II-b1, II-b2, II-c, II-c1, II-c2, II-d, II-d1, or II-d2:

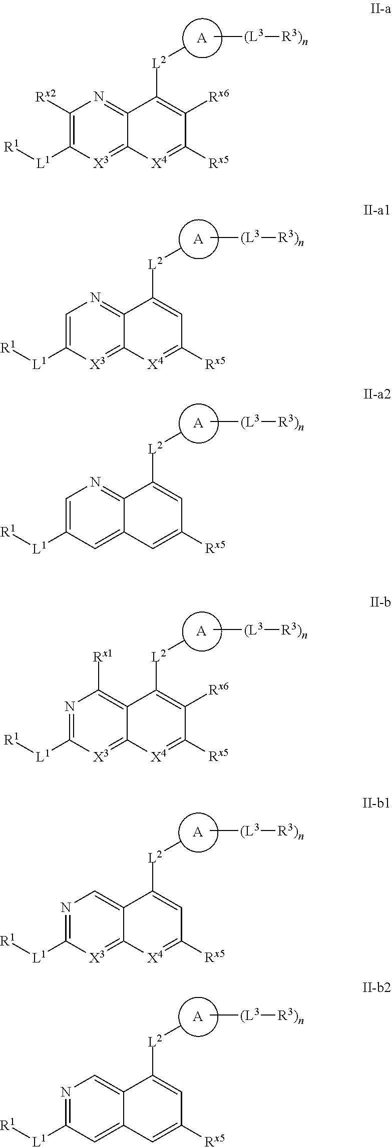

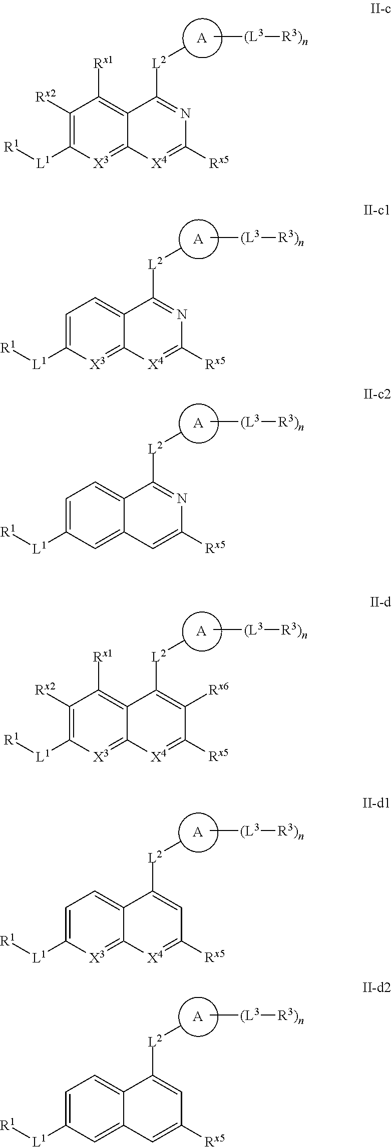

or a pharmaceutically acceptable salt thereof, wherein each of LI, L², X³, X⁴, R¹, Ring A, L³, R³, n, R^{x1}, R^{x2}, R^{x5}, and R^{x6} is as defined above and described herein.

In some embodiments, the present disclosure provides a compound of Formulae III-a, III-a1, III-a2, III-b, III-b1, III-b2, III-c, III-c1, III-c2, III-d, III-d1, or III-d2:

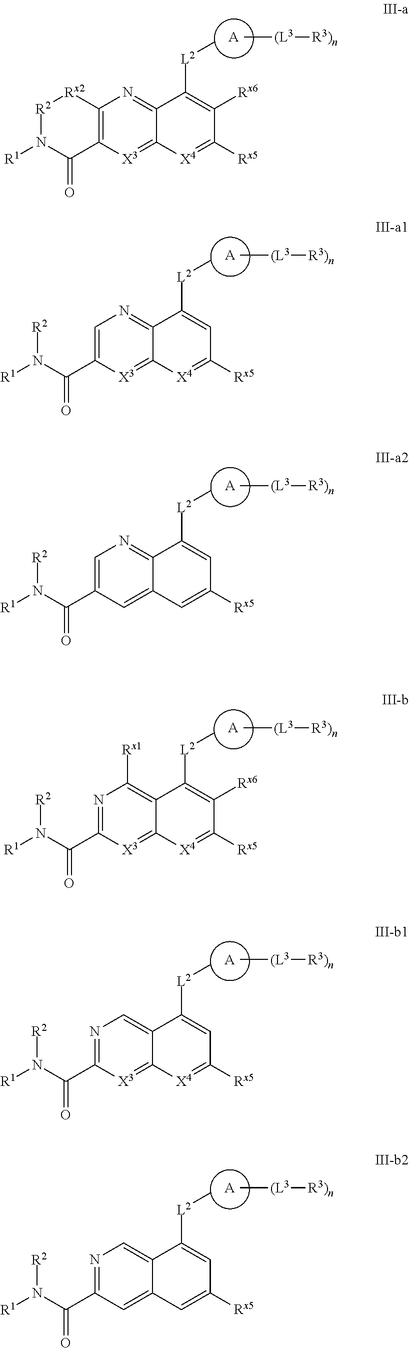

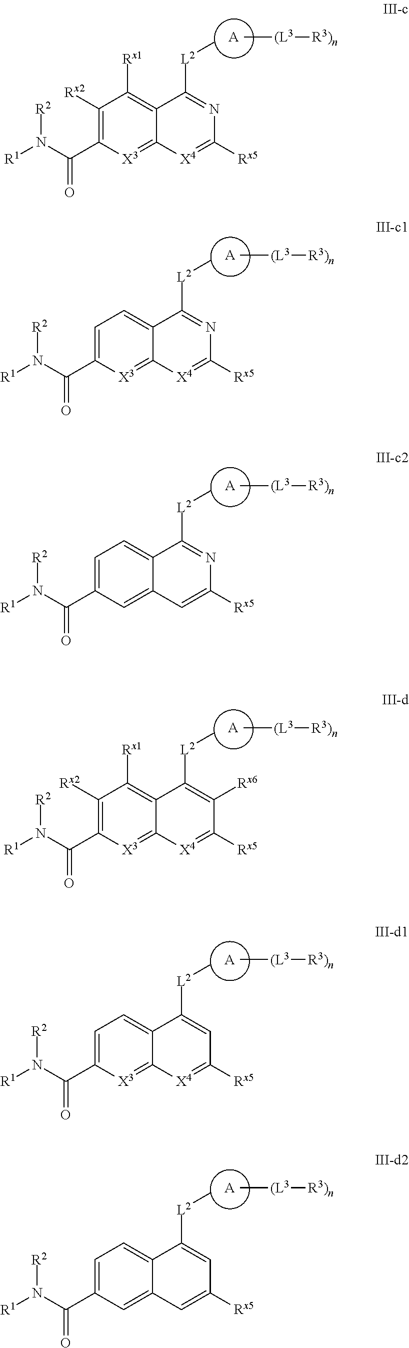

or a pharmaceutically acceptable salt thereof, wherein each of L², X³, X⁴, R¹, R², Ring A, L³, R³, n, R^{x1}, R^{x2}, R^{x5}, and R^{x6} is as defined above and described herein.

In some embodiments, the present disclosure provides a compound of Formulae IV-a, IV-a1, IV-a2, IV-b, IV-b1, IV-b2, IV-c, IV-c1, IV-c2, IV-d, IV-d1, or IV-d2:

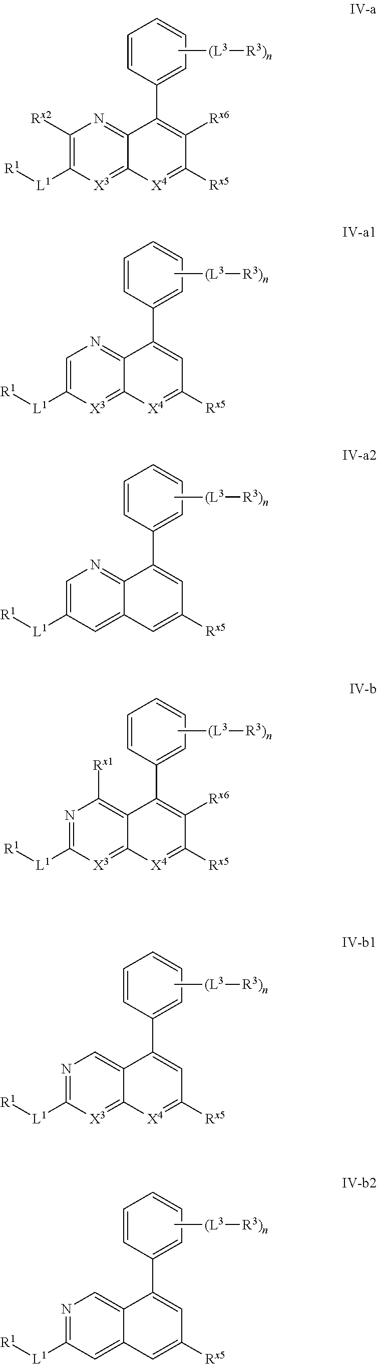

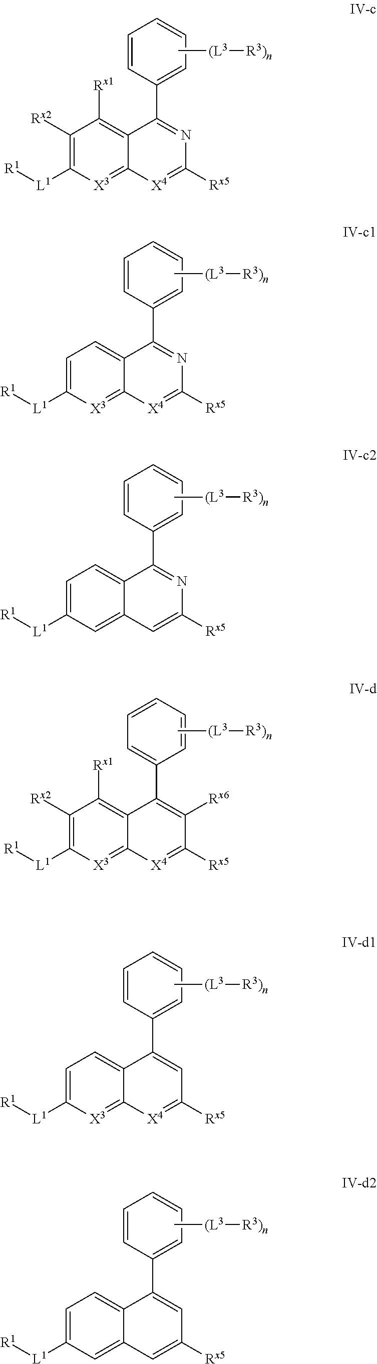

or a pharmaceutically acceptable salt thereof, wherein each of Li, X³, X⁴, R¹, L³, R³, n, R^{x1}, R^{x2}, R^{x5}, and R^{x6} is as defined above and described herein.

In some embodiments, the present disclosure provides a compound of Formulae V-a, V-a1, V-a2, V-b, V-b1, V-b2, V-c, V-c1, V-c2, V-d, V-d1, or V-d2:

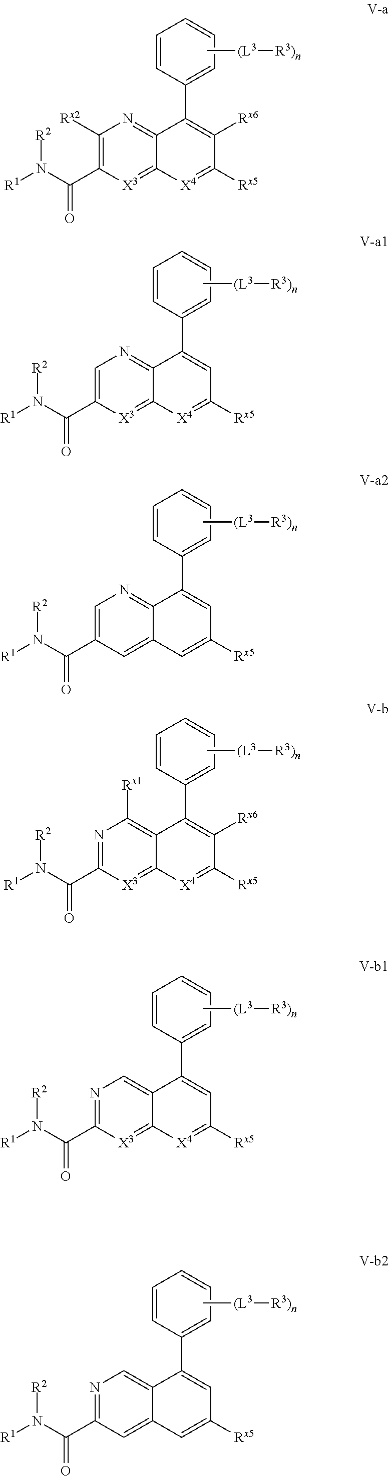

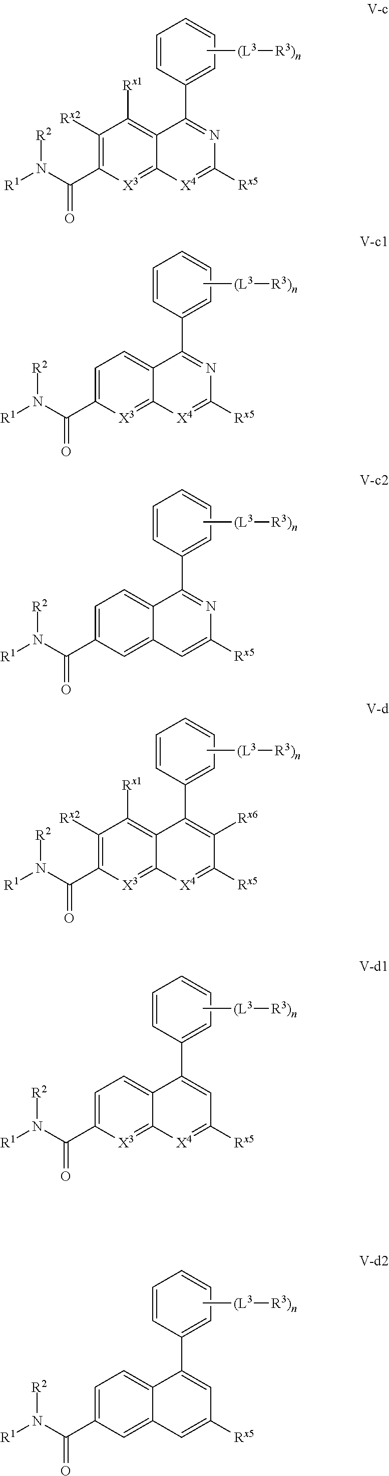

or a pharmaceutically acceptable salt thereof, wherein each of X³, X⁴, R¹, R², L³, R³, n, R^{x1}, R^{x2}, R^{x5}, and R^{x6} is as defined above and described herein.

In some embodiments, the present disclosure provides a compound of Formulae VI-a, VI-a1, VI-a2, VI-b, VI-b1, VI-b2, VI-c, VI-c1, VI-c2, VI-d, VI-d1, or VI-d2:

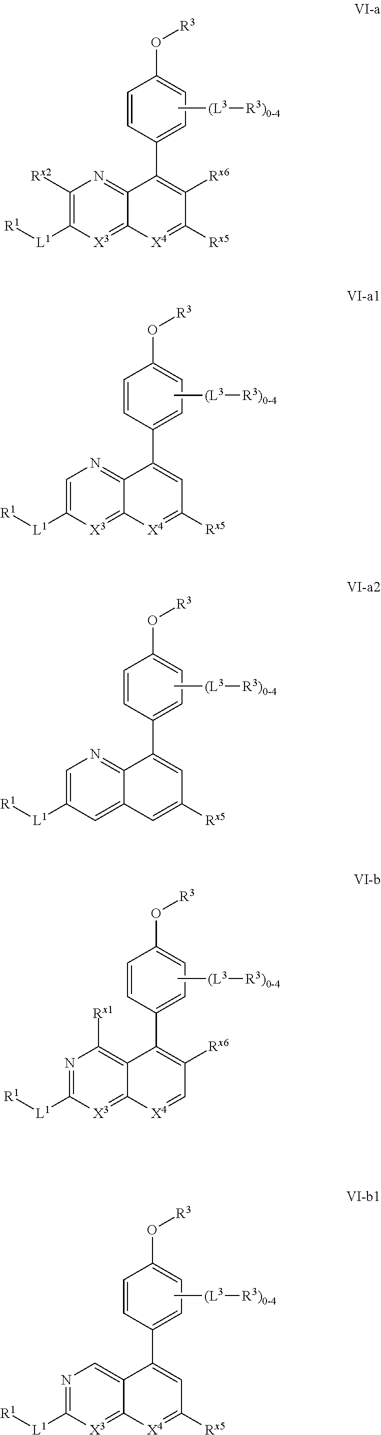

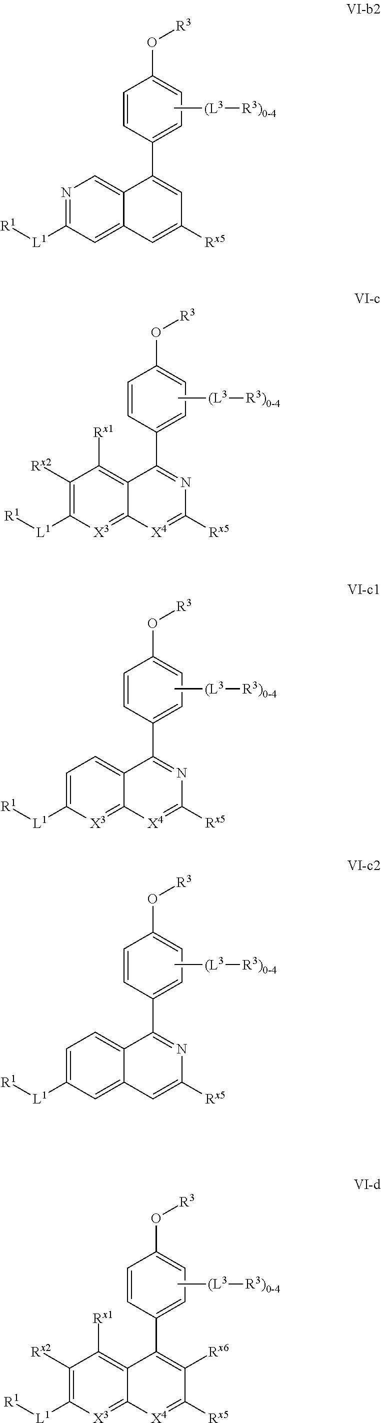

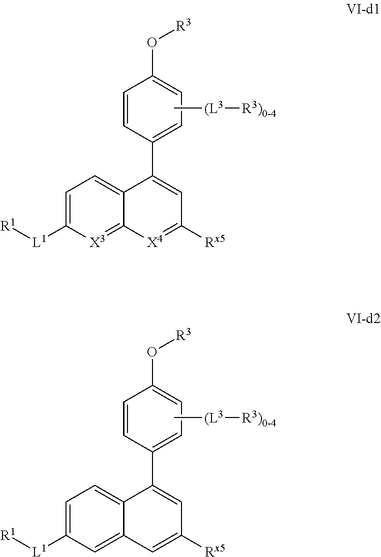

or a pharmaceutically acceptable salt thereof, wherein each of L, X³, X⁴, R¹, L³, R³, R^{x1}, R^{x2}, R^{x5}, and R^{x6} is as defined above and described herein.

In some embodiments, the present disclosure provides a compound of Formulae VII-a, VII-a1, VII-a2, VII-b, VII-b1, VII-b2, VII-c, VII-c1, VII-c2, VII-d, VII-d1, or VII-d2:

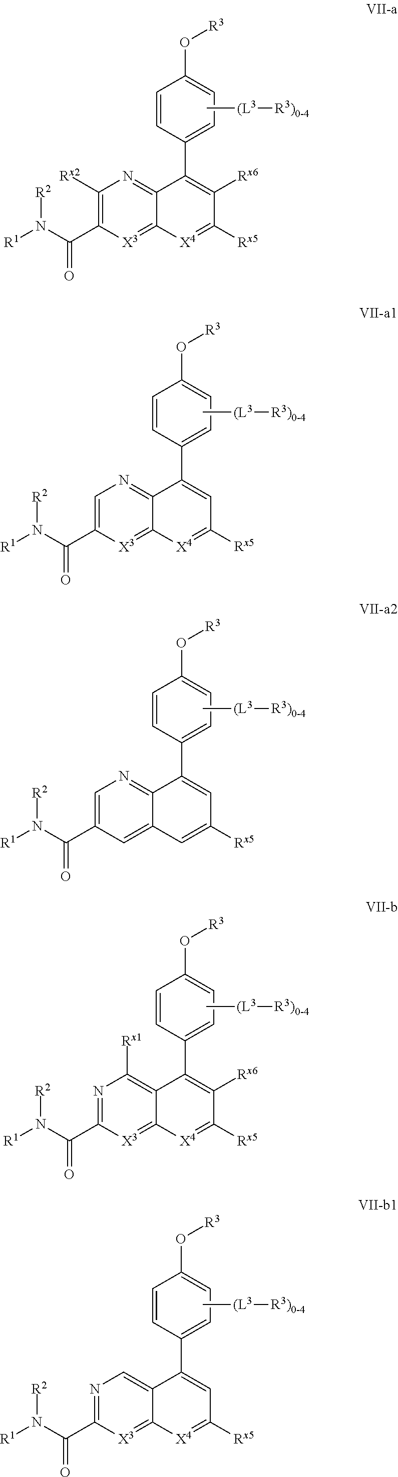

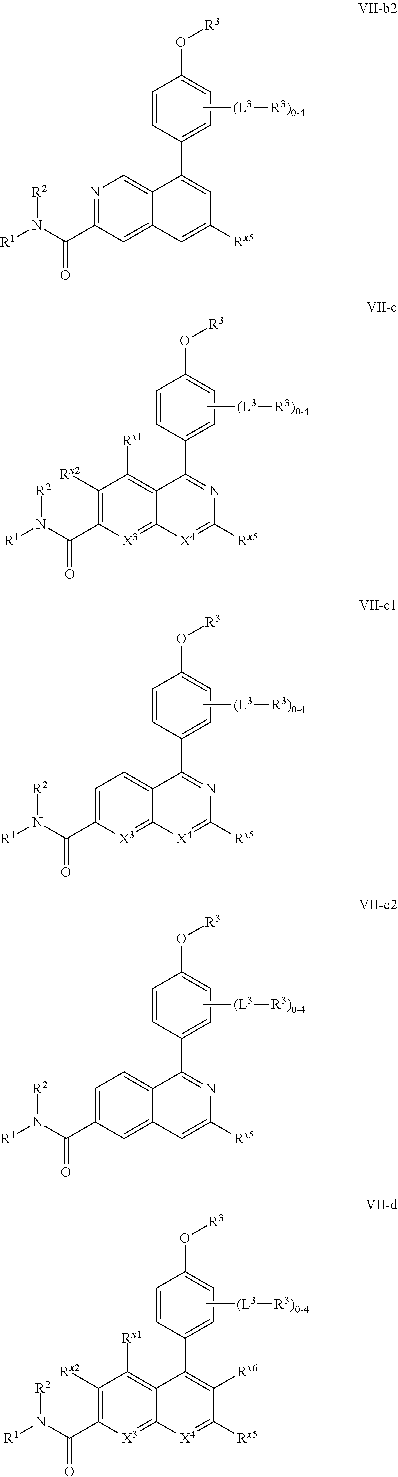

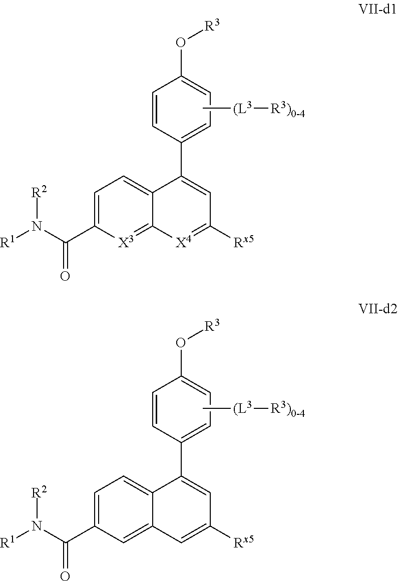

or a pharmaceutically acceptable salt thereof, wherein each of X³, X⁴, R¹, R², L³, R³, R^{x1}, R^{x2}, R^{x5}, and R^{x6} is as defined above and described herein.

In some embodiments, the present disclosure provides a compound of Formulae VIII-a, VIII-a1, VIII-a2, VIII-b, VIII-b1, VIII-b2, VIII-c, VIII-c1, VIII-c2, VIII-d, VIII-d1, or VIII-d2:

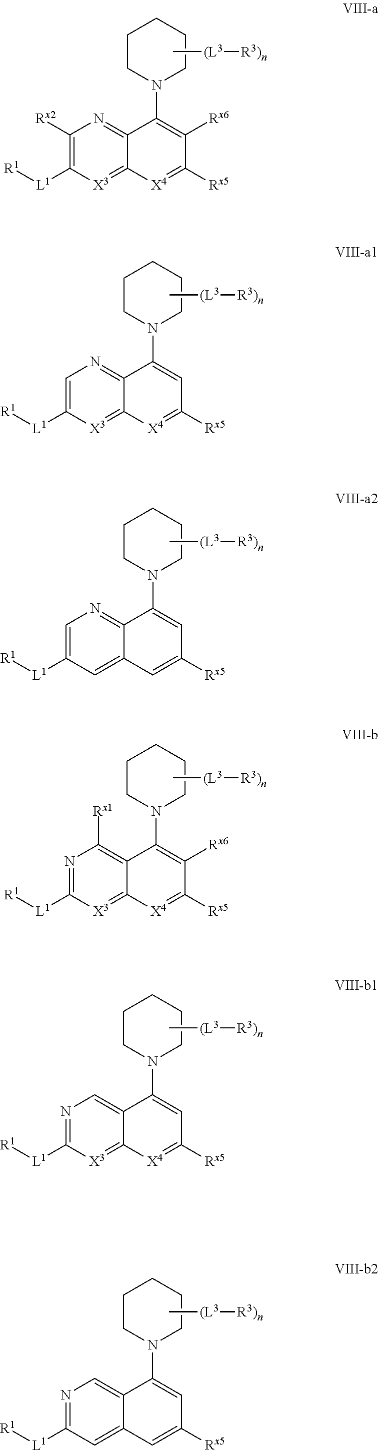

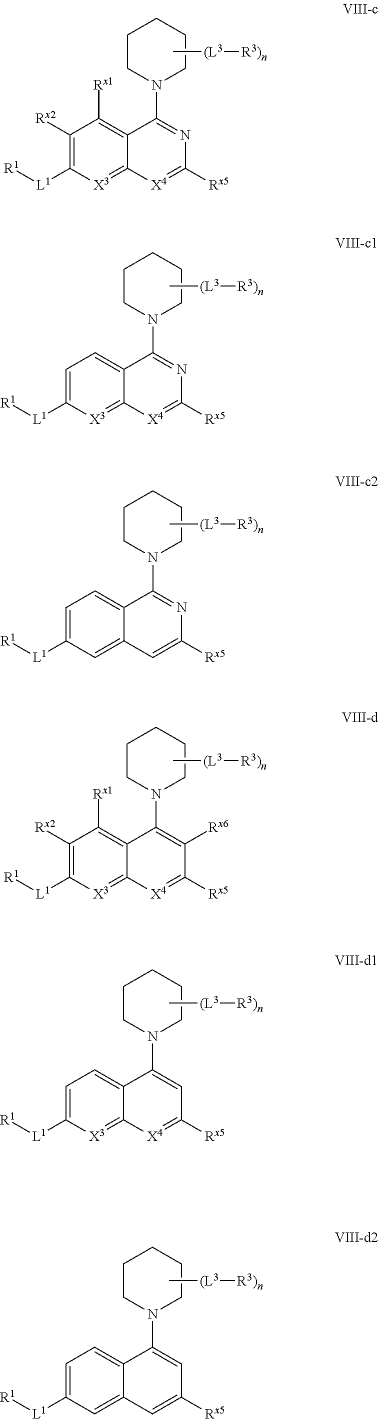

or a pharmaceutically acceptable salt thereof, wherein each of L, X³, X⁴, R¹, L³, R³, n, R^{x1}, R^{x2}, R^{x5}, and R^{x6} is as defined above and described herein.

In some embodiments, the present disclosure provides a compound of Formulae IX-a, IX-a1, IX-a2, IX-b, IX-b1, IX-b2, IX-c, IX-c1, IX-c2, IX-d, IX-d1, or IX-d2:

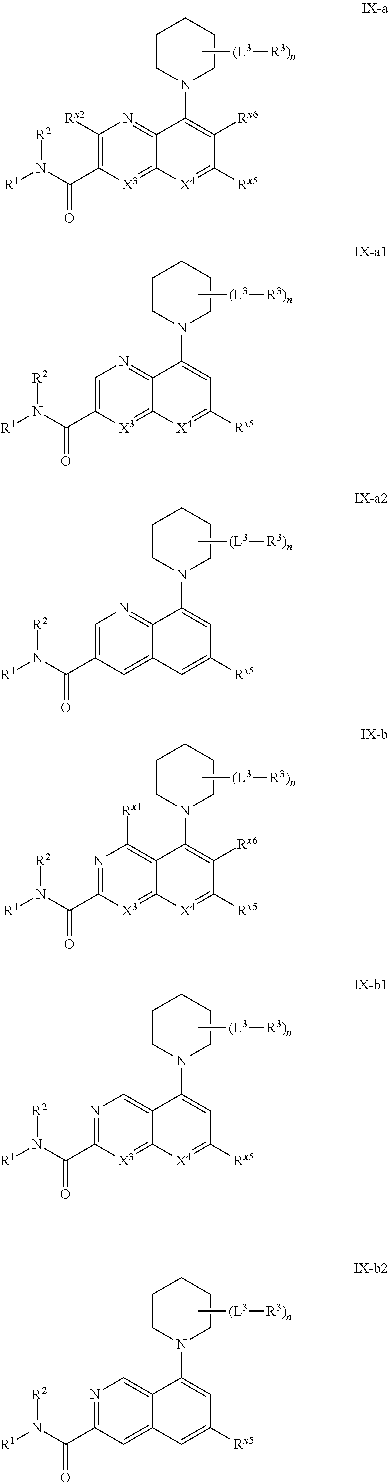

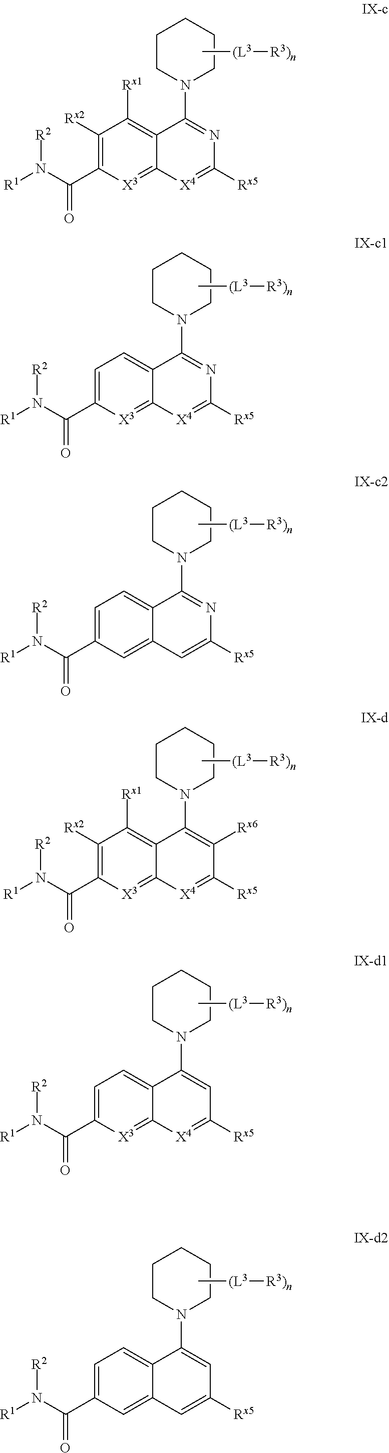

or a pharmaceutically acceptable salt thereof, wherein each of X³, X⁴, R¹, R², L³, R³, n, R^{x1}, R^{x2}, R^{x5}, and R^{x6} is as defined above and described herein.

In some embodiments, the present disclosure provides a compound of Formulae X-a, X-a1, X-a2, X-b, X-b1, X-b2, X-c, X-c1, X-c2, X-d, X-d1, or X-d2:

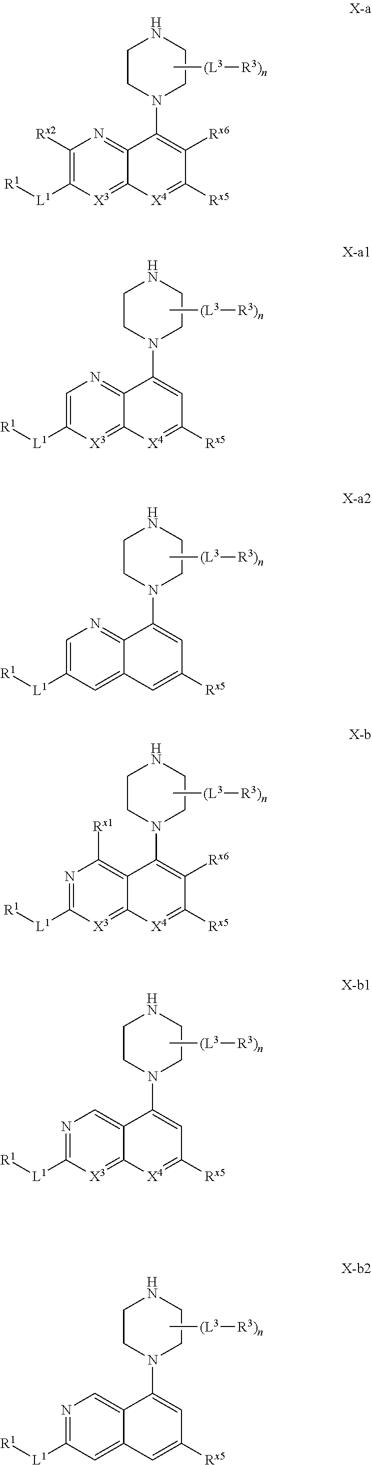

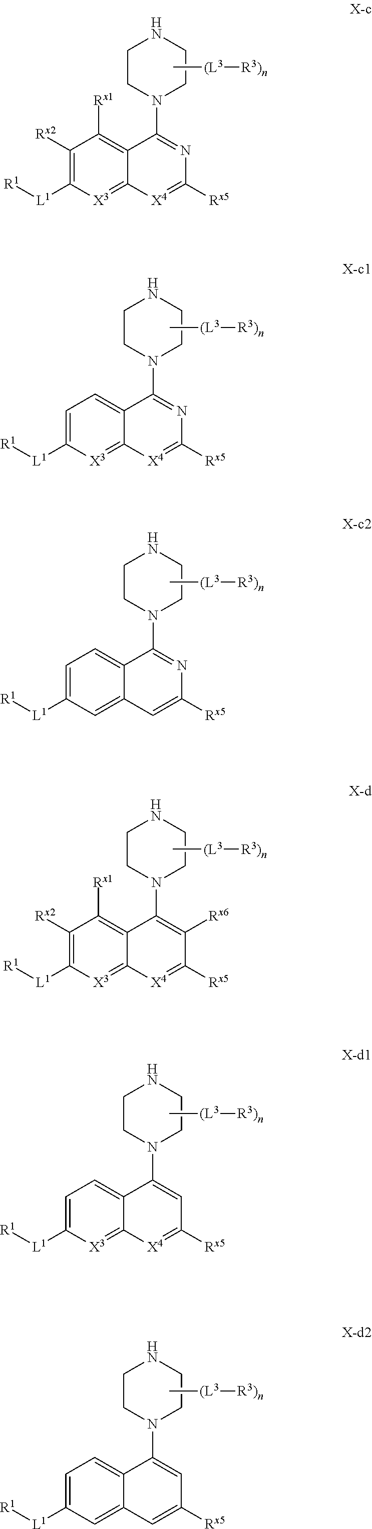

or a pharmaceutically acceptable salt thereof, wherein each of LI, X³, X⁴, R¹, L³, R³, n, R^{x1}, R^{x2}, R^{x5}, and R^{x6} is as defined above and described herein.

In some embodiments, the present disclosure provides a compound of XI-a, XI-a1, XI-a2, XI-b, XI-b1, XI-b2, XI-c, XI-c1, XI-c2, XI-d, XI-d1, or XI-d2:

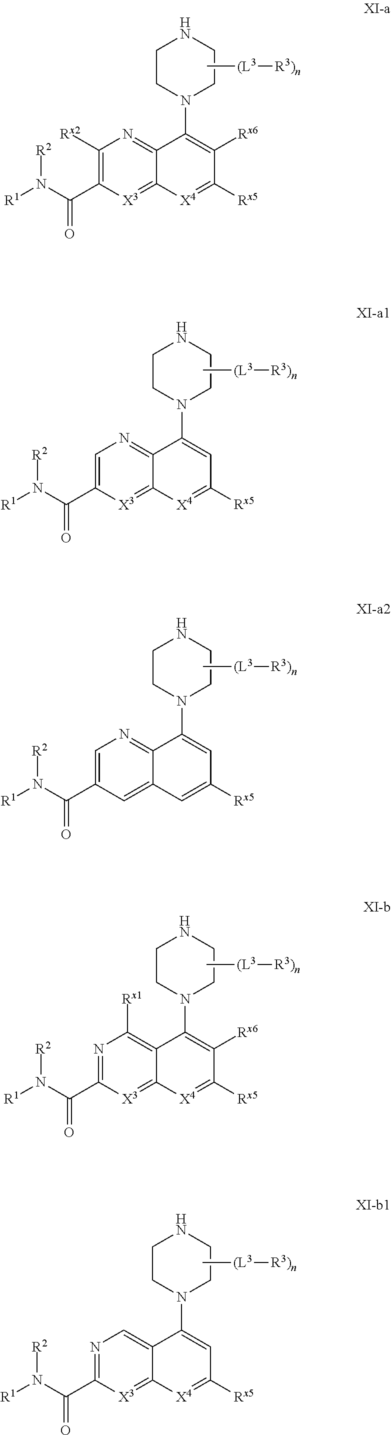

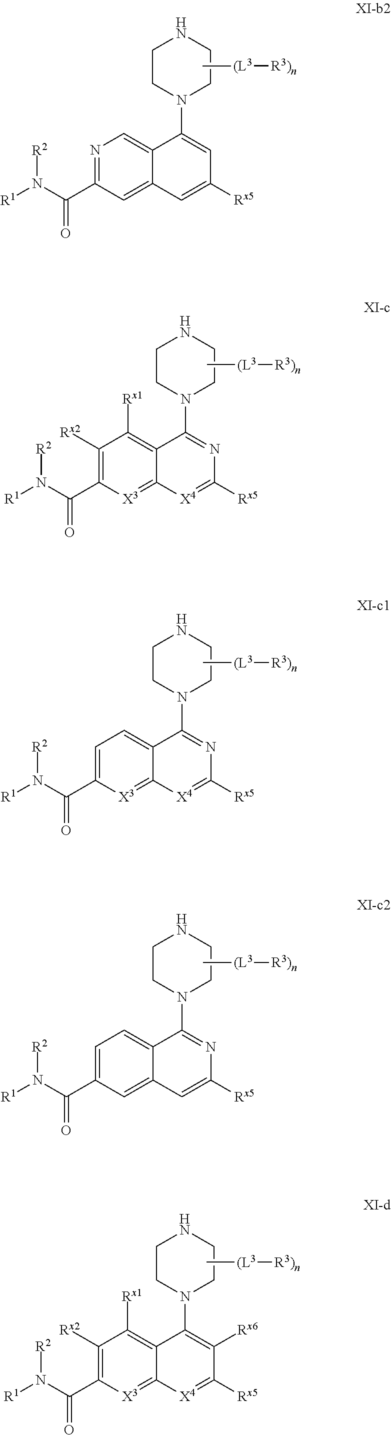

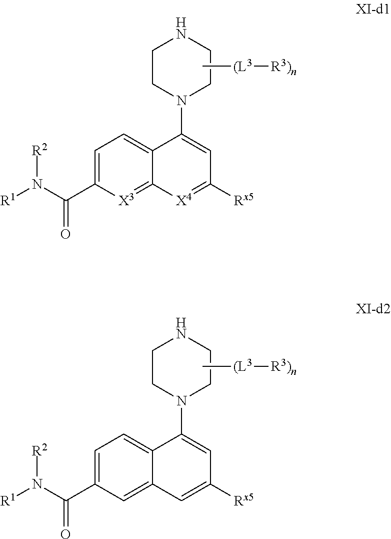

or a pharmaceutically acceptable salt thereof, wherein each of X³, X⁴, R¹, R², L³, R⁴, n, R^{x1}, R^{x2}, R^{x5}, and R^{x6} is as defined above and described herein.

In some embodiments, the present disclosure provides a compound of XII-a, XII-a1, XII-a2, XII-b, XII-b1, XII-b2, XII-c, XII-c1, XII-c2, XII-d, XII-d1, or XII-d2:

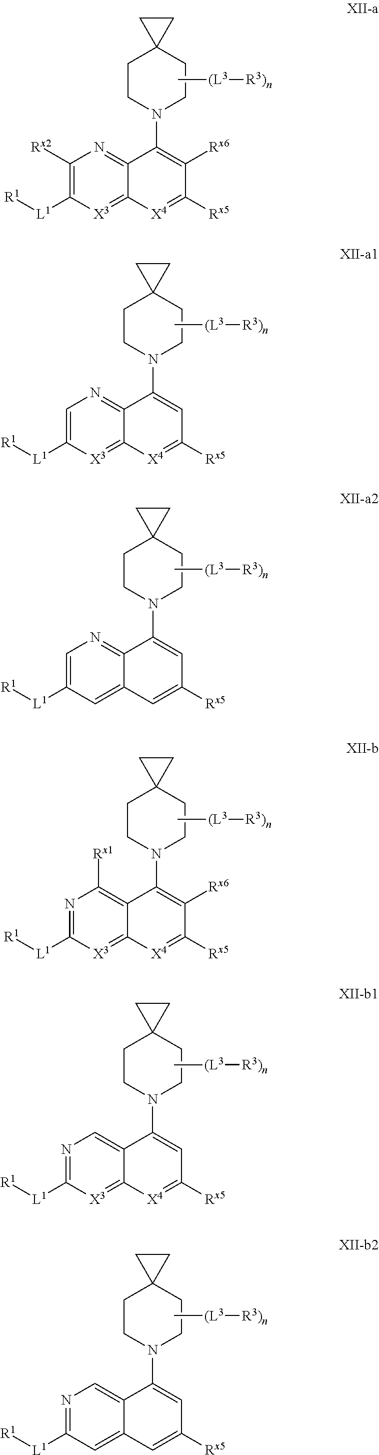

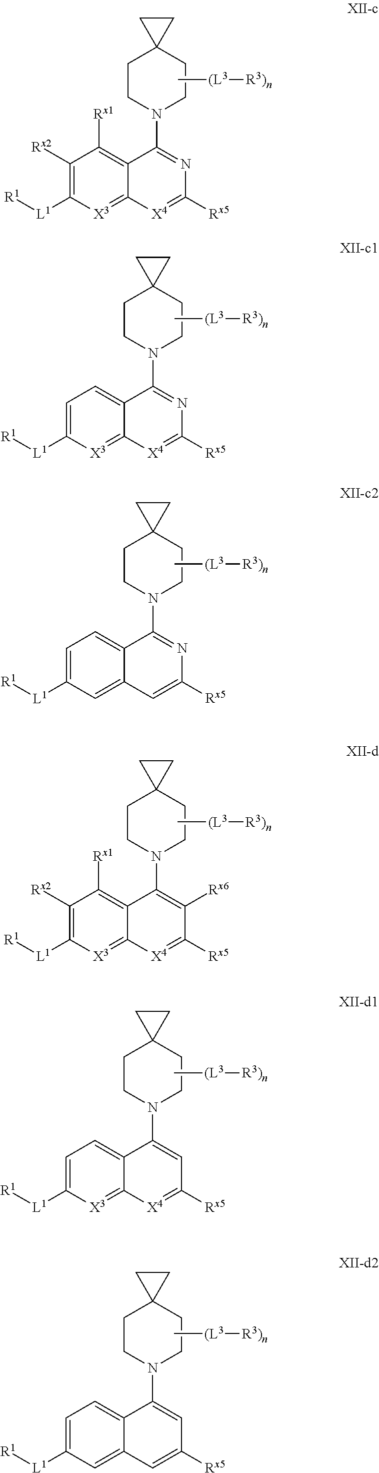

or a pharmaceutically acceptable salt thereof, wherein each of L, X³, X⁴, R¹, L³, R³, n, R^{x1}, R^{x2}, R^{x5}, and R^{x6} is as defined above and described herein.

In some embodiments, the present disclosure provides a compound of Formulae XIII-a, XIII-a1, XIII-a2, XIII-b, XIII-b1, XIII-b2, XIII-c, XIII-c1, XIII-c2, XIII-d, XIII-d1, or

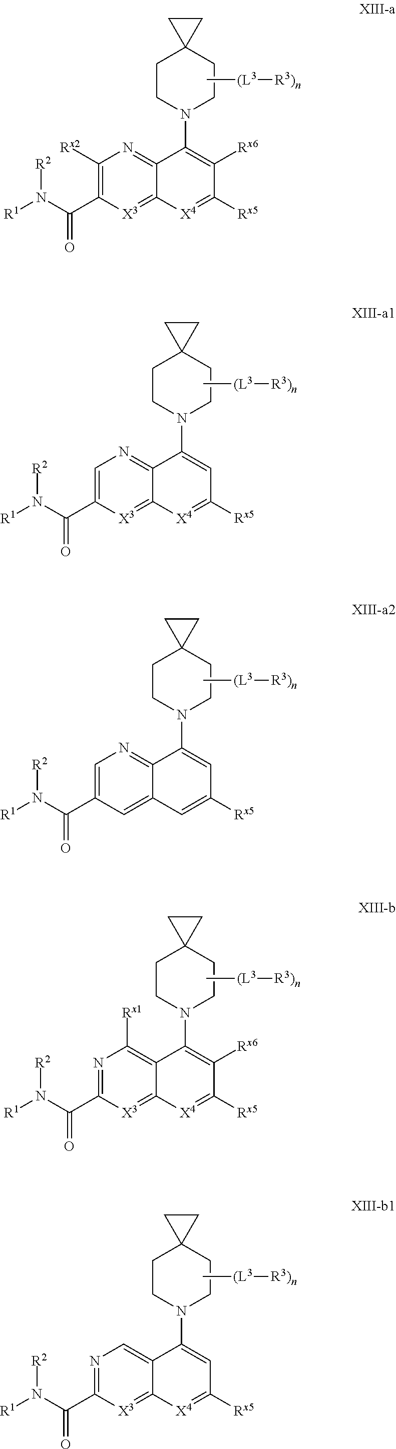

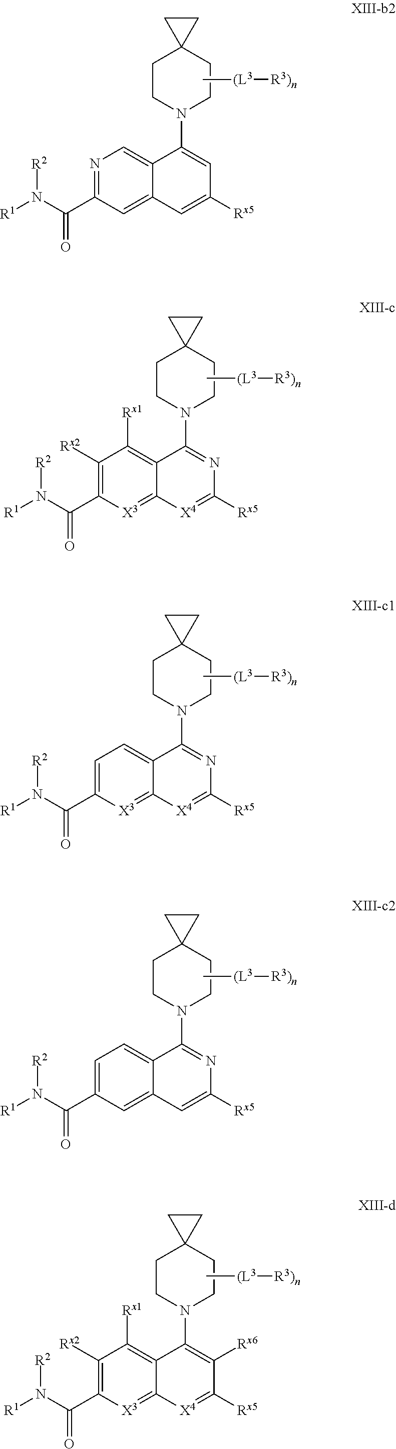

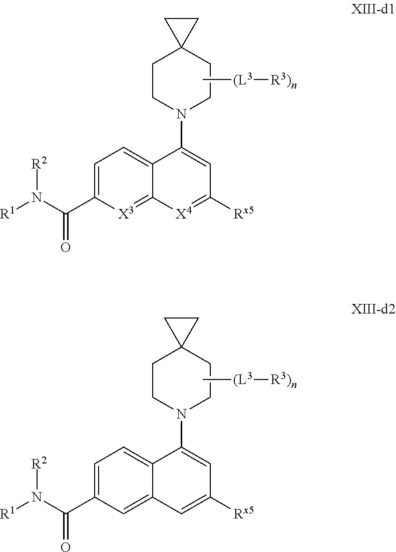

or a pharmaceutically acceptable salt thereof, wherein each of X³, X⁴, R¹, R², L³, R³, n, R^{x1}, R^{x2}, R^{x5}, and R^{x6} is as defined above and described herein.

In some embodiments, the present disclosure provides a compound of Formulae XIV-a, XIV-a1, XIV-a2, XIV-b, XIV-b1, XIV-b2, XIV-c, XIV-c1, XIV-c2, XIV-d, XIV-d1, or XIV-d2:

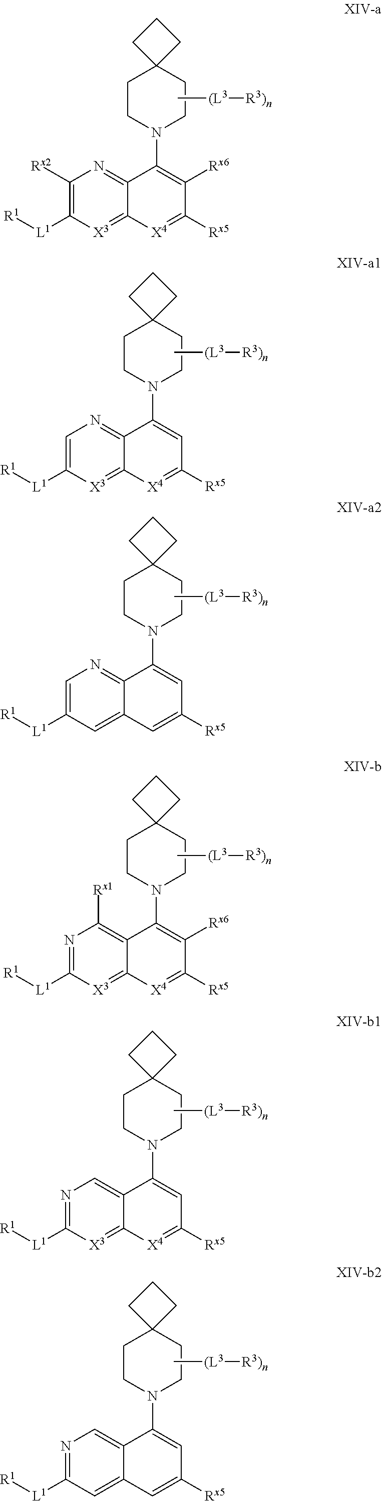

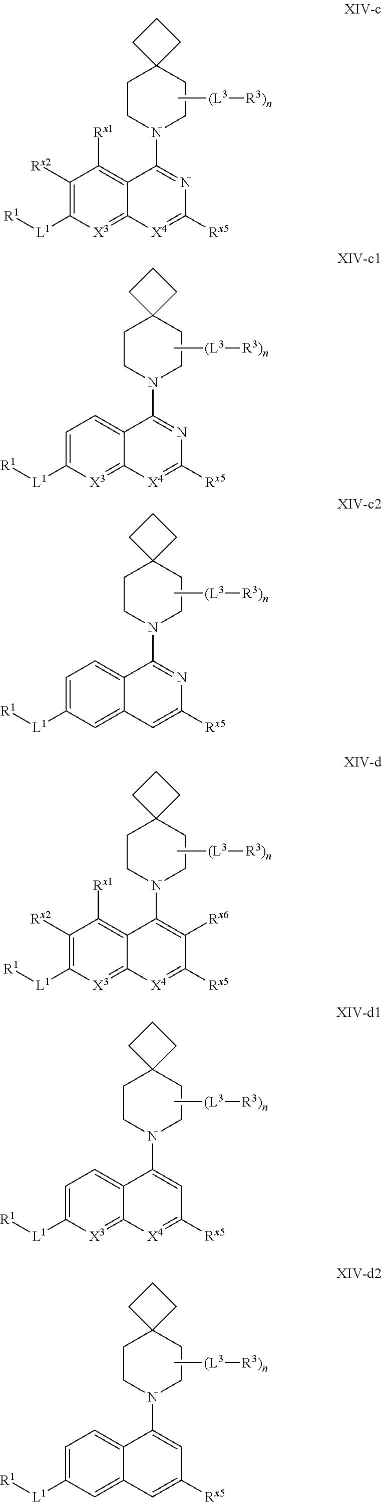

or a pharmaceutically acceptable salt thereof, wherein each of L, X³, X⁴, R¹, L³, R³, n, R^{x1}, R^{x2}, R^{x5}, and R^{x6} is as defined above and described herein.

In some embodiments, the present disclosure provides a compound of Formulae XV-a, XV-a1, XV-a2, XV-b, XV-b1, XV-b2, XV-c, XV-c1, XV-c2, XV-d, XV-d1, or XV-d2:

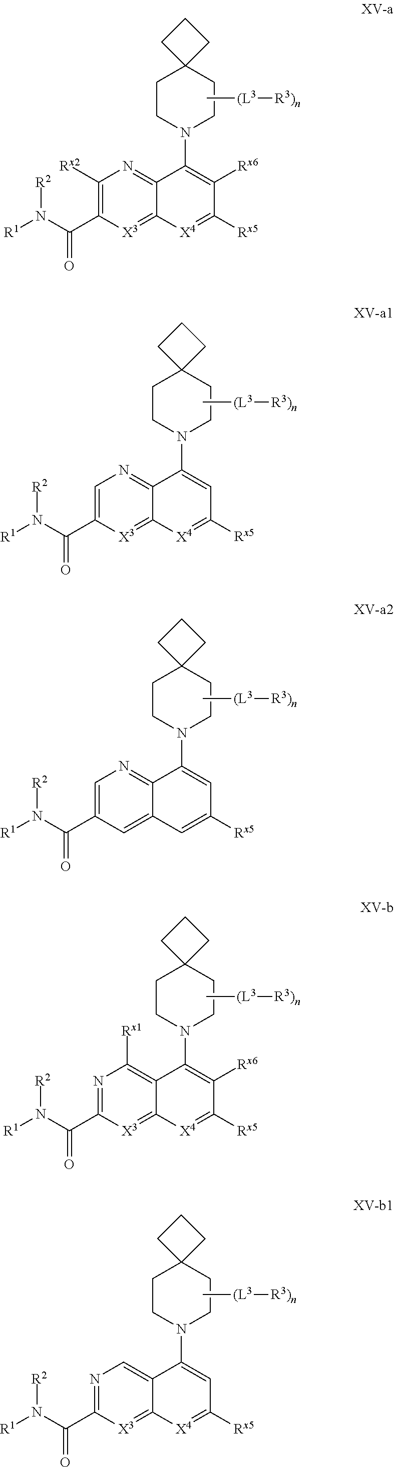

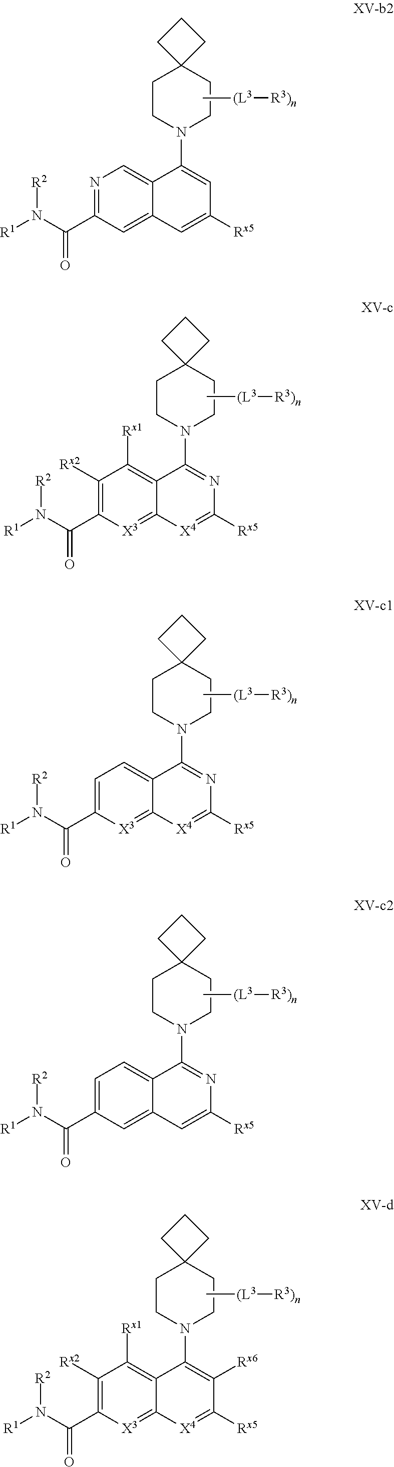

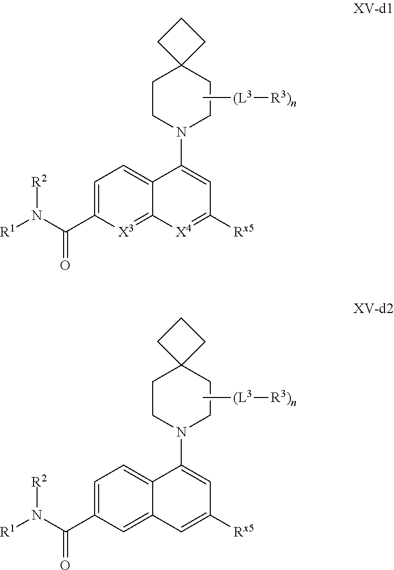

or a pharmaceutically acceptable salt thereof, wherein each of X³, X⁴, R¹, R², L³, R³, n, R^{x1}, R^{x2}, R^{x5}, and R^{x6} is as defined above and described herein.

In some embodiments, the present disclosure provides a compound of Formulae XVI-a, XVI-a1, XVI-a2, XVI-b, XVI-b1, XVI-b2, XVI-c, XVI-c1, XVI-c2, XVI-d, XVI-d1, or XVI-d2:

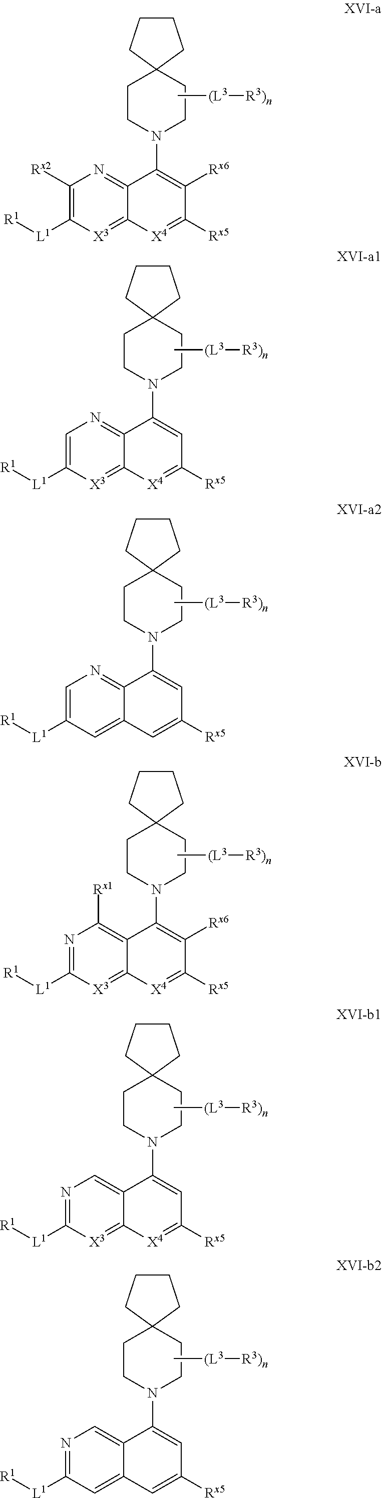

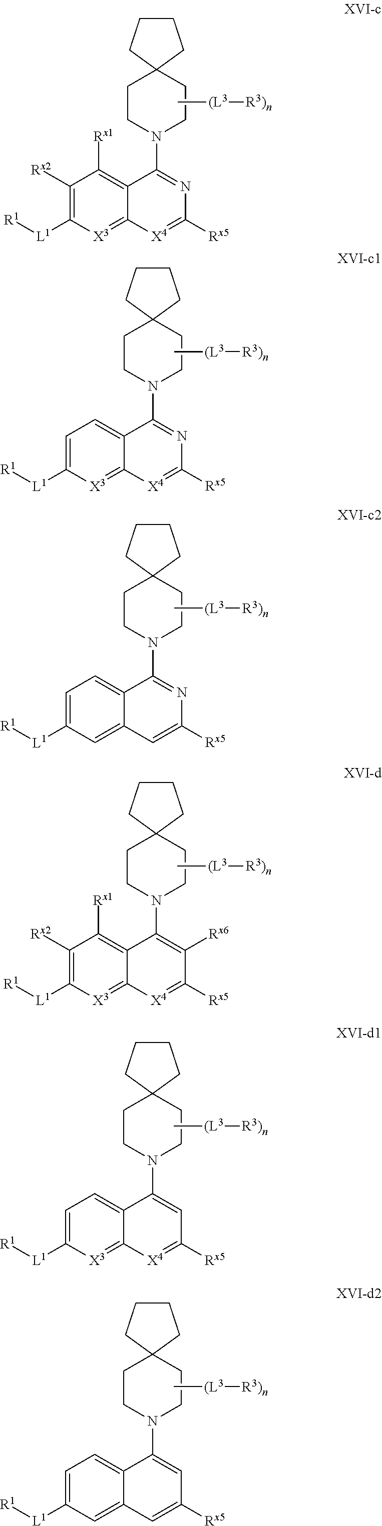

or a pharmaceutically acceptable salt thereof, wherein each of L, X³, X⁴, R¹, L³, R³, n, R^{x1}, R^{x2}, R^{x5}, and R^{x6} is as defined above and described herein.

In some embodiments, the present disclosure provides a compound of Formulae XVII-a, XVII-a1, XVII-a2, XVII-b, XVII-b1, XVII-b2, XVII-c, XVII-c1, XVII-c2, XVII-d, XVII-dl, or XVII-d2:

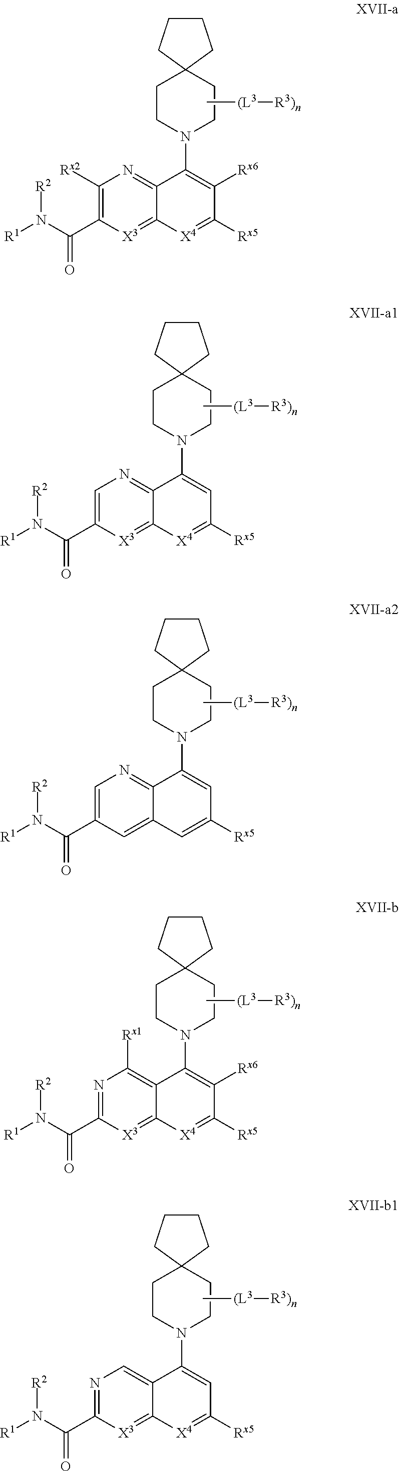

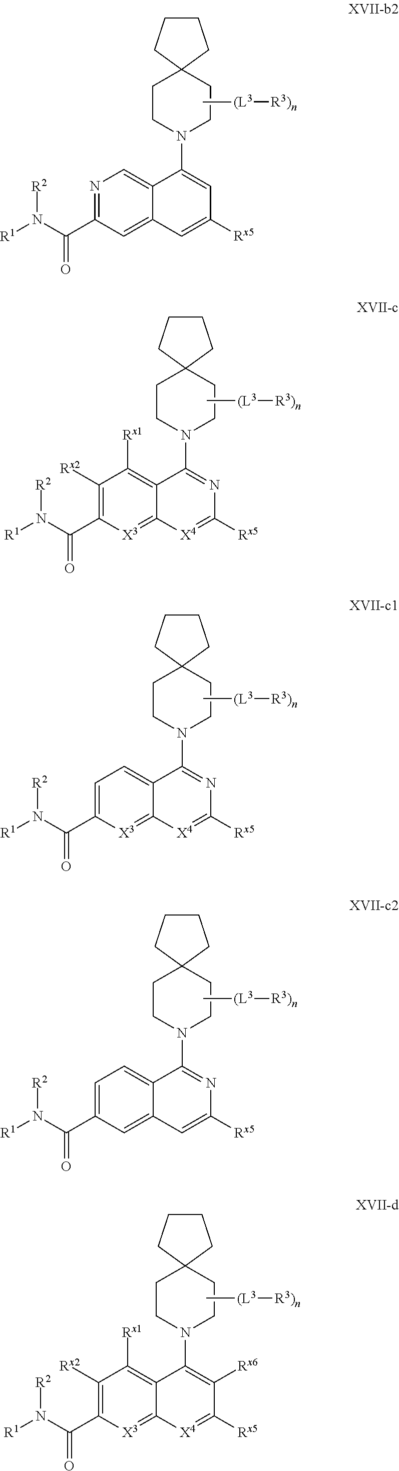

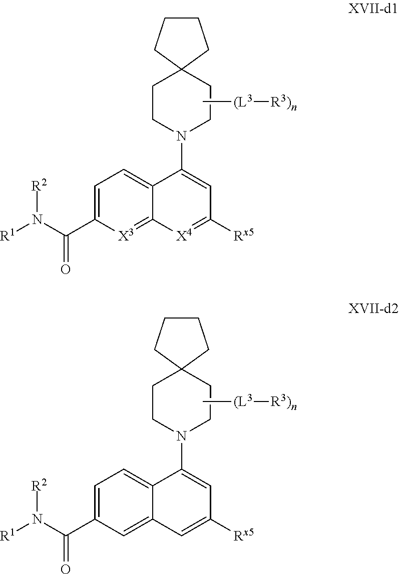

or a pharmaceutically acceptable salt thereof, wherein each of X³, X⁴, R¹, R², L³, R³, n, R^{x1}, R^{x2}, R^{x5}, and R^{x6} is as defined above and described herein.

It will be understood that, unless otherwise specified or prohibited by the foregoing definitions of Formulae II-a through XVII-d2, embodiments of variables as defined above and described in classes and subclasses herein also apply to compounds of Formulae II-a through XVII-d2, mutatis mutandis, both singly and in combination.

In some embodiments, the present disclosure provides a compound of Formulae XVIII-d, XVIII-d1, XVIII-d2, XIX-d, XIX-d1, or XIX-d2:

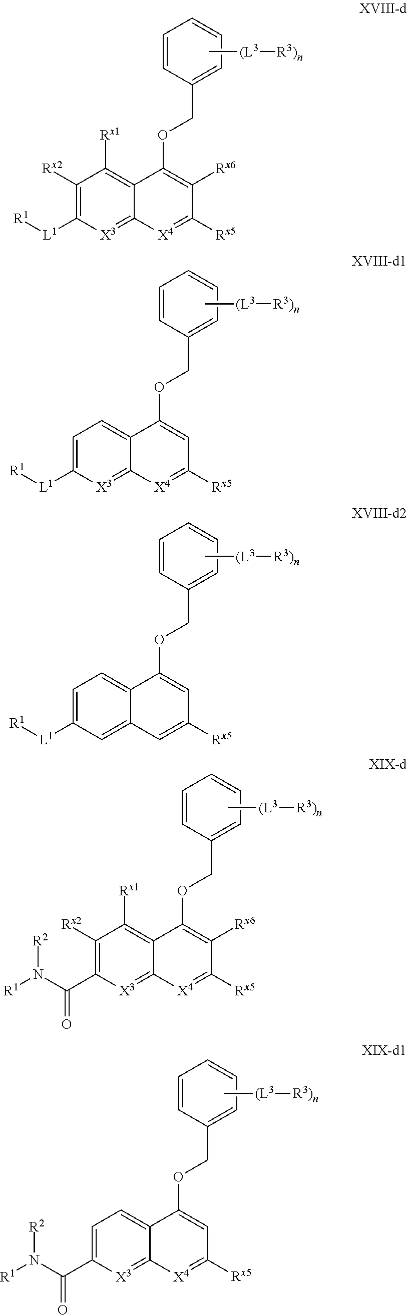

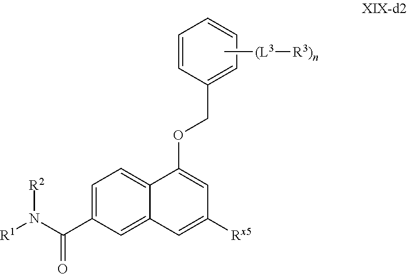

or a pharmaceutically acceptable salt thereof, wherein each of X³, X⁴, Li, R¹, R², L³, R³, n, R^{x1}, R^{x2}, R^{x5}, and R^{x6} is as defined above and described herein.

In some embodiments, the present disclosure provides a compound of Formulae XX-a, XX-a1, XX-a2, XX-d, XX-d1, or XX-d2:

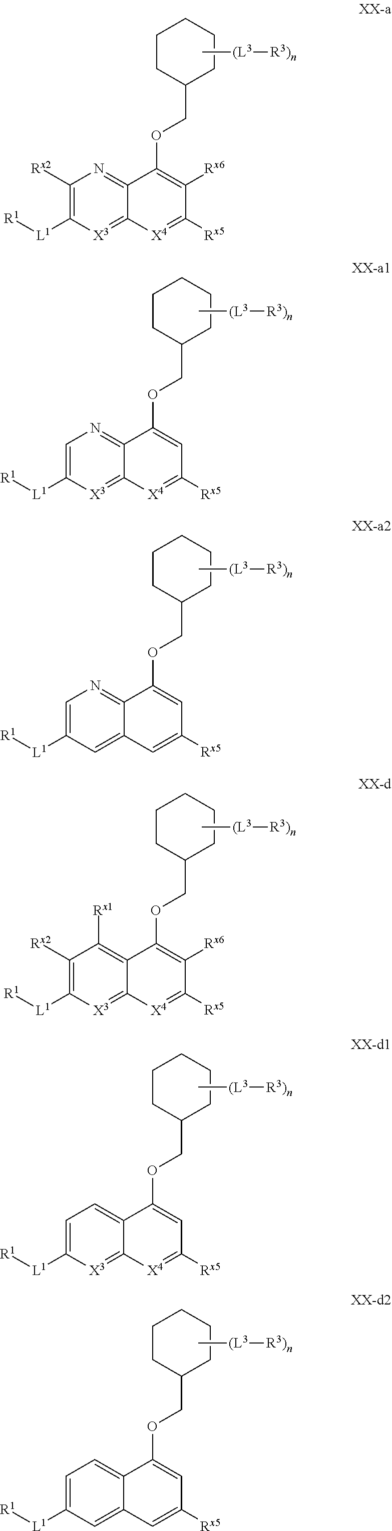

or a pharmaceutically acceptable salt thereof, wherein each of X³, X⁴, Li, R¹, L³, R³, n, R^{x1}, R^{x2}, R^{x5}, and R^{x6} is as defined above and described herein.

In some embodiments, the present disclosure provides a compound of Formulae XXI-a, XXI-a1, XXI-a2, XXI-d, XXI-d1, or XXI-d2:

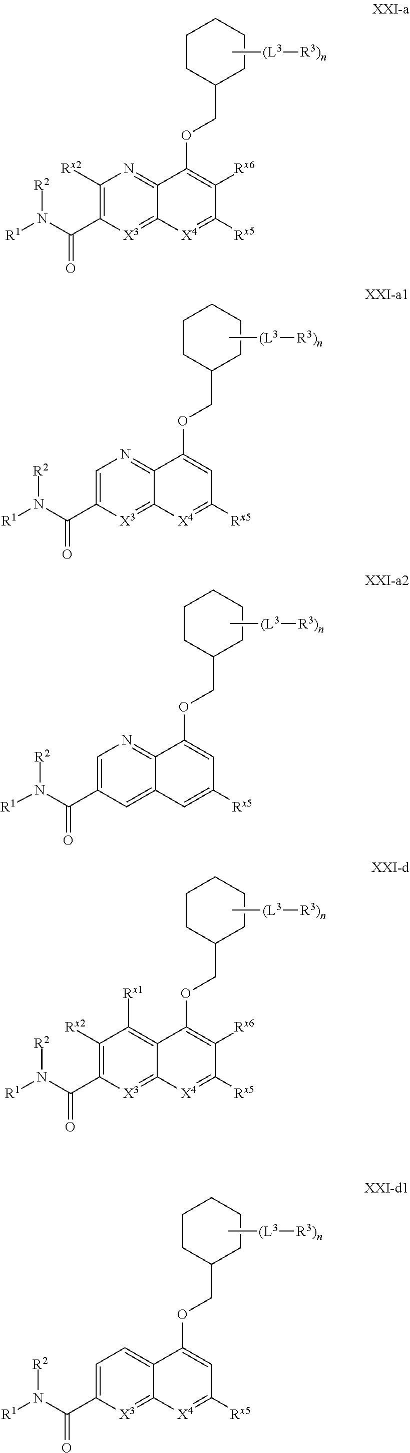

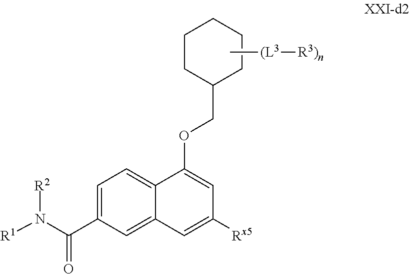

or a pharmaceutically acceptable salt thereof, wherein each of X³, X⁴, R¹, R², L³, R³, n, R^{x1}, R^{x2}, R^{x5}, and R^{x6} is as defined above and described herein.

It will be understood that, unless otherwise specified or prohibited by the foregoing definitions of Formulae XVIII-d through XXI-d2, embodiments of variables as defined above and described in classes and subclasses herein also apply to compounds of Formulae XVIII-d through XXI-d2, mutatis mutandis, both singly and in combination.

In some embodiments, the present disclosure provides a compound of Formula I′:

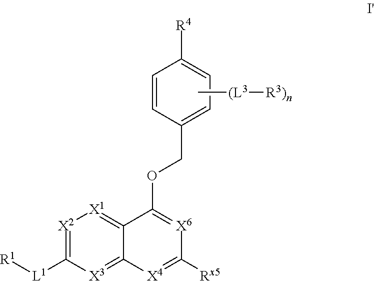

or a pharmaceutically acceptable salt thereof, wherein:
each of X¹, X², X³, X⁴, R^{x5}, X⁶, R¹, L¹, L³, and R³ is as defined above and described herein;
R⁴ is —CN or C₁₋₆ aliphatic optionally substituted with —OR, wherein R is as defined above and described herein; and
m is 0-4.

As defined generally above, R⁴ is —CN or C₁₋₆ aliphatic optionally substituted with —OR. In some embodiments, R⁴ is —CN. In some embodiments, R⁴ is C₁₋₆ aliphatic optionally substituted with —OR. In some embodiments, R⁴ is C₁₋₆ aliphatic optionally substituted with —OH or —OCH₃. In some embodiments, R⁴ is C₁₋₆ aliphatic optionally substituted with —OH. In some embodiments, R⁴ is C₁₋₄ aliphatic optionally substituted with —OR. In some embodiments, R⁴ is C₁₋₄ aliphatic optionally substituted with —OH or —OCH₃. In some embodiments, R⁴ is C₁₋₄ aliphatic optionally substituted with —OH. In some embodiments, R⁴ is selected from the group consisting of methyl, ethyl, n-propyl, isopropyl, t-butyl, —CN,

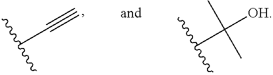

In some embodiments, R⁴ is selected from the group consisting of t-butyl, —CN,

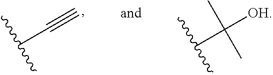

In some embodiments, m is 0-4. In some embodiments, m is 0-3. In some embodiments, m is 0-2. In some embodiments, m is 1-2. In some embodiments, m is 1-3. In some embodiments, m is 2-3. In some embodiments, m is 0. In some embodiments, m is 1. In some embodiments, m is 2. In some embodiments, m is 3. In some embodiments, m is 4.

In some embodiments of Formula I′, R^{x5} is hydrogen or —OMe.

In some embodiments of Formula I′, R¹ is:

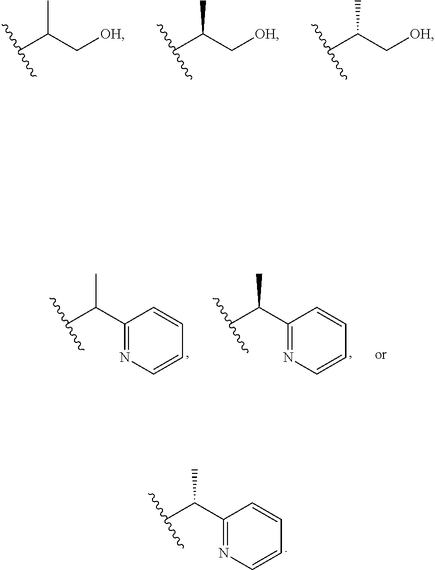

In some embodiments of Formula I′, R¹ is

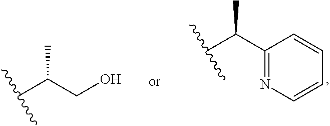

In some embodiments, the present disclosure provides a compound of Formulae II′-a, II′-a1, II′-a2, III′-a, III′-a1, III′-a2, IV′-a, IV′-a1, or IV′-a2:

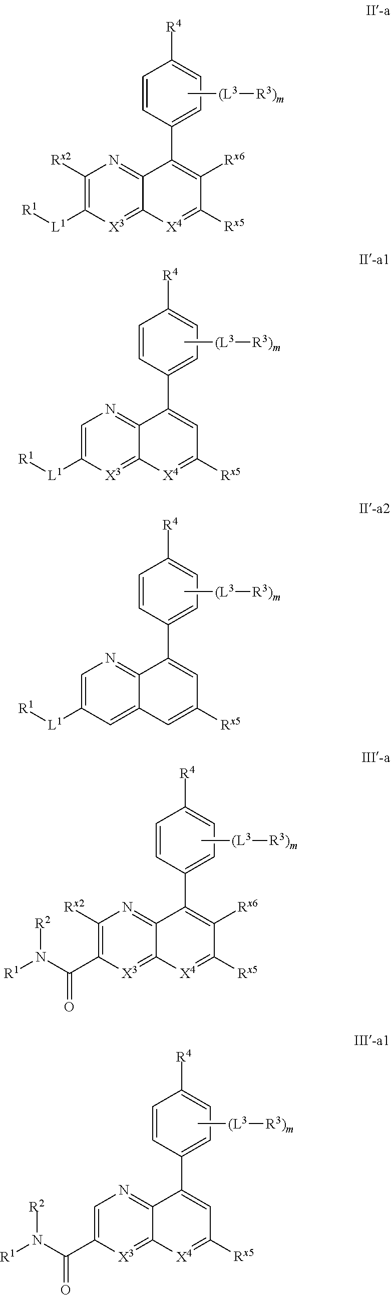

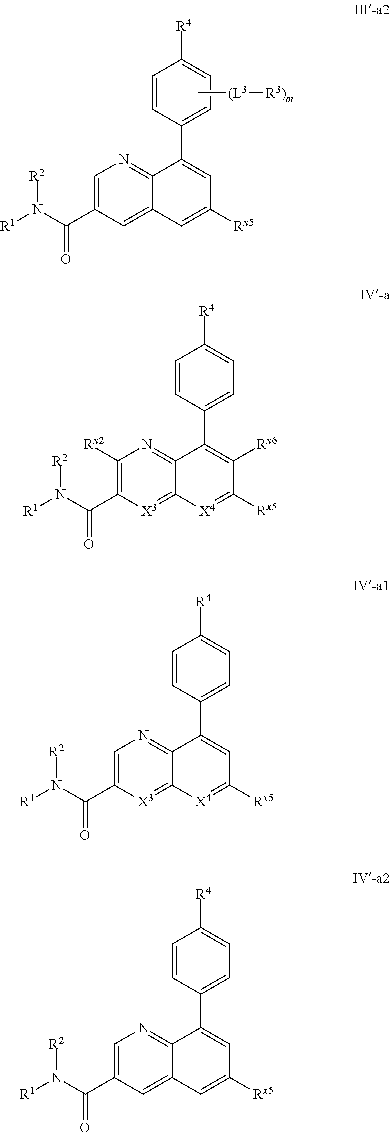

or a pharmaceutically acceptable salt thereof, wherein each of X³, X⁴, L¹, R¹, R², L³, R³, m, R⁴, R^{x2}, R^{x5}, and R^{x6} is as defined above and described herein.

It will be understood that, unless otherwise specified or prohibited by the foregoing definitions of Formulae II′-a through IV′-a2, embodiments of variables as defined above and described in classes and subclasses herein also apply to compounds of Formulae II′-a through IV′-a2, mutatis mutandis, both singly and in combination.

In some embodiments, the present disclosure provides a compound of Formula I″:

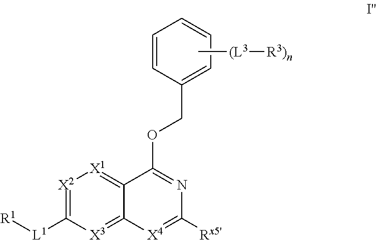

or a pharmaceutically acceptable salt thereof, wherein:
each of X¹, X², X³, X⁴, R¹, L¹, L³, R³, and n is as defined above and described herein; and
R^{x5′} is selected from —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur.

As defined generally above, R^{x5′} is selected from —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur. In some embodiments, R^{x5′} is selected from —OR or —N(R)₂. In some embodiments, R^{x5′} is —OR. In some embodiments, R^{x5′} is —OH or —OCH₃. In some embodiments, R^{x5′} is —OH. In some embodiments, R^{x5′} is —OCH₃.

In some embodiments of Formula I″, R¹ is:

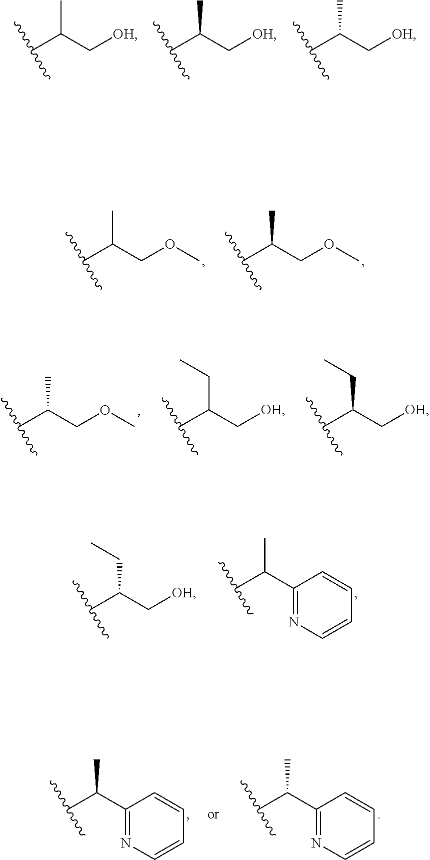

In some embodiments of Formula I″, R¹ is:

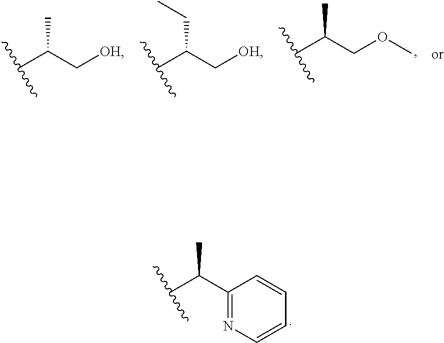

In some embodiments of Formula I″, L³ is a covalent bond and R³ is —CF₃. In some embodiments of Formula I″, R³ is —CF₃.

In some embodiments, the present disclosure provides a compound of Formulae II″-c1 II″-c2 III″-c, III″-c1, III″-c2, IV″-c, IV″-c1, IV″-c2, V″-c, V″-c1, or V″-c2:

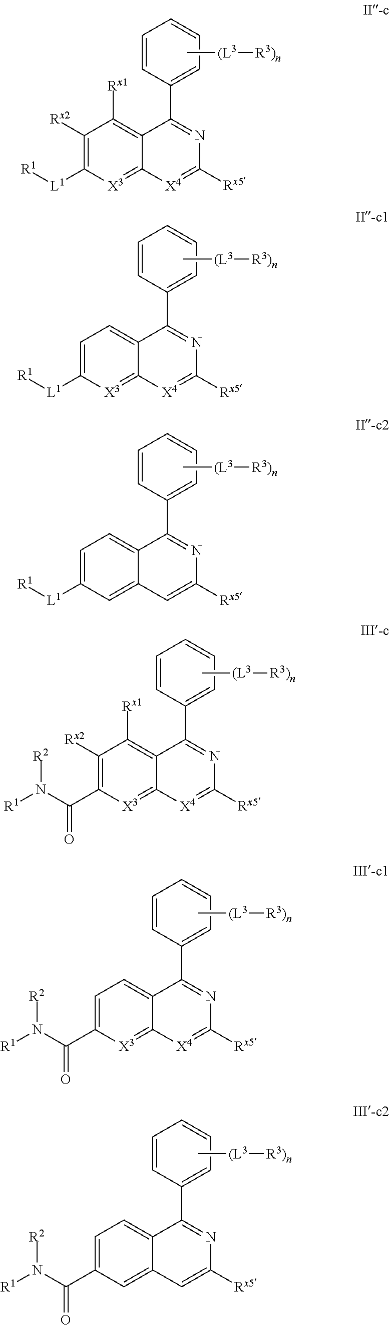

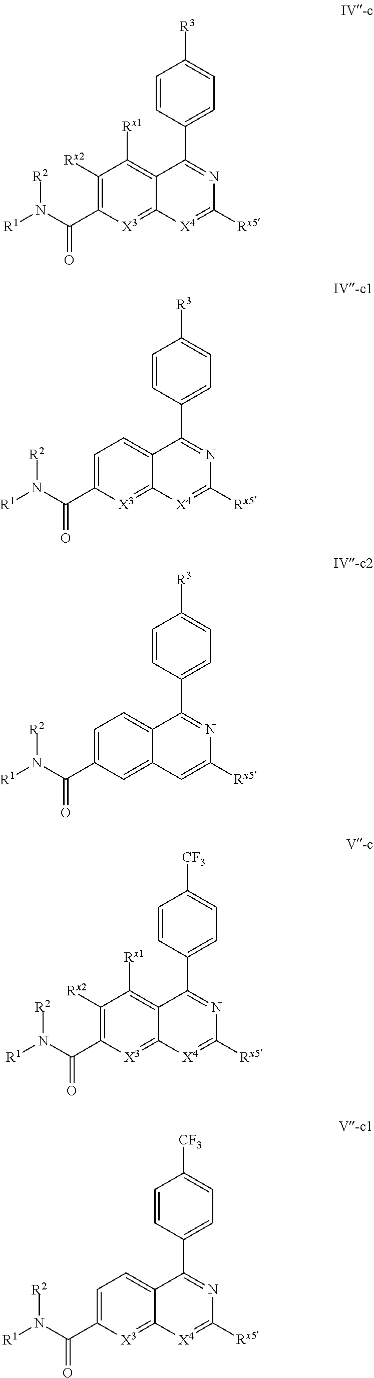

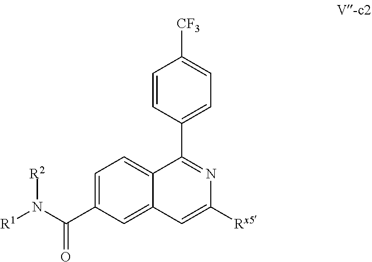

or a pharmaceutically acceptable salt thereof, wherein each of X³, X⁴, LI, R¹, R², L³, R³, n, R^{x1}, R^{x2}, and R^{x5′} is as defined above and described herein.

It will be understood that, unless otherwise specified or prohibited by the foregoing definitions of Formulae II″-c1 through V″-c2, embodiments of variables as defined above and described in classes and subclasses herein also apply to compounds of Formulae II″-c1 through V″-c2, mutatis mutandis, both singly and in combination.

In some embodiments, a compound of Formula I is selected from the group consisting of:

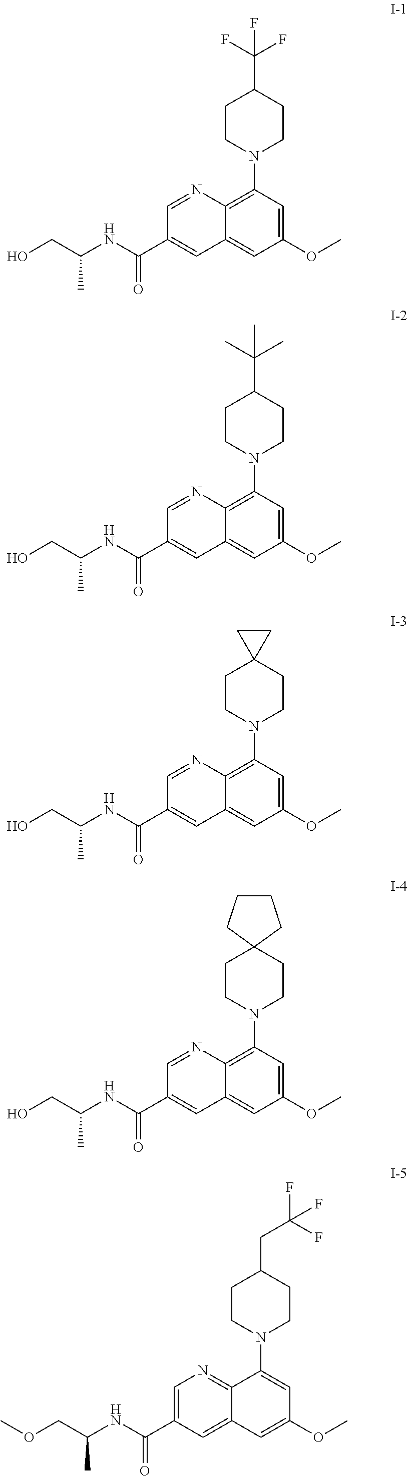

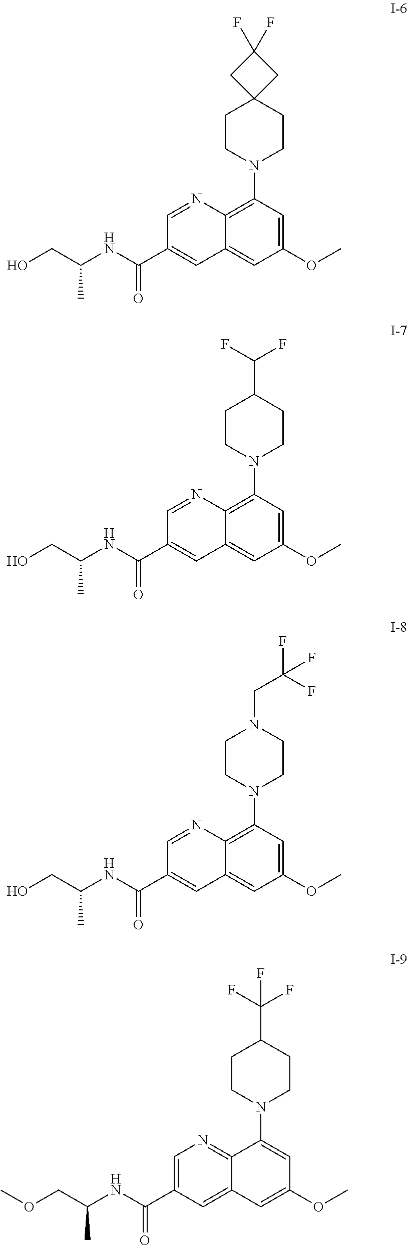

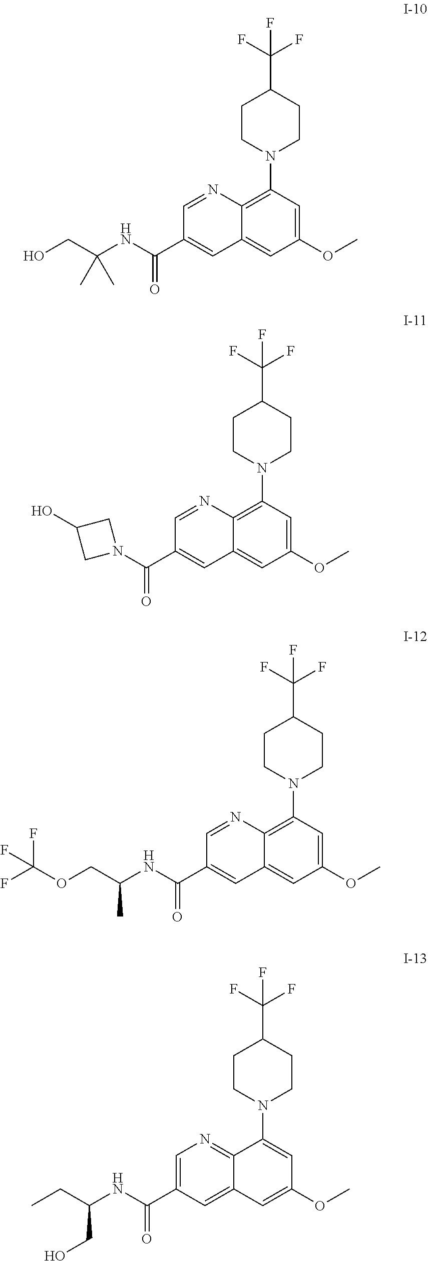

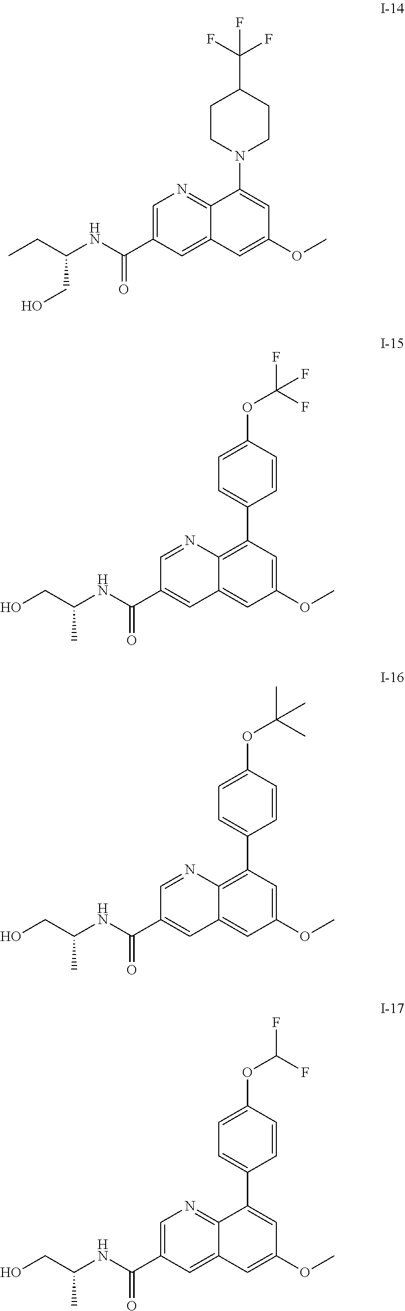 [SMILES: CC[C@@H](CO)NC(=O)c1cnc2c(N3CCC(C(F)(F)F)CC3)cc(OC)cc2c1 ; COc1cc(-c2ccc(OC(F)(F)F)cc2)c2ncc(C(=O)N[C@H](C)CO)cc2c1 ; COc1cc(-c2ccc(OC(C)(C)C)cc2)c2ncc(C(=O)N[C@H](C)CO)cc2c1 ; COc1cc(-c2ccc(OC(F)F)cc2)c2ncc(C(=O)N[C@H](C)CO)cc2c1]

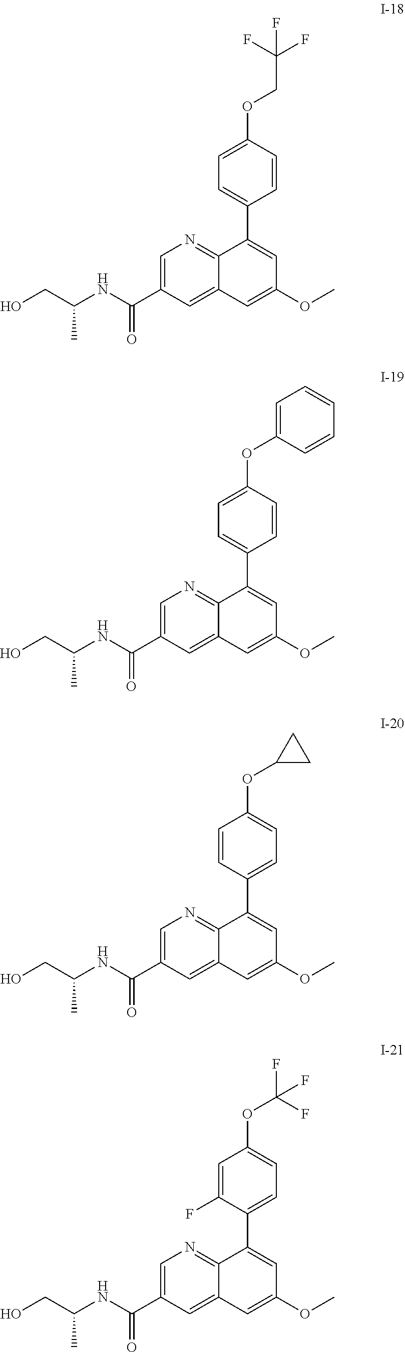 [SMILES: COc1cc(-c2ccc(OCC(F)(F)F)cc2)c2ncc(C(=O)N[C@H](C)CO)cc2c1 ; COc1cc(-c2ccc(Oc3ccccc3)cc2)c2ncc(C(=O)N[C@H](C)CO)cc2c1 ; COc1cc(-c2ccc(OC3CC3)cc2)c2ncc(C(=O)N[C@H](C)CO)cc2c1 ; COc1cc(-c2ccc(OC(F)(F)F)cc2F)c2ncc(C(=O)N[C@H](C)CO)cc2c1]

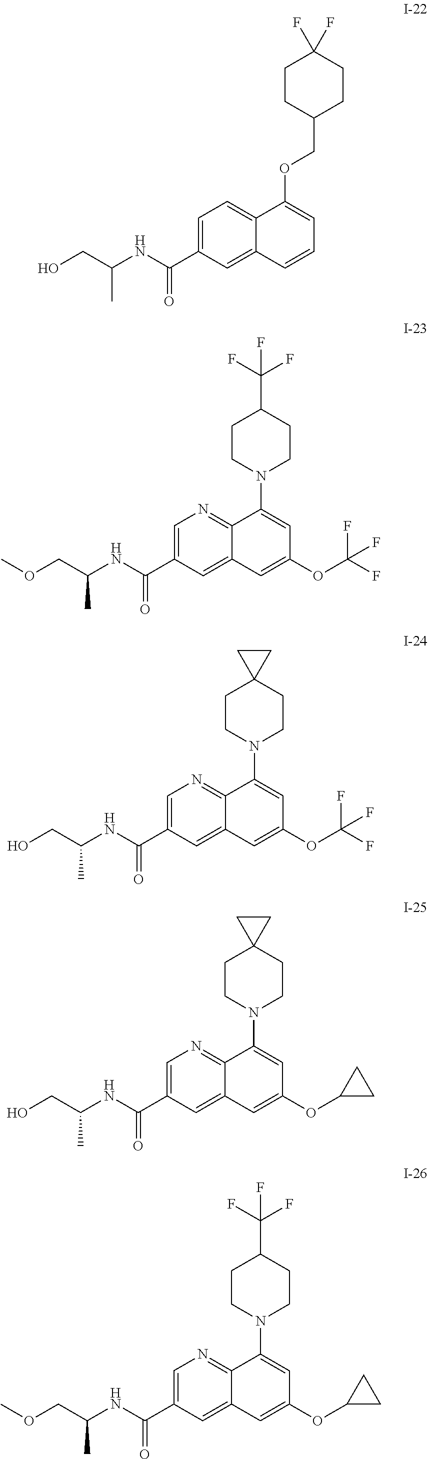 [SMILES: CC(CO)NC(=O)c1ccc2c(OCC3CCC(F)(F)CC3)cccc2c1 ; COC[C@H](C)NC(=O)c1cnc2c(N3CCC(C(F)(F)F)CC3)cc(OC(F)(F)F)cc2c1 ; C[C@H](CO)NC(=O)c1cnc2c(N3CCC4(CC3)CC4)cc(OC(F)(F)F)cc2c1 ; C[C@H](CO)NC(=O)c1cnc2c(N3CCC4(CC3)CC4)cc(OC3CC3)cc2c1 ; COC[C@H](C)NC(=O)c1cnc2c(N3CCC(C(F)(F)F)CC3)cc(OC3CC3)cc2c1]

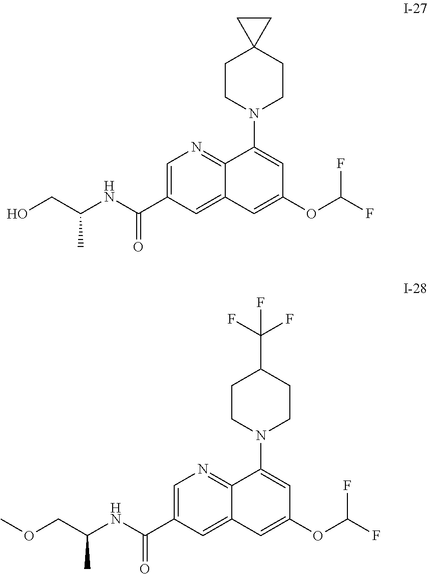 [SMILES: C[C@H](CO)NC(=O)c1cnc2c(N3CCC4(CC3)CC4)cc(OC(F)F)cc2c1 ; COC[C@H](C)NC(=O)c1cnc2c(N3CCC(C(F)(F)F)CC3)cc(OC(F)F)cc2c1]

or a pharmaceutically acceptable salt thereof.

In some embodiments, a compound provided by this disclosure is selected from the group consisting of:

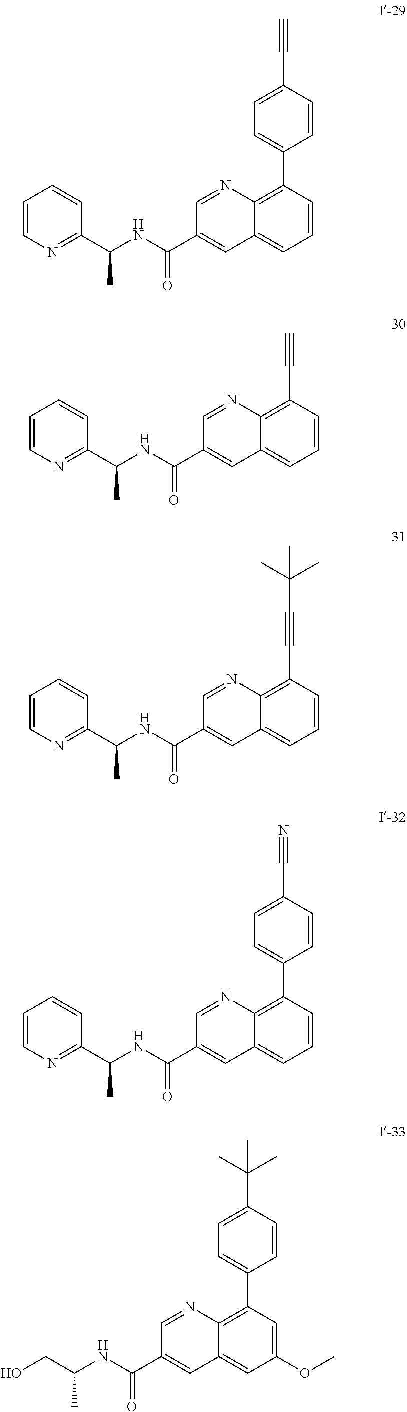 [SMILES: C#Cc1ccc(-c2cccc3cc(C(=O)N[C@@H](C)c4ccccn4)cnc23)cc1 ; C#Cc1cccc2cc(C(=O)N[C@@H](C)c3ccccn3)cnc12 ; C[C@H](NC(=O)c1cnc2c(C#CC(C)(C)C)cccc2c1)c1ccccn1 ; C[C@H](NC(=O)c1cnc2c(-c3ccc(C#N)cc3)cccc2c1)c1ccccn1 ; COc1cc(-c2ccc(C(C)(C)C)cc2)c2ncc(C(=O)N[C@H](C)CO)cc2c1]

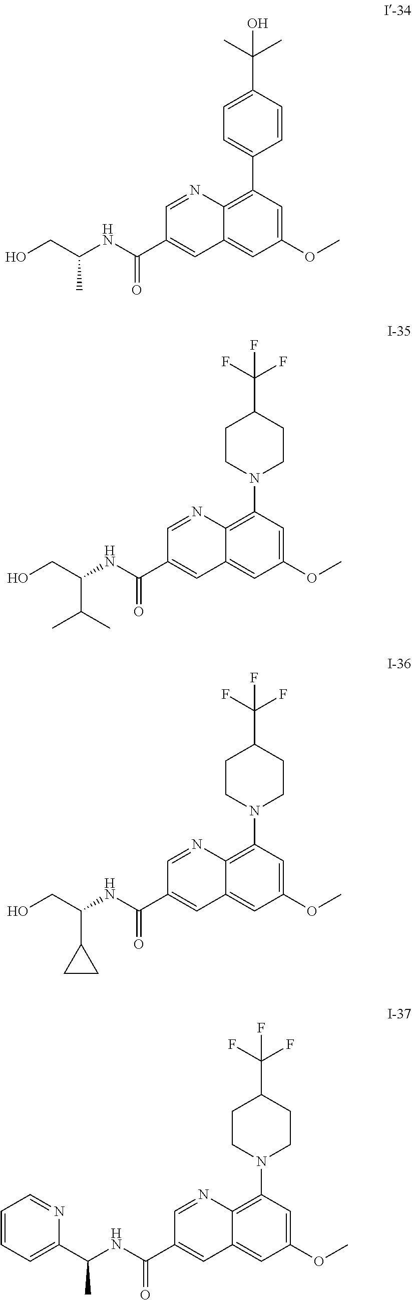 [SMILES: COc1cc(-c2ccc(C(C)(C)O)cc2)c2ncc(C(=O)N[C@H](C)CO)cc2c1 ; COc1cc(N2CCC(C(F)(F)F)CC2)c2ncc(C(=O)N[C@@H](CO)C(C)C)cc2c1 ; COc1cc(N2CCC(C(F)(F)F)CC2)c2ncc(C(=O)N[C@@H](CO)C3CC3)cc2c1 ; COc1cc(N2CCC(C(F)(F)F)CC2)c2ncc(C(=O)N[C@@H](C)c3ccccn3)cc2c1]

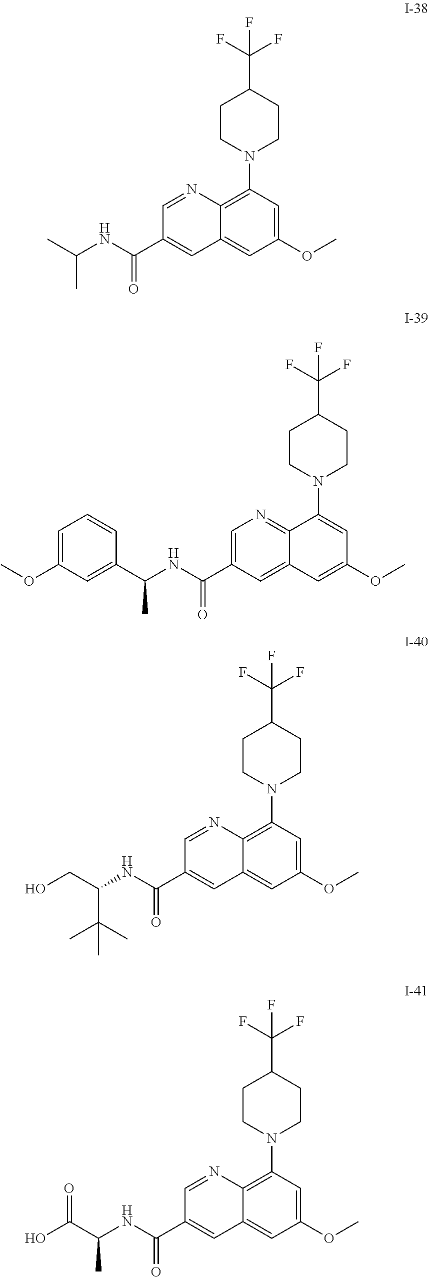

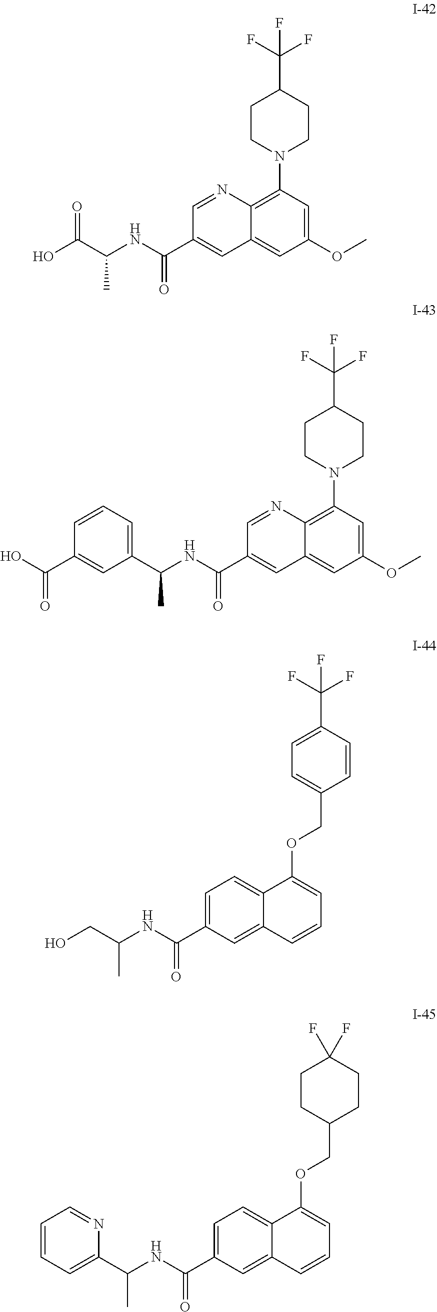 [SMILES: COc1cc(N2CCC(C(F)(F)F)CC2)c2ncc(C(=O)N[C@H](C)C(=O)O)cc2c1 ; COc1cc(N2CCC(C(F)(F)F)CC2)c2ncc(C(=O)N[C@@H](C)c3cccc(C(=O)O)c3)cc2c1 ; CC(CO)NC(=O)c1ccc2c(OCc3ccc(C(F)(F)F)cc3)cccc2c1 ; CC(NC(=O)c1ccc2c(OCC3CCC(F)(F)CC3)cccc2c1)c1ccccn1]

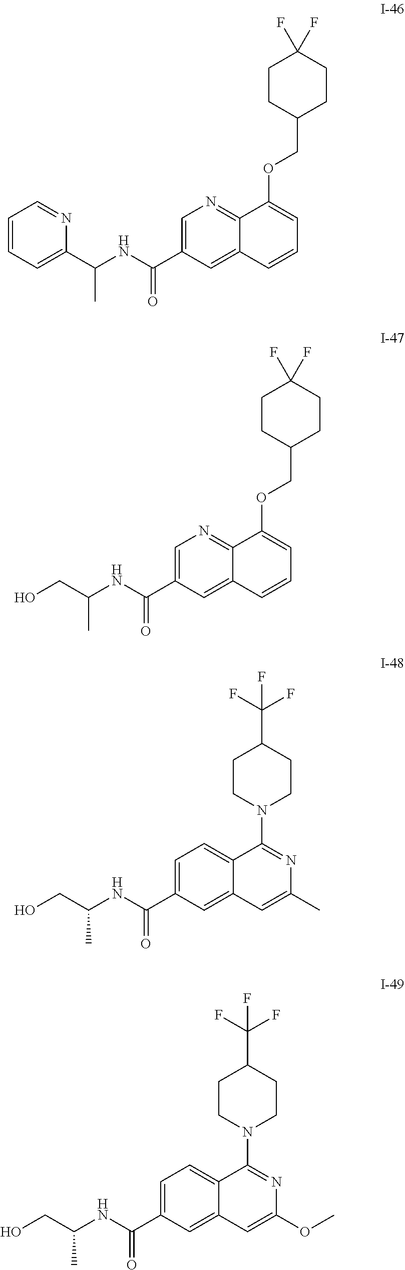 [SMILES: CC(NC(=O)c1cnc2c(OCC3CCC(F)(F)CC3)cccc2c1)c1ccccn1 ; CC(CO)NC(=O)c1cnc2c(OCC3CCC(F)(F)CC3)cccc2c1 ; Cc1cc2cc(C(=O)N[C@H](C)CO)ccc2c(N2CCC(C(F)(F)F)CC2)n1 ; COc1cc2cc(C(=O)N[C@H](C)CO)ccc2c(N2CCC(C(F)(F)F)CC2)n1]

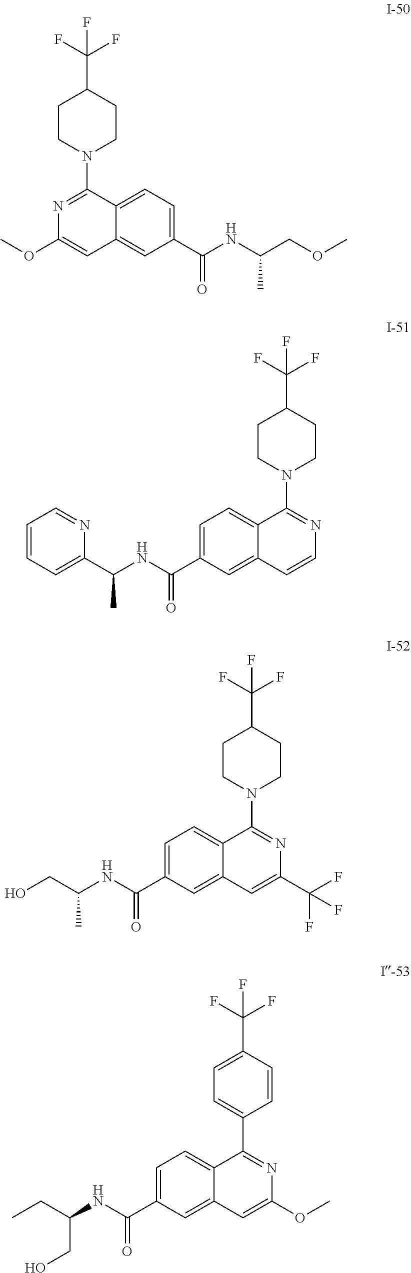

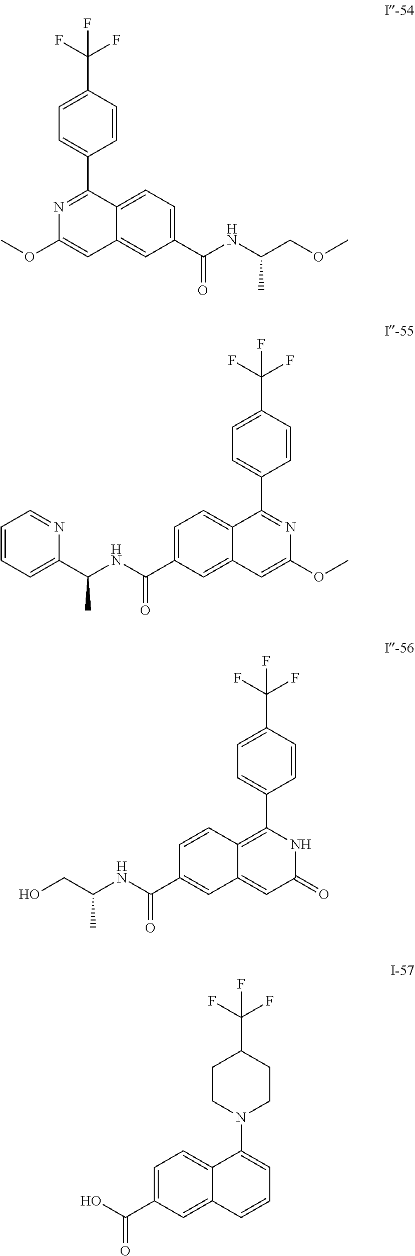

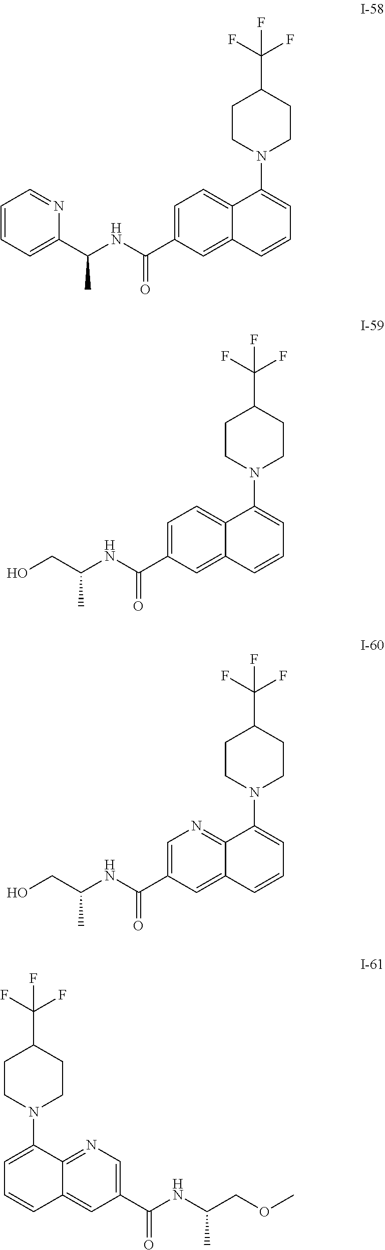 [SMILES: C[C@H](NC(=O)c1ccc2c(N3CCC(C(F)(F)F)CC3)cccc2c1)c1ccccn1 ; C[C@H](CO)NC(=O)c1ccc2c(N3CCC(C(F)(F)F)CC3)cccc2c1 ; C[C@H](CO)NC(=O)c1cnc2c(N3CCC(C(F)(F)F)CC3)cccc2c1 ; COC[C@H](C)NC(=O)c1cnc2c(N3CCC(C(F)(F)F)CC3)cccc2c1]

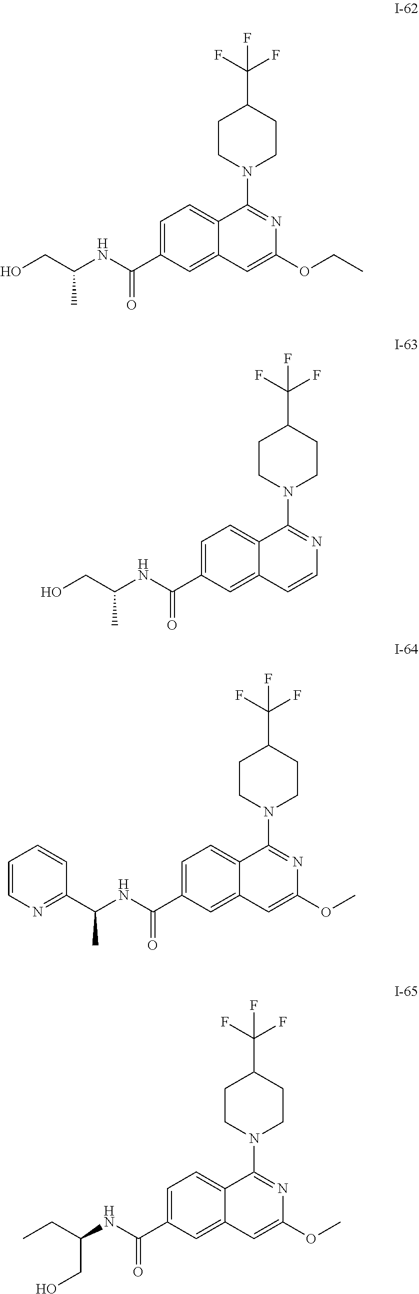

or a pharmaceutically acceptable salt thereof.

In some embodiments, a compound provided by this disclosure is selected from the group consisting of:

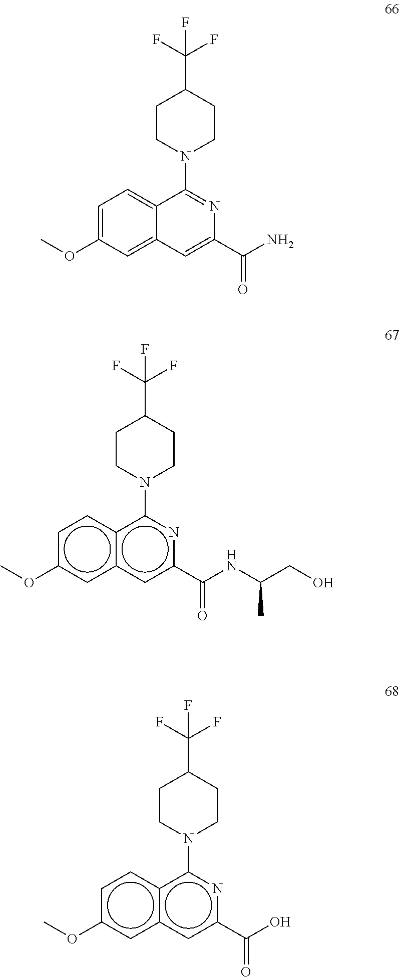

or a pharmaceutically acceptable salt thereof.

### 4. Uses, Formulation, and Administration:

Pharmaceutically Acceptable Compositions

According to another embodiment, the present disclosure provides a composition comprising a compound described herein or a pharmaceutically acceptable salt thereof and a pharmaceutically acceptable carrier, adjuvant, or vehicle. In certain embodiments, the amount of compound in compositions described herein is such that it is effective to measurably inhibit activity of a TEAD transcription factor, or a mutant thereof, in a biological sample or in a patient. In certain embodiments, a composition described herein is formulated for administration to a patient in need of such composition. In some embodiments, a composition described herein is formulated for oral administration to a patient.

Compounds and compositions, according to method of the present disclosure, are administered using any amount and any route of administration effective for treating or lessening the severity of a disorder provided herein (i.e., a TEAD-mediated disease or disorder). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternallyor via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Uses of Compounds and Pharmaceutically Acceptable Compositions

### The Hippo Signaling Pathway

The Hippo signaling pathway (also known as the Salvador/Warts/Hippo (SWH) pathway) is a key regulator of cell proliferation, death, and differentiation. In one aspect, a key function of the Hippo signaling pathway is the regulation of transcriptional co-activators Yes-associated protein (YAP; also known as YAP1 or YAP65) and its paralog, PDZ-binding motif (TAZ; also known as WWTR1). For example, the Hippo signaling pathway phosphorylates and inhibits YAP/TAZ activity by promoting their cytoplasmic retention and degradation, thereby inhibiting the growth promoting function regulated by YAP/TAZ. In an un-phosphorylated/de-phosphorylated state, YAP, together with TAZ, are transported into the nucleus where they interact with the TEAD family of transcriptions factors to upregulate genes that promote proliferation and migration, and inhibit apoptosis. Without wishing to be bound by a particular theory, in some instances, unregulated upregulation of these genes involved in proliferation, migration, and anti-apoptosis leads to the development of a disease, disorder, or condition (e.g., cancer). In some embodiments, overexpression of YAP/TAZ is associated with a disease, disorder, or condition (e.g., cancer).

Additional key members of the Hippo signaling pathway include the serine/threonine kinases MST1/2 (homologues of Hippo/Hpo of Drosophilia), Lats1/2 (homologues of Warts/Wts) and their adaptor proteins Sav1 (homologue of Salvador/Sav) and Mob (MOBKL1A and MOBKL1B1; homologues of Mats), respectively. In general, MST1/2 kinases complex with scaffold protein Sav1, which in turn phosphorylate and activate Lats1/2 kinase. Lats1/2 is also activated by the scaffold protein Mob. The activated Lats1/2 then phosphorylates and inactivates YAP or its paralog TAZ. The phosphorylation of YAP/TAZ leads to their nuclear export, retention within the cytoplasm, and degradation by the ubiquitin proteasome system.

In some instances, Lats1/2 phosphorylates YAP at the [HXRXXS] (SEQ ID NO: 5) consensus motifs, wherein X denotes any amino acid residue. YAP comprises five [HXRXXS](SEQ ID NO: 5) consensus motifs. In some instances, Lats1/2 phosphorylates YAP at one or more of the consensus motifs. In some instances, Lats1/2 phosphorylates YAP at all five of the consensus motifs. In some instances, Lats1/2 phosphorylates YAP at S127. In one aspect, the phosphorylation of YAP S127 promotes 14-3-3 protein binding and results in cytoplasmic sequestration of YAP. Mutation of YAP at the S127 position thereby disrupts its interaction with 14-3-3 and subsequently promotes nuclear translocation.

Additional phosphorylation occurs at S381 of YAP. Phosphorylation of YAP at S381 and on the corresponding site in TAZ primes both proteins for further phosphorylation events by CK1δ/ε in the degradation motif, which then signals for interaction with the β-TRCP E3 ubiquitin ligase, leading to polyubiquitination and degradation of YAP.

In some instances, Lats1/2 phosphorylates TAZ at the [HXRXXS] (SEQ ID NO: 5) consensus motifs, wherein X denotes any amino acid residue. TAZ comprises four [HXRXXS](SEQ ID NO: 5) consensus motifs. In some instances, Lats1/2 phosphorylates TAZ at one or more of the consensus motifs. In some instances, Lats1/2 phosphorylates TAZ at all four of the consensus motifs. In some instances, Lats1/2 phosphorylates TAZ at S89. In one aspect, the phosphorylation of TAZ S89 promotes 14-3-3 protein binding and results in cytoplasmic sequestration of TAZ. Mutation of TAZ at the S89 position thereby disrupts its interaction with 14-3-3 and subsequently promotes nuclear translocation.

In some embodiments, phosphorylated YAP/TAZ accumulates in the cytoplasm, and undergoes SCF^{β-TRCP}-mediated ubiquitination and subsequent proteasomal degradation. In some instances, the Skp, Cullin, F-box containing complex (SCF complex) is a multi-protein E3 ubiquitin ligase complex that comprises a F-box family member protein (e.g., Cdc4), Skp1, a bridging protein, and RBX1, which contains a small RING Finger domain which interacts with E2-ubiquitin conjugating enzyme. In some cases, the F-box family comprises more than 40 members, in which exemplary members include F-box/WD repeat-containing protein IA (FBXWIA, β-TrCPI, Fbxwl, hsSlimb, plkappaBalpha-E3 receptor subunit) and S-phase kinase-associated proteins 2 (SKP2). In some embodiments, the SCF complex (e.g., SCF^{β-TRCP}) interacts with an E1 ubiquitin-activating enzyme and an E2 ubiquitin-conjugating enzyme to catalyze the transfer of ubiquitin to the YAP/TAZ substrate. Exemplary E1 ubiquitin-activating enzymes include those encoded by the following genes: UBA1, UBA2, UBA3, UBA5, UBA6, UBA7, ATG7, NAEI, and SAEI. Exemplary E2 ubiquitin-conjugating enzymes include those encoded by the following genes: UBE2A, UBE2B, UBE2C, UBE2D1, UBE2D2, UBE2D3, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2M, UBE2N, UBE2O, UBE2Q1, UBE2Q2, UBE2R1, UBE2R2, UBE2S, UBE2T, UBE2U, UBE2V1, UBE2V2, UBE2Z, ATG2, BIRC5, and UFCI. In some embodiments, ubiquitinated YAP/TAZ further undergoes the degradation process through the 26S proteasome.

In some embodiments, the Hippo signaling pathway is regulated upstream by several different families of regulators. For example, in some instances, the Hippo signaling pathway is regulated by the G-protein and its coupled receptors, the Crumbs complex, regulators upstream of the MST kinases, and the adherens junction.

In some embodiments, the Hippo signaling pathway is regulated by G protein-coupled receptors (GPCR) and G protein (also known as guanine nucleotide-binding proteins) family of proteins. G proteins are molecular switches that transmit extracellular stimuli into the cell through GPCRs. In some instances, there are two classes of G proteins: monomeric small GTPases and heterotrimeric G protein complexes. In one aspect, the heterotrimeric G protein complexes comprise alpha (G_{α}), beta (G_{β}), and gamma (G_{γ}) subunits. In other aspects, there are several classes of Gα subunits: e.g., G_{q/11}α, G_{12/13}α, G_{i/o}α (G inhibitory, G other), and Gₛα (stimulatory).

In some instances, G_{q/11}α, G_{12/13}α, Gᵢα, and Gₒα coupled GPCRs activate YAP/TAZ and promote nuclear translocation. In other instances, Gₛα coupled GPCRs suppress YAP/TAZ activity, leading to YAP/TAZ degradation. In some instances, G_{q/11}α, G_{12/13}α, Gᵢα, and Gₒα coupled GPCRs activate YAP/TAZ through inhibition of Lats1/2 activity. In other instances, Gₛα coupled GPCRs promotes or induces Lats1/2 activity, thereby leading to YAP/TAZ degradation. See Yu et al., Cell. (2012) 150, 780-791.

In some embodiments, the Hippo signaling pathway is regulated by the Crumbs (Crb) complex. The Crumbs complex is a key regulator of cell polarity and cell shape. In some instances, the Crumbs complex comprises transmembrane CRB proteins that assemble multi-protein complexes that function in cell polarity. In some instances, CRB complexes recruit members of the Angiomotin (AMOT) family of adaptor proteins that interact with the Hippo signaling pathway. In some instances, AMOT directly binds to YAP, promotes YAP phosphorylation, and inhibits its nuclear localization. Zhao et al., Genes & Dev. (2011) 25, 51-63.

In some instances, the Hippo signaling pathway is regulated by other components (e.g., TAO kinases and cell polarity kinase PAR-1) that modulate the activity of MST kinases. MST kinases monitor actin cytoskeletal integrity.

In some instances, the Hippo signaling pathway is regulated by molecules of the adherens junction. In some instances, E-Cadherin (E-cad) suppresses YAP nuclear localization and activity through regulating MST activity. In some embodiments, E-cad-associated protein a-catenin regulates YAP through sequestering YAP/14-3-3 complexes in the cytoplasm. In other instances, Ajuba protein family members interact with Lats1/2 kinase activity, thereby preventing inactivation of YAP/TAZ.

In some embodiments, additional proteins that interact with YAP/TAZ either directly or indirectly include, but are not limited to, Merlin, protocadherin Fat 1, MASK1/2, HIPK2, PTPN14, RASSF, PP2A, Salt-inducible kinases (SIKs), Scribble (SCRIB), the Scribble associated proteins Discs large (Dlg), KIBRA, PTPN14, NPHP3, LKB1, Ajuba, and ZO1/2.

In some instances, it has been shown that in BRAF-mutant tumor cells, YAP acts as a parallel survival input to promote resistance to RAF and MEK inhibitor therapy. See Lin et al Nat. Genet. (2015) 47, 250-256. It has been shown that combined YAP and RAF or MEK inhibition is lethal in several BRAF-mutant tumor types and also RAS-mutant tumors. See Lin et al Nat. Genet. (2015) 47, 250-256. Additionally or alternatively, silencing either TEAD2 or TEAD4 had the same phenotypic effect as YAP1 suppression on sensitivity of RAF and MEK inhibitors in HCC364 cells. See Lin et al Nat. Genet. (2015) 47, 250-256. A directed interaction between MAPK signaling and TEAD stability has also been shown, such that knockdown of YAP combined with MEK inhibition results in robust inhibition of tumor cell growth in Hippo dysreulgated tumors. Pham et al. Cancer Discovery (2021) 11, 778-793.

In addition, it has been demonstrated that certain cancer cells treated with epidermal growth factor receptor (EGFR) tyrosine kinase inhibitors enter a drug-tolerant dormant state characterized by high YAP/TEAD activity. Kurppa et al Cancer Cell (2020) 37, 104-122. By engaging with transcription factor SLUG to directly repress pro-apopototic BMF, YAP/TEAD is able to limit drug-induced apoptosis. Co-inhibition of YAP and TEAD, or genetic deletion of YAP1, depleted dormant cells by enhancing EGFR/MEK inhibition-induced apoptosis. Without wishing to be bound to a particular theory, it is hypothesized that targeting YAP/TEAD could enhance drug-induced apoptosis (e.g., through EGFR/MEK inhibition) and reduce residual disease and/or drug resistance.

### TEAD

In some embodiments, un-phosphorylated and/or dephosphorylated YAP/TAZ accumulates in the nucleus. In one aspect, once within the nucleus, YAP/TAZ interacts with the TEAD family of transcriptions factors (e.g., human TEAD1 (UniProt KB ID P28347-1 (SEQ IDNO: 1)); human TEAD2 (UniProtKB ID Q15562 (SEQ IDNO: 2)); human TEAD3 (UniProtKB ID Q99594 (SEQ ID NO: 3)); and human TEAD4 (UniProtKB ID Q15561 (SEQ ID NO: 4)) to activate genes that promote proliferation and migration, and inhibit apoptosis, such as, e.g., CTFG, Cyr61, and FGF1. In one aspect, without wishing to be bound by a particular theory, since TEAD is a downstream transcription factor of the Hippo pathway, inhibiting the function of TEAD is an attractive therapeutic strategy to reduce aberrant Hippo signaling and gene transcription.

TEAD1-4 are composed of a highly conserved TEA DNA binding domain and YAP binding domain, which is separated by a proline rich region. Despite the high homology shared between human TEAD1-4, the individual TEAD proteins are differentially expressed in a tissue- and development-dependent manner. For example, in some instances, TEAD1 is required for heart biogenesis, TEAD2 for embryonic development, TEAD4 for activating skeletal muscle genes, and TERAD3 has been shown to be specifically expressed in the placenta and several embryonic tissues during development. Holden et al. Cancers (2018) 10, 81, 1-15.

Proteomic and biochemical studies have shown that the TEAD family of transcription factors are palmitoylated at evolutionarily conserved cysteine residues. Three cysteine residues were found that are evolutionarily conserved and mutated to serine in human TEAD1 (C53S, C327S and C359S) to test whether the mutation affects TEAD1 palmitoylation. The C359S mutant showed the greatest loss of palmitoylation, and C327S and C53S also showed decreased palmitoylation. These results suggest that C359 plays a key role in TEAD1 palmitoylation. Furthermore, combination mutation of all three cysteine residues, C53/327/359S (3CS), completely ablated TEAD1 palmitoylation, indicating that these residues are involved in TEAD1 palmitoylation. In one aspect, it has been found that TEADs undergo PAT-independent autopalmitoylation, under physiological concentrations of palmitoyl-CoA. Furthermore, autopalmitoylation plays key roles in regulating TEAD-YAP association and their physiological functions in vitro and in vivo. Chan, et al. Nature Chem. Biol. (2016) 12, 282-289; Noland, et al. Structure, (2016) 24, 1-8; Gibault et al. J. Med. Chem. (2018) 61, 5057-5072. Therefore, in one aspect, palmitoylation of TEADs play important roles in regulating Hippo signaling pathway transcriptional complexes.

It will be understood that the term “YAP/TAZ” refers to YAP, TAZ, or both YAP and TAZ.

In some embodiments, compounds disclosed herein modulate the interaction between YAP/TAZ and TEAD. In some embodiments, compounds disclosed herein bind to TEAD and/or prevent interaction between YAP/TAZ and TEAD.

In some embodiments, compounds disclosed herein irreversibly bind to a TEAD transcription factor (e.g., TEAD1, TEAD2, TEAD3, or TEAD4). In some embodiments, compounds disclosed herein covalently binds to a TEAD transcription factor (e.g., TEAD1, TEAD2, TEAD3, or TEAD4). In some embodiments, compounds disclosed covalently inhibit the activity of a TEAD transcription factor (e.g., TEAD1, TEAD2, TEAD3, or TEAD4). In some embodiments, compounds disclosed irreversibly inhibit the activity of a TEAD transcription factor (e.g., TEAD1, TEAD2, TEAD3, or TEAD4).

In some embodiments, compounds disclosed herein bind to TEAD1 at C53. In some embodiments, compounds disclosed herein bind to TEAD1 at C327. In some embodiments, compounds disclosed herein bind to TEAD1 at C359. In some embodiments, compounds disclosed herein bind to TEAD1 at C405. In some embodiments, compounds disclosed herein bind to TEAD1 at C53 and C327. In some embodiments, compounds disclosed herein bind to TEAD1 at C53 and C359. In some embodiments, compounds disclosed herein bind to TEAD1 at C53 and C405. In some embodiments, compounds disclosed herein bind to TEAD1 at C327 and C359. In some embodiments, compounds disclosed herein bind to TEAD1 at C327 and C405. In some embodiments, compounds disclosed herein bind to TEAD1 at C359 and C405. In some embodiments, compounds disclosed herein bind to TEAD1 at C53, C327, and C359. In some embodiments, compounds disclosed herein bind to TEAD1 at C53, C327, and C405. In some embodiments, compounds disclosed herein bind to TEAD1 at C53, C359, and C405. In some embodiments, compounds disclosed herein bind to TEAD1 at C327, C359, and C405. In some embodiments, compounds disclosed herein bind to TEAD1 at C53, C327, C359, and C405.

In some embodiments, compounds disclosed herein bind to TEAD2 at C368. In some embodiments, compounds disclosed herein bind to TEAD2 at C380. In some embodiments, compounds disclosed herein bind to TEAD2 at C368 and C380

In some embodiments, compounds disclosed herein bind to TEAD3 at C368. In some embodiments, compounds disclosed herein bind to TEAD3 at C371. In some embodiments, compounds disclosed herein bind to TEAD3 at C368 and C368.

In some embodiments, compounds disclosed herein bind to TEAD4 at C367.

In some embodiments, compounds disclosed herein bind to a TEAD transcription factor (e.g., TEAD1, TEAD2, TEAD3, or TEAD4) and disrupt or inhibit the interaction between YAP/TAZ and the TEAD transcription factor. In some embodiments, compounds disclosed herein bind to TEAD1 and disrupt or inhibit the interaction between YAP/TAZ and TEAD1. In some embodiments, compounds disclosed herein bind to TEAD2 and disrupt or inhibit the interaction between YAP/TAZ and TEAD2. In some embodiments, compounds disclosed herein bind to TEAD3 and disrupt or inhibit the interaction between YAP/TAZ and TEAD3. In some embodiments, compounds disclosed herein bind to TEAD4 and disrupt or inhibit the interaction between YAP/TAZ and TEAD4.

In some embodiments, compounds disclosed herein bind to TEAD1 at C53, and disrupt or inhibit the interaction between YAP/TAZ and TEAD1. In some embodiments, compounds disclosed herein bind to TEAD1 at C327, and disrupt or inhibit the interaction between YAP/TAZ and TEAD1. In some embodiments, compounds disclosed herein bind to TEAD1 at C359, and disrupt or inhibit the interaction between YAP/TAZ and TEAD1. In some embodiments, compounds disclosed herein bind to TEAD1 at C405, and disrupt or inhibit the interaction between YAP/TAZ and TEAD1. In some embodiments, compounds disclosed herein bind to TEAD1 at C53 and C327, and disrupt or inhibit the interaction between YAP/TAZ and TEAD1. In some embodiments, compounds disclosed herein bind to TEAD1 at C53 and C359, and disrupt or inhibit the interaction between YAP/TAZ and TEAD1. In some embodiments, compounds disclosed herein bind to TEAD1 at C53 and C405, and disrupt or inhibit the interaction between YAP/TAZ and TEAD1. In some embodiments, compounds disclosed herein bind to TEAD1 at C327 and C359, and disrupt or inhibit the interaction between YAP/TAZ and TEAD1. In some embodiments, compounds disclosed herein bind to TEAD1 at C327 and C405, and disrupt or inhibit the interaction between YAP/TAZ and TEAD1. In some embodiments, compounds disclosed herein bind to TEAD1 at C359 and C405, and disrupt or inhibit the interaction between YAP/TAZ and TEAD1. In some embodiments, compounds disclosed herein bind to TEAD1 at C53, C327, and C359, and disrupt or inhibit the interaction between YAP/TAZ and TEAD1. In some embodiments, compounds disclosed herein bind to TEAD1 at C53, C327, and C405, and disrupt or inhibit the interaction between YAP/TAZ and TEAD1. In some embodiments, compounds disclosed herein bind to TEAD1 at C53, C359, and C405, and disrupt or inhibit the interaction between YAP/TAZ and TEAD1. In some embodiments, compounds disclosed herein bind to TEAD1 at C327, C359, and C405, and disrupt or inhibit the interaction between YAP/TAZ and TEAD1. In some embodiments, compounds disclosed herein bind to TEAD1 at C53, C327, C359, and C405, and disrupt or inhibit the interaction between YAP/TAZ and TEAD1.

In some embodiments, compounds disclosed herein bind to TEAD2 at C368, and disrupt or inhibit the interaction between YAP/TAZ and TEAD2. In some embodiments, compounds disclosed herein bind to TEAD2 at C380, and disrupt or inhibit the interaction between YAP/TAZ and TEAD2. In some embodiments, compounds disclosed herein bind to TEAD2 at C368 and C380, and disrupt or inhibit the interaction between YAP/TAZ and TEAD2.

In some embodiments, compounds disclosed herein bind to TEAD3 at C368, and disrupt or inhibit the interaction between YAP/TAZ and TEAD3. In some embodiments, compounds disclosed herein bind to TEAD3 at C371, and disrupt or inhibit the interaction between YAP/TAZ and TEAD3. In some embodiments, compounds disclosed herein bind to TEAD3 at C368 and C368, and disrupt or inhibit the interaction between YAP/TAZ and TEAD3.

In some embodiments, compounds disclosed herein bind to TEAD4 at C367, and disrupt or inhibit the interaction between YAP/TAZ and TEAD4.

In some embodiments, compounds disclosed herein bind to a TEAD transcription factor (e.g., TEAD1, TEAD2, TEAD3, or TEAD4) and prevent TEAD transcription palmitoylation. In some embodiments, compounds disclosed herein bind to TEAD1 and prevent TEAD1 palmitoylation. In some embodiments, compounds disclosed herein bind to TEAD1 and prevent TEAD1 palmitoylation at C53. In some embodiments, compounds disclosed herein bind to TEAD1 and prevent TEAD1 palmitoylation at C327. In some embodiments, compounds disclosed herein bind to TEAD1 and prevent TEAD1 palmitoylation at C359. In some embodiments, compounds disclosed herein bind to TEAD1 and prevent TEAD1 palmitoylation at C405. In some embodiments, compounds disclosed herein bind to TEAD1 and prevent TEAD1 palmitoylation at C53 and C327. In some embodiments, compounds disclosed herein bind to TEAD1 and prevent TEAD1 palmitoylation at C53 and C359. In some embodiments, compounds disclosed herein bind to TEAD1 and prevent TEAD1 palmitoylation at C53 and C459. In some embodiments, compounds disclosed herein bind to TEAD1 and prevent TEAD1 palmitoylation at C327 and C359. In some embodiments, compounds disclosed herein bind to TEAD1 and prevent TEAD1 palmitoylation at C327 and C405. In some embodiments, compounds disclosed herein bind to TEAD1 and prevent TEAD1 palmitoylation at C359 and C405. In some embodiments, compounds disclosed herein bind to TEAD1 and prevent TEAD1 palmitoylation at C53, C327, and C359. In some embodiments, compounds disclosed herein bind to TEAD1 and prevent TEAD1 palmitoylation at C53, C327, and C405. In some embodiments, compounds disclosed herein bind to TEAD1 and prevent TEAD1 palmitoylation at C327, C359, and C405. In some embodiments, compounds disclosed herein bind to TEAD1 and prevent TEAD1 palmitoylation at C53, C327, C359, and C405.

In some embodiments, compounds disclosed herein bind to TEAD2 and prevent TEAD2 palmitoylation at C368. In some embodiments, compounds disclosed herein bind to TEAD2 and prevent TEAD2 palmitoylation at C380. In some embodiments, compounds disclosed herein bind to TEAD2 and prevent TEAD2 palmitoylation at C368 and C380.

In some embodiments, compounds disclosed herein bind to TEAD3 and prevent TEAD3 palmitoylation at C368. In some embodiments, compounds disclosed herein bind to TEAD3 and prevent TEAD3 palmitoylation at C371. In some embodiments, compounds disclosed herein bind to TEAD3 and prevent TEAD3 palmitoylation at C368 and C371.

In some embodiments, compounds disclosed herein bind to TEAD4 and prevent TEAD4 palmitoylation at C367.

In some embodiments, compounds disclosed herein bind to a TEAD transcription factor (e.g., TEAD1, TEAD2, TEAD3, or TEAD4), prevent TEAD transcription factor palmitoylation, and disrupt or inhibit the interaction between YAP/TAZ and the TEAD transcription factor. In some embodiments, compounds disclosed herein bind to TEAD1, prevent TEAD1 palmitoylation, and disrupt or inhibit the interaction between YAP/TAZ and TEAD1. In some embodiments, compounds disclosed herein bind to TEAD1, prevent TEAD1 palmitoylation at C53, and disrupt or inhibit the interaction between YAP/TAZ and TEAD1. In some embodiments, compounds disclosed herein bind to TEAD1, prevent TEAD1 palmitoylation at C327, and disrupt or inhibit the interaction between YAP/TAZ and TEAD1. In some embodiments, compounds disclosed herein bind to TEAD1, prevent TEAD1 palmitoylation at C359, and disrupt or inhibit the interaction between YAP/TAZ and TEAD1. In some embodiments, compounds disclosed herein bind to TEAD1, prevent TEAD1 palmitoylation at C405, and disrupt or inhibit the interaction between YAP/TAZ and TEAD1. In some embodiments, compounds disclosed herein bind to TEAD1, prevent TEAD1 palmitoylation at C53 and C327, and disrupt or inhibit the interaction between YAP/TAZ and TEAD1. In some embodiments, compounds disclosed herein bind to TEAD1, prevent TEAD1 palmitoylation at C53 and C359, and disrupt or inhibit the interaction between YAP/TAZ and TEAD1. In some embodiments, compounds disclosed herein bind to TEAD1, prevent TEAD1 palmitoylation at C53 and C459, and disrupt or inhibit the interaction between YAP/TAZ and TEAD1. In some embodiments, compounds disclosed herein bind to TEAD1, prevent TEAD1 palmitoylation at C327 and C359, and disrupt or inhibit the interaction between YAP/TAZ and TEAD1. In some embodiments, compounds disclosed herein bind to TEAD1, prevent TEAD1 palmitoylation at C327 and C405, and disrupt or inhibit the interaction between YAP/TAZ and TEAD1. In some embodiments, compounds disclosed herein bind to TEAD1, prevent TEAD1 palmitoylation at C359 and C405, and disrupt or inhibit the interaction between YAP/TAZ and TEAD1. In some embodiments, compounds disclosed herein bind to TEAD1, prevent TEAD1 palmitoylation at C53, C327, and C359, and disrupt or inhibit the interaction between YAP/TAZ and TEAD1. In some embodiments, compounds disclosed herein bind to TEAD1, prevent TEAD1 palmitoylation at C53, C327, and C405, and disrupt or inhibit the interaction between YAP/TAZ and TEAD1. In some embodiments, compounds disclosed herein bind to TEAD1, prevent TEAD1 palmitoylation at C327, C359, and C405, and disrupt or inhibit the interaction between YAP/TAZ and TEAD1. In some embodiments, compounds disclosed herein bind to TEAD1, prevent TEAD1 palmitoylation at C53, C327, C359, and C405, and disrupt or inhibit the interaction between YAP/TAZ and TEAD1.

In some embodiments, compounds disclosed herein bind to TEAD2, prevent TEAD2 palmitoylation at C368, and disrupt or inhibit the interaction between YAP/TAZ and TEAD1. In some embodiments, compounds disclosed herein bind to TEAD2, prevent TEAD2 palmitoylation at C380, and disrupt or inhibit the interaction between YAP/TAZ and TEAD1. In some embodiments, compounds disclosed herein bind to TEAD2, prevent TEAD2 palmitoylation at C368 and C380, and disrupt or inhibit the interaction between YAP/TAZ and TEAD1.

In some embodiments, compounds disclosed herein bind to TEAD3, prevent TEAD3 palmitoylation at C368, and disrupt or inhibit the interaction between YAP/TAZ and TEAD1. In some embodiments, compounds disclosed herein bind to TEAD3, prevent TEAD3 palmitoylation at C371, and disrupt or inhibit the interaction between YAP/TAZ and TEAD1. In some embodiments, compounds disclosed herein bind to TEAD3, prevent TEAD3 palmitoylation at C368 and C371, and disrupt or inhibit the interaction between YAP/TAZ and TEAD1.

In some embodiments, compounds disclosed herein bind to TEAD4, prevent TEAD4 palmitoylation at C367, and disrupt or inhibit the interaction between YAP/TAZ and TEAD1.

The activity of a compound described herein as an inhibitor of TEAD (e.g., TEAD1, TEAD2, TEAD3, and/or TEAD4), or a variant or mutant thereof, can be assayed in vitro, in vivo, or in a cell line. In vitro assays include assays that determine inhibition of TEAD (e.g., TEAD1, TEAD2, TEAD3, and/or TEAD4), or a variant or mutant thereof. Alternate in vitro assays quantitate the ability of the inhibitor to bind to TEAD (e.g., TEAD1, TEAD2, TEAD3, and/or TEAD4) or a variant or mutant thereof. Detailed conditions for assaying a compound described herein as an inhibitor of TEAD (e.g., TEAD1, TEAD2, TEAD3, and/or TEAD4), or a variant or mutant thereof, are set forth in the Examples below. See, for example, Example 2.

The provided compounds are inhibitors of TEAD (e.g., TEAD1, TEAD2, TEAD3, and/or TEAD4) and are therefore useful for treating one or more disorders associated with activity of TEAD (e.g., TEAD1, TEAD2, TEAD3, and/or TEAD4). Thus, in some aspects and embodiments, the present disclosure provides a method for treating a TEAD-mediated disease, disorder, or condition comprising the step of administering to a patient in need thereof a compound of the present disclosure, or pharmaceutically acceptable composition thereof.

In some embodiments, the present disclosure provides a method of inhibiting TEAD (e.g., TEAD1, TEAD2, TEAD3, and/or TEAD4) comprising contacting a cell with a compound of formula I.

As used herein, the term “TEAD-mediated” disorders or conditions as used herein means any disease or other deleterious condition in which TEAD (e.g., TEAD1, TEAD2, TEAD3, and/or TEAD4), or a mutant thereof, is known to play a role. Accordingly, another embodiment of the present disclosure relates to treating or lessening the severity of one or more diseases in which TEAD (e.g., TEAD1, TEAD2, TEAD3, and/or TEAD4), or a mutant thereof, is known to play a role.

In some embodiments, the present disclosure provides methods of treating, reducing the severity of, delaying the onset of, or inhibiting the progress of a disease or disorder, or one or more symptoms thereof of a disease or disorder characterized by or associated with increased TEAD (e.g., TEAD1, TEAD2, TEAD3, and/or TEAD4) expression and/or increased TEAD (e.g., TEAD1, TEAD2, TEAD3, and/or TEAD4) activity, comprising the step of administering to a patient in need thereof a therapeutically effective a compound of the present disclosure, or pharmaceutically acceptable composition thereof. In some embodiments, the present disclosure provides methods of treating, reducing the severity of, delaying the onset of, or inhibiting the progress of a disease or disorder, or one or more symptoms thereof of a disease or disorder in which inhibition or antagonizing of TEAD (e.g., TEAD1, TEAD2, TEAD3, and/or TEAD4) activity is beneficial comprising the step of administering to a patient in need thereof a compound described herein, or pharmaceutically acceptable composition thereof. In some aspects and embodiments, provided herein are methods of treating, reducing the severity of, delaying the onset of, or inhibiting the progress of a disease or disorder, or one or more symptoms thereof of a disease or disorder in which inhibition or antagonizing of the Hippo signaling pathway is beneficial comprising the step of administering to a patient in need thereof a therapeutically effective compound of the present disclosure, or pharmaceutically acceptable composition thereof.

In some aspects and embodiments, the present disclosure provides a method for treating one or more disorders, diseases, and/or conditions wherein the disorder, disease, or condition includes, but is not limited to, a cellular proliferative disorder, comprising administering to a patient in need thereof, a TEAD inhibitor compound as described herein, or a pharmaceutical salt or composition thereof. In some embodiments, a cellular proliferative disorder is cancer. In some embodiments, the cancer is characterized by increased TEAD (e.g., TEAD1, TEAD2, TEAD3, and/or TEAD4) expression and/or increased TEAD (e.g., TEAD1, TEAD2, TEAD3, and/or TEAD4) activity.

In some embodiments, provided methods include the co-administration of a provided compound and at least one mitogen-activated protein kinase (MAPK) inhibitor. In some embodiments, provided methods include the co-administration of a provided compound and at least one inhibitor of the RAS/MAPK pathway. In some embodiments, provided methods include the co-administration of a provided compound and at least one epidermal growth factor receptor (EGFR) inhibitor. In some embodiments, an inhibitor of the RAS/MAPK pathway is a KRAS inhibitor, RAF inhibitor (e.g., a BRAF monomer or RAF dimer inhibitor), a MEK inhibitor, an ERK inhibitor, an EGFR inhibitor, or a MAPK inhibitor, or a combination thereof. In some embodiments, an inhibitor of the RAS/MAPK pathway is an EGFR inhibitor or a MAPK inhibitor, or a combination thereof. Examples of EGFR inhibitors, MAPK inhibitors, and/or RAS/MAPK pathway inhibitors are disclosed in Moore A. R. Rosenberg, S. C., McCormock, F. et al. Nat. Rev. Discov. (2020) and include, e.g., Osimertinib (TAGRISSO®, AstraZeneca), sotorasib (AMG 510 from Amgen), MRTX849 (from Mirati Therapeutics), JNJ-74699157/ARS-3248 (from J&J Wellspring Biosciences), LY3499446 (from Eli Lilly), GDCBI 1701963 (from Boehringer Ingelheim), mRNA-5671 (from Moderna Therapeutics), G12D inhibitor (from Mirati Therapeutics), RAS(ON) inhibitors (from Revolution Medicines), BBP-454 (from BridgeBio Pharma), SP600125, PLX4032, GW5074, AZD6244, PD98059, simvastatin, alisertib, teriflunomide, NSC95397, PD325901, PD98059, lovastatin, sorafenib (NEXAVAR®, Bayer Labs), vermurafenib (ZELBORAF®, Hoffman La Roche Inc.), dabrafenib (TAFLINAR®, Novartis Pharmaceuticals Corporation), selumetinib (KOSELUGO™, AstraZeneca Pharmaceuticals LP), trametinib (MEKINIST®, Novartis Pharmaceuticals Corporation), uxliertinib, silimarin, sirolimus (RAPAMUNE®, PV Prism CV), lapatinib (TYKERB®/TYVERB®, GlaxoSmithKline), crizotinib (XALKORI®, PF Prism CV), taselisib (Roche), PF-0491502, pF502, enterolactone, PLX4720, PD0325901, PD184352, SC-514, alisterib (MLN8237), SB415286, PLX4720, obtaoclax (GX15-070), pimasterib, venetoclax (ABT-199/VENCLEXTA®/VENCLYXTO®), eprenetapopt (APR-246), gemcitabine (GEMZAR®), birinapant (TL32711), pexmetinib (ARRY-614), afuresertib, ralimetinib (LY2228820, Eli Lilly), cobimetinib (COTELLIC®, Exelixis/Genentech), prexasertib (LY2606368), erlotinib (TARCEVA®, OSI Pharmaceuticals), bevacizumab (AVASTIN®, Genentech), belvarafenib (Hanmi Pharm./Genentech, Inc.) and binimetinib (MEKTOVI®, Array Biopharma Inc.).

As used herein, the terms “increased expression” and/or “increased activity” of a substance, such as TEAD, in a sample or cancer or patient refers to an increase in the amount of the substance, such as TEAD, of about 5%, about I 0%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount of the substance, such as TEAD, in a control sample or control samples, such as an individual or group of individuals who are not suffering from the disease or disorder (e.g., cancer) or an internal control, as determined by techniques known in the art. A subject can also be determined to have an “increased expression” or “increased activity” of TEAD if the expression and/or activity of TEAD is increased by one standard deviation, two standard deviations, three standard deviations, four standard deviations, five standard deviations, or more, relative to the mean (average) or median amount of TEAD in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

In some embodiments, the present disclosure provides a method for treating or lessening the severity of a cancer including, without limitation, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer. In some embodiments, the cancer is or has metastasized. In some embodiments, the cancer is relapsed or refractory cancer. In some embodiments, the cancer is a relapsed or refractory solid tumor. In some embodiments, the cancer is a relapsed or refractory hematological malignancy. In some embodiments, the cancer is or has been characterized by or has been established to have one or more genetic alterations in the Hippo pathway (e.g., NF2, LATS1/2, AMOTL2, SAV1, TAOK1-3, etc.). In some embodiments, the cancer is or has been characterized by or has been established to have one or more genetic alterations that affect or alter the stability of Hippo pathway components (e.g., BAP1, SOCS6, etc.). In some embodiments, the cancer is or has been characterized by or has been established to have a YAP/TAZ gene translocation (e.g., WWTR1(TAZ)-CAMTA1, YAP1-TFE3, etc.). In some embodiments, the cancer is selected from those disclosed in WO 2019/113236, the entire contents of which are hereby incorporated by reference.

In some embodiments, the cancer is mediated by activation YAP/TAZ. In some embodiments of the methods and uses described herein, the cancer is mediated by modulation of the interaction of YAP/TAZ with TEAD (e.g., TEADI, TEAD2, TEAD3, and/or TEAD4). In some embodiments, the cancer is characterized by or associated with increased TEAD (e.g., TEAD1, TEAD2, TEAD3, and/or TEAD4) expression and/or increased TEAD (e.g., TEAD1, TEAD2, TEAD3, and/or TEAD4) activity. In some embodiments, the cancer being treated is a cancer in which YAP/TAZ is localized in the nucleus of the cancer cells. In some embodiments, the cancer being treated is or has been characterized or established by one or more YAP/TAZ genetic amplifications or mutations.

In some embodiments, the cancer is characterized by a mutant Gα-protein. In some embodiments, a mutant Gα-protein is G₁₂, G₁₃, G_{q}, G₁₁, Gᵢ, Gₒ, or Gₛ. In some embodiments, a mutant Gα-protein is G₁₂. In some embodiments, a mutant Gα-protein is G₁₃. In some embodiments, a mutant Gα-protein is G_{q}. In some embodiments, a mutant Gα-protein is G₁₁. In some embodiments, a mutant Gα-protein is Gᵢ. In some embodiments, a mutant Ga-protein is Go. In some embodiments, a mutant Gα-protein is Gₛ.

In some embodiments, the cancer is lung cancer, thyroid cancer, ovarian cancer, colorectal cancer, prostate cancer, cancer of the pancreas, cancer of the esophagus, liver cancer, breast cancer, skin cancer, or mesothelioma. In some embodiments, the cancer is mesothelioma, such as malignant mesothelioma. In some embodiments, the cancer is leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell-involved cancers (including cervical squamous cell carcinoma, lung squamous cell carcinoma, esphageal squamous cell carcinoma, head and neck squamous cell carcinoma, bladder urothelial carcinoma), basal cell carcinoma, adenocarcinoma, sweat gland carcinoma, sebaceous gland carcmoma, papillary carcmoma, papillary adenocarcinomas, cystadenocarcinoma, medullary carcinoma, bronchogenic carcinoma, renal cell carcinoma, hepatoma, bile duct carcinoma (i.e. cholangiocarcinoma), choriocarcinoma, seminoma, embryonal carcinoma, Wilm's tumor, cervical cancer, endometrial/uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, epithelioid hemangioendothelioma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g., Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

In some embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcmoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcmoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcmoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is being treated adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells; as well as virus-associated tumors in gastric cancer, nasopharyngeal carcinoma, cervical cancer, vaginal cancer, vulvar cancer, squamous cell carcinoma of the head and neck, and Merkel cell carcinoma.

In some embodiments, the cancer is melanoma cancer. In some embodiments, the cancer is breast cancer. In some embodiments, the cancer is lung cancer. In some embodiments, the cancer is small cell lung cancer (SCLC). In some embodiments, the cancer is non-small cell lung cancer (NSCLC).

### EXEMPLIFICATION

As depicted in the Examples below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Example 1. Synthesis of Exemplary Compounds

### Example 1.1. Synthesis of (R)-N-(1-hydroxypropan-2-yl)-6-methoxy-8-(4-(trifluoromethyl)piperidin-1-yl)quinoline-3-carboxamide (I-1)

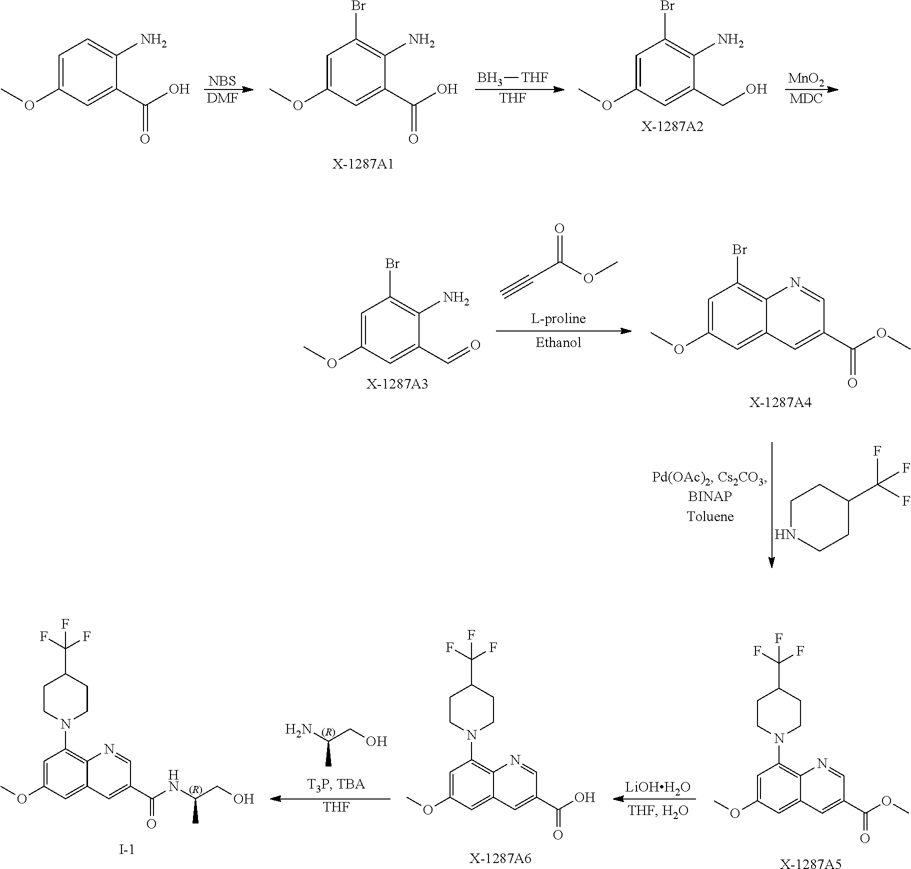

2-Amino-3-bromo-5-methoxybenzoic acid (X-1287A1). To a stirred solution of 2-amino-5-methoxybenzoic acid (20.0 g, 119.7 mmol) in DMF (400 mL) was added N-bromosuccinimide (21.3 g, 119.7 mmol) portion wise at 0° C. under nitrogen and the resulting mixture was stirred at room temperature for 16 h. Reaction mixture was directly purified by reverse phase (C-18) silica gel column chromatography, using acetonitrile-water=0:1→1:0 as gradient, to afford 2-amino-3-bromo-5-methoxybenzoic acid (X-1287A1) (9.0 g, 34%) as a purple solid. MS: [MH]⁺245.9/[MH+2]⁺247.9.

(2-Amino-3-bromo-5-methoxyphenyl)methanol (X-1287A2). To a stirred solution of 2-amino-3-bromo-5-methoxybenzoic acid (9.0 g, 36.73 mmol) in THF (17 mL) was added BH₃.THF (105 mL, 110.20 mmol) at −5° C. under nitrogen. After 15 min of stirring at the same temperature, the reaction temperature was slowly brought to 70° C. and stirred for 16 h at the same temperature. Reaction mixture was cooled to room temperature, quenched with MeOH (500 mL) and concentrated under reduced pressure. The obtained residue was dissolved in ethyl acetate (300 mL), washed with water (100 mL×3), dried over anhydrous Na₂SO₄, filtered, and concentrated under reduced pressure to afford (2-amino-3-bromo-5-methoxyphenyl) methanol (X-1287A2) (6.2 g, 73% (crude)) as an off-white solid. The compound was pure enough to proceed to the next step without further purification. MS: [MH]⁺231.9/[MH+2]⁺233.9.

2-Amino-3-bromo-5-methoxybenzaldehyde (X-1287A3). To a stirred solution of (2-amino-3-bromo-5-methoxyphenyl) methanol (X-1287A2) (6.2 g, 26.83 mmol) in DCM (125 mL) was added MnO₂ (23.4 g, 268.30 mmol) at 0° C. and the resulting mixture was stirred at room temperature for 16 h. Reaction mixture was filtered through a celite bed and filtrate was concentrated under reduced pressure to afford 2-amino-3-bromo-5-methoxybenzaldehyde (X-1287A3) (5.5 g, 89% (crude)) as a brown solid, which was used in next step without further purification. MS: [MH]⁺229.8/[MH+2]⁺231.8.

Methyl 8-bromo-6-methoxyquinoline-3-carboxylate (X-1287A4). To a stirred solution of 2-amino-3-bromo-5-methoxybenzaldehyde (X-1287A3) (5.5 g, 23.93 mmol) in ethanol (60 mL) were added methyl propiolate (3.0 g, 35.89 mmol) and L-proline (1.38 g, 11.96 mmol) at room temperature and the resulting mixture was heated at 80° C. for 16 h. After cooling to room temperature, the reaction mixture was slowly poured into n-hexane (500 mL) and the resulting precipitate was collected by filtration. Obtained solid residue was washed with n-hexane (500 mL), and dried under high vacuum to afford methyl 8-bromo-6-methoxyquinoline-3-carboxylate (X-1287A4) (4.5 g, 64%) as an off-white solid. MS: [MH]⁺295.9/[MH+2]⁺297.9.

Methyl 6-methoxy-8-(4-(trifluoromethyl)piperidin-1-yl)quinoline-3-carboxylate (X-1287A5). To a stirred solution of methyl 8-bromo-6-methoxyquinoline-3-carboxylate (X-1287A4) (1.20 g, 4.06 mmol) in a toluene (10 mL) were added 4-(trifluoromethyl)piperidine (1.86 g, 12.20 mmol), cesium carbonate (7.95 g, 24.40 mmol), and rac-BINAP (0.505 g, 0.81 mmol) sequentially at room temperature under nitrogen. The reaction mixture was degassed (purging with nitrogen) for 20 min followed by the addition of Pd(OAc)₂ (0.091 g, 0.40 mmol) and the resulting mixture was heated at 100° C. for 16 h. Reaction mixture was cooled to room temperature, diluted with water (300 mL), and was extracted with ethyl acetate (300 mL×3). Combined organic extracts were dried over anhydrous Na₂SO₄ and concentrated under reduce pressure. Obtained crude product was purified by silica gel column chromatography, using ethyl acetate-hexane=1:9→1:4 as gradient, to afford ethyl 6-methoxy-8-(4-(trifluoromethyl)piperidin-1-yl)quinoline-3-carboxylate (X-1287A5) (1.00 g, 66%) as an off-white solid. MS: [MH]⁺369.1.

6-Methoxy-8-(4-(trifluoromethyl)piperidin-1-yl)quinoline-3-carboxylic acid (X-1287A6). To a stirred solution of methyl 6-methoxy-8-(4-(trifluoromethyl)piperidin-1-yl)quinoline-3-carboxylate (X-1287A5) (1.00 g, 2.71 nmol) in a mixture of THF-water (3:1; 30 mL) was added lithium hydroxide monohydrate (0.342 g, 8.15 mmol) at room temperature and the resulting mixture was heated at 70° C. for 1h. After cooling to room temperature, the reaction mixture was concentrated under reduced pressure. Obtained crude was diluted with water (50 mL) and was extracted with ethyl acetate (60 mL×2) to remove unwanted organic impurities. Aqueous part was acidified (pH ˜2-3) with an aqueous solution of 1N HCl and the resulting precipitate was collected by filtration. Obtained crude residue was washed with cold water until the pH of the filtrate became neutral (pH ˜6-7), and dried under high vacuum to afford 6-methoxy-8-(4-(trifluoromethyl)piperidin-1-yl)quinoline-3-carboxylic acid (X-1287A6) (0.900 g, 93%) as a white solid. MS: [MH]⁺355.02.

(R)-N-(1-hydroxypropan-2-yl)-6-methoxy-8-(4-(trifluoromethyl)piperidin-1-yl)quinoline-3-carboxamide (I-1). To a solution of 6-methoxy-8-(4-(trifluoromethyl)piperidin-1-yl)quinoline-3-carboxylic acid (X-1287A6) (0.300 g, 0.84 mmol) and (R)-2-aminopropan-1-ol (0.190 g, 2.54 mmol) in THF (5 mL) were added TEA (0.430 g, 4.23 mmol) and T₃P (0.400 g, 1.27 mmol) sequentially at room temperature under nitrogen and stirred for 1 h at the same temperature. Reaction mixture was diluted with water (20 mL) and was extracted with ethyl acetate (20 mL×3). Combined organic extracts were dried over anhydrous Na₂SO₄ and concentrated under reduced pressure. Obtained crude was purified by reverse phase (C-18) silica gel column chromatography, using acetonitrile-water=0:1→1:0 as gradient, to afford (R)-N-(1-hydroxypropan-2-yl)-6-methoxy-8-(4-(trifluoromethyl)piperidin-1-yl)quinoline-3-carboxamide (I-1) (0.130 g, 39%) as an off-white solid. ¹H NMR (400 MHz, DMSO-d₆)*δ 9.05-9.04 (d, J=2.0 Hz, 1H), 8.60-8.60 (d, J=2.0 Hz, 1H), 8.40-8.38 (d, J=8.0 Hz, 1H), 7.01-7.01 (d, J=2.4 Hz, 1H), 6.79-6.78 (d, J=2.8 Hz, 1H), 4.78-4.76 (t, J=6.0 Hz, 1H), 4.08-4.00 (m, 3H), 3.86 (s, 3H), 3.52-3.46 (m, 1H), 3.40-3.35 (m, 1H), 2.78-2.72 (t, J=11.2 Hz, 2H), 1.95-1.92 (d, J=11.2 Hz, 2H), 1.80-1.74 (m, 2H), 1.17-1.15 (d, J=6.4 Hz, 3H). MS: [MH]⁺412.02. *(one proton merged in DMSO-d₆ peak).

The following compounds were prepared in a manner analogous to the procedures described above for (R)-N-(1-hydroxypropan-2-yl)-6-methoxy-8-(4-(trifluoromethyl)piperidin-1-yl)quinoline-3-carboxamide (I-1):

(R)-8-(4-(tert-butyl)piperidin-1-yl)-N-(1-hydroxypropan-2-yl)-6-methoxyquinoline-3-carboxamide (I-2) (0.050 g, 22%) as a yellow solid. ¹H NMR (400 MHz, DMSO-d₆) δ 9.02 (s, 1H), 8.58 (s, 1H), 8.38-8.36 (d, J=8.0 Hz, 1H), 6.96 (s, 1H), 6.74-6.74 (d, J=1.6 Hz, 1H), 4.78-4.76 (t, J=5.2 Hz, 1H), 4.07-3.98 (m, 3H), 3.85 (s, 3H), 3.50-3.46 (m, 1H), 3.30-3.34 (m, 1H; merged with the peak of DMSO-d₆), 2.62-2.56 (m, 3H), 1.77-1.74 (m, 2H), 1.52-1.50 (m, 2H), 1.17-1.15 (d, J=6.8 Hz 3H), 0.90 (s, 9H). MS: [MH]⁺400.12.

(R)-N-(1-hydroxypropan-2-yl)-6-methoxy-8-(6-azaspiro[2.5]octan-6-yl)quinoline-3-carboxamide (I-3) (0.120 g, 40%) as a yellow solid. ¹H NMR (400 MHz, DMSO-d₆) δ 9.05-9.04 (d, J=2.0 Hz, 1H), 8.59-8.58 (d, J=2.4 Hz, 1H), 8.38-8.36 (d, J=8.0 Hz, 1H), 6.98-6.97 (d, J=2.4 Hz, 1H), 6.78-6.77 (d, J=2.4 Hz, 1H), 4.78-4.75 (t, J=6.0 Hz, 1H), 4.08-4.04 (m, 1H), 3.86 (s, 3H), 3.50-3.46 (m, 1H), 3.39-3.35 (m, 1H), 3.33 (4H; merged with moisture peak from DMSO-d₆), 1.57 (brs, 4H), 1.17-1.15 (d, J=6.8 Hz, 3H), 0.35 (s, 4H). MS: [MH]⁺370.22.

(R)-N-(1-hydroxypropan-2-yl)-6-methoxy-8-(8-azaspiro[4.5]decan-8-yl)quinoline-3-carboxamide (I-4) (0.100 g, 71%) as a yellow solid. ¹H NMR (400 MHz, DMSO-d₆) δ 9.02 (s, 1H), 8.57 (s, 1H), 8.38-8.36 (d, J=7.6 Hz, 1H), 6.96 (s, 1H), 6.75 (s, 1H), 4.78-4.75 (t, J=5.2 Hz, 1H), 4.08-4.04 (m, 1H), 3.85 (s, 3H), 3.50-3.46 (m, 1H), 3.27 (s, 4H), 1.64-1.61 (m, 8H), 1.48 (s, 4H), 1.28-1.23 (m, 1H), 1.17-1.15 (d, J=6.4 Hz, 3H). MS: [MH]⁺398.3.

(S)-6-methoxy-N-(1-methoxypropan-2-yl)-8-(4-(2,2,2-trifluoroethyl)piperidin-1-yl)quinoline-3-carboxamide (I-5) (0.11 g, 46%) as a yellow solid. ¹H NMR (400 MHz, DMSO-d₆) δ 9.02-9.01 (d, J=2.0 Hz, 1H), 8.58-8.58 (d, J=2.0 Hz, 1H), 8.51-8.49 (d, J=8.0 Hz, 1H), 6.98-6.98 (d, J=2.0 Hz, 1H), 6.77-6.76 (d, J=2.4 Hz, 1H), 4.28-4.21 (quin, J=6.4 Hz 1H), 3.92-3.89 (d, J=12.0 Hz, 2H), 3.86 (s, 3H), 3.46-3.42 (m, 1H), 3.34-3.30 (m, 1H); merged with moisture from DMSO-d₆), 3.28 (s, 3H), 2.75-2.66 (m, 2H), 2.38-2.28 (m, 2H), 1.86-1.83 (m, 3H), 1.65-1.60 (m, 2H), 1.18-1.16 (d, J=6.4 Hz, 3H). MS: [MH]⁺440.2.

(R)-8-(2,2-difluoro-7-azaspiro[3.5]nonan-7-yl)-N-(1-hydroxypropan-2-yl)-6-methoxyquinoline-3-carboxamide (I-6) (0.110 g, 60%) as an off white solid. ¹H NMR (400 MHz, DMSO-d₆) δ 9.04-9.03 (d, J=2.0 Hz, 1H), 8.59-8.58 (d, J=2.0 Hz, 1H), 8.39-8.37 (d, J=8.0 Hz, 1H), 6.98-6.98 (d, J=2.0 Hz, 1H), 6.76-6.75 (d, J=2.4 Hz, 1H), 4.78-4.76 (t, J=5.6 Hz, 1H), 4.09-4.03 (quin, J=6.8 Hz, 1H), 3.86 (s, 3H), 3.52-3.46 (m, 1H), 3.39-3.36 (m, 1H; merged with moisture from DMSO-d₆), 3.25 (brs, 4H), 2.49-2.41 (t, J=12.8 Hz, 4H; merged with DMSO-d₆), 1.83 (brs, 4H), 1.17-1.15 (d, J=8.0 Hz, 3H). MS: [MH]⁺420.1.

(R)-8-(4-(difluoromethyl)piperidin-1-yl)-N-(1-hydroxypropan-2-yl)-6-methoxyquinoline-3-carboxamide (I-7) (0.070 g, 35%) as a yellow solid. ¹H NMR (400 MHz, DMSO-d₆) δ 9.04-9.03 (d, J=1.2 Hz, 1H), 8.59 (s, 1H), 8.39-8.37 (d, J=8.0 Hz, 1H), 7.00-6.99 (d, J=2.0 Hz, 1H), 6.78-6.77 (d, J=2.4 Hz, 1H), 6.14-5.86 (dt, J=52.8 Hz, 4.4 Hz, 1H), 4.78-4.75 (t, J=8.0 Hz, 1H), 4.09-4.04 (quin, J=6.0 Hz, 1H), 3.99-3.96 (d, J=11.2 Hz, 2H), 3.86 (s, 3H), 3.52-3.46 (m, 1H), 3.40-3.38 (m, 1H; merged with moisture from DMSO-d₆), 2.74-2.66 (m, 2H), 2.01-1.98 (m, 1H), 1.82-1.79 (m, 2H), 1.72-1.63 (m, 2H), 1.17-1.15 (d, J=6.8 Hz, 3H), MS: [MH]+394.1.

(R)-N-(1-hydroxypropan-2-yl)-6-methoxy-8-(4-(2,2,2-trifluoroethyl)piperazin-1-yl)quinoline-3-carboxamide (I-8) (0.2 g, 69%) as a yellow solid. ¹H NMR (400 MHz, DMSO-d₆) δ 9.03-9.02 (d, J=2.4 Hz, 1H), 8.60-8.60 (d, J=2.0 Hz, 1H), 8.39-8.37 (d, J=8.0 Hz, 1H), 7.01-7.00 (d, J=2.4 Hz, 1H), 6.77-6.76 (d, J=2.4 Hz, 1H), 4.78-4.75 (t, J=6.0 Hz, 1H), 4.08-4.03 (m, 1H), 3.86 (s, 3H), 3.52-3.46 (m, 1H), 3.40-13 (m, 5H; peaks merged in moisture from DMSO-d₆), 3.30-3.23 (m, 2H), 2.87 (brs, 4H), 1.18-1.15 (d, J=6.0 Hz, 3H), MS: [MH]⁺427.1.

(S)-6-methoxy-N-(1-methoxypropan-2-yl)-8-(4-(trifluoromethyl)piperidin-1-yl)quinoline-3-carboxamide(I-9) (0.200 g, 48%) as a yellow solid. ¹H NMR (400 MHz, DMSO-d6) δ 9.04-9.03 (d, J=2.0 Hz, 1H), 8.60-8.59 (d, J=2.0 Hz, 1H), 8.53-8.51 (d, J=8.0 Hz, 1H), 7.02-7.01 (d, J=2.4 Hz, 1H), 6.79-6.78 (d, J=2.4 Hz, 1H), 4.26-4.22 (m, 1H), 4.02-4.01 (d, J=4.8 Hz, 2H), 3.87 (s, 3H), 3.46-3.42 (m, 1H), 3.36-3.33 (1H, peak merged with moisture from DMSO-d₆), 3.28 (s, 3H), 2.78-2.72 (t, J=11.6 Hz, 2H), 2.50 (1H, peak merged with DMSO-d₆), 1.95-1.92 (d, J=11.2 Hz, 2H), 1.80-1.74 (m, 2H), 1.18-1.16 (t, J=6.8 Hz, 3H). MS: [MH]⁺426.1.

N-(1-hydroxy-2-methylpropan-2-yl)-6-methoxy-8-(4-(trifluoromethyl)piperidin-1-yl)quinoline-3-carboxamide (I-10) (0.120 g, 66%) as a yellow solid. ¹H NMR (400 MHz, DMSO-d6) δ 9.00-8.99 (d, J=2.0 Hz, 1H), 8.56-8.55 (d, J=2.0 Hz, 1H), 7.85 (s, 1H), 7.01-7.00 (d, J=2.4 Hz, 1H), 6.78-6.77 (d, J=2.4 Hz, 1H), 4.90-4.87 (t, J=6.0 Hz, 1H), 4.03-4.00 (d, J=11.6 Hz, 2H), 3.86 (s, 3H), 3.56-3.54 (d, J=6.0 Hz, 2H), 3.32-3.28 (m, 1H, peak merged with moisture from DMSO-d₆), 2.78-2.72 (t, J=11.2 Hz, 2H), 1.95-1.92 (d, J=11.2 Hz, 2H), 1.80-1.74 (m, 2H), 1.34 (s, 6H). MS: [MH]⁺426.2.

(3-hydroxyazetidin-1-yl)(6-methoxy-8-(4-(trifluoromethyl)piperidin-1-yl)quinolin-3-yl)methanone (I-11) (0.120 g, 35%) as an yellow solid. ¹H NMR (400 MHz, DMSO-d₆) δ 8.84-8.83 (d, J=2.4 Hz, 1H), 8.45-8.44 (d, J=2.0 Hz, 1H), 7.10-7.09 (d, J=2.4 Hz, 1H). 6.80-6.79 (d, J=2.4 Hz, 1H), 5.84-5.82 (d, J=6.0 Hz, 1H), 4.57-4.55 (m, 2H), 4.31-4.29 (m, 1H), 4.18-4.16 (d, J=4.4 Hz, 1H). 4.00-3.97 (d, J=11.2 Hz, 2H), 3.86 (s, 3H), 3.83 (brs. 1H), 2.77-2.72 (t, J=11.6 Hz, 2H), 2.50 (2H, merged with DMSO-d₆), 1.95-1.92 (d, J=10.8 Hz, 2H), 1.79-1.73 (m, 2H). MS: [MH]⁺410.1.

(S)-6-methoxy-N-(1-(trifluoromethoxy)propan-2-yl)-8-(4-(trifluoromethyl)piperidin-1-yl)quinoline-3-carboxamide (I-12) (0.120 g, 60%) as a yellow solid. ¹H NMR (400 MHz, DMSO-d₆) δ 9.04-9.03 (d, J=2.0 Hz, 1H), 8.73-8.71 (d, J=8.0 Hz, 1H), 8.61-8.60 (d, J=2.0 Hz, 1H), 7.03-7.02 (d, J=2.0 Hz, 1H). 6.80-6.79 (d, J=2.0 Hz, 1H), 4.38-4.35 (m, 1H), 4.15-4.13 (d, J=5.6 Hz, 2H), 4.03-4.02 (d, J=12.0 Hz, 2H), 3.87 (s, 3H), 2.79-2.73 (t, J=11.2 Hz, 2H), 1.96-1.93 (d, J=11.2 Hz, 2H), 1.80-1.72 (m, 2H), 1.26-1.24 (d, J=6.8 Hz, 3H) (1H could be merged with DMSO-d₆). MS: [MH]⁺ 480.1.

(R)-N-(1-hydroxybutan-2-yl)-6-methoxy-8-(4-(trifluoromethyl)piperidin-1-yl)quinoline-3-carboxamide (I-13) (0.080 g, 44%) as an off-white solid. ¹H NMR (400 MHz, DMSO-d₆) δ 9.06-9.05 (d, J=2.0 Hz, 1H), 8.62-8.61 (d, J=1.6 Hz, 1H), 8.31-8.29 (d, J=8.4 Hz, 1H), 7.02-7.01 (d, J=2.0 Hz, 1H), 6.79-6.78 (d, J=2.0 Hz, 1H), 4.73-4.70 (t, J=5.6 Hz. 1H), 4.03-4.00 (d, J=11.2 Hz, 2H), 3.92-3.91 (m, 1H), 3.87 (s. 3H), 3.51-3.41 (m. 2H), 2.79-2.73 (t, =11.2 Hz, 2H), 1.96-1.93 (d, J=11.2 Hz, 2H), 1.80-1.67 (m, 3H), 1.49-1.46 (m, 1H), 0.92-0.89 (t, J=7.6 Hz, 3H) (1H could be merged with DMSO-d₆). MS: [MH]⁺ 426.1.

(S)-N-(1-hydroxybutan-2-yl)-6-methoxy-8-(4-(trifluoromethyl)piperidin-1-yl)quinoline-3-carboxamide (I-14) (0.100 g, 55%) as an off-white solid. ¹H NMR (400 MHz, DMSO-d₆) δ 9.06-9.05 (d, J=2.0 Hz, 1H), 8.62-8.61 (d, J=2.0 Hz, 1H), 8.31-8.29 (d, J=8.4 Hz, 1H), 7.02-7.01 (d, J=2.0 Hz, 1H), 6.79-6.78 (d, J=2.0 Hz, 1H), 4.73-4.70 (t, J=5.6 Hz, 1H), 4.03-4.00 (d, J=11.2 Hz, 2H), 3.92-3.91 (m, 1H), 3.87 (s, 3H), 3.51-3.36 (i, 2H), 2.79-2.72 (t, J=11.6 Hz, 2H), 1.96-1.93 (d, J=11.6 Hz, 2H), 1.81-1.66 (m, 3H), 1.51-1.44 (m, 1H), 0.92-0.89 (t, J=7.6 Hz, 3H) (1H could be merged with DMSO-d₆). MS: [MH]⁺426.1.

### Example 1.2. Synthesis of (R)-N-(1-hydroxypropan-2-yl)-6-methoxy-8-(4-(trifluoromethoxy)phenyl)quinoline-3-carboxamide (I-15)

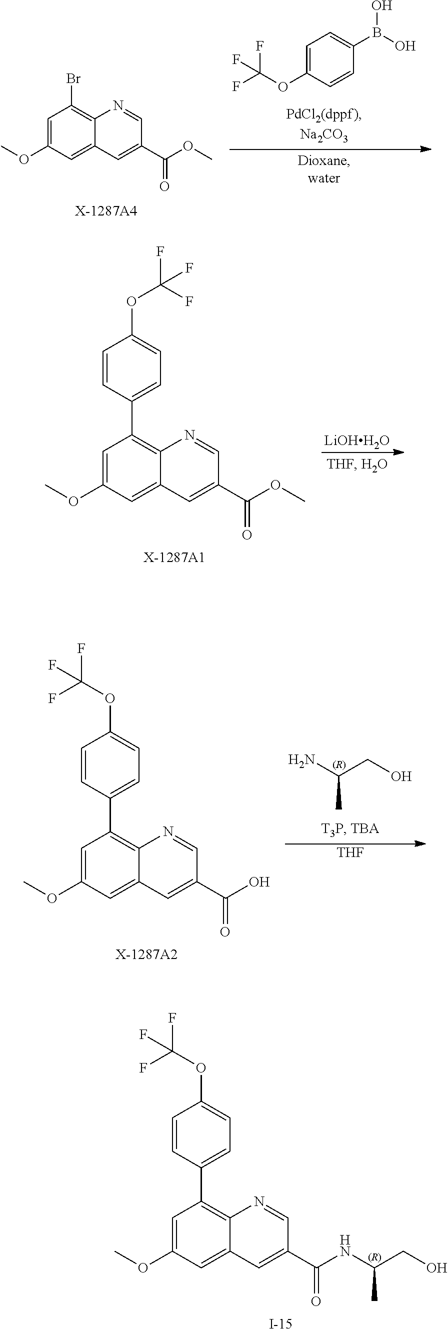

Methyl 6-methoxy-8-(4-(trifluoromethoxy)phenyl)quinoline-3-carboxylate (X-1289A1). To a stirred solution of methyl 8-bromo-6-methoxyquinoline-3-carboxylate (X-1287A4) (0.300 g, 1.01 mmol) in a mixture of 1,4-dioxane-water (3:1, 5 mL) were added (4-(trifluoromethoxy)phenyl)boronic acid (0.251 g, 1.22 mmol) and Na₂CO₃ (0.215 g, 2.03 mmol) sequentially at room temperature under nitrogen. The reaction mixture was degassed (purging with nitrogen) for 20 min followed by the addition of PdCl₂(dppf) (0.037 g, 0.05 mmol) and the resulting mixture was heated at 100° C. for 2 h. Reaction mixture was cooled to room temperature, diluted with water (30 mL) and was extracted with ethyl acetate (30 mL×3). Combined organic extracts were dried over anhydrous Na₂SO₄ and concentrated under reduce pressure to afford crude mass, which was purified by silica gel CombiFlash column chromatography, using ethyl acetate-hexane=1:19→1:3 as eluent, to afford methyl 6-methoxy-8-(4-(trifluoromethoxy)phenyl)quinoline-3-carboxylate (X-1289A1) (0.295 g, 77%) as an off-white solid. MS: [MH]⁺378.0.

6-Methoxy-8-(4-(trifluoromethoxy)phenyl)quinoline-3-carboxylic acid (X-1289A2). To a stirred solution of methyl 6-methoxy-8-(4-(trifluoromethoxy)phenyl)quinoline-3-carboxylate (X-1289A1) (0.295 g. 0.78 mmol) in a mixture of THF-water (2:1; 4.0 mL) was added lithium hydroxide monohydrate (0.082 g, 1.95 mmol) at room temperature and the resulting mixture was heated at 70° C. for 1 h. After cooling to room temperature, the reaction mixture was concentrated under reduced pressure, obtained crude was diluted with water (10 mL), and was extracted with ethyl acetate (10 mL×2) to remove unwanted organic impurities. Aqueous part was acidified (pH ˜2-3) with an aqueous solution of 1N HCl and the resulting precipitate was collected by filtration. Crude residue was washed with cold water until the pH of the filtrate became neutral (pH ˜6-7). Obtained solid was dried under high vacuum to afford to afford 6-methoxy-8-(4-(trifluoromethoxy)phenyl)quinoline-3-carboxylic acid (X-1289A2) (0.250 g, 88%) as a white solid, which was pure enough to proceed to the next step. MS: [MH]⁺363.97.

(R)-N-(1-hydroxypropan-2-yl)-6-methoxy-8-(4-(trifluoromethoxy)phenyl)quinoline-3-carboxamide (I-15). To a stirred solution of 6-methoxy-8-(4-(trifluoromethoxy)phenyl)quinoline-3-carboxylic acid (X-1289A2) (0.250 g, 0.68 mmol) and (R)-2-aminopropan-1-ol (0.103 g, 1.37 mmol) in THF (3 mL) were added TEA (0.347 g, 3.44 mmol) and T₃P (0.328 g, 1.03 mmol) sequentially at room temperature under nitrogen and stirred for 1h at the same temperature. The resulting reaction mixture was diluted with water (20 mL) and was extracted with ethyl acetate (30 mL×3). Collected organic extracts were dried over anhydrous Na₂SO₄ and concentrated under reduced pressure. The resulting crude was purified by reverse phase (C-18) silica gel column chromatography, using acetonitrile-water=0:1→1:0 as gradient, to afford (R)-N-(1-hydroxypropan-2-yl)-6-methoxy-8-(4-(trifluoromethoxy)phenyl)quinoline-3-carboxamide (I-15) (0.130 g, 45%) as an off-white solid. ¹H NMR (400 MHz, DMSO-d₆) δ 9.08-9.07 (d, J=1.2 Hz, 1H), 8.76-8.76 (d, J=1.2 Hz, 1H), 8.47-8.45 (d, J=8.0 Hz, 1H), 7.80-7.78 (d, J=8.4 Hz, 2H), 7.52 (s, 2H), 7.48-7.46 (d, J=8.0 Hz, 2H), 4.10-4.04 (quin, J=6.8 Hz, 1H), 3.95 (s, 3H), 3.52-3.48 (m, 1H), 3.41-3.36 (m, 1H), 1.18-1.16 (d, J=6.8 Hz, 3H). MS: [MH]⁺421.0.

The following compounds were prepared in a manner analogous to the procedures described above for (R)-N-(1-hydroxypropan-2-yl)-6-methoxy-8-(4-(trifluoromethoxy)phenyl)quinoline-3-carboxamide (I-15):

(R)-8-(4-(tert-butoxy)phenyl)-N-(1-hydroxypropan-2-yl)-6-methoxyquinoline-3-carboxamide (I-16) (0.150 g, 43%) as yellow solid. ¹H NMR (400 MHz, DMSO-d₆) δ 9.08 (s, 1H), 8.73 (s, 1H), 8.45-8.43 (d, J=6.8 Hz, 1H), 7.62-7.60 (d, J=7.6 Hz, 2H), 7.45 (s, 2H), 7.08-7.06 (d, J=7.2 Hz, 2H), 4.79 (s, 1H), 4.07 (brs, 1H), 3.94 (s, 3H), 3.49 (brs, 1H), 3.34 (1H, merged with moisture from DMSO-d₆), 1.37 (s, 9H), 1.18-1.16 (d, J=6 Hz, 3H). MS: [MH]⁺409.1.

(R)-8-(4-(difluoromethoxy)phenyl)-N-(1-hydroxypropan-2-yl)-6-methoxyquinoline-3-carboxamide (I-17) (0.050 g, 21%) as a white solid. ¹H NMR (400 MHz, DMSO-d₆) δ 9.07 (brs, 1H), 8.74 (brs, 1H), 8.46 (brs, 1H), 7.72 (brs, 2H), 7.49 (brs, 2H), 7.33-7.29 (m, 2H), 4.79-3.50 (t, J=228 Hz, 1H), 3.95 (s, 3H), 1.17 (s, 3H). MS: [MH]⁺403.1.

(R)-N-(1-hydroxypropan-2-yl)-6-methoxy-8-(4-(2,2,2-trifluoroethoxy)phenyl)quinoline-3-carboxamide (I-18) (0.025 g, 16%) as an off-white solid. ¹H NMR (400 MHz, DMSO-d₆) δ 9.07-9.06 (d, J=2.0 Hz, 1H), 8.73-8.73 (d, J=1.6 Hz, 1H), 8.46-8.44 (d, J=8.0 Hz, 1H), 7.67-7.65 (d, J=8.8 Hz, 2H), 7.46-7.44 (dd, J=7.2 Hz, 2.8 Hz, 2H), 7.17-7.15 (d, J=8.8 Hz, 2H), 4.88-4.78 (m, 3H), 4.09-4.05 (m, 1H), 3.94 (s, 3H), 3.51-3.47 (m, 1H), 3.41-3.34 (m, 1H; peak merged with moisture from DMSO-d₆), 1.18-1.16 (d, J=6.4 Hz, 3H). MS: [MH]⁺435.2.

(R)-N-(1-hydroxypropan-2-yl)-6-methoxy-8-(4-phenoxyphenyl)quinoline-3-carboxamide (I-19) (0.037 g, 8%) as an off-white solid. ¹H NMR (400 MHz, DMSO-d₆) δ 9.09-9.08 (d, J=2.4 Hz, 1H), 8.74-8.74 (d, J=2.0 Hz, 1H), 8.46-8.44 (d, J=8.0 Hz, 1H), 7.71-7.69 (d, J=8.4 Hz, 2H), 7.47 (s, 1H), 7.46-7.42 (t, J=8.0 Hz, 2H), 7.20-7.17 (t, J=7.6 Hz, 1H), 7.13-7.08 (m, 4H), 4.80-4.77 (t, J=6.0 Hz, 1H), 4.09-4.06 (m, 1H), 3.95 (s, 3H), 3.51-3.49 (m, 1H), 3.39-3.37 (m, 1H), 1.18-1.16 (d, J=6.8 Hz, 3H). MS: [MH]⁺429.2.

(R)-8-(4-cyclopropoxyphenyl)-N-(1-hydroxypropan-2-yl)-6-methoxyquinoline-3-carboxamide (I-20) (0.120 g, 51%) as an off white solid. ¹H NMR (400 MHz, DMSO-d₆) δ 9.07-9.06 (d, J=2.0 Hz, 1H), 8.73-8.72 (d, J=2.4 Hz, 1H), 8.45-8.43 (d, J=8.0 Hz, 1H), 7.63-7.61 (d, J=8.8 Hz, 2H), 7.44-7.42 (d, J=2.0 Hz, 2H), 7.16-7.14 (d, J=8.8 Hz, 2H), 4.79-4.76 (t, J=6.0 Hz, 1H), 4.11-4.01 (m, 1H), 3.94 (s, 3H), 3.92-3.88 (m, 1H), 3.53-3.47 (m, 1H), 3.41-3.35 (m, 1H), 1.18-1.16 (d, J=6.4 Hz, 3H) 0.84-0.80 (m, 2H), 0.72-0.68 (m, 2H). MS: [MH]⁺393.2.

(R)-8-(2-fluoro-4-(trifluoromethoxy)phenyl)-N-(1-hydroxypropan-2-yl)-6-methoxyquinoline-3-carboxamide (I-21) (0.130 g, 56%) as a white solid. ¹H NMR (400 MHz, DMSO-d₆) δ 9.02-9.02 (d, J=2.4 Hz, 1H), 8.75-8.75 (d, J=2.0 Hz, 1H), 8.45-8.43 (d, J=8.0 Hz, 1H), 7.67-7.63 (t, J=8.4 Hz, 1H), 7.58-7.57 (d, J=2.4 Hz, 1H), 7.53-7.49 (m, 2H), 7.37-7.35 (d, J=8.4 Hz, 1H), 4.78-4.75 (t, J=5.6 Hz, 1H), 4.10-4.01 (m, 1H), 3.98 (s, 3H), 3.52-3.47 (m, 1H), 3.41-3.35 (m, 1H), 1.17-1.16 (d, J=6.8 Hz, 3H). MS: [MH]⁺438.9.

### Example 1.3. Synthesis of 5-((4,4-Difluorocyclohexyl)methoxy)-N-(1-hydroxypropan-2-yl)-2-naphthamide (I-22)

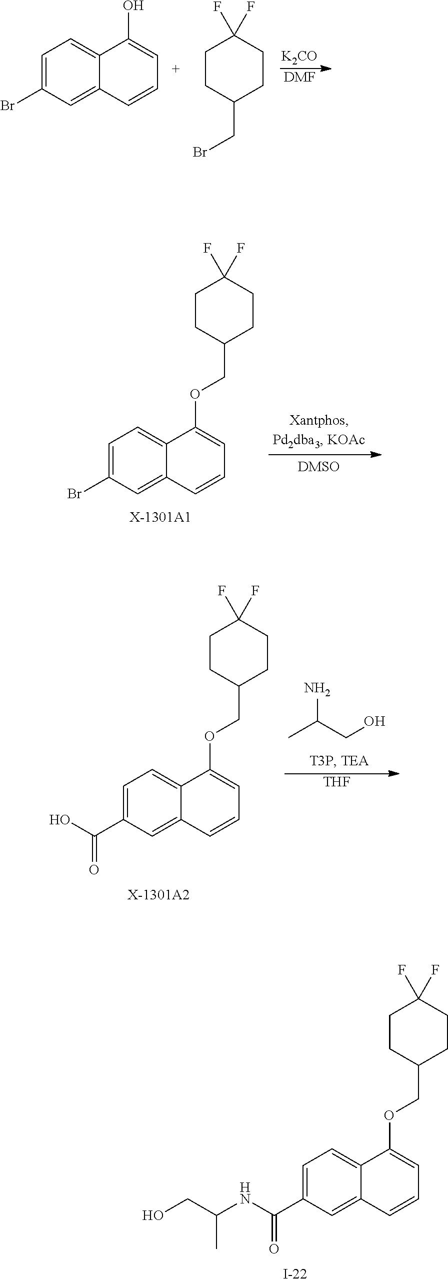

6-Bromo-1-((4,4-difluorocyclohexyl)methoxy)naphthalene (X-1301A1). To a stirred solution of 6-bromonaphthalen-1-ol (1.00 g, 4.50 mmol) in DMF (18 mL) were added potassium carbonate (2.48 g, 18.00 mmol) and 4-(bromomethyl)-1,1-difluorocyclohexane (1.11 g, 5.40 mmol) sequentially at room temperature under nitrogen, and the resulting mixture was heated at 120° C. for 16 h. Reaction mixture was cooled to room temperature, quenched with water (200 mL), and extracted with ethyl acetate (75 mL×3). Collected organics were washed with brine (150 mL), dried over anhydrous Na₂SO₄, and concentrated in vacuo. The resulting crude was purified by silica gel column chromatography, using ethyl acetate-hexane=0:1→1:4 as gradient, to afford 6-bromo-1-((4,4-difluorocyclohexyl)methoxy)naphthalene (X-1301A1) (1.40 g, 64%) as an off-white solid. ¹H NMR (400 MHz, DMSO-d₆) 8.15-8.15 (d, J=1.6 Hz, 1H), 8.09-8.07 (d, J=8.8 Hz, 1H), 7.63-7.60 (dd, J=1.6, 8.8 Hz, 1H), 7.46-7.45 (m, 2H), 7.01-6.99 (m, 1H), 4.05-4.04 (d, J=6.0 Hz, 1H), 2.06-1.84 (m, 7H), 1.47-1.41 (m, 2H).

5-((4,4-Difluorocyclohexyl)methoxy)-2-naphthoic acid (X-1301A2). To a stirred solution of 6-bromo-1-((4,4-difluorocyclohexyl)methoxy)naphthalene (X-1301A1) (0.500 g, 1.41 mmol) in DMSO (12 mL) was added potassium acetate (0.415 g, 4.23 mmol) at room temperature under nitrogen. The reaction mixture was degassed (purging with nitrogen) for 20 min followed by the addition of Xanthphos (0.082 g, 0.141 mmol) and Pd₂dba₃ (0.130 g, 0.141 mmol) sequentially, and the resulting mixture was heated at 120° C. for 16 h under CO at 50 psi in a Parr autoclave. Reaction mixture was cooled to room temperature, diluted with water (100 mL) and was extracted with ethyl acetate (75 mL×2). Combined organic extracts were dried over anhydrous Na₂SO₄ and concentrated under reduce pressure. Obtained crude was purified by reverse phase (C-18) silica gel column chromatography, using acetonitrile-water=0:1→1:0 as gradient, to afford 5-((4,4-Difluorocyclohexyl)methoxy)-2-naphthoic acid (X-1301A2) (0.33 g, 36%) as an off-white solid. ¹H NMR (400 MHz, DMSO-d₆) 13.10 (br, 1H), 8.53 (s, 1H), 8.24-8.21 (d, J=8.8 Hz, 1H), 7.98-7.95 (d, J=8.8 Hz, 1H), 7.66-7.64 (d, J=8.4 Hz, 1H), 7.51-7.47 (t, J=8.0 Hz, 1H), 7.11-7.09 (d, J=8.0 Hz, 1H), 4.07-4.04 (d, J=6 Hz, 1H), 2.08-1.82 (m, 7H), 1.47-1.39 (m, 2H).

5-((4,4-Difluorocyclohexyl)methoxy)-N-(1-hydroxypropan-2-yl)-2-naphthamide (I-22). To a stirred solution of 5-((4,4-difluorocyclohexyl)methoxy)-2-naphthoic acid (X-1301A2) (0.157 g, 0.49 mmol) in THF (8 mL) were added 2-aminopropan-1-ol (0.073 g, 0.98 mmol), triethylamine (0.148 g, 1.47 mmol), and propylphosphonic anhydride (T3P) (0.468 g, 0.73 mmol) at 0° C. under nitrogen, and the resulting mixture was stirred at room temperature for 1 h. Reaction mixture poured into ice-water (50 mL) and was extracted with ethyl acetate (20 mL×3). Combined organic extracts were washed with brine (30 mL), dried over anhydrous Na₂SO₄, and concentrated under reduced pressure. The resulting crude was purified by reverse phase (C-18) silica gel column chromatography using acetonitrile-water-0:1→1:0 as gradient, to afford 5-((4,4-difluorocyclohexyl)methoxy)-N-(1-hydroxypropan-2-yl)-2-naphthamide (I-22) (0.114 g, 62%) as a white solid. ¹H NMR (400 MHz, DMSO-d6) δ 8.39 (s, 1H), 8.28-8.26 (d, J=8.0 Hz, 1H), 8.19-8.17 (d, J=8.8 Hz, 1H), 7.92-7.89 (dd, J=1.2, 8.8 Hz, 1H), 7.56-7.54 (d, J=8.4 Hz, 1H), 7.49-7.45 (t, J=7.6 Hz, 1H), 7.05-7.4 (d, J=7.6 Hz, 1H), 4.78-4.75 (t, J=7.6 Hz, 1H), 4.07-4.05 (m, 3H), 3.51-3.47 (m, 1H), 3.39-3.36 (m, 1H), 2.07-1.86 (m, 7H), 1.45-1.42 (m, 2H), 1.17-1.15 (d, J=6.8 Hz, 3H). MS: [MH]⁺378.1.

### Example 1.4. Synthesis of (S)-N-(1-methoxypropan-2-yl)-6-(trifluoromethoxy)-8-(4-(trifluoromethyl)piperidin-1-yl)quinoline-3-carboxamide (I-23)

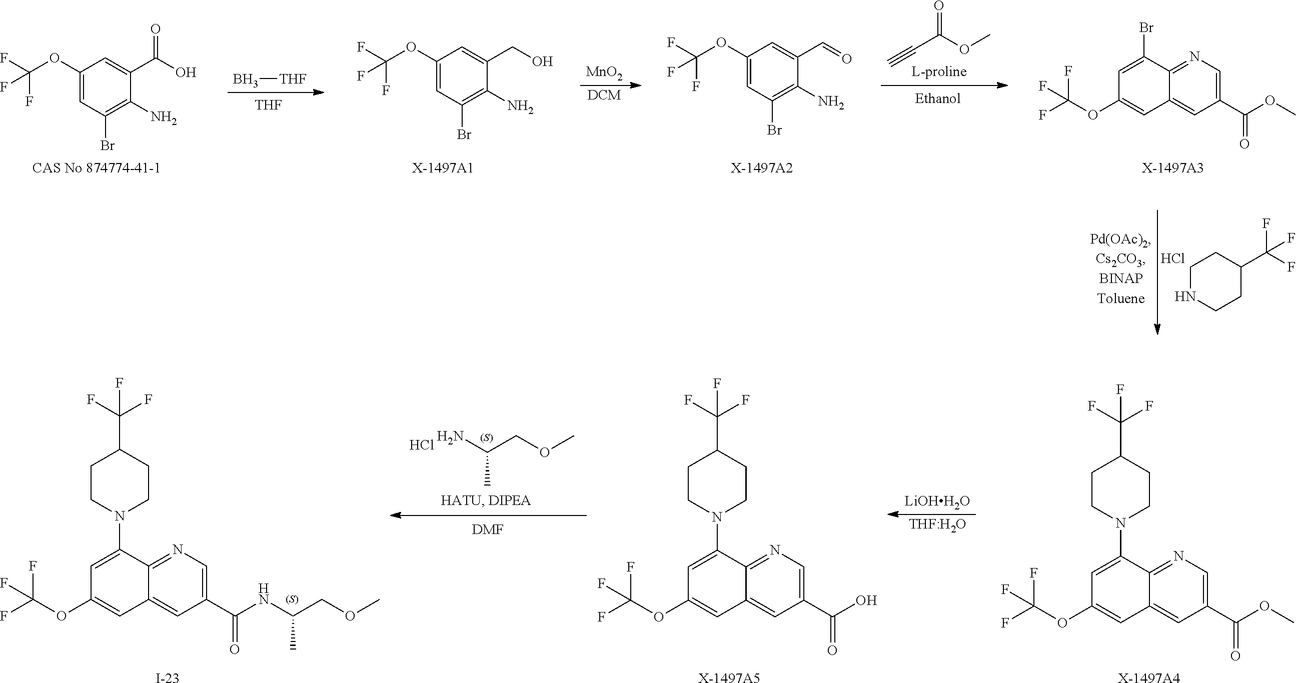

(2-Amino-3-bromo-5-(trifluoromethoxy)phenyl)methanol (X-1497A1). To a stirred solution of 2-amino-3-bromo-5-(trifluoromethoxy)benzoic acid (1.00 g, 3.33 mmol) in THF (10 mL) was added BH₃.THF (13.3 mL, 13.3 mmol) slowly at 0° C. under nitrogen. After 15 min of stirring at the same temperature, reaction temperature was brought to 70° C. and stirred for 16 h at the same temperature. After cooling to room temperature, reaction mixture was quenched with MeOH (20 mL), and volatiles were distilled off under reduced pressure. Residue was taken in ethyl acetate (3000 mL), washed with water (1000 mL×3), dried over anhydrous Na₂SO₄ and concentrated under reduced pressure to afford (2-amino-3-bromo-5-(trifluoromethoxy) phenyl) methanol (X-1497A1) (0.94 g, 98%) as an off-white solid, which was used in next step without further purification. MS: [MH]⁺ 286.1

2-Amino-3-bromo-5-(trifluoromethoxy)benzaldehyde (X-1497A2). To a stirred solution of (2-amino-3-bromo-5-(trifluoromethoxy) phenyl) methanol (X-1497A1) (0.940 g, 3.29 mmol) in DCM (15 mL) was added MnO₂ (2.80 g, 32.9 mmol) at 0° C., and the resulting mixture was stirred at room temperature for 16 h. Reaction mixture was filtered through a celite bed and filtrate was concentrated under reduced pressure to afford 2-amino-3-bromo-5-(trifluoromethoxy)benzaldehyde (X-1497A2) (0.880 g, 94%) as off-white solid, which was used in next step without further purification. ¹H NMR (400 MHz, DMSO-d₆) δ 9.84 (s, 1H), 7.86 (s, 1H), 7.79 (s, 1H), 7.30 (s, 1H).

Methyl 8-bromo-6-(trifluoromethoxy) quinoline-3-carboxylate (X-1497A3). To a stirred solution of 2-amino-3-bromo-5-(trifluoromethoxy)benzaldehyde (X-1497A2) (0.870 g, 3.20 mmol) in ethanol (10 mL) were added methyl propiolate (0.30 g, 3.60 mmol) and L-proline (0.17 g, 1.53 mmol) at room temperature, and the resulting mixture was heated at 80° C. for 16 h. After cooling to room temperature, the reaction mixture was slowly poured into n-hexane (500 mL), and the resulting precipitate was collected by filtration. Obtained solid residue was washed with n-hexane (500 mL) and dried under high vacuum to afford methyl 8-bromo-6-(trifluoromethoxy) quinoline-3-carboxylate (X-1497A3) (0.930 g, 86%) as a yellow solid, which was pure enough to proceed to the next step without further purification. MS: [MH]⁺349.70.

Methyl 6-(trifluoromethoxy)-8-(4-(trifluoromethyl)piperidin-1-yl)quinoline-3-carboxylate (X-1497A4). To a stirred solution of methyl 8-bromo-6-(trifluoromethoxy) quinoline-3-carboxylate (X-1497A3) (0.400 g, 1.14 mmol) in toluene (10 mL) were added 4-(trifluoromethyl)piperidine hydrochloride (0.640 g, 3.43 mmol), cesium carbonate (2.20 g, 6.84 mmol), and rac-BINAP (0.141 g, 0.22 mmol) sequentially at room temperature under nitrogen. The reaction mixture was degassed (purging with nitrogen) for 20 min followed by addition of Pd(OAc)₂ (0.025 g, 0.11 mmol) and was stirred at 100° C. for 3 h. Reaction mixture was cooled to room temperature, diluted with water (20 mL), and was extracted with ethyl acetate (60 mL×3). Combined organic extracts were dried over anhydrous Na₂SO₄ and concentrated under reduce pressure. Obtained crude was purified by silica gel column chromatography using ethyl acetate-hexane=3:7 as gradient to afford methyl 6-(trifluoromethoxy)-8-(4-(trifluoromethyl)piperidin-1-yl)quinoline-3-carboxylate (X-1497A4) (0.390 g, 81%) as a yellow solid. MS: [MH]⁺422.92.

6-(Trifluoromethoxy)-8-(4-(trifluoromethyl)piperidin-1-yl)quinoline-3-carboxylic acid (X-1497A5). To a stirred solution of methyl 6-(trifluoromethoxy)-8-(4-(trifluoromethyl)piperidin-1-yl)quinoline-3-carboxylate (X-1497A4) (0.390 g, 0.92 mmol) in a mixture of THF-water (3:1; 4 mL) was added lithium hydroxide monohydrate (0.071 g, 1.84 mmol) at room temperature and the resulting mixture was stirred for 1 h at the same temperature. Reaction mixture was concentrated under reduced pressure, and obtained crude was diluted with water (5 mL) and was extracted with ethyl acetate (30 mL×2) to remove unwanted organic impurities. Aqueous part was acidified (pH ˜2-3) with an aqueous solution of 1N HCl and was extracted with ethyl acetate (60 mL×2). Combined organic extracts were dried over anhydrous Na₂SO₄ and concentrated under reduce pressure to afford 6-(trifluoromethoxy)-8-(4-(trifluoromethyl)piperidin-1-yl)quinoline-3-carboxylic acid (X-1497A5) (0.350 g, 94%) as a white solid. MS: [MH]⁺409.09.

(S)-N-(1-methoxypropan-2-yl)-6-(trifluoromethoxy)-8-(4-(trifluoromethyl)piperidin-1-yl)quinoline-3-carboxamide (I-23). To a stirred solution of 6-(trifluoromethoxy)-8-(4-(trifluoromethyl)piperidin-1-yl)quinoline-3-carboxylic acid (X-1497A5) (0.340 g, 0.83 mmol) in DMF (8 mL) were added (S)-1-methoxypropan-2-amine hydrochloride (0.310 g, 2.49 mmol), DIPEA (0.71 mL, 4.16 mmol), and HATU (0.474 g, 12.4 mmol) at room temperature and stirred for 2 h at the same temperature. Reaction mixture was diluted with water (10 mL) and was extracted with ethyl acetate (40 mL×2). Organic extracts were combined, dried over anhydrous Na₂SO₄, and concentrated under reduced pressure. Obtained crude was purified by reverse phase (C-18) silica gel column chromatography, using acetonitrile-water=0:1→1:0 as gradient, to afford (S)-N-(1-methoxypropan-2-yl)-6-(trifluoromethoxy)-8-(4-(trifluoromethyl)piperidin-1-yl)quinoline-3-carboxamide (I-23) (0.200 g, 50%) as a yellow solid. ¹H NMR (400 MHz, DMSO-d₆) δ 922-9.22 (d, J=1.6 Hz, 1H), 8.80-8.79 (d, J=2.0 Hz, 1H), 8.61-8.59 (d, J=8.0 Hz, 1H), 7.61 (s. 1H), 7.07 (s. 1H), 4.26-4.23 (m, 1H), 4.11-4.08 (d, J=11.2 Hz, 2H), 3.46-3.42 (m, 1H), 3.28 (s, 3H), 2.88-2.82 (1, J=12.4 Hz, 2H), 1.97-1.94 (d, J=11.2 Hz, 2H), 1.77-1.75 (m, 2H), 1.18-1.16 (d, J=6.80 Hz, 3H). (2H merged with DMSO-d₆ residual and moisture peak) MS: [MH]⁺ 480.1. ¹H NMR (400 MHz, CH₃OH-d₄) δ 9.24-9.23 (d, J=4.0 Hz, 1H), 8.70 (s, 1H), 7.52 (s, 1H), 7.15 (s, 1H), 4.42-4.37 (m, 1H), 4.05-4.02 (d, J=1.2 Hz, 2H), 3.57-3.46 (m, 2H), 3.41 (s, 3H), 2.88-2.82 (m, 2H), 2.43-2.41 (m, 1H), 2.04-2.00 (m, 4H), 1.31-1.29 (d, J=8.0 Hz, 3H). (amide proton got exchanged) MS: [MH]⁺ 480.16.

The following compound was prepared in a manner analogous to the procedures described above for (S)-N-(1-methoxypropan-2-yl)-6-(trifluoromethoxy)-8-(4-(trifluoromethyl)piperidin-1-yl)quinoline-3-carboxamide (I-23):

(R)-N-(1-hydroxypropan-2-yl)-8-(6-azaspiro[2.5]octan-6-yl)-6-(trifluoromethoxy)quinoline-3-carboxamide (I-24) (0.130 g, 45%) as a yellow solid. ¹H NMR (400 MHz, DMSO-d₆) δ 9.23 (s, 1H), 8.79 (s, 1H), 8.47-8.45 (d, J=8.0 Hz, 1H), 7.56 (s, 1H), 7.05 (s, 1H), 4.80-4.77 (t, J=8.0 Hz, 1H), 4.09-4.05 (m, 1H), 3.52-3.48 (m, 1H), 3.47-3.37 (m, 4H), 1.59 (br. s, 4H), 1.17-1.16 (d, J=4.0 Hz, 3H), 0.37 (s, 4H). MS: [MH]⁺424.1.

### Example 1.5. Synthesis of (R)-6-cyclopropoxy-N-(1-hydroxypropan-2-yl)-8-(6-azaspiro[2.5]octan-6-yl)quinoline-3-carboxamide (I-25)

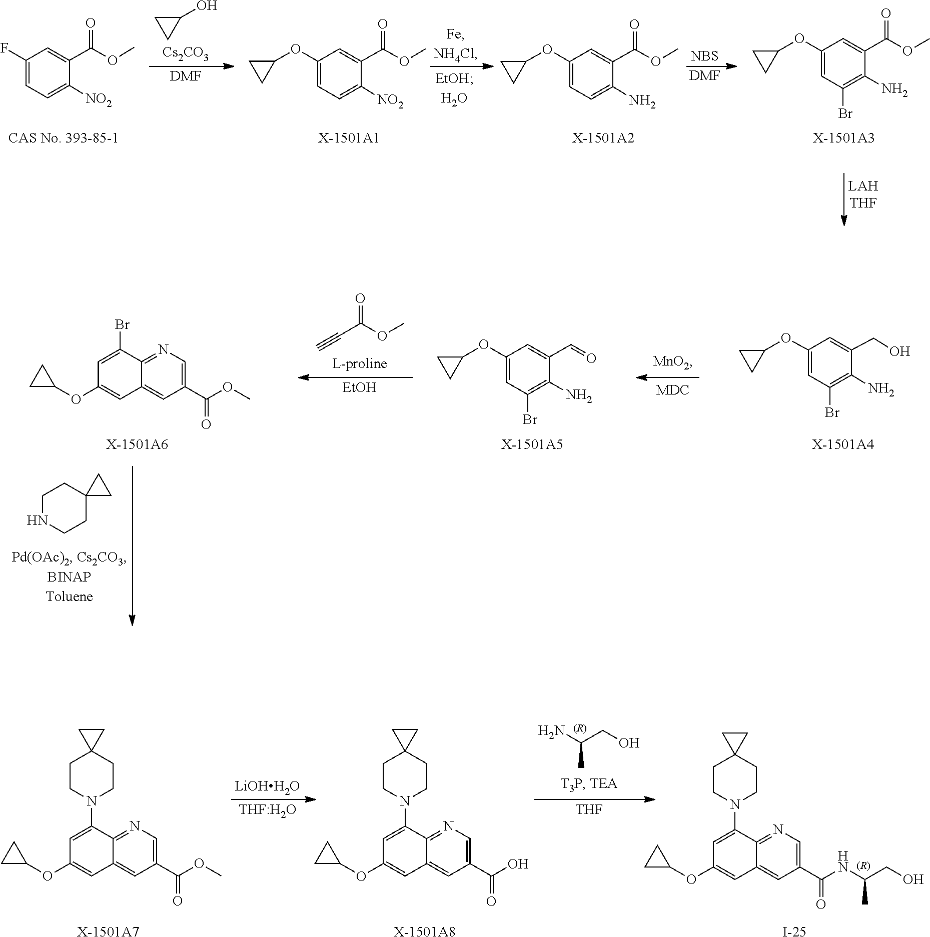

Methyl 5-cyclopropoxy-2-nitrobenzoate (X-1501A1). To a stirred solution of cyclopropanol (4.44 g, 76.55 mmol) in DMF (20 mL) were added cesium carbonate (16.86 g, 229.60 mmol) and methyl 5-fluoro-2-nitrobenzoate (15.23 g, 76.55 mmol) sequentially at room temperature under nitrogen, and the resulting mixture was stirred at 100° C. for 6 h. Reaction mixture was cooled to room temperature, diluted with water (300 mL), and extracted with DCM (200 mL×3). Combined organic extracts were dried over anhydrous Na₂SO₄ and concentrated under reduce pressure. The crude product was purified by silica gel column chromatography using ethyl acetate-hexane=0:10→1:9 as gradient, to afford methyl 5-cyclopropoxy-2-nitrobenzoate (X-1501A1) (4.5 g, 25%) as a colorless oil. ¹H NMR (400 MHz, DMSO-d₆) δ 8.16-8.13 (dd, J=7.2 Hz, 2.0 Hz, 1H), 7.38-7.35 (m, 2H), 4.10-4.05 (m, 1H), 0.88-0.84 (m, 2H), 0.79-0.71 (m, 2H).

Methyl 2-amino-5-cyclopropoxybenzoate (X-1501A2). To a stirred solution of 5-cyclopropoxy-2-nitrobenzoate (X-1501A1) (4.5 g, 18.98 mmol) in a mixture of EtOH-water (5:1, 60 mL) were added Fe powder (5.31 g, 94.68 mmol) and ammonium chloride (5.03 g, 94.68 mmol) at room temperature under nitrogen and the resulting mixture was stirred 70° C. for 16 h. Reaction mixture was filtered through a celite bed and filtrate was concentrated under reduced pressure to afford methyl 2-amino-5-cyclopropoxybenzoate (X-1501A2) (4.0 g, Quantitative (crude)) as a yellow solid, which was used in next step without further purification. MS: [MH]⁺ 208.0.

Methyl 2-amino-3-bromo-5-cyclopropoxybenzoate (X-1501A3). To a stirred solution of methyl 2-amino-5-cyclopropoxybenzoate (X-1501A2) (4.0 g (crude), 19.32 mmol) in DCM (15 mL) was added NBS (3.43 g, 19.32 mmol) at 0° C. under nitrogen, and the resulting mixture was stirred at same temperature for 40 min. Reaction mixture was quenched with an aqueous solution of saturated NaHCO₃ (50 mL) and was extracted with DCM (100 mL×3). Combined organic extracts were dried over anhydrous Na₂SO₄ and concentrated under reduced pressure. The crude product was purified by silica gel column chromatography using ethyl acetate-hexane=0:10→1:9 as gradient, to afford methyl 2-amino-3-bromo-5-cyclopropoxybenzoate (X-1501A3) (2.76 g, 73%) as a purple solid. MS: [MH]⁺285.8/[MH+2]⁺287.8.

(2-Amino-3-bromo-5-cyclopropoxyphenyl)methanol (X-1501A4). To a stirred solution of methyl 2-amino-3-bromo-5-cyclopropoxybenzoate (X-1501A3) (2.71 g, 9.50 mmol) in THF (15 mL) was added LiAlH₄ (2.0 M in THF, 5 mL, 10.0 mmol) at 0° C. under nitrogen, and the resulting mixture was stirred for 30 min at the same temperature. Reaction mixture was quenched with an aqueous solution of saturated NH₄Cl (100 mL) and was extracted with DCM (100 mL×3). Combined organic extracts were dried over anhydrous Na₂SO₄ and concentrated under reduced pressure to afford (2-amino-3-bromo-5-cyclopropoxyphenyl)methanol (X-1501A4) (2.35 g, 96% (crude)) as a brown solid, which was used in next step without further purification. MS: [MH]⁺257.8/[MH+2]⁺259.9.

2-Amino-3-bromo-5-cyclopropoxybenzaldehyde (X-1501A5). To a stirred solution of (2-amino-3-bromo-5-cyclopropoxyphenyl)methanol (X-1501A4) (2.30 g; crude, 8.94 mmol) in DCM (150 mL) was added MnO₂ (7.78 g, 89.4 mmol) at 0° C. under nitrogen, and the resulting mixture was stirred at room temperature for 1.2 h. Reaction mixture was filtered through a celite bed and filtrate was concentrated under reduced pressure to afford 2-amino-3-bromo-5-cyclopropoxybenzaldehyde (X-1501A5) (2.1 g, 94% (crude)) as a yellow solid, which was used in next step without further purification. MS: [MH]⁺255.9/[MH+2]⁺257.8.

Methyl 8-bromo-6-cyclopropoxyquinoline-3-carboxylate (X-1501A6). To a stirred solution of 2-amino-3-bromo-5-cyclopropoxybenzaldehyde (X-1501A5) (2.1 g; crude, 8.23 mmol) in ethanol (5 mL) were added methyl propiolate (1.03 g, 12.35 mmol) and L-proline (0.47 g, 4.11 mmol) sequentially at room temperature, and the resulting mixture was heated at 80° C. for 16 h. Reaction mixture was concentrated under reduced pressure, crude was diluted with water (30 mL), and the resulting precipitate was collected by filtration. Obtained solid residue was washed with n-hexane (20 mL×2) and dried under high vacuum to afford methyl 8-bromo-6-cyclopropoxyquinoline-3-carboxylate (X-1501A6) (1.5 g, 57%) as a brown solid. MS: [MH]⁺321.7/[MH+2]⁺323.7.

Methyl 6-cyclopropoxy-8-(6-azaspiro[2.5]octan-6-yl)quinoline-3-carboxylate (X-1501A7). To a stirred solution of methyl 8-bromo-6-cyclopropoxyquinoline-3-carboxylate (X-1501A6) (0.600 g, 1.86 mmol) in a toluene (5 mL) were added 6-azaspiro [2.5]octane hydrochloride (0.827 g, 5.6 mmol), cesium carbonate (4.56 g, 14.0 mmol), and rac-BINAP (0.233 g, 0.37 mmol) sequentially at room temperature under nitrogen. The reaction mixture was degassed (purging with nitrogen) for 20 min followed by the addition of Pd(OAc)₂ (0.042 g, 0.18 mmol) and the resulting mixture was heated at 100° C. for 9 h. Reaction mixture was cooled to room temperature, diluted with water (30 mL), and extracted with ethyl acetate (30 mL×3). Combined organic extracts were dried over anhydrous Na₂SO₄ and concentrated under reduce pressure. The crude product was purified by silica gel column chromatography using ethyl acetate-hexane=0:10->1:9 as gradient to afford methyl 6-cyclopropoxy-8-(6-azaspiro[2.5]octan-6-yl)quinoline-3-carboxylate (X-1501A7) (0.400 g, 61%) as a yellow solid. MS: [MH]⁺353.0.

6-cyclopropoxy-8-(6-azaspiro[2.5]octan-6-yl)quinoline-3-carboxylic acid (X-1501A8). To a stirred solution of methyl 6-cyclopropoxy-8-(6-azaspiro[2.5]octan-6-yl)quinoline-3-carboxylate (X-1501A7) (0.350 g, 0.99 mmol) in a mixture of THF-water (3:1; 5.0 mL) was added lithium hydroxide monohydrate (0.126 g, 2.98 mmol) at room temperature, and the resulting mixture was heated at 70° C. for 1 h. After cooling to room temperature, the reaction mixture was concentrated under reduced pressure, and obtained crude was diluted with water (40 mL) and was extracted with ethyl acetate (40 mL×2) to remove unwanted organic impurities. Aqueous part was acidified (pH ˜2-3) with an aqueous solution of 1N HCl, and the resulting precipitate was collected by filtration. Crude residue was washed with cold water until the pH of the filtrate became neutral (pH ˜6-7). Obtained solid was dried under high vacuum to afford to afford 6-cyclopropoxy-8-(6-azaspiro[2.5]octan-6-yl)quinoline-3-carboxylic acid (X-1501A8) (0.30 g, 37%) as a yellow solid. MS: [MH]⁺353.0.

(R)-6-cyclopropoxy-N-(1-hydroxypropan-2-yl)-8-(6-azaspiro[2.5]octan-6-yl)quinoline-3-carboxamide (I-25). To a stirred solution of 6-cyclopropoxy-8-(6-azaspiro[2.5]octan-6-yl)quinoline-3-carboxylic acid (X-1501A8) (0.200 g, 0.59 mmol) in THF (5 mL) were added (R)-2-aminopropan-1-ol (0.155 g, 2.07 mmol), TEA (0.297 g, 2.95 mmol) and T₃P (0.283 g, 0.88 mmol) sequentially at 0° C. under nitrogen and the resulting reaction mixture was stirred at room temperature for 1h. Reaction mixture was diluted with water (30 mL) and was extracted with ethyl acetate (30 mL×2). Collected organic extracts were dried over anhydrous Na₂SO₄ and concentrated under reduced pressure. The resulting crude was purified by reverse phase (C-18) silica gel column chromatography, using acetonitrile-water=0:1→1:0 as gradient, to afford (R)-6-cyclopropoxy-N-(1-hydroxypropan-2-yl)-8-(6-azaspiro[2.5]octan-6-yl)quinoline-3-carboxamide (I-25) (0.07 g, 30%) as an off-white solid. ¹H NMR (400 MHz, DMSO-d₆) δ 9.05-9.04 (d, J=2.0 Hz, 1H), 8.63-8.62 (d, J=2.0 Hz, 1H), 8.39-8.38 (d, J=8.0 Hz, 1H), 7.21-7.20 (d, J=2.0 Hz, 1H), 6.78-6.77 (d, J=2.0 Hz, 1H), 4.78-4.75 (t, J=6.0 Hz, 1H), 4.10-4.05 (m, 1H), 3.97-3.94 (m, 1H), 3.51-3.48 (m, 1H), 3.39-3.34 (m, 1H), 3.27 (s, 4H; merged with moisture from DMSO-d₆), 1.57 (brs, 4H). 1.17-1.16 (d, J=6.4 Hz, 3H). 0.87-0.85 (m, 2H), 0.73 (brs, 2H), 0.35 (s, 41-), MS: [MH]⁺ 396.1. (four proton merged with moisture peak).

The following compound was prepared in a manner analogous to the procedures described above for (R)-6-cyclopropoxy-N-(1-hydroxypropan-2-yl)-8-(6-azaspiro[2.5]octan-6-yl)quinoline-3-carboxamide (I-25):

(S)-6-cyclopropoxy-N-(1-methoxypropan-2-yl)-8-(4-(trifluoromethyl)piperidin-1-yl)quinoline-3-carboxamide (I-26) (0.1 g, 42%) as a yellow solid. ¹H NMR (400 MHz, DMSO-d₆) δ 9.04-9.04 (d, J=1.6 Hz, 1H), 8.63-8.62 (d, J=1.6 Hz, 1H), 8.54-8.52 (d, J=8.0 Hz, 1H), 7.25-7.25 (d, J=1.6 Hz, 1H), 6.77-6.77 (d, J2.0 Hz, 1H), 4.28-4.21 (m, 1H), 4.02-3.96 (m, 3H), 3.46-3.42 (m, 1H), 3.33-3.29 (m, 1H; merged with moisture from DMSO-d₆), 3.28 (s, 3H), 2.77-2.72 (t, J=11.6 Hz, 2H). 2.49 (1H; merged with DMSO-d₆), 1.95-1.92 (d, J=11.2 Hz, 2H), 1.79-1.71 (m, 2H), 1.18-1.16 (d, J=6.8 Hz, 3H), 0.86-0.85 (m, 2H), 0.729 (s, 2H) MS: [MH]⁺ 452.0.

### Example 1.6. Synthesis of (R)-6-(difluoromethoxy)-N-(1-hydroxypropan-2-yl)-8-(6-azaspiro[2.5]octan-6-yl)quinoline-3-carboxamide (I-27)

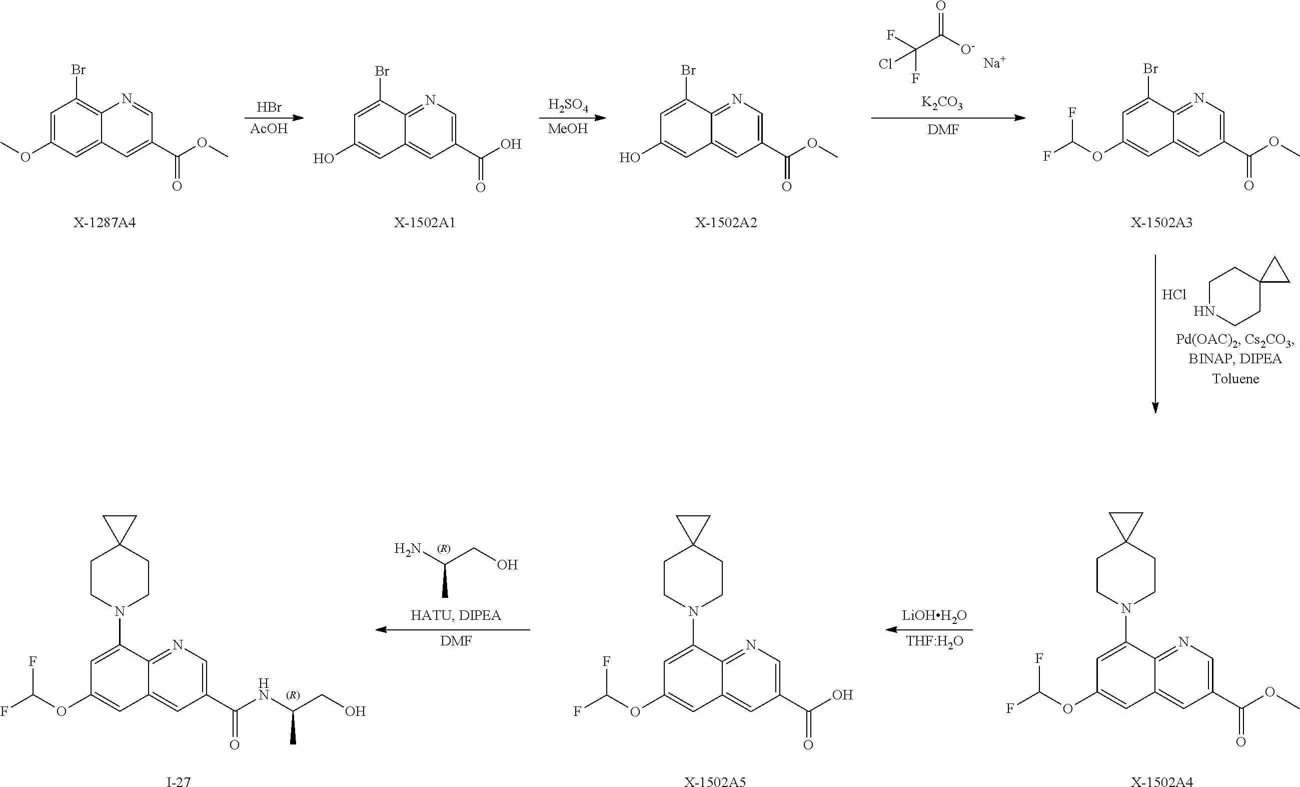

8-Bromo-6-hydroxyquinoline-3-carboxylic acid (X-1502A1). To a stirred solution of methyl 8-bromo-6-methoxyquinoline-3-carboxylate (X-1287A4) (4.0 g, 13.5 mmol) in AcOH (32 mL) was added HBr (23.0 g, 288.0 mmol) at room temperature under nitrogen, and the reaction mixture was heated at 120° C. for 48 h. After cooling to room temperature, reaction mixture was slowly poured into ice-water (400 mL), and the resulting precipitate was collected by filtration. Obtained solid residue was washed with water (100 mL) and dried under high vacuum to afford 8-bromo-6-hydroxyquinoline-3-carboxylic acid (X-1502A1) (4.66 g, quantitative (crude)) as a yellow solid, which was taken to next step without further purification. MS: [MH]⁺267.9/[MH]+269.9.

Methyl 8-bromo-6-hydroxyquinoline-3-carboxylate (X-1502A2). To a stirred solution of 8-bromo-6-hydroxyquinoline-3-carboxylic acid (X-1502A1) (4.66 g, 17.40 mmol) in methanol (300 mL) was added concentrated H₂SO₄ (0.9 mL) at room temperature, and the resulting mixture was heated at 70° C. for 16 h. After cooling to room temperature, the reaction mixture was concentrated under reduced pressure. Residue was dissolved in ethyl acetate (200 mL) and washed with an aqueous solution of saturated NaHCO₃ (120 mL). The organic extract was dried over anhydrous Na₂SO₄, filtered, and concentrated under reduced pressure to afford methyl 8-bromo-6-hydroxyquinoline-3-carboxylate (X-1502A2) (2.18 g, 44%) as a white solid. MS: [MH]+281.8/[MH+2]⁺283.8.

Methyl 8-bromo-6-(difluoromethoxy)quinoline-3-carboxylate (X-1502A3). To a stirred solution of methyl 8-bromo-6-hydroxyquinoline-3-carboxylate (X-1502A2) (2.15 g, 7.65 mmol) in DMF (7 mL) were added potassium carbonate (6.33 g, 45.9 mmol) and sodium 2-chloro-2,2-difluoroacetate (5.81 g, 38.2 mmol) sequentially at room temperature under nitrogen, and the resulting mixture was stirred at 80° C. for 6 h. The reaction mixture was poured into ice-water (100 mL) and was extracted with ethyl acetate (120 mL×3). Combined organic extracts were dried over anhydrous Na₂SO₄ and concentrated under reduced pressure. Obtained crude product was purified by silica gel column chromatography using ethyl acetate-hexane=1:94→2:8 as gradient to afford methyl 8-bromo-6-(difluoromethoxy)quinoline-3-carboxylate (X-1502A3) (1.27 g, 49%) as a white solid. MS: [MH]⁺331.9/[MH+2]⁺333.8.

Methyl 6-(difluoromethoxy)-8-(6-azaspiro[2.5]octan-6-yl)quinoline-3-carboxylate (X-1502A4). To a stirred solution of methyl 8-bromo-6-(difluoromethoxy)quinoline-3-carboxylate (X-1502A3) (0.400 g, 1.20 mmol) in a toluene (8 mL) were added 6-azaspiro[2.5]octane hydrochloride (0.266 g, 1.81 mmol), cesium carbonate (2.75 g, 8.45 mmol), and rac-BINAP (0.150 g, 0.24 mmol) at room temperature under nitrogen. The reaction mixture was degassed (purging with nitrogen) for 20 min followed by addition of Pd(OAc)₂ (0.027 g, 0.12 mmol), and the reaction mixture was heated at 110° C. for 48 h. Reaction mixture was cooled to room temperature, diluted with water (40 mL), and extracted with ethyl acetate (40 mL×3). Combined organic extracts were dried over anhydrous Na₂SO₄ and concentrated under reduce pressure. The crude product was purified by silica gel column chromatography using ethyl acetate-hexane=0:10→1:9 as gradient to afford methyl 6-(difluoromethoxy)-8-(6-azaspiro[2.5]octan-6-yl)quinoline-3-carboxylate (X-1502A4) (0.400 g, 61%) as a yellow solid. MS: [MH]⁺363.2.

6-(Difluoromethoxy)-8-(6-azaspiro[2.5]octan-6-yl)quinoline-3-carboxylic acid (X-1502A5). To a stirred solution of methyl 6-(difluoromethoxy)-8-(6-azaspiro[2.5]octan-6-yl)quinoline-3-carboxylate (X-1502A4) (0.170 g, 0.46 mmol) in a mixture of THF-water (3:1; 4.0 mL) was added lithium hydroxide monohydrate (0.059 g, 1.40 mmol) at room temperature, and the resulting mixture was stirred for 1 h at the same temperature. Reaction mixture was concentrated under reduced pressure, and obtained crude was diluted with water (40 mL) and extracted with ethyl acetate (40 mL×2) to remove unwanted organic impurities. Aqueous part was acidified (pH ˜2-3) with an aqueous solution of 1N HCl, and the resulting precipitate was collected by filtration. Crude residue was washed with cold water until the pH of the filtrate became neutral (pH ˜6-7). Obtained solid was dried under high vacuum to afford 6-(difluoromethoxy)-8-(6-azaspiro[2.5]octan-6-yl)quinoline-3-carboxylic acid (X-1502A5) [0.155 g, 95% (crude) as a yellow solid. The crude product is pure enough to taken to next step without further purification. MS: [MH]⁺349.0.

(R)-6-(difluoromethoxy)-N-(1-hydroxypropan-2-yl)-8-(6-azaspiro[2.5]octan-6-yl)quinoline-3-carboxamide (I-27). To a stirred solution of 6-(difluoromethoxy)-8-(6-azaspiro[2.5]octan-6-yl)quinoline-3-carboxylic acid (X-1502A5) (0.08 g, 0.22 mmol) in DMF (4 mL) were added (R)-2-aminopropan-1-ol (0.026 g, 0.34 mmol), DIPEA (0.118 g, 0.91 mmol), and HATU (0.130 g, 0.34 mmol) sequentially at 0° C. under nitrogen, and the resulting reaction mixture was stirred at room temperature for 1 h. Reaction mixture was diluted with water (30 mL) and was extracted with ethyl acetate (30 mL×2). Organic extracts were combined, dried over anhydrous Na₂SO₄, and concentrated under reduced pressure. The resulting crude was purified by reverse phase (C-18) silica gel column chromatography using acetonitrile-water=0:1→1:0 as gradient to afford (R)-6-(difluoromethoxy)-N-(1-hydroxypropan-2-yl)-8-(6-azaspiro[2.5]octan-6-yl)quinoline-3-carboxamide (I-27) (0.028 g, 30%) as an off-white solid. ¹H NMR (400 MHz, DMSO-d) δ 9.16-9.16 (d, J=2.0 Hz, 1H), 8.69-8.69 (d, J=2.0 Hz, 1H), 8.44-8.42 (d, J=7.6 Hz, 1H), 7.58-7.21 (s, J=73.6 Hz, 1H), 7.27 (s, 1H), 6.96-6.95 (d, J2.4 Hz, 1H), 4.79-4.76 (t, J=5.6 Hz 1H), 4.08-4.05 (m, 1H), 3.52-3.46 (m, 2H), 3.40-3.34 (m, 4H). 1.58 (brs, 4H), 1.17-1.15 (d, J=6.8, 3H), 0.37 (s, 41). MS: [MH]⁺406.0.

The following compound was prepared in a manner analogous to the procedures described above for (R)-6-(difluoromethoxy)-N-(1-hydroxypropan-2-yl)-8-(6-azaspiro[2.5]octan-6-yl)quinoline-3-carboxamide (I-27):

(S)-6-(difluoromethoxy)-N-(1-methoxypropan-2-yl)-8-(4-(trifluoromethyl)piperidin-1-yl)quinoline-3-carboxamide (I-28) (0.11 g, 48%) as a yellow solid. ¹H NMR (400 MHz, DMSO-d₆) δ 9.16-9.16 (d, J=1.6 Hz 1H), 8.70-8.69 (d, J=1.6 Hz, 1H), 8.58-8.56 (d, J=8.0 Hz, 1H), 7.58-7.21 (s, J=74.0 Hz, 1H), 7.32 (s, 1H), 6.97-6.96 (d, J=2.0 Hz, 1H), 4.26-4.23 (m, 1H), 4.10-4.07 (d, J=11.6 Hz, 2H), 3.46-3.42 (m, 1H), 3.32 (1H, merged with moisture from DMSO-d₆), 3.28 (s, 3H), 2.86-2.80 (t, J=11.6, 2H), 2.49 (1H, merged with DMSO-d₆), 1.97-1.94 (d, J=12.4, 2H), 1.81-1.75 (m, 2H), 1.18-1.17 (d, J=6.4, 3H). MS: [MH]⁺462.0.

### Example 1.7. Synthesis of (S)-8-(4-ethynylphenyl)-N-(1-(pyridin-2-yl)ethyl)quinoline-3-carboxamide (I′-29)

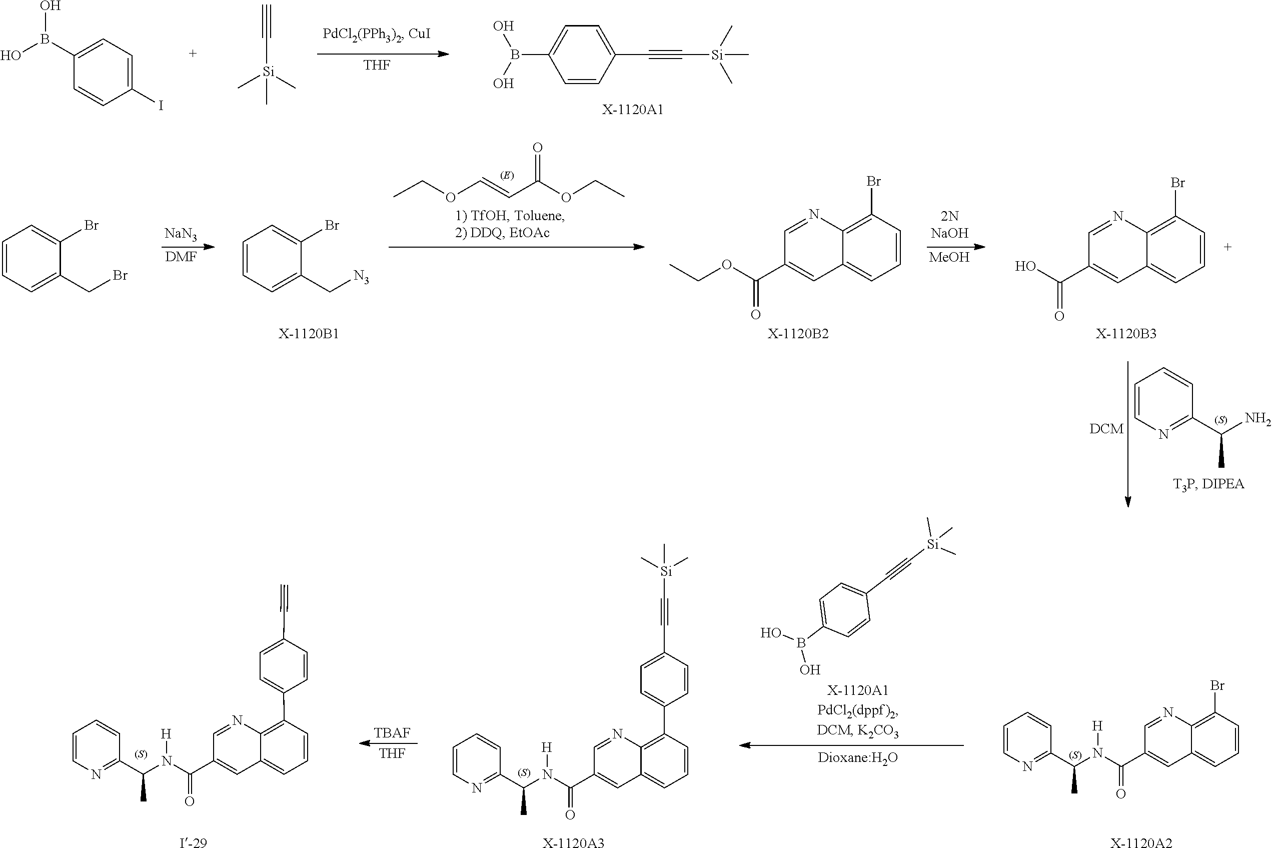

1-(Azidomethyl)-2-bromobenzene (X-1120B1). To a stirred solution of 1-bromo-2-(bromomethyl)benzene (10.0 g, 0.040 mmol) in DMF (100 mL) were added sodium azide (5.2 g, 0.080 mmol) at room temperature under nitrogen, and the resulting mixture was stirred at room temperature for 3h. The reaction mixture quenched with water (600 mL) and was extracted with ethyl acetate (500 mL×3). Collected organics were washed with brine (150 mL), dried over anhydrous Na₂SO₄, and concentrated in vacuo to afford 1-(azidomethyl)-2-bromobenzene (X-1120B1) (8.4 g, 99%) as a pale yellow oil, which was used in next step without further purification.

Ethyl 8-bromoquinoline-3-carboxylate (X-1120B2). To a stirred solution of 1-(azidomethyl)-2-bromobenzene (X-1120B1) (8.4 g, 0.039 mmol) in toluene were added trifluoromethanesulfonic acid (5.9 g, 0.039 mmol) at 0° C. under nitrogen, and the resulting mixture was stirred at room temperature for 10 min followed by addition of ethyl (E)-3-ethoxyacrylate. The resulting mixture was stirred at 80° C. for 3 h. The reaction mixture quenched with aq. NaHCO₃ (600 mL) and was extracted with ethyl acetate (500 mL×3). Collected organics were washed with brine (150 mL), dried over anhydrous Na₂SO₄, and concentrated in vacuo. The reaction mixture was diluted with ethyl acetate followed by addition of DDQ. The resulting mixture was stirred at room temperature for 16 h. The reaction mixture was concentrated under reduced pressure. The crude product was purified by silica gel column chromatography, using ethyl acetate-hexane=0:1→3:7 as gradient, to afford ethyl 8-bromoquinoline-3-carboxylate (X-1120B2) (2.5 g, 22%) as an white solid. MS: [MH]⁺280.1.

8-Bromoquinoline-3-carboxylic acid (X-1120B3). To a stirred solution of ethyl 8-bromoquinoline-3-carboxylate (X-1120B2) (2.5 g, 0.0089 mmol) in methanol (10 mL) were added 2N sodium hydroxide (3 mL) at room temperature under nitrogen, and the resulting mixture was stirred at room temperature for 3h. The reaction mixture was concentrated under reduced pressure, and the crude mass was diluted with water (200 mL) and extracted with ethyl acetate (200×2 mL) to remove unwanted organic impurities. The aqueous layer was acidified (pH ˜2-3) with an aqueous 1 N HCl, and the resulting precipitate was extracted with ethyl acetate (100 mL×3). Collected organics were washed with brine (150 mL), dried over anhydrous Na₂SO₄, and concentrated in vacuo, to afford 8-bromoquinoline-3-carboxylic acid (X-1120B3) (1.5 g, 71%) as an off-white solid. MS: [MH]⁺252.0.

(4-((Trimethylsilyl)ethynyl)phenyl)boronic acid (X-1120A1). To a stirred solution of (4-iodophenyl)boronic acid (1.0 g, 2.01 mmol) in THF (20.0 mL) were added ethynyltrimethylsilane (0.780 g, 4.03 mmol) and triethylamine (0.487 g, 4.80 mmol), followed by addition of CuI (0.382 g, 1.0 mmol) at room temperature under nitrogen. The reaction mixture was degassed (purging with nitrogen) for 20 min followed by the addition of PdCl₂(PPh₃)₂(0.282 g, 0.20 mmol), and the reaction mixture was heated at room temperature for 3h. The reaction mixture was cooled to room temperature, diluted with water (50 mL) and was extracted with ethyl acetate (100 mL×2). Combined organic extracts were dried over anhydrous Na₂SO₄, filtered and concentrated under reduced pressure. Obtained crude was purified by silica gel column chromatography, using ethyl acetate-hexane=0:1→2:8 as gradient, to afford methyl (4-((trimethylsilyl)ethynyl)phenyl)boronic acid (X-1120A1) (0.600 g, 68%) as an white solid (X-1120A1). ¹H NMR (400 MHz, DMSO-d6) δ 8.18 (s, 2H), 7.78-7.76 (d, J=7.6 Hz, 2H), 7.42-7.40 (d, J=7.6 Hz, 2H), 0.23 (s, 9H).

8-Bromoquinoline-3-carboxylic acid (X-1120A2). To a stirred solution of 8-bromoquinoline-3-carboxylic acid (X-1120B3) (1.5 g, 5.90 mmol) in DCM (20.0 mL) were added N, N-diisopropylethylamine (2.31 g, 11.94 mmol), (S)-1-(pyridin-2-yl)ethan-1-amine (0.874 g, 7.17 mmol), and propylphosphonic anhydride (3.79 g, 11.94 mmol) sequentially at 0° C. under nitrogen, and the resulting mixture was stirred at room temperature for 1h. Reaction mixture was diluted with water (100 mL) and was extracted with ethyl acetate (150 mL×3). Combined organic extracts were dried over anhydrous Na₂SO₄ and concentrated under reduce pressure to provide a crude mass, which was purified by silica gel column chromatography, using methanol-DCM=0:1→0.2:8.8 as gradient, to afford 8-bromoquinoline-3-carboxylic acid (X-1120A2) (1.1 g, 51%) as an white solid. MS: [MH]⁺356.2.

(S)-N-(1-(pyridin-2-yl)ethyl)-8-(4-((trimethylsilyl)ethynyl)phenyl)quinoline-3-carboxamide (X-1120A3). To a stirred solution of (S)-8-bromo-N-(1-(pyridin-2-yl)ethyl)quinoline-3-carboxamide (X-1120A2) (0.250 g, 0.42 mmol) in a mixture of dioxane:water (4:2, 6 mL) were added (4-((trimethylsilyl)ethynyl)phenyl)boronic acid (X-1120A1) (0.184 g, 0.84 mmol) and K₂CO₃ (0.174 g, 1.26 mmol) at room temperature. The reaction mixture was degassed (purging with nitrogen) for 20 min followed by addition of PdCl₂(dppf)₂.DCM (0.034 g, 0.040 mmol), and the reaction mixture was heated at 90° C. for 2 h. The reaction mixture was cooled to room temperature, quenched with water (100 mL), and was extracted with ethyl acetate (100 mL×2). The combined organic extracts were dried over anhydrous Na₂SO₄ and concentrated under reduced pressure. The crude product was purified by silica gel column chromatography, using ethyl acetate-Hexane=0:1→3:7 as gradient, to afford (S)-N-(1-(pyridin-2-yl)ethyl)-8-(4-((trimethylsilyl)ethynyl)phenyl)quinoline-3-carboxamide (X-1120A3) (0.130 g, 41%) as an brown solid. MS: [MH]⁺449.6

(S)-8-(4-ethynylphenyl)-N-(1-(pyridin-2-yl)ethyl)quinoline-3-carboxamide (I′-29). To a stirred solution of (S)-N-(1-(pyridin-2-yl)ethyl)-8-(4-((trimethylsilyl)ethynyl)phenyl)quinoline-3-carboxamide (X-1120A3) (0.100 g, 0.22 mmol) in THF (5 mL) was added TBAF (0.5 mL, 0.55 mmol) at 0° C. Then reaction mixture stirred at room temperature for 1h. The reaction mixture was quenched by aqueous NaHCO₃ solution and was extracted by ethyl acetate (100 ml×2). The combined organic extracts were dried over anhydrous Na₂SO₄ and concentrated under reduce pressure, to afford (S)-8-(4-ethynylphenyl)-N-(1-(pyridin-2-yl)ethyl)quinoline-3-carboxamide (I′-29) (0.060 g, 71%) as an brown solid. MS: [MH]⁺378.1 ¹H NMR (400 MHz, DMSO-d6) δ 9.30 (s, 1H), 9.26-9.24 (d, J=7.6 Hz, 1H), 8.96 (s, 1H), 8.55-8.54 (d, J=4.4 Hz, 1H), 8.17-8.15 (d, J=8.0 Hz, 1H), 7.91-7.89 (d, J=7.2 Hz, 1H), 7.80-7.78 (t, J=7.6 Hz, 2H), 7.72-7.70 (d, J=8.0 Hz, 2H), 7.61-7.59 (d, J=8.0 Hz, 2H), 7.49-7.47 (d, J=8.0 Hz, 1H), 7.29-7.26 (t, J=5.2 Hz, 1H), 5.29-5.27 (m, 1H), 4.28 (s, 1H), 1.57-1.59 (d, J=7.2 Hz, 3H). MS: [MH]⁺378.19.

### Example 1.8. Synthesis of (S)-8-ethynyl-N-(1-(pyridin-2-yl)ethyl)quinoline-3-carboxamide (30)

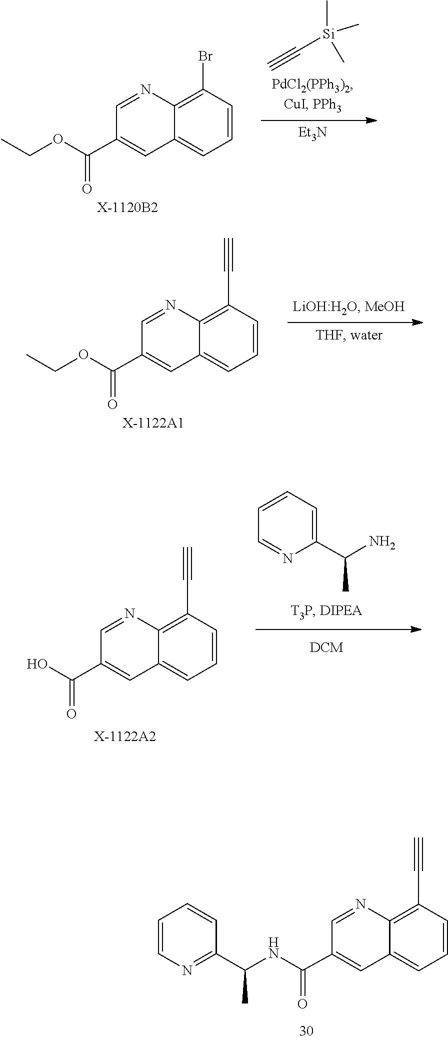

The synthetic procedure of ethyl 8-bromoquinoline-3-carboxylate (X-1120B2) described in Example 1.7.

Ethyl 8-ethynylquinoline-3-carboxylate (X-1122A1). To a stirred solution of ethyl 8-bromoquinoline-3-carboxylate (X-1120B2) (0.400 g, 1.43 mmol) in trimethylamine (5 mL) were added ethynyltrimethylsilane (0.702 g, 7.16 mmol) and CuI (0.013 g, 0.071 mmol) sequentially at room temperature under nitrogen. The reaction mixture was degassed (purging with nitrogen) for 20 min followed by the addition of PdCl₂(PPh₃)₂(0.010 g, 0.040 mmol), and the resulting mixture was heated at 110° C. for 1.5 h. The reaction mixture was cooled to room temperature, was diluted with water (100 mL), and was extracted with ethyl acetate (100 mL×3). Combined organic extracts were dried over anhydrous Na₂SO₄ and concentrated under reduce pressure. The crude product was combined with identically prepared one more batch and the combined crude product was purified by reverse phase (C-18) silica gel column chromatography, using acetonitrile-water-=0:1→5:5 as gradient, to afford ethyl 8-ethynylquinoline-3-carboxylate (X-1122A1) (0.240 g, 38%) as an off-white solid. MS: [MH]⁺278.9.

8-Ethynylquinoline-3-carboxylic acid (X-1122A2). To a stirred solution of ethyl 8-ethynylquinoline-3-carboxylate (X-1122A1) (0.150 g, 0.66 nmol) in a mixture of THF-water (2:1; 3.0 mL) was added lithium hydroxide monohydrate (0.083 g, 1.99 mmol) at room temperature, and the resulting mixture was stirred at room temperature for 2 h. The reaction mixture was concentrated under reduced pressure, and the obtained crude was diluted with water (10 mL) and was extracted with ethyl acetate (10 mL×2) to remove unwanted organic impurities. The aqueous part was acidified (pH ˜6) with an aqueous solution of 1 N HCl, and the resulting precipitate was collected by filtration. The obtained solid was dried under high vacuum to afford 8-ethynylquinoline-3-carboxylic acid (X-1122A2) (0.100 g, 86%) as an off-white solid. MS: [MH]⁺197.0.

(S)-8-ethynyl-N-(1-(pyridin-2-yl)ethyl)quinoline-3-carboxamide (30). To a stirred solution of (S)-1-(pyridin-2-yl)ethan-1-amine (0.083 g, 0.68 mmol) in DCM (3 mL) were added triethylamine (0.098 g, 1.37 mmol), 8-ethynylquinoline-3-carboxylic acid (X-1122A2) (0.090 g, 0.45 mmol) and propylphosphonic anhydride (0.290 g, 0.91 mmol) sequentially at 0° C. under nitrogen, and the resulting mixture was stirred at room temperature for 1 h. The reaction mixture was diluted with water (100 mL) and was extracted with ethyl acetate (100 mL×3). Combined organic extracts were dried over anhydrous Na₂SO₄ and concentrated under reduce pressure to provide a crude mass, which was purified by reverse phase (C-18) silica gel column chromatography using acetonitrile-water=0:1→2:8 as gradient, to afford (S)-8-ethynyl-N-(1-(pyridin-2-yl)ethyl)quinoline-3-carboxamide (30) (0.060 g, 44%) as an white solid. ¹H NMR (400 MHz, DMSO-d₆) δ 9.365-9.360 (d, J=2.0 Hz, 1H), 9.28-9.26 (d, J=7.6 Hz, 1H), 8.965-8.961 (d, J=1.6 Hz, 1H), 8.56-8.55 (d, J=4.4 Hz, 1H), 8.17-8.15 (d, J=8.0 Hz, 1H), 8.07-8.06 (d, J=6.8 Hz, 1H), 7.81-7.77 (t, J=7.6 Hz, 1H), 7.71-7.67 (t, J=7.6 Hz, 1H), 7.50-7.48 (d, J=8.0 Hz, 1H), 7.30-7.27 (t, J=5.2 Hz, 1H), 5.31-5.24 (m, 1H), 4.55 (s, 1H), 1.58-1.56 (d, J=7.2 Hz, 3H). MS: [MH]⁺ 302.06.

### Example 1.9. Synthesis of (S)-8-(3,3-dimethylbut-1-yn-1-yl)-N-(1-(pyridin-2-yl)ethyl)quinoline-3-carboxamide (31)

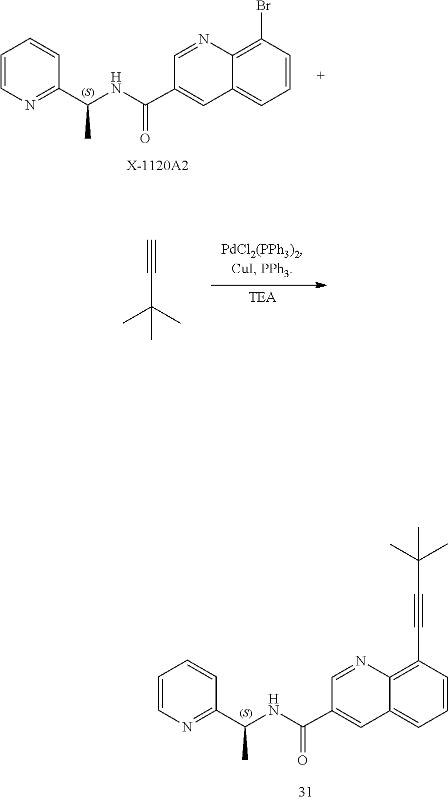

The synthetic procedure of (S)-8-bromo-N-(1-(pyridin-2-yl)ethyl)quinoline-3-carboxamide (X-1120A2) described in Example 1.7.

To a stirred solution of (S)-8-bromo-N-(1-(pyridin-2-yl)ethyl)quinoline-3-carboxamide (X-1120A2) (0.150 g, 0.42 mmol) in trimethylamine (3.0 mL) were added 3,3-dimethylbut-1-yne (0.052 g, 0.63 mmol) and CuI (0.040 g, 0.021 mmol) sequentially at room temperature under nitrogen. The reaction mixture was degassed (purging with nitrogen) for 20 min followed by the addition of PdCl₂(PPh₃)₂(0.029 g, 0.042 mmol), and the resulting mixture was heated at 100° C. for 1 h. The reaction mixture was cooled to room temperature, was diluted with water (100 mL), and was extracted with ethyl acetate (100 mL×3). Combined organic extracts were dried over anhydrous Na₂SO₄ and concentrated under reduce pressure to afford crude mass, which was purified by reverse phase (C-18) silica gel column chromatography, using acetonitrile-water=0:1→3:7 as gradient, to afford (S)-8-(3,3-dimethylbut-1-yn-1-yl)-N-(1-(pyridin-2-yl)ethyl)quinoline-3-carboxamide (31) (0.030 g, 16%) as a white solid. ¹H NMR (400 MHz, DMSO-d₆) δ 9.38 (s, 1H), 9.22-9.21 (d, J=7.6 Hz, 1H), 8.90 (s, 1H), 8.55 (s, 1H), 8.08-8.06 (d, J=7.6 Hz, 1H), 7.92-7.90 (d, J=6.8 Hz, 1H), 7.80-7.77 (t, J=7.2 Hz, 1H), 7.65-7.62 (t, J=7.6 Hz, 1H), 7.50-7.48 (d, J=7.6 Hz, 1H), 7.28 (br. s, 1H), 5.28-5.25 (t, J=7.2 Hz, 1H), 1.57-1.55 (d, J=6.8 Hz, 3H), 1.38 (s, 9H). MS: [MH]⁺358.7.

### Example 1.10. Synthesis of (S)-8-(4-cyanophenyl)-N-(1-(pyridin-2-yl)ethyl)quinoline-3-carboxamide (I′-32)

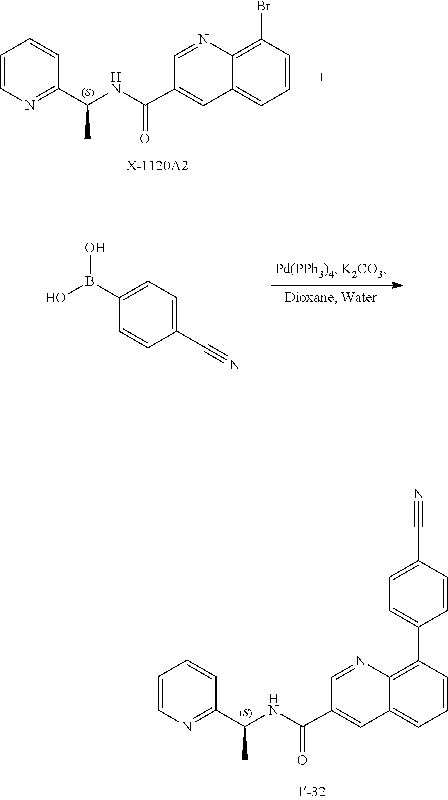

The synthetic procedure of (S)-8-bromo-N-(1-(pyridin-2-yl)ethyl)quinoline-3-carboxamide (X-1120A2) described in Example 1.7.

To a stirred solution of (S)-8-bromo-N-(1-(pyridin-2-yl)ethyl)quinoline-3-carboxamide (X-1120A2) (0.150 g, 0.42 mmol) in a mixture of dioxane:water (2:1, 3 mL) were added (4-cyanophenyl)boronic acid (0.099 g, 0.67 mmol) and K₂CO₃ (0.205 g, 1.47 mmol) sequentially at room temperature under nitrogen. The reaction mixture was degassed (purging with nitrogen) for 20 min followed by the addition of Pd(PPh₃)₄(0.073 g, 0.06 mmol) and the resulting mixture was heated at 90° C. for 16h. Reaction mixture was cooled to room temperature, diluted with water (100 mL) and was extracted with ethyl acetate (100 mL×3). Combined organic extracts were dried over anhydrous Na₂SO₄ and concentrated under reduce pressure to afford crude mass, which was purified by reverse phase (C-18) silica gel column chromatography, using acetonitrile-water=0:1→8:2 as gradient, to afford (S)-8-(4-cyanophenyl)-N-(1-(pyridin-2-yl)ethyl)quinoline-3-carboxamide (I′-32) (0.040 g, 25%) as a white solid. ¹H NMR (400 MHz, DMSO-d₆) δ 9.30-9.30 (m, 2H), 9.01-9.00 (d, J=1.6 Hz, 1H), 8.55-8.54 (d, J=4.4 Hz, 1H), 8.22-8.20 (d, J=8.0 Hz, 1H), 7.98-7.96 (t, J=8.4 Hz, 3H), 7.90-7.88 (d, J=8.0 Hz, 2H), 7.84-7.82 (m, 2H), 7.49-7.47 (d, J=7.6 Hz, 1H), 7.29-7.28 (t, J=5.2 Hz, 1H), 5.29-5.27 (m, 1H), 1.57-1.55 (d, J=7.2 Hz, 3H), 0.92-0.88 (t, J=8.4 Hz, 3H). MS: [MH]⁺379.2.

### Example 1.11. Synthesis of (R)-8-(4-(tert-butyl)phenyl)-N-(1-hydroxypropan-2-yl)-6-methoxyquinoline-3-carboxamide (I′-33)

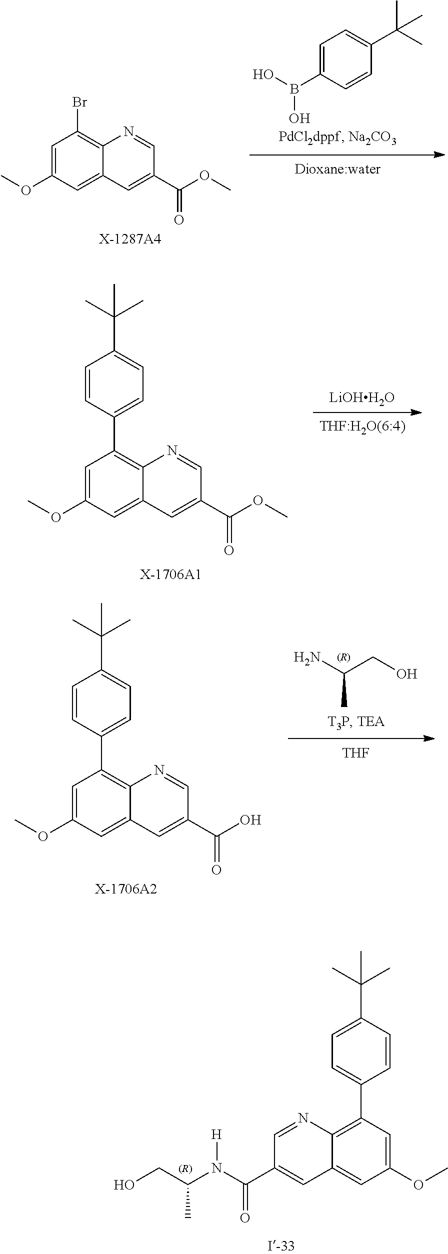

The following compound was prepared in a manner analogous to the procedures mentioned in Example 1.2:

(R)-8-(4-(tert-butyl)phenyl)-N-(1-hydroxypropan-2-yl)-6-methoxyquinoline-3-carboxamide (I′-33) (0.030 g, 10%) as an off-white solid. ¹H NMR (400 MHz, DMSO-d₆) δ 9.06 (s, 1H), 8.74 s, (1H), 8.45-8.43 (d, J=7.6 Hz, 1H), 7.61-7.59 (d, J=8.4 Hz, 2H), 7.51-7.49 (d, J=8.0 Hz, 2H), 7.46 (s, 2H), 4.79-4.78 (t, J=5.6 Hz, 1H), 4.10-4.07 (quint, J=6.4 Hz, 1H), 3.95 (s, 3H), 3.53-3.49 (m, 1H), 3.42-3.33 (m, 1H), 1.36 (s, 9H), 1.19-1.18 (d, J=6.8 Hz, 3H). MS: [MH]+393.2.

### Example 1.12. Synthesis of (R)-N-(1-hydroxypropan-2-yl)-8-(4-(2-hydroxypropan-2-yl) phenyl)-6-methoxyquinoline-3-carboxamide (I′-34)

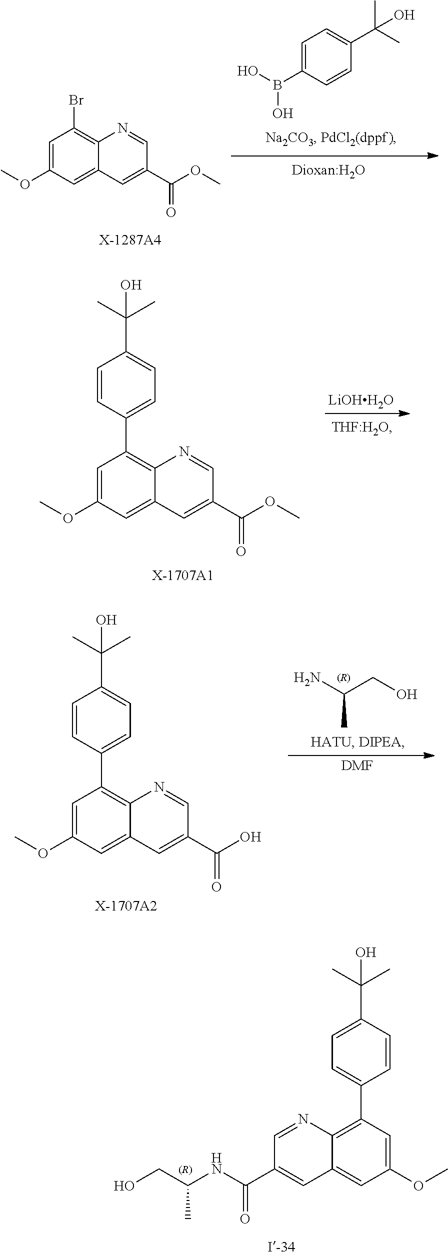

Synthetic procedure of 6-methoxy-8-(4-(trifluoromethyl)piperidin-1-yl)quinoline-3-carboxylic acid (X-1287A4) described in Example 1.1.

Methyl 8-(4-(2-hydroxypropan-2-yl)phenyl)-6-methoxyquinoline-3-carboxylate (X-1707A1). To a stirred solution of methyl 8-bromo-6-methoxyquinoline-3-carboxylate (X-1287A4) (0.800 g, 2.71 mmol) in a mixture of 1,4-dioxane-water (1:4, 15 mL) were added (4-(2-hydroxypropan-2-yl)phenyl)boronic acid (0.732 g, 4.06 mmol) and Na₂CO₃ (0.861 g, 8.13 mmol) sequentially at room temperature under nitrogen. The reaction mixture was degassed (purging with nitrogen) for 20 min followed by the addition of PdCl₂(dppf) (0.099 g, 0.013 mmol) and the resulting mixture was heated at 100° C. for 1 h. The reaction mixture was cooled to room temperature, quenched with water (100 mL), and extracted with ethyl acetate (300 mL×2). The combined organic extracts were dried over anhydrous Na₂SO₄ and concentrated under reduced pressure. The crude product was purified by silica gel column chromatography, using ethyl acetate-hexane=0:1→2:8 as gradient, to afford methyl 8-(4-(2-hydroxypropan-2-yl) phenyl)-6-methoxyquinoline-3-carboxylate (X-1707A1) (1.30 g, 94%) as an off-white solid. MS: [MH]⁺352.2.

8-(4-(2-Hydroxypropan-2-yl)phenyl)-6-methoxyquinoline-3-carboxylic acid (X-1707A2). To a stirred solution of methyl 8-(4-(2-hydroxypropan-2-yl) phenyl)-6-methoxyquinoline-3-carboxylate (X-1707A1) (1.0 g, 2.84 mmol) in a mixture of THF-water (3:1; 11.5 mL) was added lithium hydroxide monohydrate (0.357 g, 8.52 mmol) at room temperature under nitrogen, and the resulting mixture was heated at 70° C. for 1 h. After cooling to room temperature, the reaction mixture was concentrated under reduced pressure, the obtained crude was diluted with water (30 mL) and acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was triturated using n-pentane and dried under high vacuum to afford 8-(4-(2-hydroxypropan-2-yl)phenyl)-6-methoxyquinoline-3-carboxylic acid (X-1707A2) (0.600 g, 69%) as a white solid. MS: [MH]⁺338.15.

(R)-N-(1-hydroxypropan-2-yl)-8-(4-(2-hydroxypropan-2-yl) phenyl)-6-methoxyquinoline-3-carboxamide (I′-34). To a stirred solution of 8-(4-(2-hydroxypropan-2-yl)phenyl)-6-methoxyquinoline-3-carboxylic acid (X-1707A2) (0.300 g, 0.89 mmol) in DMF (5 mL) were added DIPEA (0.74 mL, 3.56 mmol) and HATU (0.507 g, 1.33 mmol) at 0° C. under nitrogen. After stirring for 10 min at the same temperature, (R)-2-aminopropan-1-ol (0.200 g, 2.67 mmol) was added, and the resulting reaction mixture was stirred at room temperature for 30 min. The reaction mixture was poured into ice water (30 mL), and the solid product was precipitated, which was collected by filtration and dried under reduced pressure. The resulting crude material was triturated with n-pentane (20 mL×3) and dried over high vacuum to afford (R)-N-(1-hydroxypropan-2-yl)-8-(4-(2-hydroxypropan-2-yl) phenyl)-6-methoxyquinoline-3-carboxamide (I′-34) (0.200 g, 57%) as a white solid. ¹H NMR (400 MHz, DMSO-d₆) δ 9.07-9.06 (d, J=2.4 Hz, 1H), 8.747-8.742 (d, J=2.0 Hz, 1H), 8.46-8.44 (d, J=8.0 Hz, 1H), 7.61-7.55 (m, 4H), 7.47-7.45 (m, 2H), 5.07 (s, 1H), 4.80-4.77 (t, J=5.6 Hz, 1H), 4.12-4.05 (m, 1H), 3.95 (s, 3H), 3.53-3.49 (m, 1H), 3.42-3.37 (m, 1H), 1.50 (s, 6H), 1.19-1.18 (d, J=6.4 Hz, 3H). MS: [MH]⁺395.4.

### Example 1.13. Synthesis of (R)-N-(1-hydroxy-3-methylbutan-2-yl)-6-methoxy-8-(4-(trifluoromethyl)piperidin-1-yl) quinolone-3-carboxamide (I-35)

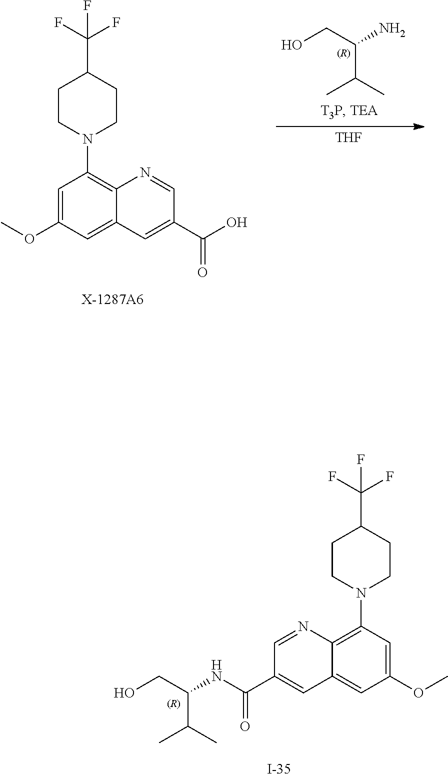

Synthetic procedure of 6-methoxy-8-(4-(trifluoromethyl)piperidin-1-yl)quinoline-3-carboxylic acid (X-1287A6) described in Example 1.1.

To a stirred solution of 6-methoxy-8-(4-(trifluoromethyl)piperidin-1-yl)quinoline-3-carboxylic acid (X-1287A6) (0.380 g, 1.07 mmol) in THF (5.0 mL) were added triethylamine (0.460 g, 3.22 mmol) and propanephosphonic acid anhydride (0.700 g, 2.14 mmol) at 0° C. under nitrogen. After 10 min of stirring at the same temperature, (R)-2-amino-3-methylbutan-1-ol (0.170 g, 1.61 mmol) was added, and the resulting mixture was stirred at room temperature for 2 h. The reaction mixture was diluted with water (100 mL) and was extracted with ethyl acetate (100 mL×3). Combined organic extracts were washed with brine (150 mL), dried over anhydrous Na₂SO₄, and concentrated under reduced pressure. Obtained crude was purified by reverse phase (C-18) silica gel column chromatography using acetonitrile-water=0:1→5:5 as gradient, to afford (R)-N-(1-hydroxy-3-methylbutan-2-yl)-6-methoxy-8-(4-(trifluoromethyl)piperidin-1-yl) quinolone-3-carboxamide (I-35) (0.050 g, 10%) as yellow solid. ¹H NMR (400 MHz, DMSO-d6) δ 9.07-9.06 (d, J=2.4 Hz, 1H), 8.63-8.62 (d, J=2.0 Hz, 1H), 8.27-8.25 (d, J=8.8 Hz, 1H), 7.03-7.02 (d, J=2.8 Hz, 1H), 6.80-6.79 (d, J=2.8 Hz, 1H), 4.64-4.62 (t, J=5.6 Hz, 1H), 4.05-4.02 (d, J=11.6 Hz, 2H), 3.91 (s, 3H), 3.92-3.89 (m, 1H) 3.58-3.51 (m, 2H), 2.80-2.77 (t, J=12.0 Hz, 2H) 2.00-1.98 (m, 3H) 1.89-1.72 (m, 2H) 0.95-0.93 (t, J=7.6 Hz, 6H). (one proton merged with DMSO-d₆ solvent peak). MS: [MH]⁺340.1.

The following compound was prepared in a manner analogous to the procedures described above for (R)-N-(1-hydroxy-3-methylbutan-2-yl)-6-methoxy-8-(4-(trifluoromethyl)piperidin-1-yl) quinolone-3-carboxamide (I-35):

(R)-N-(1-cyclopropyl-2-hydroxyethyl)-6-methoxy-8-(4-methylpiperidin-1-yl)quinoline-3-carboxamide (I-36) (0.025 g, 8%) as off-white solid. ¹H NMR (400 MHz, DMSO-d) δ 9.07-9.06 (d, J=2.0 Hz, 1H), 8.63-8.62 (d, J=2.0 Hz, 1H), 8.45-8.43 (d, J=8.0 Hz, 1H), 7.03-7.02 (d, J=2.4 Hz, 1H), 6.80-6.79 (d, J=2.4 Hz, 1H), 4.73-4.72 (t, J=5.6 Hz, 1H), 4.04-4.01 (d, J=11.6 Hz, 2H), 3.88 (s, 3H), 3.62-3.51 (m, 3H), 2.80-2.74 (t, J=11.2 Hz, 2H) 1.97-1.94 (d, J=10.8 Hz, 2H) 1.79-1.75 (m, 2H) 1.04-1.02 (m, 1H) 0.49-0.47 (m, 1H) 0.39-0.33 (m, 2H) 0.28-0.26 (m, 1H). (one proton merged with DMSO-d₆ solvent peak). MS: [MH]⁺438.1.

### Example 1.14. Synthesis of (S)-6-methoxy-N-(1-(pyridin-2-yl)ethyl)-8-(4-(trifluoromethyl)piperidin-1-yl)quinoline-3-carboxamide (I-37)

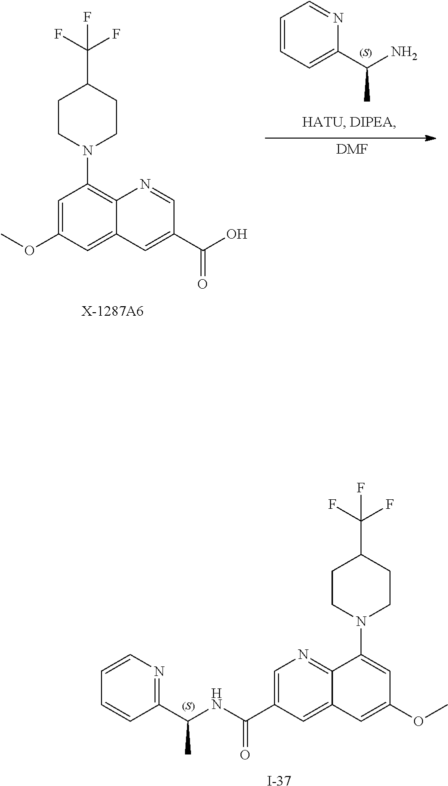

Synthetic procedure of 6-methoxy-8-(4-(trifluoromethyl)piperidin-1-yl)quinoline-3-carboxylic acid (X-1287A6) described in Example 1.1.

To a stirred solution of 6-methoxy-8-(4-(trifluoromethyl)piperidin-1-yl)quinoline-3-carboxylic acid (X-1287A6) (0.250 g, 0.70 mmol) in a DMF (5 mL) were added DIPEA (0.273 g, 2.11 mmol) and HATU (0.402 g, 1.05 mmol) sequentially at 0° C. under nitrogen. After stirring for 10 min at the same temperature, (S)-1-(pyridin-2-yl)ethan-1-amine (0.129 g, 1.05 mmol) was added at room temperature. The reaction mixture was stirred at room temperature for 16 h. The reaction mixture was slowly poured into ice-water (100 mL) and was extracted with ethyl acetate (100 mL×3). The combined organic extracts were washed with brine (100 mL), dried over anhydrous Na₂SO₄, and concentrated in vacuo. The resulting crude was purified by reverse phase (C-18) silica gel column chromatography, using acetonitrile/water=0:1→4:6 as a gradient, to afford (S)-6-methoxy-N-(1-(pyridin-2-yl)ethyl)-8-(4-(trifluoromethyl)piperidin-1-yl)quinoline-3-carboxamide (I-37) (0.200 g, 62%) as an off white solid. ¹H NMR (400 MHz, DMSO-d6) δ 9.15-9.13 (d, J=7.6 Hz, 1H), 9.10-9.10 (d, J=2.0 Hz, 1H), 8.69-8.69 (d, J=2.0 Hz, 1H), 8.54-8.53 (d, J=4.4 Hz, 1H), 7.80-7.75 (m, 1H), 7.48-7.46 (d, J=8.0 Hz, 1H), 7.29-7.26 (m, 1H), 7.04-7.03 (d, J=2.4 Hz, 1H), 6.81-6.80 (d, J=2.4 Hz, 1H), 5.29-5.21 (m, 1H), 4.04-4.01 (d, J=1.2 Hz, 2H), 3.88 (s, 3H), 2.80-2.74 (t, J=11.6 Hz, 2H), 1.96-1.94 (d, J=11.2 Hz, 2H) 1.81-1.73 (m, 2H), 1.55-1.54 (d, J=7.2 Hz, 3H) (one proton merged with DMSO-d6 solvent peak) MS: [MH]⁺459.4.

The following compounds were prepared in a manner analogous to the procedures described above for (S)-6-methoxy-N-(1-(pyridin-2-yl)ethyl)-8-(4-(trifluoromethyl)piperidin-1-yl)quinoline-3-carboxamide (I-37).

N-isopropyl-6-methoxy-8-(4-(trifluoromethyl)piperidin-1-yl)quinoline-3-carboxamide (I-38) (0.060 g, 36%) as a white solid. ¹H NMR (400 MHz, DMSO-d₆) δ 9.05-9.04 (d, J=2.0 Hz, 1H), 8.60-8.59 (d, J=2.0 Hz, 1H), 8.52-8.50 (d, J=7.6 Hz, 1H), 7.03-7.02 (d, J=2.0 Hz, 1H), 6.80-6.79 (d, J=2.4 Hz, 1H), 4.19-4.11 (m, 1H), 4.03-4.0 (d, J=1.2 Hz, 2H), 3.87 (s, 3H), 2.79-2.73 (t, J=11.2 Hz, 2H), 1.96-1.94 (d, J=11.2 Hz, 2H) 1.81-1.73 (m, 2H), 1.22-1.20 (d, J=6.8 Hz, 6H). (one proton merged with DMSO-d₆ solvent peak) MS: [MH]⁺396.0

(S)-6-methoxy-N-(1-(3-methoxyphenyl)ethyl)-8-(4-(trifluoromethyl)piperidin-1-yl)quinoline-3-carboxamide (I-39) (0.080 g, 50%) as a white solid. ¹H NMR (400 MHz, DMSO-d₆) δ 9.09-9.08 (d, J=2.0 Hz, 1H), 9.05 (s, 1H), 8.65-8.64 (d, J=2.0 Hz, 1H), 7.28-7.26 (t, J=4.0 Hz, 1H), 7.05-7.0 (m, 3H), 6.83-6.82 (d, J=2.4 Hz, 1H), 6.80 (s, 1H), 5.22-5.15 (m, 1H), 4.03-4.01 (d, J=11.2 Hz, 2H), 3.87 (s, 3H), 3.75 (s, 3H), 2.79-2.73 (t, J=11.2 Hz, 2H), 1.96-1.93 (d, J=10.8 Hz, 2H), 1.81-1.72 (m, 2H), 1.51-1.49 (d, J=6.8 Hz, 3H) (one proton merged with DMSO-d₆ solvent peak). MS: [MH]⁺488.4.

(R)-N-(1-hydroxy-3,3-dimethylbutan-2-yl)-6-methoxy-8-(4-(trifluoromethyl)piperidin-1-yl)quinoline-3-carboxamide (I-40) (0.120 g, 48%) as a white solid. ¹H NMR (400 MHz, DMSO-d6) δ 9.07-9.06 (d, J=2.4 Hz, 1H), 8.62-8.62 (d, J=2.0 Hz, 1H), 8.18-8.15 (d, J=9.2 Hz, 1H), 7.04-7.03 (d, J=2.4 Hz, 1H), 6.80-6.79 (d, J=2.4 Hz, 1H), 4.52-4.49 (t, J=5.6 Hz, 1H), 4.03-4.00 (m, 2H), 3.98-3.92 (m, 1H), 3.88 (s, 3H), 3.73-3.68 (m, 1H), 3.53-3.49 (m, 1H), 2.79-2.73 (t, J=9.6 Hz, 2H), 1.96-1.94 (d, J=10.8 Hz, 2H) 1.82-1.76 (m, 2H), 0.95 (s, 9H) (one proton merged with DMSO-d₆ solvent peak). MS: [MH]⁺454.5.

### Example 1.15. Synthesis of (6-methoxy-8-(4-(trifluoromethyl)piperidin-1-yl)quinoline-3-carbonyl)-L-alanine (I-41)

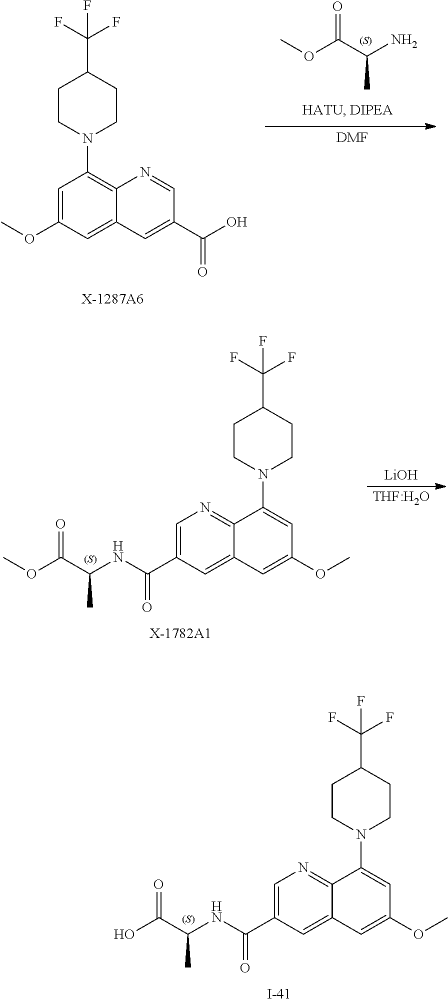

Synthetic procedure of 6-methoxy-8-(4-(trifluoromethyl)piperidin-1-yl)quinoline-3-carboxylic acid (X-1287A6) described in Example 1.1.

(6-methoxy-8-(4-(trifluoromethyl)piperidin-1-yl)quinoline-3-carbonyl)-L-alaninate (X-1782A1) prepared in an analogous manner as (S)-6-methoxy-N-(1-(pyridin-2-yl)ethyl)-8-(4-(trifluoromethyl)piperidin-1-yl)quinoline-3-carboxamide (I-37) described in Example 1.14.

(6-Methoxy-8-(4-(trifluoromethyl)piperidin-1-yl)quinoline-3-carbonyl)-L-alanine (I-41). To a stirred solution of methyl (6-methoxy-8-(4-(trifluoromethyl)piperidin-1-yl)quinoline-3-carbonyl)-L-alaninate (X-1782A1) (0.200 g, 0.45 mmol) in a mixture of THF-water (3:1; 5.0 mL) was added lithium hydroxide monohydrate (0.032 g, 0.77 mmol) at room temperature. The reaction mixture was stirred at 70° C. for 2 h. After cooling to room temperature, the reaction mixture was concentrated under reduced pressure, the obtained crude was acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was dried under reduced pressure, and the resulting crude was purified by (C-18) silica gel column chromatography, using acetonitrile: water=0:1→4:6 as gradient, to afford (6-methoxy-8-(4-(trifluoromethyl)piperidin-1-yl)quinoline-3-carbonyl)-L-alanine (I-41) (0.050 g, 29%) as an off white solid. ¹H NMR (400 MHz, DMSO-d6) δ 12.6 (br. s, 1H), 9.076-9.071 (d, J=2.0 Hz, 1H), 8.97-8.95 (d, J=9.6 Hz, 1H), 8.66-8.65 (d, J=2.0 Hz, 1H), 7.04-7.03 (d, J=2.4 Hz, 1H), 6.82-6.81 (d, J=2.4 Hz, 1H), 4.49-4.45 (t, J=9.2 Hz, 1H), 4.03-4.01 (d, J=10.0 Hz, 2H), 3.88 (s, 3H), 2.80-2.74 (t, J=12.0 Hz, 2H), 1.99-1.96 (m, J=9.6 Hz, 2H), 1.81-1.76 (m, 2H), 1.45-1.43 (s, 3H). (one proton merged with DMSO-d₆ solvent peak) MS: [MH]⁺426.5.

The following compounds were prepared in a manner analogous to the procedures described above for (6-Methoxy-8-(4-(trifluoromethyl)piperidin-1-yl)quinoline-3-carbonyl)-L-alanine (I-41):

(6-Methoxy-8-(4-(trifluoromethyl)piperidin-1-yl)quinoline-3-carbonyl)-D-alanine (I-42) (0.160 g, 55%) as a white solid. ¹H NMR (400 MHz, DMSO-d₆) δ 12.5 (s, 1H), 9.098-9.092 (d, J=2.4 Hz, 1H), 9.01-9.00 (d, J=7.2 Hz, 1H), 8.725-8.721 (d, J=1.6 Hz, 1H), 7.11-7.10 (d, J=2.0 Hz, 1H), 6.91 (s, 1H), 4.50-4.46 (m, 1H), 3.99-3.96 (d, J=10.4 Hz, 2H), 3.89 (s, 3H), 2.86-2.80 (t, J=11.6 Hz, 2H), 1.99-1.91 (m, 2H), 1.86-1.80 (m, 2H), 1.45-1.43 (s, 3H) (one proton merged with DMSO-d₆ solvent peak). MS: [MH]⁺426.5.

(S)-3-(1-(6-methoxy-8-(4-(trifluoromethyl)piperidin-1-yl)quinoline-3-carboxamido)ethyl)benzoic acid (I-43) (0.170 g, 87%) as a white solid. ¹H NMR (400 MHz, DMSO-d₆) δ 13.04-12.97 (br. s, 1H), 9.20-9.18 (d, J=7.6 Hz, 1H), 9.09-9.08 (d, J=2.0 Hz, 1H), 8.65-8.64 (d, J=2.0 Hz, 1H), 8.03 (s, 1H), 7.84-7.82 (d, J=8.0 Hz, 1H), 7.69-7.67 (d, J=7.6 Hz, 1H), 7.49-7.09 (t, J=7.6 Hz, 1H), 7.04-7.03 (d, J=2.4 Hz, 1H), 6.806-6.800 (d, J=2.4 Hz, 1H), 5.28-5.25 (m, 1H), 4.04-4.00 (t, J=9.2 Hz, 2H), 3.87 (s, 3H), 2.78-2.74 (t, J=10.0 Hz, 2H), 1.96-1.93 (d, J=11.2 Hz, 2H), 1.78-1.75 (d, J=9.6 Hz, 2H), 1.55-1.53 (d, J=6.8 Hz, 3H) (one proton merged with DMSO-d₆ solvent peak). MS: [MH]⁺502.4.

### Example 1.16. Synthesis of N-(1-hydroxypropan-2-yl)-5-((4-(trifluoromethyl)benzyl)oxy)-2-naphthamide (I-44

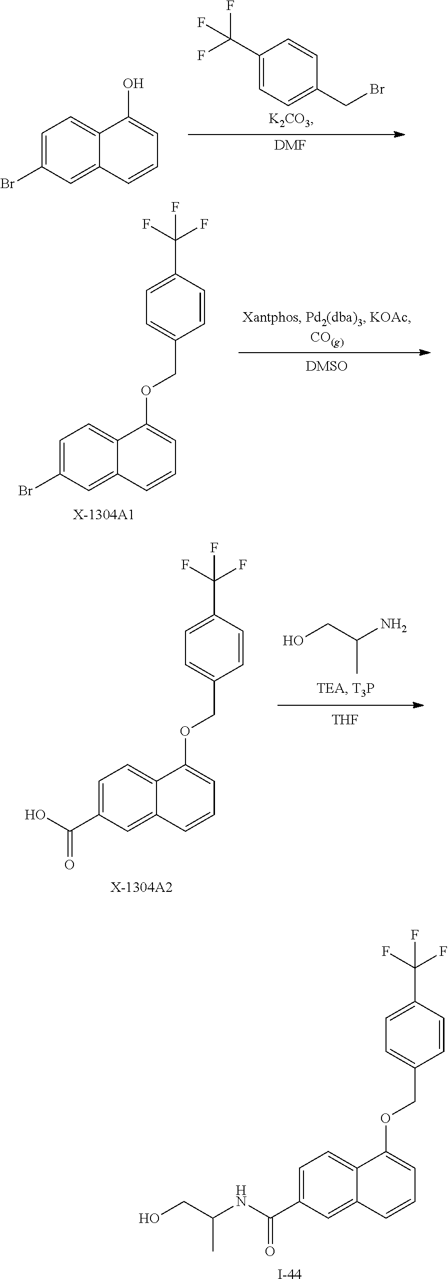

This compound was prepared in a manner analogous to the procedures described in Example 1.3.

N-(1-hydroxypropan-2-yl)-5-((4-(trifluoromethyl)benzyl)oxy)-2-naphthamide (I-44) (0.250 g, 12%) as an off white solid. ¹H NMR (400 MHz, DMSO-d₆) δ 8.41 (s, 1H), 8.30-8.28 (d, J=8.4 Hz, 1H), 8.27-8.25 (d, J=8.8 Hz, 1H), 7.95-7.93 (d, J=8.8 Hz, 1H), 7.82-7.78 (m, 4H), 7.61-7.59 (d, J=8.0 Hz, 1H), 7.51-7.47 (t, J=7.6 Hz, 1H), 7.16-7.14 (d, J=7.6 Hz, 1H), 5.45 (s, 2H), 4.78-4.75 (t, J=5.6 Hz, 1H), 4.08-4.05 (m, 1H), 3.51-3.49 (m, 1H), 3.40-3.33 (m, 1H), 1.17-1.16 (d, J=6.8 Hz, 3H). MS: [MH+] 404.2.

### Example 1.17. Synthesis of 5-((4,4-difluorocyclohexyl)methoxy)-N-(1-(pyridin-2-yl)ethyl)-2-naphthamide (I-45)

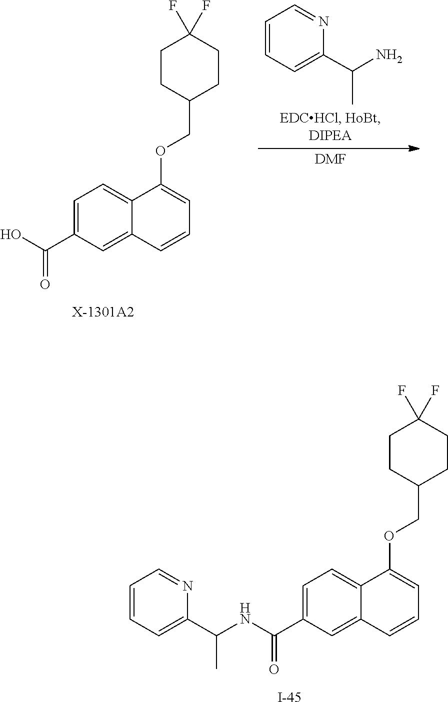

Synthetic procedure of 5-((4,4-difluorocyclohexyl)methoxy)-2-naphthoic acid (X-1301 A2) described in Example 1.3.

To a solution of 5-((4,4-difluorocyclohexyl)methoxy)-2-naphthoic acid (X-1301A2) (0.120 g, 0.37 mmol) in DMF (8 mL), were added DIPEA (0.145 g, 1.12 mmol), EDC.HCl (0.123 g, 0.64 mmol), and HOBt (0.097 g, 0.64 mmol) at 0° C. The mixture was stirred at 0° C. for 10 min. 1-(pyridin-2-yl)ethan-1-amine (0.055 g, 0.45 mmol) was added at 0° C., and the reaction mixture was stirred at room temperature for 1.5 h. The mixture was diluted with water (30 mL) and was extracted with ethyl acetate (40 mL×3). The combined organic layers were dried over Na₂SO₄, filtered, and concentrated under reduced pressure. The crude product was purified by (C-18) silica gel column chromatography, using acetonitrile-water=0:1→6:4 as a gradient, to afford 5-((4,4-difluorocyclohexyl)methoxy)-N-(1-(pyridin-2-yl)ethyl)-2-naphthamide (I-45) (0.110 g, 69%) as a white solid. ¹H NMR (400 MHz, DMSO-d₆) δ 9.02-9.00 (d, J=7.6 Hz, 1H), 8.53-8.52 (d, J=4.0 Hz, 1H), 8.48 (s, 1H), 8.21-8.19 (d, J=8.4 Hz, 1H), 7.96-7.94 (d, J=8.4 Hz, 1H), 7.78-7.75 (t, J=7.2 Hz, 1H), 7.59-7.57 (d, J=8.0 Hz, 1H), 7.51-7.44 (m, 2H), 7.27-7.24 (t, J=5.6 Hz, 1H), 7.07-7.05 (d, J=7.6 Hz, 1H), 5.26-5.24 (t, J=7.2 Hz, 1H), 4.07-4.06 (d, J=6.0 Hz, 2H), 2.08-1.82 (m, 7H), 1.55-1.53 (d, J=6.8 Hz, 3H), 1.48-1.45 (m, 2H). MS: [MH]⁺425.2

### Example 1.18. Synthesis of 8-((4,4-Difluorocyclohexyl)methoxy)-N-(1-(pyridin-2-yl)ethyl)quinoline-3-carboxamide (I-46)

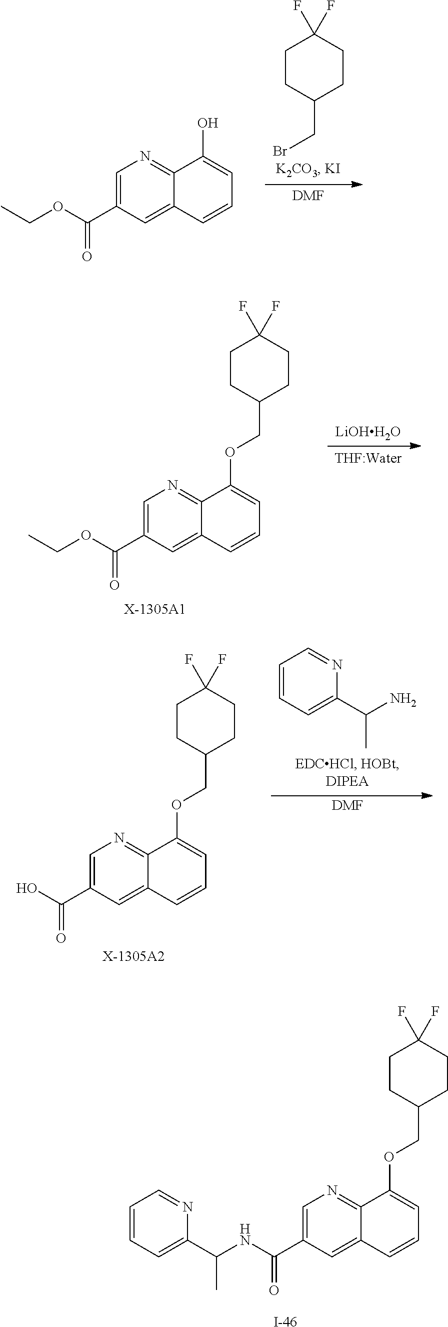

Ethyl 8-((4,4-difluorocyclohexyl)methoxy)quinoline-3-carboxylate (X-1305A1). To a stirred solution of ethyl 8-hydroxyquinoline-3-carboxylate (0.600 g, 2.76 mmol) in DMF (5 mL) were added 4-(bromomethyl)-1,1-difluorocyclohexane (0.706 g, 3.316 mmol), anhydrous K₂CO₃ (1.144 g, 8.292 mmol), and KI (0.045 g, 0.276 mmol) sequentially at room temperature under nitrogen, and the resulting mixture was heated at 100° C. for 16 h. The reaction mixture was cooled to room temperature, quenched with water (100 mL) and was extracted with ethyl acetate (75 mL×3). Collected organics were washed with brine (150 mL), dried over anhydrous Na₂SO₄, and concentrated in vacuo. The resulting crude was purified by silica gel column chromatography, using ethyl acetate-hexane=0:1→1:9 as gradient, to afford ethyl 8-((4,4-difluorocyclohexyl)methoxy)quinoline-3-carboxylate (X-1305A1) (0.265 g, 40%) as white solid. MS: [MH]⁺350.0.

8-((4,4-Difluorocyclohexyl)methoxy)quinoline-3-carboxylic acid (X-1305A2).

To a stirred solution of 8-((4,4-difluorocyclohexyl)methoxy)quinoline-3-carboxylate (X-1305A1) (0.265 g, 0.759 mmol) in a mixture of THF-water (8:2; 3.0 mL) was added lithium hydroxide monohydrate (0.095 g, 2.277 mmol) at room temperature, and the resulting mixture was stirred at 70° C. for 1 h. After cooling to room temperature, the reaction mixture was concentrated under reduced pressure, and the obtained crude was diluted with water (40 mL) and was extracted with ethyl acetate (40 mL×2) to remove unwanted organic impurities. The aqueous part was acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was dried under high vacuum to afford 8-((4,4-difluorocyclohexyl)methoxy)quinoline-3-carboxylic acid (X-1305A2) (0.240 g, quantitative) as an off white solid. MS: [MH]⁺322.5.

8-((4,4-Difluorocyclohexyl)methoxy)-N-(1-(pyridin-2-yl)ethyl)quinoline-3-carboxamide (I-46). To a stirred solution of 8-((4,4-difluorocyclohexyl)methoxy)quinoline-3-carboxylic acid (X-1303A2) (0.110 g, 0.342 mmol) in DMF (2 mL) were added diisopropylethylamine (0.132 g, 1.027 mmol), EDC.HCl (0.078 g, 0.513 mmol), HOBT (0.077 g, 0.513 mmol), and 1-(pyridin-2-yl)ethan-1-amine (0.050 g, 0.411 mmol), sequentially at 0° C. under nitrogen, and the resulting mixture was stirred at 50° C. for 45 min. The reaction mixture was cooled to room temperature, quenched with water (100 mL), and extracted with ethyl acetate (75 mL×3). Collected organics were washed with brine (150 mL), dried over anhydrous Na₂SO₄, and concentrated in vacuo. The resulting crude was purified by silica gel column chromatography using methanol-dichoromethane 0:1→0.5:9.5 as gradient to afford 8-((4,4-difluorocyclohexyl)methoxy)-N-(1-(pyridin-2-yl)ethyl)quinoline-3-carboxamide (I-46) (0.110 g, 75%) as a white solid. ¹H NMR (400 MHz, DMSO-d6) δ 9.24-9.23 (d, J=2.4 Hz, 1H), 9.20-9.17 (d, J=7.6 Hz, 1H), 8.84-8.83 (d, J=2.4 Hz, 1H), 8.54-8.53 (d, J=4.4 Hz, 1H), 7.79-7.75 (dt, J=8.0 Hz, 2.0 Hz, 1H), 7.62-7.55 (m, 2H), 7.48-7.46 (d, J=8.0 Hz, 1H), 7.31-7.25 (m, 2H), 5.27-5.23 (m, 1H), 4.08-4.06 (d, J=6.0 Hz, 2H), 2.07-1.93 (m, 5H), 1.89-1.82 (m, 2H) 1.56-1.54 (d, J=6.8 Hz, 3H) 1.45-1.35 (m, 2H). MS: [MH]⁺426.8.

### Example 1.18. Synthesis of 8-((4,4-difluorocyclohexyl)methoxy)-N-(1-hydroxypropan-2-yl)quinoline-3-carboxamide (I-47)

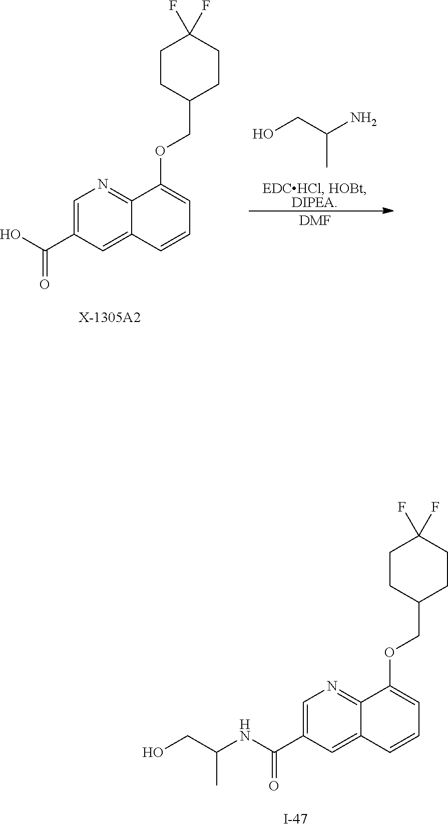

The synthetic procedure of 8-((4,4-difluorocyclohexyl)methoxy)quinoline-3-carboxylic acid (X-1305A2) described in Example 1.17.

To a stirred solution of 8-((4,4-difluorocyclohexyl)methoxy)quinoline-3-carboxylic acid (X-1305A2) (0.220 g, 0.685 mmol) in DMF (4 mL) was added diisopropylethylamine (0.265 g, 2.055 mmol), EDC.HCl (0.155 g, 1.027 mmol), HOBT (0.157 g, 1.027 mmol), and 2-aminopropan-1-ol (0.067 g, 0.890 mmol), sequentially at 0° C. under nitrogen, and the resulting mixture was heated at 50° C. for 1 h. The reaction mixture was cooled to room temperature, quenched with water (100 mL), and extracted with ethyl acetate (75 mL×3). Collected organics were washed with brine (150 mL), dried over anhydrous Na₂SO₄, and concentrated in vacuo. The resulting crude was purified by silica gel column chromatography using methanol-dichloromethane→0:1→0.5:9.5 as gradient to afford 8-((4,4-difluorocyclohexyl)methoxy)-N-(1-hydroxypropan-2-yl)quinoline-3-carboxamide (I-47) (0.130 g, 52%) as a white solid. ¹H NMR (400 MHz, DMSO-d6) δ 9.207-9.202 (d, J=2.0 Hz, 1H), 8.74-8.73 (d, J=2.0 Hz, 1H), 8.47-8.45 (d, J=8.0 Hz, 1H), 7.59-7.54 (m, 2H), 7.29-7.27 (dd, J=2.8 Hz, J=6.8 Hz, 1H), 4.80-4.78 (t, J=5.6 Hz, 1H), 4.09-4.06 (m, 3H), 3.51-3.49 (m, 1H), 3.41-3.34 (m, 1H), 2.07-1.98 (m, 5H), 1.93-1.85 (m, 2H), 1.41-1.38 (m, 2H) 1.18-1.16 (m, J=6.8 Hz, 3H). MS: [MH⁺] 379.6.

### Example 1.19. Synthesis of (R)-N-(1-hydroxypropan-2-yl)-3-methyl-1-(4-(trifluoromethyl)piperidin-1-yl)isoquinoline-6-carboxamide (I-48)

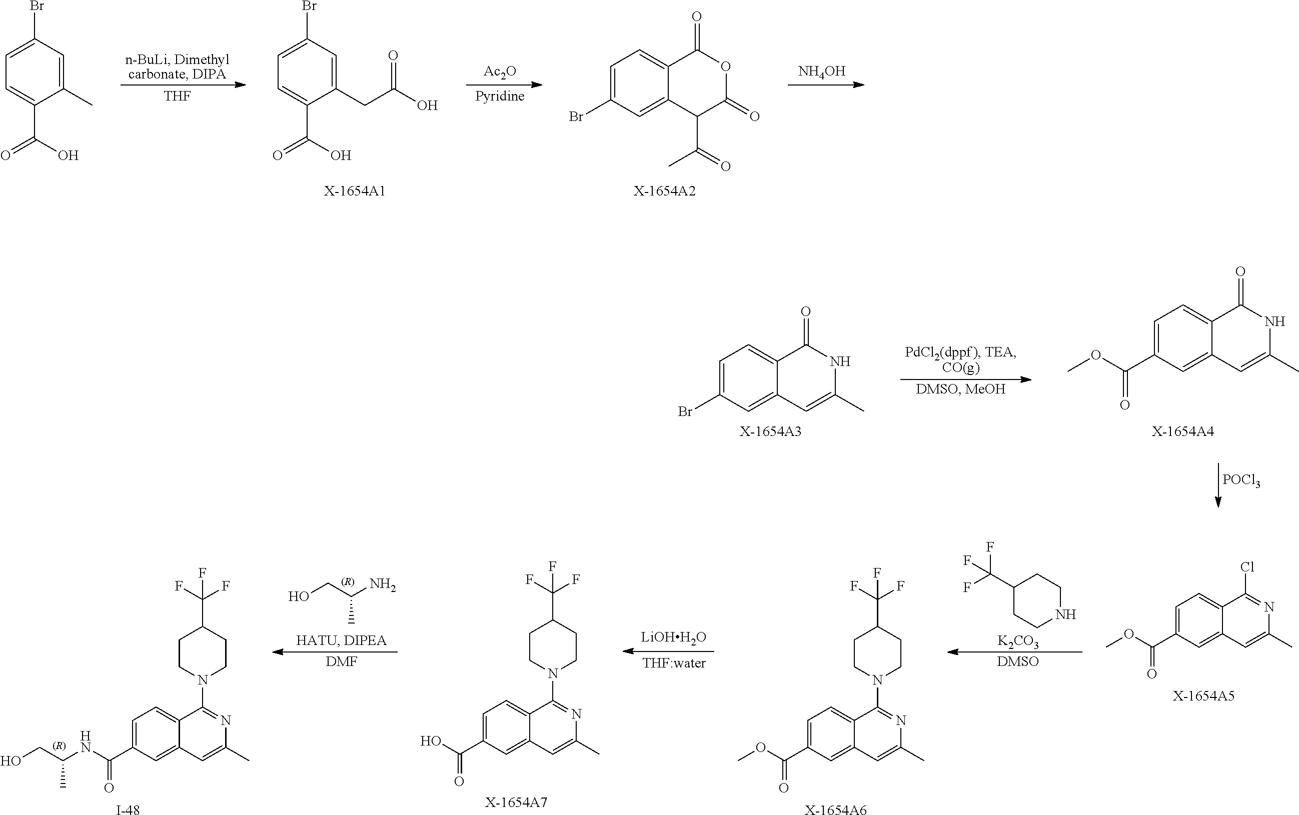

4-Bromo-2-(carboxymethyl)benzoic acid (X-1654A1). To a stirred solution of DIPA (28.1 gm g, 279.06 mmol) in THF (100 mL) was added n-BuLi (1.6 M in hexane 17.4 ml, 279.06 mmol) at −78° C. temperature (in first round bottom flask). In second round bottom flask, to 4-bromo-2-methylbenzoic acid (15.0 g, 69.76 mmol) in THF (75 mL) was added dimethyl carbonate (12.5 g, 139.53 mmol) at room temperature, and the resulting mixture was added in first round bottom flask in dropwise manner at −78° C. The reaction mixture was stirred at room temperature for 2 h. The reaction mixture was quenched with water and stirred for overnight, and the resulting mixture form a homogenous solution. The aqueous layer was separated and acidify by HCl, and the product was extracted by ethyl acetate. The combined organic extracts were dried over anhydrous Na₂SO₄ and concentrated under reduced pressure to afford 4-bromo-2-(carboxymethyl)benzoic acid (X-1654A1) (16 g, 89%) as a white solid. MS: [MH]⁺258.8.

4-Acetyl-6-bromoisochromane-1,3-dione (X-1654A2). To a stirred solution of 4-bromo-2-(carboxymethyl)benzoic acid (X-1654A1) (6.0 g, 23.25 mmol) in acetic anhydride (25 mL) was added pyridine (8 mL) at 0° C. temperature under nitrogen, and the reaction mixture was stirred at room temperature for 30 min. The reaction mixture was slowly poured in water (100 mL), the obtained precipitates were filtered, and the residue was washed with water (100 mL). Solid precipitate was dried under reduced pressure to afford 4-acetyl-6-bromoisochromane-1,3-dione (X-1654A2) (4.5 g, 69%; crude) as a brown solid, which was carried to the next step without purification.

6-Bromo-3-methylisoquinolin-1(2H)-one (X-1654A3). 4-acetyl-6-bromoisochromane-1,3-dione (X-1654A2) (3.50 g, 12.4 mmol) and NH₄OH (20 ml) were stirred in a sealed tube, and the resulting mixture was heated at 100° C. for 1 h. The reaction mixture was cooled to room temperature and was slowly poured into water (500 mL), and the resulting precipitate was collected by filtration, washed with water (500 mL), and dried under reduced pressure to afford 6-bromo-3-methylisoquinolin-1(2H)-one (X-1654A3) (1.50 g, 51%) as a white solid. MS: [MH]⁺237.8.

Methyl 3-methyl-1-oxo-1,2-dihydroisoquinoline-6-carboxylate (X-1654A4).

To a stirred solution of 6-bromo-3-methylisoquinolin-1(2H)-one (X-1654A3) (0.500 g, 2.10 mmol) in MeOH (6.0 mL) was added TEA (0.639 g, 6.32 mmol). The reaction mixture was degassed (by purging nitrogen) for 30 min followed by the addition of PdCl₂(dppf) (0.108 g, 0.147 mmol). The reaction mixture was purged with CO (g) for 30 min and the reaction mixture stirred for 16 h at 70° C. temperature. The reaction mixture was cooled to room temperature, slowly poured into water (50 mL), and extracted with ethyl acetate (50 mL×3). The combined organic extracts were dried over anhydrous Na₂SO₄ and concentrated under reduced pressure to afford methyl 3-methyl-1-oxo-1,2-dihydroisoquinoline-6-carboxylate (X-1654A4) (0.350 g, 76%) as a solid. Crude was use in next step without further purifications. MS: [MH]⁺217.8

Methyl 1-chloro-3-methylisoquinoline-6-carboxylate (X-1654A5). A solution of methyl 3-methyl-1-oxo-1,2-dihydroisoquinoline-6-carboxylate (X-1654A4) (0.350 g, 1.612 mmol) in POCl₃ (2 mL) was heated for 1 h at 120° C. The reaction mixture was cooled to room temperature, slowly poured into water (50 mL), and extracted with ethyl acetate (50 mL×3). The combined organic extracts were dried over anhydrous Na₂SO₄ and concentrated under reduced pressure. The crude product was purified by silica gel column chromatography, ethyl acetate-hexane=0:1→1:9 as gradient, to afford methyl 1-chloro-3-methylisoquinoline-6-carboxylate (X-1654A5) (0.090 g, 24%) as a white solid.

Methyl 3-methyl-1-(4-(trifluoromethyl)piperidin-1-yl)isoquinoline-6-carboxylate (X-1654A6). To a stirred solution of methyl 1-chloro-3-methylisoquinoline-6-carboxylate (X-1654A5) (0.090 g, 0.382 mmol) in DMSO (1.5 mL) was added 4-(trifluoromethyl)piperidine (0.117 g, 0.765 mmol) and K₂CO₃ (0.080 g, 0.574 mmol) at room temperature, and the resulting mixture was heated at 120° C. for 16 h. After cooling the reaction mixture to room temperature, the reaction mixture was slowly poured into water (50 mL), and the resulting precipitate was collected by filtration. Obtained solid residue was washed with water (50×2 mL) and dried under high vacuum to afford methyl 3-methyl-1-(4-(trifluoromethyl)piperidin-1-yl)isoquinoline-6-carboxylate (X-1654A6) (0.080 g, 59%) as a brown solid. MS: [MH]⁺352.8.

3-Methyl-1-(4-(trifluoromethyl)piperidin-1-yl)isoquinoline-6-carboxylic acid (X-1654A7). To a stirred solution of methyl 3-methyl-1-(4-(trifluoromethyl)piperidin-1-yl)isoquinoline-6-carboxylate (X-1654A6) (0.200 g, 0.568 mmol) in a mixture of THF: water (3:1; 3.0 mL) was added lithium hydroxide monohydrate (71.6 g, 1.704 nmol) at room temperature, and the resulting mixture was heated at 70° C. for 1 h. After cooling to room temperature, the reaction mixture was concentrated under reduced pressure. The obtained crude was diluted with water (40 mL) and was extracted with ethyl acetate (40 mL×2) to remove unwanted organic impurities. The aqueous part was acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was dried under high vacuum to afford 3-methyl-1-(4-(trifluoromethyl)piperidin-1-yl)isoquinoline-6-carboxylic acid (X-1654A7) (0.180 g, 94%) as a white solid. MS: [MH]⁺338.

(R)-N-(1-hydroxypropan-2-yl)-3-methyl-1-(4-(trifluoromethyl)piperidin-1-yl)isoquinoline-6-carboxamide (I-48). To a stirred solution of 3-methyl-1-(4-(trifluoromethyl)piperidin-1-yl)isoquinoline-6-carboxylic acid (X-1654A7) (0.100 g, 0.295 mmol) in DMF (3 mL) were added DIPEA (0.114 g, 0.887 mmol) and HATU (0.225 g, 0.591 mmol) sequentially at 0° C. The resulting reaction mixture was stirred for 15 min, and (R)-2-aminopropan-1-ol (0.111 g, 1.47 mmol) was added under nitrogen and stirred for 1 h at room temperature. The reaction mixture was diluted with water (50 mL), and the resulting precipitate was collected by filtration. Obtained solid residue was washed with water (50×2 mL) and dried under high vacuum to afford (R)-N-(1-hydroxypropan-2-yl)-3-methyl-1-(4-(trifluoromethyl)piperidin-1-yl)isoquinoline-6-carboxamide (I-48) (0.100 g, 86%) as a white solid. ¹H-NMR (400 MHz, DMSO-d6) δ 8.34-8.32 (d, J=7.6 Hz, 1H), 8.24 (s, 1H), 8.05-8.03 (d, J=8.8 Hz, 1H), 7.89-7.87 (d, J=8.4 Hz, 1H), 7.27 (s, 1H), 4.77-4.74 (t, J=6.0 Hz, 1H), 4.07-4.04 (m, 1H), 3.84-3.81 (d, J=12.4 Hz, 2H), 3.51-3.48 (m, 1H), 3.41-3.48 (m, 1H), 2.97-2.91 (t, J=12.0 Hz, 2H), 2.59-2.55 (m, 1H; merged in DMSO-d₆), 1.98-1.95 (d, J=11.2 Hz, 2H), 1.85-1.79 (m, 2H), 1.77-1.16 (d, J=6.8 Hz, 3H). (one aromatic —CH₃ protons merged with DMSO-d₆ solvent peak that appeared at 2.55 ppm in CD₃OD). MS: [MH]⁺395.8.

### Example 1.20. Synthesis of (R)-N-(1-hydroxypropan-2-yl)-3-methoxy-1-(4-(trifluoromethyl)piperidin-1-yl)isoquinoline-6-carboxamide (I-49)

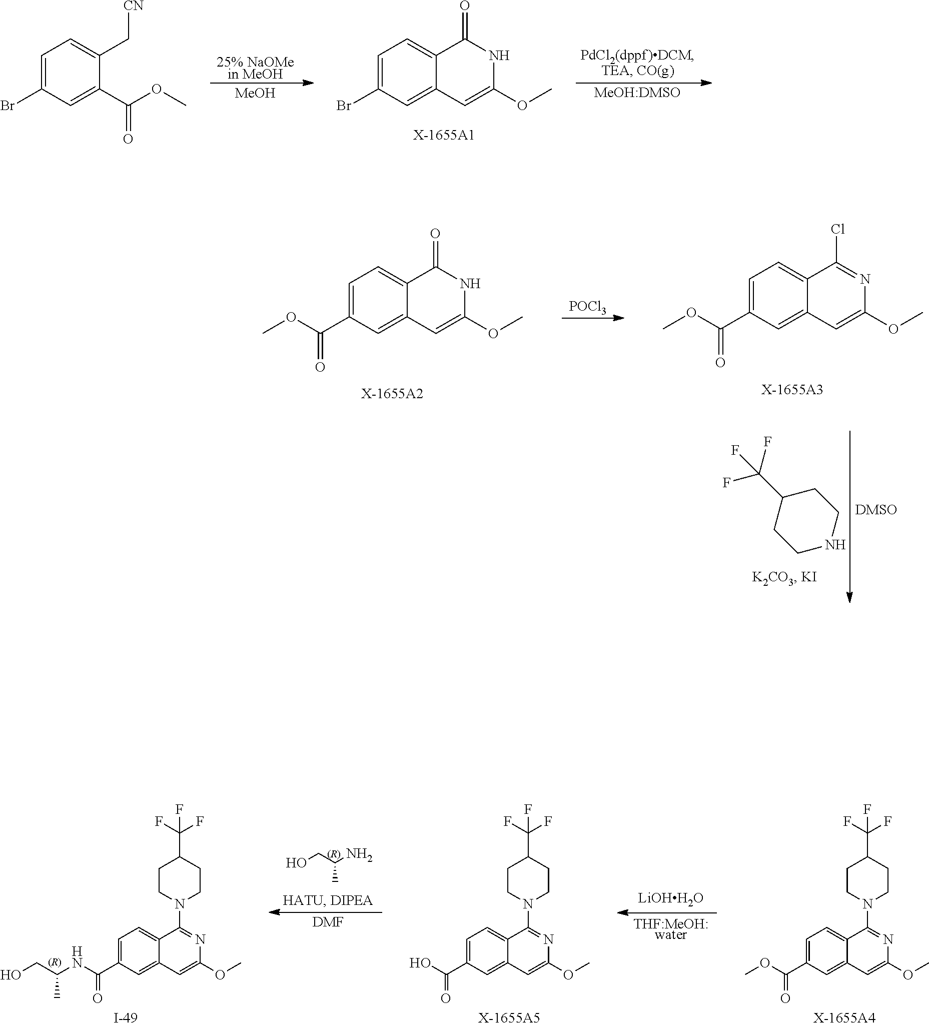

6-Bromo-3-methoxyisoquinolin-1(2H)-one (X-1655A1). To a stirred methyl 4-bromo-2-(cyanomethyl)benzoate (2.0 g, 7.90 mmol) in MeOH (25 mL) was added 25% NaOMe in MeOH (4.17 mL), and the resulting reaction mixture was heated at 80° C. for 2 h. After cooling to room temperature, the reaction mixture was concentrated under reduced pressure. Crude was diluted with water and acidified (pH ˜3-4) with 1 N HCl. The resulting precipitate was collected by filtration. Obtained solid residue was washed with water (100×3 mL) and dried under high vacuum to afford 6-bromo-3-methoxyisoquinolin-1(2H)-one (X-1655A1) (1.80 g, 90%) as a white solid, which was taken to next step without further purification. MS: [MH]⁺253.7/[MH+2]⁺ 255.7.

Methyl 3-methoxy-1-oxo-1,2-dihydroisoquinoline-6-carboxylate (X-1655A2).

To a stirred solution of 6-bromo-3-methoxyisoquinolin-1(2H)-one (X-1655A1) (1.30 g, 5.13 mmol) in MeOH (25 mL) was added trimethylamine (2.07 g, 20.5 mmol), and the reaction mixture was degassed (purging with nitrogen) for 10 min followed by addition of PdCl₂(dppf). DCM (0.419 g, 0.51 mmol) sequentially at room temperature under nitrogen then resulting mixture was stirred at 80° C. under 150 psi CO gas pressure for 16 h. The reaction mixture combined with an identically prepared 1 more batch of 0.5 g, combined crude was filtered and washed with EtOAc (3×50 mL), and the solid product was dried over high vacuum to afford methyl 3-methoxy-1-oxo-1,2-dihydroisoquinoline-6-carboxylate (X-1656A2) (0.650 g, 40%) as an off-white solid. MS: [MH]⁺233.90.

Methyl 1-chloro-3-methoxyisoquinoline-6-carboxylate (X-1655A3). A solution of methyl 3-methoxy-1-oxo-1,2-dihydroisoquinoline-6-carboxylate (X-1655A2) (0.650 g, 2.78 mmol) in POCl₃ (6 mL) was heated at 90° C. for 2 h. After completion of the reaction, the reaction mixture was slowly poured into ice-water (80 mL), and the resulting precipitate was collected by filtration. Obtained solid residue was washed with water (100×3 mL) and dried under high vacuum to afford methyl 1-chloro-3-methoxyisoquinoline-6-carboxylate (X-1655A3) (0.48 g, 69%) as a yellow solid. MS: [MH]⁺251.80/[MH+2]⁺253.7.

Methyl 3-methoxy-1-(4-(trifluoromethyl)piperidin-1-yl)isoquinoline-6-carboxylate (X-1655A4). To a stirred solution of methyl 1-chloro-3-methoxyisoquinoline-6-carboxylate (X-1655A4) (0.480 g, 1.91 mmol) in DMSO (6 mL) were added 4-(trifluoromethyl)piperidine (0.585 g, 3.82 mmol), K₂CO₃ (0.791 g, 5.73 mmol), and potassium iodide (0.063 g, 0.382 mmol) sequentially at room temperature under nitrogen, and the resulting reaction mixture was heated at 120° C. for 1 h. The reaction mixture was diluted with water (40 mL) and extracted with ethyl acetate (80 mL×2). Organic extracts were combined, dried over anhydrous Na₂SO₄, and concentrated under reduced pressure to afford methyl 3-methoxy-1-(4-(trifluoromethyl)piperidin-1-yl)isoquinoline-6-carboxylate (X-1655A4) (0.440 g, 63%) as a yellow solid, which was taken to next step without further purification. MS: [MH]⁺369.0.

3-Methoxy-1-(4-(trifluoromethyl)piperidin-1-yl)isoquinoline-6-carboxylic acid (X-1655A5). To a stirred solution of methyl 3-methoxy-1-(4-(trifluoromethyl)piperidin-1-yl)isoquinoline-6-carboxylate (X-1655A4) (0.440 g. 11.9 mmol) in a mixture of THF: MeOH: water (3:2:1; 8.0 mL) was added lithium hydroxide monohydrate (0.100 g, 2.39 mmol) at room temperature, and the resulting mixture was heated at 70° C. for 1 h. After cooling to room temperature, the reaction mixture was concentrated under reduced pressure, and the obtained crude was diluted with water (40 mL) and extracted with ethyl acetate (40 mL×2) to remove unwanted organic impurities. The aqueous part was acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was dried under high vacuum to afford 3-methoxy-1-(4-(trifluoromethyl)piperidin-1-yl)isoquinoline-6-carboxylic acid (X-1655A5) (0.27 g, 64%) as a white solid, which was taken to next step without further purification. MS: [MH]⁺355.00.

(R)-N-(1-hydroxypropan-2-yl)-3-methoxy-1-(4-(trifluoromethyl)piperidin-1-yl)isoquinoline-6-carboxamide (I-49). To a stirred solution of 3-methoxy-1-(4-(trifluoromethyl)piperidin-1-yl)isoquinoline-6-carboxylic acid (X-1655A5) (0.25 g, 0.70 mmol) in DMF (5 mL) were added DIPEA (0.364 g, 2.82 mmol) and HATU (0.402 g, 1.05 mmol) sequentially at 0° C. under nitrogen. After stirring for 10 min at the same temperature, (R)-2-aminopropan-1-ol (0.161 g, 2.14 mmol) in DMF (1 mL) was added and stirring was continued at room temperature for 2 h. The reaction mixture was diluted with water (20 mL) and extracted with ethyl acetate (60×2 mL). Combined organic extracts were washed with brine (100 ml), dried over anhydrous Na₂SO₄, and concentrated under reduced pressure. The resulting crude was purified by reverse phase (C-18) silica gel column chromatography, using acetonitrile-water=3:7→4:6 as gradient to afford (R)-N-(1-hydroxypropan-2-yl)-3-methoxy-1-(4-(trifluoromethyl)piperidin-1-yl)isoquinoline-6-carboxamide (I-49) (0.110 g, 38%) as a yellow solid. ¹H NMR (400 MHz, DMSO-d₆) δ 8.30-8.28 (d, J=8.0 Hz, 1H), 8.19 (s, 1H). 7.99-7.97 (d, J=8.8 Hz, 1H), 7.71-7.70 (dd, J=8.8 Hz, 1.20 Hz, 1H), 6.75 (s, 1H) 4.76-4.73 (t, J=6.0 Hz, 1H), 4.10-4.00 (m, 1H), 3.94-3.90 (m, 2H), 3.90 (s, 3H), 3.52-3.46 (m, 1H), 3.40-3.35 (m. 1H), 3.04-2.98 (t, J=12.4 Hz, 2H), 2.68-2.61 (s, 1H), L97-1.94 (d, J=10.8 Hz, 2H), 1.84-1.76 (m, 2H), 1.17-1.15 (d, J=6.8 Hz, 3H). MS: [MH]⁺ 412.4.

The following compounds were prepared in a manner analogous to the procedures described above for (R)-N-(1-hydroxypropan-2-yl)-3-methoxy-1-(4-(trifluoromethyl)piperidin-1-yl)isoquinoline-6-carboxamide (I-49):

(S)-3-Methoxy-N-(1-methoxypropan-2-yl)-1-(4-(trifluoromethyl)piperidin-1-yl)isoquinoline-6-carboxamide (I-50) (0.140 g, 73%) as white solid. ¹H NMR (400 MHz, DMSO-d₆) δ 8.44-8.42 (d, J=8.0 Hz, 1H), 8.178-8.175 (d, J=1.2 Hz, 1H), 7.99-7.97 (d, J=8.8 Hz, 1H), 7.70-7.67 (dd, J=8.8, 1.6 Hz, 1H), 6.75 (s, 1H), 4.26-4.19 (m, 1H), 3.93-3.89 (m, 2H), 3.89 (s, 3H), 3.45-3.41 (m, 1H), 3.34-3.29 (m, 1H), 3.28 (s, 3H), 3.04-2.98 (t, J=12.0 Hz, 2H), 2.65-2.58 (m, 1H), 1.96-1.94 (d, J=10.8 Hz, 2H), 1.84-1.75 (m, 2H), 1.17-1.15 (d, J=6.8 Hz, 3H). MS: [MH]⁺426.4.

(S)-N-(1-(Pyridin-2-yl)ethyl)-1-(4-(trifluoromethyl)piperidin-1-yl)isoquinoline-6-carboxamide (I-51) (0.700 g, 91%) as off white solid. ¹H NMR (400 MHz, DMSO-d4) δ 9.12-9.10 (d, J=7.6 Hz, 1H), 8.54-8.53 (m, 1H), 8.455-8.452 (d, J=1.2 Hz, 1H), 8.18-8.17 (d, J=5.6 Hz, 1H), 8.14-8.12 (d, J=8.8 Hz, 1H), 8.03-8.00 (dd, J=8.8, 1.6 Hz, 1H), 7.80-7.75 (m, 1H), 7.50-7.44 (m, 2H), 7.29-7.25 (m, 1H), 5.26-5.23 (m, 1H), 3.86-3.83 (d, J=12.4 Hz, 2H), 3.00-2.93 (t, J=12.0 Hz, 2H), 1.98-1.95 (d, J=10.4 Hz, 2H), 1.86-1.80 (m, 2H), 1.55-1.54 (d, J=6.8 Hz, 3H). (1H merged with DMSO-d₆ moisture) MS: [MH]⁺429.4.

### Example 1.21. Synthesis of (R)-N-(1-hydroxypropan-2-yl)-3-(trifluoromethyl)-1-(4-(trifluoromethyl)piperidin-1-yl)isoquinoline-6-carboxamide (I-52)

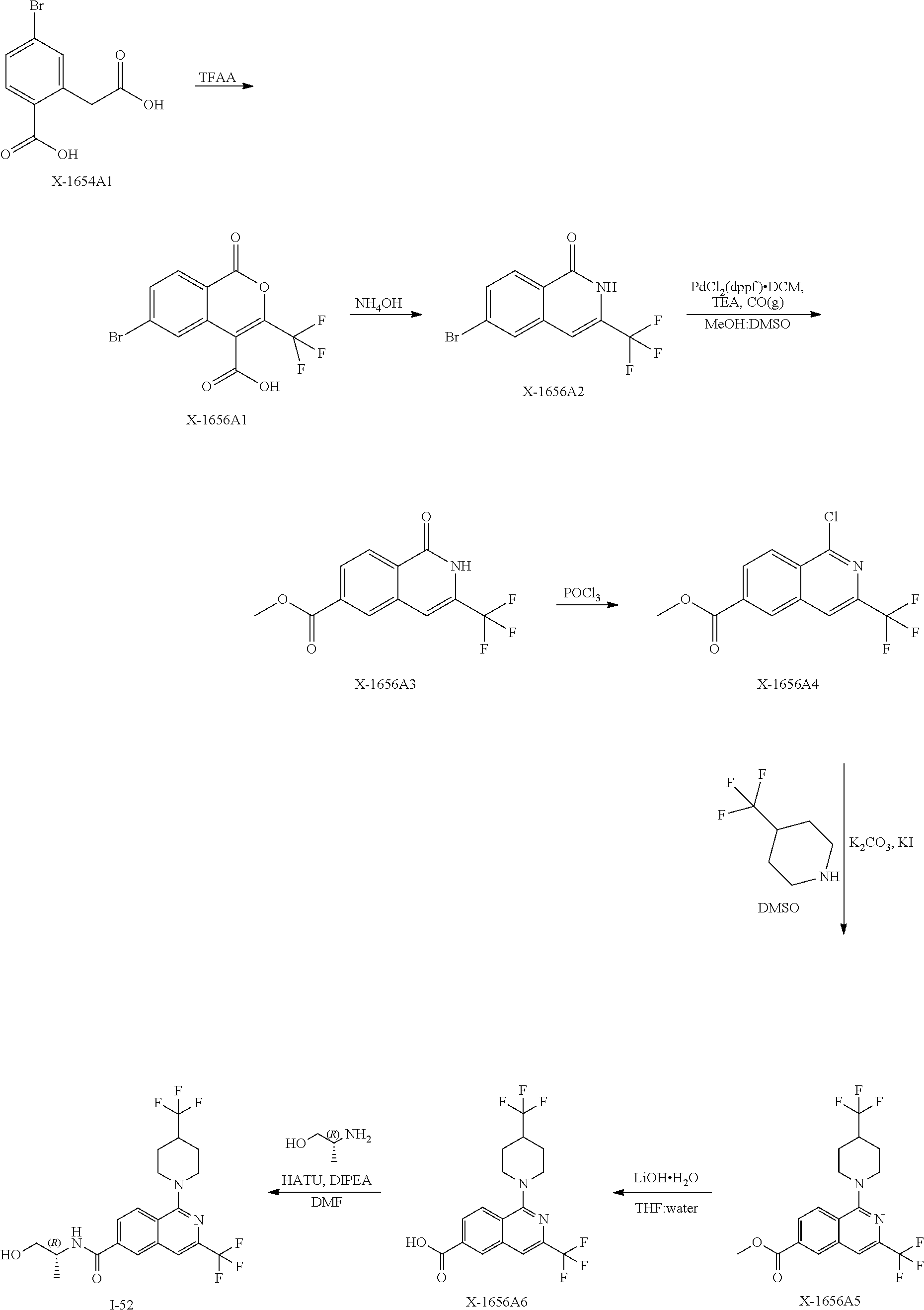

6-Bromo-1-oxo-3-(trifluoromethyl)-1H-isochromene-4-carboxylic acid (X-1656A1). The stirred solution of 4-bromo-2-(carboxymethyl)benzoic acid (X-1654A1) (3.0 g, 11.6 mmol) in TFAA (15 mL) was heated at 140° C. for 16 h. After cooling to room temperature, the reaction mixture was slowly poured into ice-water (100 mL), and the resulting precipitate was collected by filtration. Obtained solid residue was washed with water (100×3 mL), dried under high vacuum, which was combined with another identically prepared batch, and the combined batches were concentrated under reduced pressure to afford 6-bromo-1-oxo-3-(trifluoromethyl)-1H-isochromene-4-carboxylic acid (X-1656A1) (4.7 g, 51%) as a white solid, which was taken to next step without further purification. MS: [MH]⁺672.8.

6-Bromo-3-(trifluoromethyl)isoquinolin-1(2H)-one (X-1656A2). A solution of 6-bromo-1-oxo-3-(trifluoromethyl)-1H-isochromene-4-carboxylic acid (X-1656A1) (2.90 g, 8.63 mmol) in a NH₄OH (16 mL) was stirred at room temperature, and the resulting mixture was heated at 120° C. for 16 h in sealed tube. After cooling to room temperature, the reaction mixture was slowly poured into water (100 mL), and the resulting precipitate was collected by filtration. Obtained solid residue was washed with water (80×3 mL) and dried under high vacuum to afford 6-bromo-3-(trifluoromethyl)isoquinolin-1(2H)-one (X-1656A2) (1.90 g, 76%) as a white solid. MS: [MH]⁺291.6/[MH+2]⁺293.6.

Methyl 1-oxo-3-(trifluoromethyl)-1,2-dihydroisoquinoline-6-carboxylate (X-1656A3). To a stirred solution of 6-bromo-3-(trifluoromethyl)isoquinolin-1(2H)-one (X-1656A2) (1.50 g, 5.53 mmol) in MeOH (33 mL) was added trimethylamine (2.23 g, 22.1 mmol), and the reaction mixture was degassed (purging with nitrogen) for 10 min followed by addition of PdCl₂(dppf).DCM (0.36 g, 0.44 mmol) sequentially at room temperature under nitrogen. The resulting mixture was degassed (purging with CO gas) for 10 min and stirred at 80° C. under CO gas pressure for 5 h. The reaction mixture was filtered and washed with EtOAc (3×50 mL). The solid product was further dissolved in 20% MeOH: DCM, and the filtrate was concentrated under reduced pressure to afford methyl 1-oxo-3-(trifluoromethyl)-1,2-dihydroisoquinoline-6-carboxylate (X-1656A3) (0.50 g, 36%) as an off-white solid. MS: [MH]⁺271.6.

Methyl 1-chloro-3-(trifluoromethyl)isoquinoline-6-carboxylate (X-1656A4).

A solution of methyl 1-oxo-3-(trifluoromethyl)-1,2-dihydroisoquinoline-6-carboxylate (X-1656A3) (0.500 g, 1.84 mmol) in POCl₃ (4 mL) was stirred and heated at 90° C. for 5 h. After completion of reaction, the reaction mixture was slowly poured into ice-water (100 mL), and the resulting precipitate was collected by filtration. Obtained solid residue was washed with water (100×3 mL) and dried under high vacuum to afford methyl 1-chloro-3-(trifluoromethyl)isoquinoline-6-carboxylate (X-1656A4) (0.35 g, 66%) as an off-white solid. MS: [MH]⁺290.20.

Methyl 3-(trifluoromethyl)-1-(4-(trifluoromethyl)piperidin-1-yl)isoquinoline-6-carboxylate (X-1656A5). To a stirred solution of methyl 1-chloro-3-(trifluoromethyl)isoquinoline-6-carboxylate (X-1656A4) (0.350 g, 1.21 mmol) in DMSO (5 mL) were added 4-(trifluoromethyl)piperidine (0.37 g, 2.42 mmol), K₂CO₃ (0.501 g, 3.63 mmol), and potassium iodide (0.040 g, 0.242 mmol) sequentially at room temperature under nitrogen, and the resulting reaction mixture was heated at 120° C. for 1 h. After cooling to room temperature, the reaction mixture was diluted with water (40 mL) and extracted with ethyl acetate (80 mL×2). The combined organic extracts were, dried over anhydrous Na₂SO₄, and concentrated under reduced pressure to afford methyl 3-(trifluoromethyl)-1-(4-(trifluoromethyl)piperidin-1-yl)isoquinoline-6-carboxylate (X-1656A5) (0.450 g, 91%) as a yellow solid, which was taken to next step without further purification. MS: [MH]⁺406.71.

3-(Trifluoromethyl)-1-(4-(trifluoromethyl)piperidin-1-yl)isoquinoline-6-carboxylic acid (X-1656A6). To a stirred solution of methyl 3-(trifluoromethyl)-1-(4-(trifluoromethyl)piperidin-1-yl)isoquinoline-6-carboxylate (X-1656A5) (0.35 g, 0.862 mmol) in a mixture of THF-water (3:1; 8.0 mL) was added lithium hydroxide monohydrate (0.072 g, 1.72 mmol) at room temperature, and the resulting mixture was stirred for 2 h at the same temperature. The reaction mixture was concentrated under reduced pressure, and the obtained crude was diluted with water (40 mL) and extracted with ethyl acetate (40 mL×2) to remove unwanted organic impurities. The aqueous part was acidified (pH ˜2-3) with an aqueous solution of 1 N HCl, and the resulting precipitate was collected by filtration. Crude residue was washed with cold water until the pH of the filtrate became neutral (pH ˜6-7). Obtained solid was dried under high vacuum to afford 3-(trifluoromethyl)-1-(4-(trifluoromethyl)piperidin-1-yl)isoquinoline-6-carboxylic acid (X-1656A6) (0.28 g, 83%) as an off-white solid, which was taken to next step without further purification. MS: [MH]⁺392.7.

(R)-N-(1-hydroxypropan-2-yl)-3-(trifluoromethyl)-1-(4-(trifluoromethyl)piperidin-1-yl)isoquinoline-6-carboxamide (I-52). To a stirred solution of 3-(trifluoromethyl)-1-(4-(trifluoromethyl)piperidin-1-yl)isoquinoline-6-carboxylic acid (X-1656A6) (0.280 g, 0.714 mmol) in DMF (6 mL) were added DIPEA (0.368 g, 2.85 mmol) and HATU (0.407 g, 1.07 mmol) sequentially at 0° C. under nitrogen. After stirring for 10 min at the same temperature, (R)-2-aminopropan-1-ol (0.16 g, 2.14 mmol) in DMF (1 mL) was added and stirring was continued at room temperature for 2 h. The reaction mixture was poured into ice-water (15 mL), and the resulting precipitate was collected by filtration. Obtained solid residue was washed with water (50×3 mL) and dried under high vacuum. The crude product was triturated with diethyl ether (3×10 mL) and dried over high vacuum to afford (R)-N-(1-hydroxypropan-2-yl)-3-(trifluoromethyl)-1-(4-(trifluoromethyl)piperidin-1-yl)isoquinoline-6-carboxamide (I-52) (0.050 g, 16%) as a yellow solid. H NMR (400 MHz, DMSO-d₆) δ 8.543-8.541 (d, J=0.80 Hz, 1H), 8.43-8.41 (d, J=8.0 Hz, 1H), 8.21-8.19 (d, J=8.80 Hz, 1H), 8.13-8.11 (dd, J=8.8 Hz, 1.20 Hz, 1H), 7.98 (s, 1H), 4.79-4.76 (t, J=6.0 Hz, 1H), 4.09-4.04 (m, 1H), 3.98-3.95 (d, J=12.0 Hz, 1H), 3.53-3.48 (m, 1H), 3.42-3.36 (m, 2-). 3.08-3.02 (t, J=12.0 Hz, 2H), 2.67-2.65 (m, 1H), 2.00-. 97 (d, J=11.2 Hz, 2H), 1.86-1.78 (m, 2H), 1.18-1.17 (d, J=6.8 Hz. 3H). MS: [MH]⁺ 449.7.

### Example 1.22. Synthesis of (R)-N-(1-hydroxybutan-2-yl)-3-methoxy-1-(4-(trifluoromethyl)phenyl)isoquinoline-6-carboxamide (I″-53)

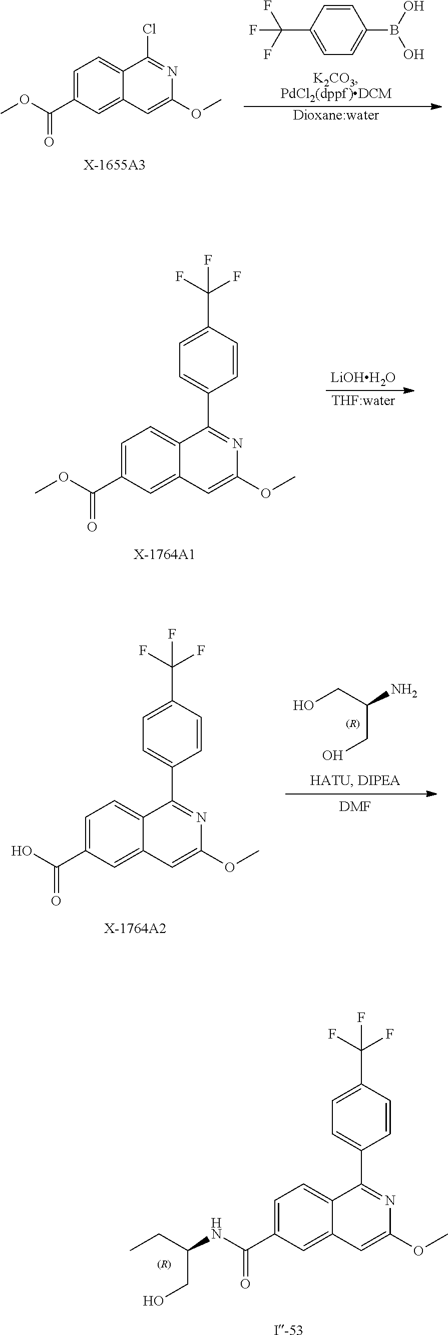

Methyl 3-methoxy-1-(4-(trifluoromethyl)phenyl)isoquinoline-6-carboxylate (X-1764A1). To the stirred solution of methyl 1-chloro-3-methoxyisoquinoline-6-carboxylate (X-1655A3) (2.50 g, 9.96 mmol) in dioxane: H₂O (7:3 30 mL) was added (4-(trifluoromethyl)phenyl)boronic acid (2.83 g, 14.94 mmol) and K₂CO₃ (4.12 g, 0.498 mmol), and the reaction mixture was degassed (purging with nitrogen) for 10 min followed by addition of PdCl₂(dppf).DCM (0.406 g, 29.88 mmol) sequentially at room temperature under nitrogen. The resulting reaction mixture was heated at 100° C. for 2 h. After cooling to room temperature, the reaction mixture was diluted with water (400 mL) and extracted with ethyl acetate (400 mL×2). The combined organic extracts were dried over anhydrous Na₂SO₄ and concentrated under reduced pressure. The crude product was purified by silica gel column chromatography, using ethyl acetate-hexane=1:9→4:6 as gradient, to afford methyl 3-methoxy-1-(4-(trifluoromethyl)phenyl)isoquinoline-6-carboxylate (X-1764A1) (2.40 g, 67%) as an off-white solid. MS: [MH]⁺362.3.

3-Methoxy-1-(4-(trifluoromethyl)phenyl)isoquinoline-6-carboxylic acid (X-1764A2). To a stirred solution of methyl 3-methoxy-1-(4-(trifluoromethyl)phenyl)isoquinoline-6-carboxylate (X-1764A1) (2.40 g, 6.64 mmol) in a mixture of THF-water (3:1; 13 mL) was added lithium hydroxide monohydrate (0.837 g, 19.94 mmol) at room temperature, and the resulting mixture was heated at 70° C. for 2 h. The reaction mixture was concentrated under reduced pressure, and the obtained crude was diluted with water (100 mL) and was extracted with ethyl acetate (150 mL) to remove unwanted organic impurities. The aqueous part was acidified (pH ˜2-3) with an aqueous solution of 1 N HCl and the resulting precipitate was collected by filtration. Crude residue was washed with cold water until the pH of the filtrate became neutral (pH ˜6-7). Obtained solid was dried under high vacuum to afford 3-methoxy-1-(4-(trifluoromethyl)phenyl)isoquinoline-6-carboxylic acid (X-1764A2) (2.0 g, 87%) as an off-white solid, which was taken to next step without further purification. MS: [MH]⁺348.3.

(R)-N-(1-hydroxybutan-2-yl)-3-methoxy-1-(4-(trifluoromethyl)phenyl)isoquinoline-6-carboxamide (I″-53). To a solution of 3-methoxy-1-(4-(trifluoromethyl)phenyl)isoquinoline-6-carboxylic acid (X-1764A2) (0.200 g, 0,576 nmol)) in DMF (6 mL) were added DIPEA (0.074 g, 0.576 mmol) and HATU (0.439 g, 1.15 mmol) sequentially at 0° C. under nitrogen. The resulting reaction mixture was stirred at room temperature for 10 min. (R)-2-aminobutan-1-ol (0.076 g, 0.864 mmol) in DMF (10 mL) was added at 0° C., and the resulting reaction mixture was stirred at room temperature for 2 h. The reaction mixture was poured into ice water (150 mL), and the solid product was precipitated, which was collected by filtration and dried under reduced pressure. The resulting crude material was triturated with diethyl ether and pentane (30 mL×3) and dried over high vacuum to afford (R)-N-(1-hydroxybutan-2-yl)-3-methyoxy-1-(4-(trifluoromethyl)phenyl)isoquinoline-6-carboxamide (I″-53) (0.150 g, 63%) as a white solid. ¹H NMR (400 MHz, DMSO-d₆) δ 8.418-8.415 (d, J=1.2 Hz, 1H), 8.33-8.31 (d, J=8.4 Hz, 1H), 7.98-7.94 (m, 5H), 7.80-7.78 (dd, J=9.2, 2.0 Hz, 1H), 7.39 (s, 1H), 7.74-4.71 (t, J=5.6 Hz, 1H), 4.00 (s, 3H), 3.93-3.90 (m, 1H), 3.51-3.41 (m, 2H), 1.73-1.71 (m, 1H), 1.52-1.45 (m, 1H), 0.92-0.89 (t, J=7.6 Hz, 3H). MS: [MH]⁺419.5.

The following compounds were prepared in a manner analogous to the procedures described above for (R)-N-(1-hydroxybutan-2-yl)-3-methoxy-1-(4-(trifluoromethyl)phenyl)isoquinoline-6-carboxamide (I″-53):

(S)-3-Methoxy-N-(1-methoxypropan-2-yl)-1-(4-(trifluoromethyl)phenyl)isoquinoline-6-carboxamide (I″-54) (0.200 g, 83%) as an off-white solid. ¹H NMR (400 MHz, DMSO-d₆) δ 8.56-8.54 (d, J=8.0 Hz, 1H), 8.396-8.393 (d, J=1.2 Hz, 1H), 7.98-7.94 (m, 5H), 7.79-7.76 (dd, J=8.8, 1.6 Hz, 1H), 7.397 (s, 1H), 4.27-4.23 (m, 1H), 4.00 (s, 3H), 3.47-3.43 (m, 1H), 3.29 (s, 3H), 1.18-1.17 (d, J=6.8 Hz, 3H). (1H merged with DMSO-d₆ moisture) MS: [MH]⁺419.4.

(S)-3-methoxy-N-(1-(pyridin-2-yl)ethyl)-1-(4-(trifluoromethyl)phenyl)isoquinoline-6-carboxamide (I″-55) (0.150 g. 57%) as an off-white solid. ¹H NMR (400 MHz, DMSO-d₆) (9.16-9.14 (d, J=8.0 Hz, 1H), 8.55-8.54 (m, 1H), 8.50-8.49 (d, J=1.6 Hz, 1H), 8.00-7.94 (m, 5H), 7.84-7.76 (m, 2H), 7.47-7.45 (m, J=8.4 Hz, 1H), 7.41 (s, 1H), 7.29-7.26 (m, 1H), 5.26 (m, 1H), 4.01 (s, 3H), 1.56-1.54 (d, J=7.2 Hz, 3H). MS: [MH]⁺452.4.

### Example 1.23. Synthesis of (R)-N-(1-hydroxypropan-2-yl)-3-oxo-1-(4-(trifluoromethyl)phenyl)-2,3,7,8-tetrahydroisoquinoline-6-carboxamide (I″-56)

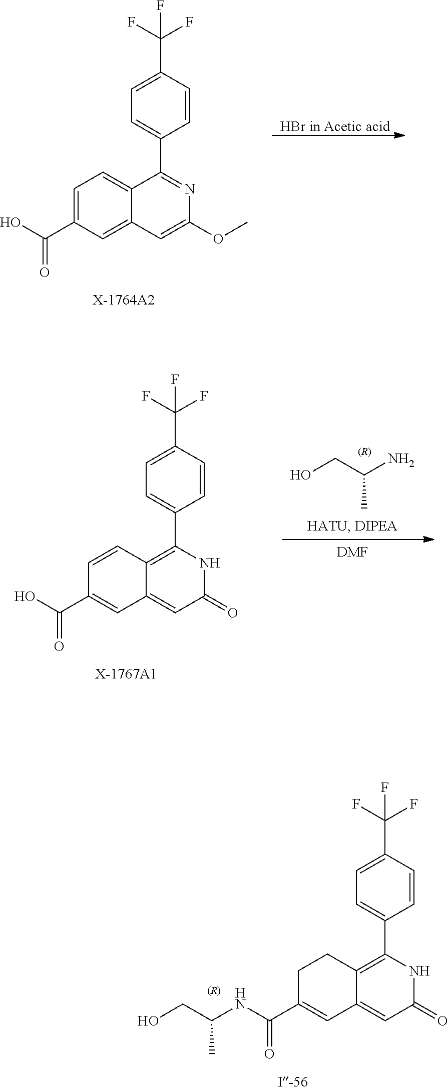

3-Oxo-1-(4-(trifluoromethyl)phenyl)-2,3-dihydroisoquinoline-6-carboxylic acid (X-1767A1). A solution of 3-methoxy-1-(4-(trifluoromethyl)phenyl)isoquinoline-6-carboxylic acid (X-1764A2) (0.200 g, 0.576 mmol) in HBr in acetic acid (4 mL) was heated at 120° C. for 3 h. After cooling to room temperature, the reaction mixture was slowly quenched with an aqueous solution of saturated NaHCO₃ and was extracted with ethyl acetate (100 mL×3). Combined organic extracts were dried over anhydrous Na₂SO₄ and concentrated under reduce pressure to afford 3-oxo-1-(4-(trifluoromethyl)phenyl)-2,3-dihydroisoquinoline-6-carboxylic acid (X-1767A1) (0.165 g, 86%) as an off-white solid. MS: [MH]⁺334.3.

(R)-N-(1-hydroxypropan-2-yl)-3-oxo-1-(4-(trifluoromethyl)phenyl)-2,3,7,8-tetrahydroisoquinoline-6-carboxamide (I″-56). To a stirred solution of 3-oxo-1-(4-(trifluoromethyl)phenyl)-2,3-dihydroisoquinoline-6-carboxylic acid (X-1767A1) (0.150 g, 0.450 mmol) in DMF (3 mL) were added DIPEA (0.174 g, 1.35 mmol) and HATU (0.343 g, 0.900 mmol) sequentially at 0° C. under nitrogen. After stirring for 10 min at the same temperature, was added (R)-2-aminopropan-1-ol (0.050 g, 0.675 mmol) and stirring was continued at room temperature for 1 h. The reaction mixture was diluted with water (60 mL) and extracted with ethyl acetate (60 mL×2). Combined organic extracts were washed with brine (100 ml), dried over anhydrous Na₂SO₄, and concentrated under reduced pressure. The resulting crude was purified by reverse phase (C-18) silica gel column chromatography, using acetonitrile-water=3:7→4:6 as gradient to afford (R)-N-(1-hydroxypropan-2-yl)-3-oxo-1-(4-(trifluoromethyl)phenyl)-2,3,7,8-tetrahydroisoquinoline-6-carboxamide (I″-56) (0.035 g, 20%) as a yellow solid. ¹H NMR (400 MHz, DMSO-d₆) δ 11.10 (br. s, 1H), 8.37-8.35 (d, J=8.0 Hz, 1H), 8.302 (br. s, 1H), 7.95-7.84 (m, 5H), 7.69-7.67 (d, J=8.4 Hz, 1H), 7.09 (br. s, 1H), 4.77 (br. s, 1H), 4.09-4.02 (m, 1H), 3.52-3.48 (m, 1H), 3.39-3.34 (m, 1H), 1.17-1.15 (d, J=6.8 Hz, 3H). MS: [MH]⁺391.4.

### Example 1.24. Synthesis of 5-(4-(Trifluoromethyl)piperidin-1-yl)-2-naphthoic acid (I-57)

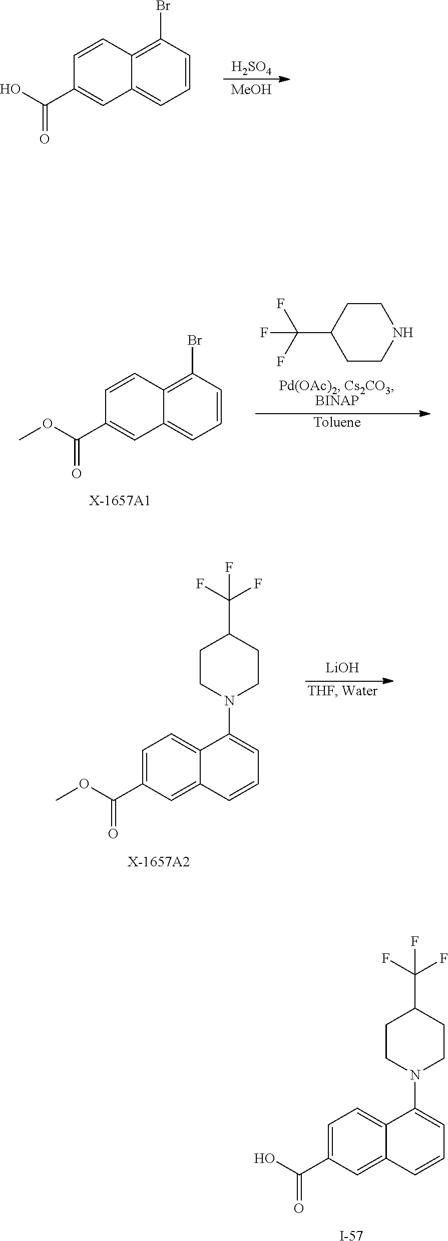

Methyl 5-bromo-2-naphthoate (X-1657A1). Concentrated H₂SO₄ (5 mL) was added to a stirred suspension of 5-bromo-2-naphthoic acid (15.40 g, 61.35 mmol) in methanol (270 mL) at room temperature, and the resulting mixture was heated at 40° C. for 18 h. After cooling to room temperature, the reaction mixture was slowly poured into an aqueous solution of saturated NaHCO₃ (700 mL) and was extracted with ethyl acetate (1000 mL×3). Combined organic extracts were dried over anhydrous Na₂SO₄ and concentrated under reduced pressure to afford methyl 5-bromo-2-naphthoate (X-1657A1) (15.80 g, 97%). ¹H NMR (400 MHz, DMSO-d6) δ 8.67 (s, 1H), 8.20-8.18 (d, J=8.8 Hz, 2H), 8.11-8.09 (dd, J=9.2, 1.6 Hz, 1H), 7.55-7.51 (t, J=8.0 Hz, 1H), 3.93 (s, 3H).

Methyl 5-(4-(trifluoromethyl)piperidin-1-yl)-2-naphthoate (X-1657A2). To a stirred solution of methyl 5-bromo-2-naphthoate (X-1657A1) (3.0 g, 11.367 mmol) in toluene (150.0 mL), 4-(trifluoromethyl)piperidine (2.60 g, 17.051 mmol) and Cs₂CO₃ (22.16 g, 68.20 mmol) were added at room temperature under nitrogen. The reaction mixture was degassed (purging with nitrogen) for 20 min followed by addition of BINAP (1.414 g, 2.27 mmol) and Pd(OAc)₂ (0.254 g, 1.13 mmol), and the resulting mixture was heated at 100° C. for 2 h. The reaction mixture was cooled to room temperature and filtered through a celite bed, and the filtrate was concentrated under reduced pressure. Obtained crude was diluted with water (200 mL) and was extracted with ethyl acetate (320 mL×3). Combined organic extracts were dried over anhydrous Na₂SO₄ and concentrated under reduced pressure. Obtained crude mass was purified by silica gel column chromatography using ethyl acetate: hexane=0:1→5:5 as gradient, to afford methyl 5-(4-(trifluoromethyl)piperidin-1-yl)-2-naphthoate (X-1657A2) (3.20 g, 83%). MS: [MH]⁺337.9.

5-(4-(Trifluoromethyl)piperidin-1-yl)-2-naphthoic acid (I-57). To a stirred solution of methyl 5-(4-(trifluoromethyl)piperidin-1-yl)-2-naphthoate (X-1657A2) (3.20 g, 9.49 mmol) in a mixture of THF-water (8:3; 26.0 mL) was added lithium hydroxide (1.19 g, 28.48 mmol) at room temperature, and the reaction mixture was heated at 60° C. for 2 h. After cooling to room temperature, the reaction mixture was concentrated under reduced pressure, diluted with water (100 mL), and extracted with ethyl acetate (50×2 mL) to remove unwanted organic impurities. The aqueous layer was acidified (pH ˜2-3) with aqueous 1 N HCl, and the resulting precipitate was collected by filtration and washed with cold water until the pH of the filtrate became neutral (pH ˜6-7). The obtained solid was dried in vacuum to afford 5-(4-(trifluoromethyl)piperidin-1-yl)-2-naphthoic acid (I-57) (2.9 g, 94%) as a white solid. ¹H NMR (400 MHz, DMSO-d6) δ 13.10 (br. s, 1H), 8.565-8.561 (d, J=1.6 Hz, 1H), 8.18-8.16 (d, J=8.8 Hz, 1H), 8.00-7.97 (dd, J=8.8 J=1.6 Hz, 1H), 7.80-7.78 (d, J=8.4 Hz, 1H), 7.54-7.50 (t, J=7.6 Hz, 1H), 7.26-7.25 (d, J=7.2 Hz, 1H), 3.40-3.34 (t, J=12.0 Hz, 2H), 2.83-2.77 (t, J=10.4 Hz, 2H), 2.57-2.56 (m, 1H), 2.00-1.98 (m, 2H), 1.90-1.79 (m, 2H). MS: [MH]⁺323.9.

### Example 1.25. Synthesis of (S)-N-(1-(pyridin-2-yl)ethyl)-5-(4-(trifluoromethyl)piperidin-1-yl)-2-naphthamide (I-58)

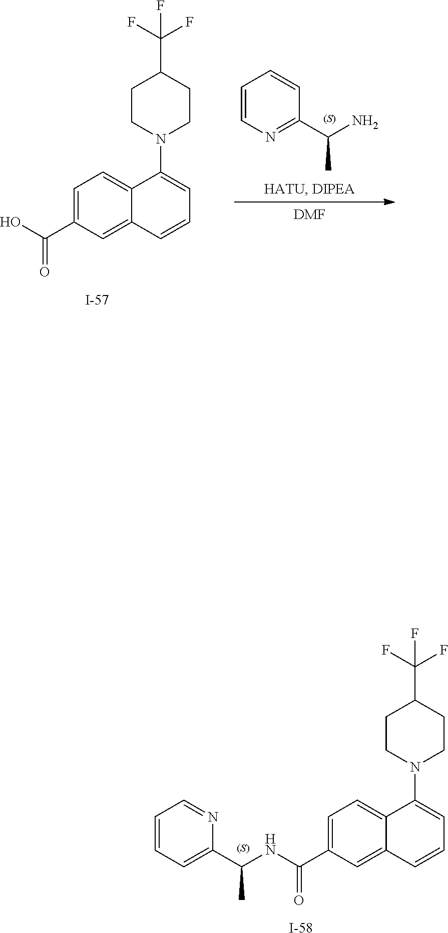

To a stirred solution of 5-(4-(trifluoromethyl)piperidin-1-yl)-2-naphthoic acid (I-57) (3.0 g, 9.28 mmol) in DMF (30 mL) were added diisopropylethylamine (4.8 g, 37.13 mmol) and HATU (5.29 g, 13.90 mmol) sequentially at 0° C. After stirring for 10 min at the same temperature, (S)-1-(pyridin-2-yl)ethan-1-amine (3.4 g, 27.85 mmol) was added under nitrogen and stirred for 1 h at room temperature. The reaction mixture was diluted with water (100 mL) and was extracted with ethyl acetate (100 mL×3). Combined organic extracts were dried over anhydrous Na₂SO₄ and concentrated under reduced pressure. Obtained crude was purified by silica gel column chromatography, using ethyl acetate-hexane=0:1→8:2 as gradient, to afford (S)-N-(1-(pyridin-2-yl)ethyl)-5-(4-(trifluoromethyl)piperidin-1-yl)-2-naphthamide (I-58) (2.1 g, 53%) as a white solid. ¹H NMR (400 MHz, DMSO-d₆) δ* 8.99-8.97 (d, J=7.6 Hz, 1H), 8.55-8.53 (m, 1H), 8.516-8.512 (d, J=1.6 Hz, 1H), 8.15-8.13 (d, J=8.8 Hz, 1H), 7.99-7.96 (dd, J=8.8, 1.6 Hz, 1H), 7.79-7.75 (m, 1H), 7.73-7.71 (d, J=8.4 Hz, 1H), 7.53-7.49 (t, J=7.6 Hz, 1H), 7.47-7.45 (d, J=7.6 Hz, 1H), 7.28-7.23 (m, 2H), 5.29-5.22 (m, 1H), 3.41-3.37 (m, 2H), 2.83-2.77 (t, J=12.0 Hz, 2H), 2.56-2.54 (m, 1H), 2.01-1.98 (d, J=10.8 Hz, 2H), 1.90-1.83 (m, 2H), 1.56-1.54 (d, J=6.8 Hz, 3H). MS: [MH]⁺428.1. *(one proton merged in DMSO-d₆ peak)

The following compound was prepared in a manner analogous to the procedures described above for (S)-N-(1-(pyridin-2-yl)ethyl)-5-(4-(trifluoromethyl)piperidin-1-yl)-2-naphthamide (I-58):

(R)-N-(1-hydroxypropan-2-yl)-5-(4-(trifluoromethyl)piperidin-1-yl)-2-naphthamide (I-59) (0.120 g, 51%) as an off-white solid. ¹H NMR (400 MHz, DMSO-d₆) δ 8.42 (s, 1H), 8.29-8.27 (d, J=8.0 Hz, 1H), 8.13-8.11 (d, J=8.8 Hz, 1H), 7.95-7.92 (d, J=8.8 Hz, 1H), 7.70-7.68 (d, J=8.0 Hz, 1H), 7.52-7.48 (t, J=7.6 Hz, 1H), 7.21-7.19 (d, J=7.6 Hz, 1H), 4.79-4.76 (t, J=5.2 Hz, 1H), 4.09-4.05 (m, 1H), 3.53-3.48 (m, 1H), 3.40-3.35 (m, 3H), 2.82-2.76 (t, J=12.0 Hz, 3H), 2.00-1.98 (d, J=10.4 Hz, 2H), 1.89-1.83 (m, 2H), 1.18-1.16 (d, J=6.8 Hz, 3H). MS: [MH]⁺381.1.

### Example 1.26. Synthesis of (R)-N-(1-hydroxypropan-2-yl)-8-(4-(trifluoromethyl) piperidin-1-yl)quinoline-3-carboxamide (I-60)

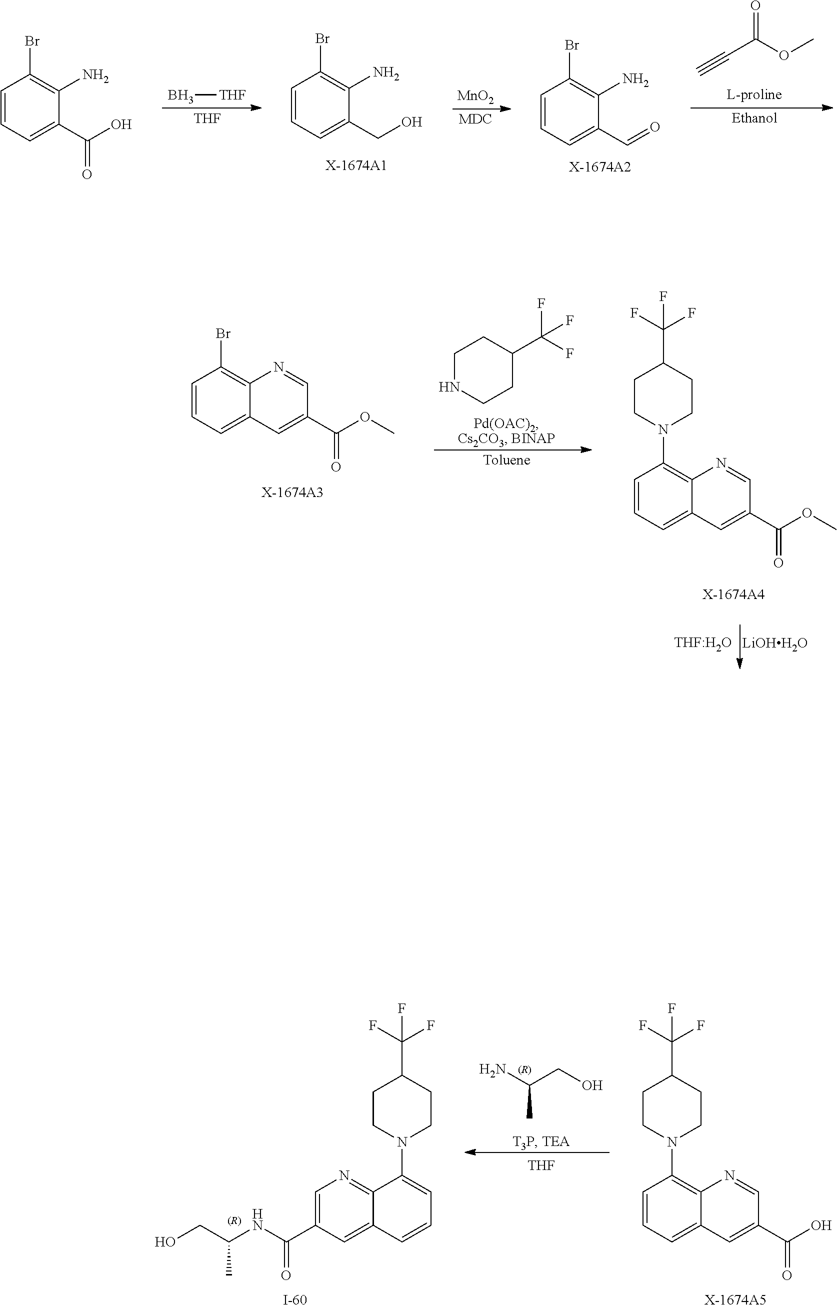

(2-Amino-3-bromophenyl)methanol (X-1674A1). To a stirred solution of 2-amino-3-bromobenzoic acid (25.0 g, 115.7 mmol) in THF (150 mL) was added BH₃-THF (30.9 g, 1.0 M in THF, 347.2 mmol) at 0° C. under nitrogen, and the resulting reaction mixture was stirred at 0° C. for 1 h. The reaction mixture was stirred at room temperature for 1 h and then heated at 70° C. for 16 h. The reaction mixture was slowly poured into methanol (1000 mL), stirred at rt for 1 h, and concentrated under reduced pressure. Obtained crude product was mixed with another prepared batch of (25.0 g) and was diluted with water, and the resulting precipitate was filtered and dried under reduce pressure to afford (2-amino-3-bromophenyl)methanol (X-1674A1) (50.0 g, quantitative) as an off-white solid. MS: [MH]⁺201.9/[MH+2]⁺203.9.

2-Amino-3-bromobenzaldehyde (X-1674A2). To a stirred solution of (2-amino-3-bromophenyl)methanol (X-1674A1) (25.0 g, 124.3 mmol) in dichloromethane (200 mL) was added MnO₂ (108.0 g, 1243.8 mmol) at 0° C., and the resulting reaction mixture was allowed to stir at room temperature for 16 h. The reaction mixture was combined with another prepared batch of (25.0 g), diluted with dichloromethane (250 mL), and filtered through a celite bed. The filtrate was concentrated under reduced pressure to afford 2-amino-3-bromobenzaldehyde (X-1674A2) as an off white solid [43.0 g, 86% (crude)], which was used in next step without further purification. MS: [MH]⁺199.8/[MH+2]⁺201.8.

Methyl 8-bromoquinoline-3-carboxylate (X-1674A3). To a stirred solution of 2-amino-3-bromobenzaldehyde (X-1674A2) (20.0 g, 100.5 mmol) in ethanol (150 mL) was added L-proline (5.77 g, 50.2 mmol) and methyl propiolate (16.8 g, 201.0 mmol) at room temperature, and the reaction mixture was stirred at 80° C. for 16 h. After cooling to room temperature, the reaction mixture combined with another prepared batch of (20.0 g) and was filtered. The precipitate was washed with water and dried under reduced pressure to afford methyl 8-bromoquinoline-3-carboxylate (X-1674A3) (35.0 g, 65%) as an off white solid, which was used in next step without further purification. [MH]⁺265.9/[MH+2]⁺267.9.

Methyl 8-(4-(trifluoromethyl)piperidin-1-yl)quinoline-3-carboxylate (X-1674A4). To a stirred solution of methyl 8-bromoquinoline-3-carboxylate (X-1674A3) (15.0 g, 56.6 mmol) and 4-(trifluoromethyl)piperidine (13.31 g, 113.2 mmol) in toluene (30 mL) was added BINAP (7.04 g, 11.32 mmol) and Cs₂CO₃ (73.5 g, 226.4 mmol) at room temperature. The reaction mixture was degassed (purging with nitrogen) for 20 min followed by addition of Pd(OAC)₂, (1.26 g, 5.66 mmol), and the reaction mixture was stirred at 100° C. for 6 h. The reaction mixture was cooled to room temperature, diluted with water (500 mL), and extracted with ethyl acetate (200 mL×3). The combined organic extracts were dried over anhydrous Na₂SO₄ and concentrated under reduced pressure. The crude product was purified by silica gel column chromatography using ethyl acetate-hexane=1:9→2:8 as gradient to afford methyl 8-(4-(trifluoromethyl)piperidin-1-yl)quinoline-3-carboxylate (X-1674A4) (14.0 g, 73%) as an off-white solid. MS: [MH]⁺339.0.

8-(4-(Trifluoromethyl)piperidin-1-yl)quinoline-3-carboxylic acid (X-1674A5). To the stirred solution of methyl 8-(4-(trifluoromethyl)piperidin-1-yl)quinoline-3-carboxylate (X-1674A4) (3.0 g, 8.87 mmol) in THF:H₂O (2:3, 30 mL) was added LiOH.H₂O (1.10 g, 26.62 mmol). The reaction mixture was stirred at 70° C. for 1 h. After completion of reaction, the reaction mixture was cooled and was concentrated under reduced pressure. The obtained crude was acidified by 1N HCl solution, and the precipitate was filtered and dried under reduced pressure to afford 8-(4-(trifluoromethyl)piperidin-1-yl)quinoline-3-carboxylic acid (X-1674A5) (2.50 g, 86%) as white solid. MS: [MH]⁺324.8.

(R)-N-(1-hydroxypropan-2-yl)-8-(4-(trifluoromethyl)piperidin-1-yl)quinoline-3-carboxamide (I-60). To a solution of (R)-2-aminopropan-1-ol (1.73 g, 23.14 mmol) in THF (20 mL) were added 8-(4-(trifluoromethyl)piperidin-1-yl)quinoline-3-carboxylic acid (2.5 g, 7.77 mmol), triethylamine (3.10 g, 30.86 mmol), and propylphosphonic anhydride (3.68 g, 11.57 mmol) sequentially at 0° C. under nitrogen, and the resulting mixture was stirred at room temperature for 10 min. The reaction mixture was poured into ice-water (100 mL) and was extracted with ethyl acetate (120 mL×3). Combined organic extracts were washed with brine (100 mL), dried over anhydrous Na₂SO₄, and concentrated under reduced pressure. The crude product was purified by silica gel column chromatography, using methanol-dichloromethane=0:1→1:9 as gradient, to afford (R)-N-(1-hydroxypropan-2-yl)-8-(4-(trifluoromethyl)piperidin-1-yl)quinoline-3-carboxamide (I-60) (1.70 g, 65%) as a white solid. ¹H NMR (400 MHz, DMSO-d6) δ 9.24-9.23 (d, J=2.4 Hz, 1H), 8.74-8.73 (d, J=2.0 Hz, 1H), 8.45-8.43 (d, J=7.6 Hz, 1H), 7.63-7.61 (d, J=8.0 Hz, 1H), 7.56-7.52 (t, J=8.0 Hz, 1H), 7.25-7.23 (d, J=7.6 Hz, 1H), 4.79-4.76 (t, J=5.6 Hz, 1H), 4.12-4.05 (m, 1H), 4.02-3.99 (d, J=11.6 Hz, 2H), 3.54-3.48 (m, 1H), 3.42-3.36 (m, 1H), 2.83-2.77 (t, J=11.6 Hz, 2H), 1.98-1.95 (d, J=11.2 Hz, 2H), 1.84-1.74 (m, 2H), 1.19-1.17 (d, J=6.8 Hz, 3H). (One proton merged with DMSO-d₆ peak). MS: [MH]⁺382.2

The following compound was prepared in a manner analogous to the procedures described above for (R)-N-(1-hydroxypropan-2-yl)-8-(4-(trifluoromethyl)piperidin-1-yl)quinoline-3-carboxamide (I-60):

(S)-N-(1-methoxypropan-2-yl)-8-(4-(trifluoromethyl)piperidin-1-yl)quinoline-3-carboxamide (I-61) (1.70 g, 40%) as an off-white solid. ¹H NMR (400 MHz, DMSO-d₆) δ 9.24-9.23 (d, J=2.4 Hz, 1H), 8.74-8.73 (d, J=2.0 Hz, 1H), 8.45-8.43 (d, J=7.6 Hz, 1H), 7.63-7.61 (d, J=8.0 Hz, 1H), 7.56-7.52 (t, J=8.0 Hz, 1H), 7.25-7.23 (dd, J=7.6, 1.2 Hz, 1H), 4.30-4.23 (t, J=5.6 Hz, 1H), 4.02-3.99 (d, J=11.6 Hz, 2H), 3.48-3.44 (m, 1H), 3.35-3.31 (m, 1H), 3.29 (s, 3H), 2.83-2.77 (t, J=11.6 Hz, 2H), 1.97-1.95 (d, J=11.2 Hz, 2H), 1.84-1.74 (m, 2H), 1.20-1.18 (d, J=6.8 Hz, 3H). (One proton merged with DMSO-d6 peak) MS: [MH]⁺396.3.

### Example 1.27. Synthesis of (S)-3-methoxy-N-(1-(pyridin-2-yl)ethyl)-1-(4-(trifluoromethyl)piperidin-1-yl)isoquinoline-6-carboxamide (I-64)

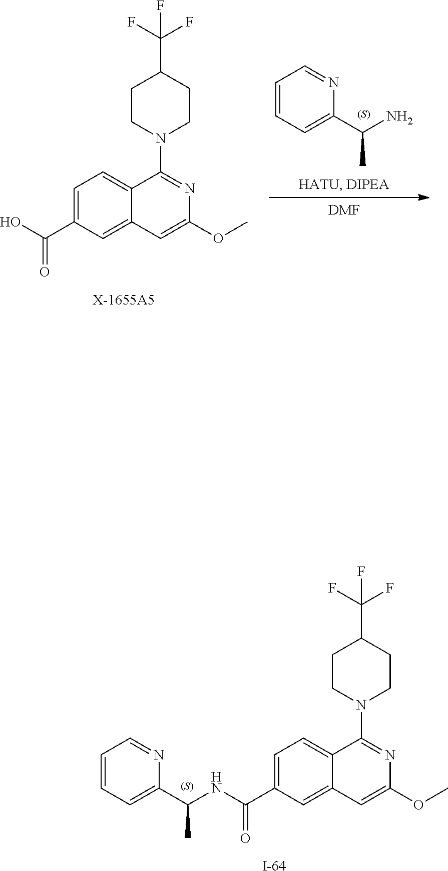

To a stirred solution of 3-methoxy-1-(4-(trifluoromethyl)piperidin-1-yl)isoquinoline-6-carboxylic acid (X-1655A5) (0.150 g, 0.42 mmol) in DMF (5 mL) were added DIPEA (0.162 g, 1.26 mmol) and HATU (0.239 g, 0.63 mmol) sequentially at 0° C. under nitrogen. After stirring for 5 min at the same temperature, was added (S)-1-(pyridin-2-yl)ethan-1-amine (0.153 g, 1.27 mmol) and stirring was continued at room temperature for 1h. Reaction mixture was poured into ice water (150 mL) during which solid was precipitated out, which was collected by filtration and dried under reduced pressure. The resulting crude material was triturated with n-pentane (15 mL×3), dried under high vacuum to afford (S)-3-methoxy-N-(1-(pyridin-2-yl)ethyl)-1-(4-(trifluoromethyl)piperidin-1-yl)isoquinoline-6-carboxamide (I-64) (0.100 g, 52%) as a yellow solid. ¹H NMR (400 MHz, DMSO-d6) δ 9.04-9.02 (d, J=7.6 Hz, 1H), 8.54-8.53 (dd, J=4.8, 0.8 Hz, 1H), 8.284-8.280 (d, J=1.6 Hz, 1H), 8.01-7.99 (d, J=8.8 Hz, 1H), 7.79-7.72 (m, 2H), 7.45-7.43 (d, J=8.0 Hz, 1H), 7.28-7.25 (m, 1H), 6.78 (s, 1H), 5.23-5.21 (m, 1H), 3.94-3.90 (m, 2H), 3.90 (s, 3H), 3.04-2.98 (t, J=12.0 Hz, 2H), 2.52-2.50 (m, 1H), 1.97-1.94 (d, J=10.8 Hz, 2H), 1.84-1.75 (m, 2H), 1.54-1.52 (d, J=7.2 Hz, 3H), MS: [MH]⁺459.4.

The following compound was prepared in a manner analogous to the procedures described above for (S)-3-methoxy-N-(1-(pyridin-2-yl)ethyl)-1-(4-(trifluoromethyl)piperidin-1-yl)isoquinoline-6-carboxamide (I-64):

(R)-N-(1-hydroxybutan-2-yl)-3-methoxy-1-(4-(trifluoromethyl)piperidin-1-yl)isoquinoline-6-carboxamide (I-65) (0.070 g, 39%) as an off-white solid. ¹H NMR (400 MHz, DMSO-d₆) δ 8.22-8.20 (d, J=9.6 Hz, 1H), 8.20 (s, 1H), 7.99-7.97 (d, J=8.4 Hz, 1H), 7.71-7.68 (dd, J=8.8 Hz, 1.6 Hz, 1H), 6.75 (s, 1H), 4.72-4.69 (t, J=5.6 Hz, 1H), 3.89-3.93 (m, 2H), 3.89 (s, 3H), 3.50-3.43 (m, 1H), 3.42-3.33 (m, 1H), 3.04-2.98 (t, J=12.4 Hz, 2H), 2.65-2.55 (m, 1H), 1.97-1.94 (d, J=11.2 Hz, 2H), 1.84-1.75 (m, 2H), 1.72-1.65 (m, 1H), 1.50-1.43 (m, 1H), 0.91-0.89 (t, J=7.2 Hz, 3H). MS: [MH]⁺426.5.

### Example 1.28. Synthesis of 6-methoxy-1-(4-(trifluoromethyl)piperidin-1-yl)isoquinoline-3-carboxylic acid (68)

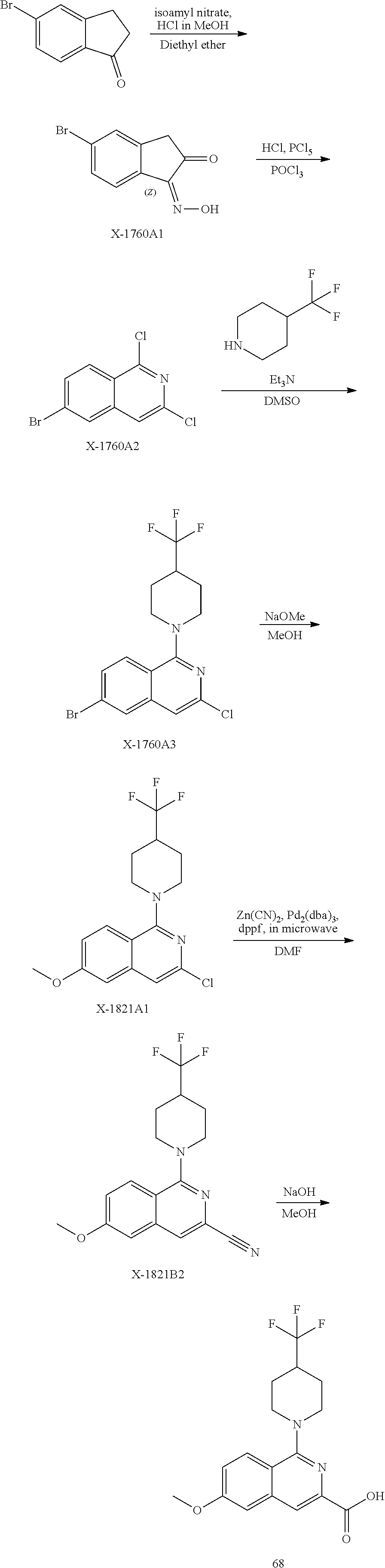

(Z)-5-bromo-1-(hydroxyimino)-1,3-dihydro-2H-inden-2-one (X-1760A1). To a stirred solution of 5-bromo-2,3-dihydro-1H-inden-1-one (50.0 g, 236.96 mmol) in a diethyl ether (500 mL) was added HCl (g) in MeOH (Freshly prepared) (100 ml) at 0° C. The reaction mixture was stirred for 20 min followed by the addition of isoamyl nitrate (41.58 g, 355.45 mmol) at the same temperature and stirring was continued for 1h at 0° C., during which a solid was precipitated out. Reaction mixture filtered over a Buchner funnel, washed the residue with diethyl ether (100 mL) and dried under reduced pressure to afforded (Z)-5-bromo-1-(hydroxyimino)-1,3-dihydro-2H-inden-2-one (X-1760A1) (45.0 g, 79%) as a white solid MS: [MH]⁺240.1/[MH+2]⁺242.1.

6-Bromo-1,3-dichloroisoquinoline (X-1760A2). HCl gas [HCl gas was generated by performing parallel reaction of solid NaCl (500 gm) with Con. H₂SO₄ (40 ml)] was purged to POCl₃ (10 mL) for 20 min at 0° C. (Z)-5-Bromo-1-(hydroxyimino)-1,3-dihydro-2H-inden-2-one (5.0 g, 20.92 mmol) was added to the mixture followed by the addition of PCl₅ (6.52 g, 31.38 mmol) portion wise over the period of 30 min at the same temperature and the resulting mixture was heated at 90° C. for 16h. [Another reaction with identical batch (5 g) was performed in parallel and mixed together prior to work-up]. After cooling to room temperature, the reaction mixture was slowly poured into ice water (500 mL) and the resulting brown solid precipitate was collected by filtration. Residue was washed with water (200 mL) and dried under reduced pressure to afford 6-bromo-1,3-dichloroisoquinoline (X-1760A2) (9.0 g, 78%; crude) as a brown solid MS: [(M+1)]⁺276.0.

6-Bromo-3-chloro-1-(4-(trifluoromethyl)piperidin-1-yl)isoquinoline (X-1760A3). To a stirred solution of 6-bromo-1,3-dichloroisoquinoline (X-1760A2) (5.0 g, 18.18 mmol) in DMSO (30 mL) were added 4-(trifluoromethyl)piperidine (4.17 g, 27.27 mmol), triethylamine (4.5 g, 45.45 mmol) at room temperature under nitrogen and the resulting reaction mixture was heated at 100° C. for 16h. Reaction mixture was cooled to room temperature, quenched with water (200 mL) and was extracted with ethyl acetate (200 mL×3). Collected organic extracts were dried over anhydrous Na₂SO₄ and concentrated under reduced pressure. The crude product was purified by column chromatography on silica gel, using ethyl acetate: hexane=0:1→1:9 as eluent, to afforded 6-bromo-3-chloro-1-(4-(trifluoromethyl)piperidin-1-yl)isoquinoline (X-1760A3) (4.0 g, 56%) as an off-white solid. MS: [MH]⁺393.3/[MH+2]⁺395.3.

3-Chloro-6-methoxy-1-(4-(trifluoromethyl)piperidin-1-yl)isoquinoline (X-1821A1). To a stirred solution of 6-bromo-3-chloro-1-(4-(trifluoromethyl)piperidin-1-yl)isoquinoline (X-1760A3) (0.200 g, 0.510 mmol) in methanol (3 mL) was added sodium methoxide (0.055 g, 1.02 mmol) portion wise over the period of 30 min at 0° C. under nitrogen. After 10 min of stirring at room temperature, the reaction mixture was heated to 90° C. and stirring was continued for another 16h at the same temperature. After cooling to room temperature, reaction mixture was concentrated under reduced pressure, obtained crude residue was taken in water (100 mL) and was extracted with ethyl acetate (100 mL×3). Combined organic extracts were dried over anhydrous Na₂SO₄ and concentrated under reduce pressure, which was combined with an identically prepared another 19 batches of 0.200 g each. Combined crude product was purified by silica gel column chromatography, using ethyl acetate-hexane=0:1→1:9 as gradient, to afforded 3-chloro-6-methoxy-1-(4-(trifluoromethyl)piperidin-1-yl)isoquinoline) (X-1821A1) (0.700 g, 20%) as an off-white solid MS: [MH]⁺345.3/[MH+2]⁺347.4.

6-Methoxy-1-(4-(trifluoromethyl)piperidin-1-yl)isoquinoline-3-carbonitrile (X-1821B2). To a stirred solution of 3-chloro-6-methoxy-1-(4-(trifluoromethyl)piperidin-1-yl)isoquinoline (X-1821B2) (0.350 g, 1.01 mmol) in DMF (10 mL) were added zinc cyanide (0.595 g, 5.08 mmol) at room temperature under nitrogen. The reaction mixture was degassed (purging with nitrogen) for 20 min followed by addition of dppf (0.281 g, 0.50 mmol) and Pd₂(dba)₃ (0.465 g, 0.50 mmol) at the same temperature and the resulting mixture was heated at 120° C. under microwave irradiation for 1h. Reaction mixture was diluted with ice-water (100 mL) and was extracted with ethyl acetate (100 mL×2). Combined organic extracts were dried over anhydrous Na₂SO₄, filtered and concentrated under reduced pressure to get a crude mass, which was combined with an identically prepared 0.350 g batch. Obtained crude mass was purified by silica gel column chromatography using ethyl acetate: hexane=0:1→1:9 as gradient, to afford 6-methoxy-1-(4-(trifluoromethyl)piperidin-1-yl)isoquinoline-3-carbonitrile (X-1821B2) (0.350 g, 51%) as an off-white solid. MS: [MH]⁺336.4

6-Methoxy-1-(4-(trifluoromethyl)piperidin-1-yl)isoquinoline-3-carboxylic acid (68). To a stirred solution of 6-methoxy-1-(4-(trifluoromethyl)piperidin-1-yl)isoquinoline-3-carbonitrile (X-1821B2) (0.200 g, 0.59 mmol) in MeOH (4 mL) was added NaOH (0.119 g, 2.98 mmol) at room temperature and the resulting mixture was stirred at 90° C. for 16 h. Reaction mixture was concentrated under reduced pressure and the obtained crude product was purification by reverse phase (C-18) silica gel column chromatography, using, acetonitrile-water=0:1→4:6 as gradient, to afford 6-methoxy-1-(4-(trifluoromethyl)piperidin-1-yl)isoquinoline-3-carboxylic acid (68) (0.050 g, 23%) as off white solid. ¹H NMR (400 MHz, DMSO-d₆) δ 7.94-7.92 (d, J=8.8 Hz, 1H), 7.86 (s, 1H), 7.29-7.28 (d, J=2.4 Hz, 1H), 7.16-7.13 (dd, J=2.4, 9.2 Hz, 1H), 3.88 (s, 3H), 3.73-3.69 (d, J=12.4 Hz, 2H), 2.94-2.88 (t, J=12.0 Hz, 2H), 1.95-1.93 (d, J=10.4 Hz, 2H), 1.84-1.74 (m, 2H). MS: [MH]⁺355.4 [One aliphatic proton merged with DMSO-d6 peak and acid proton not appeared in 1H NMR due to extra moisture from DMSO-d₆].

### Example 1.29. Synthesis of (R)-N-(1-hydroxypropan-2-yl)-6-methoxy-1-(4-(trifluoromethyl)piperidin-1-yl)isoquinoline-3-carboxamide (67)

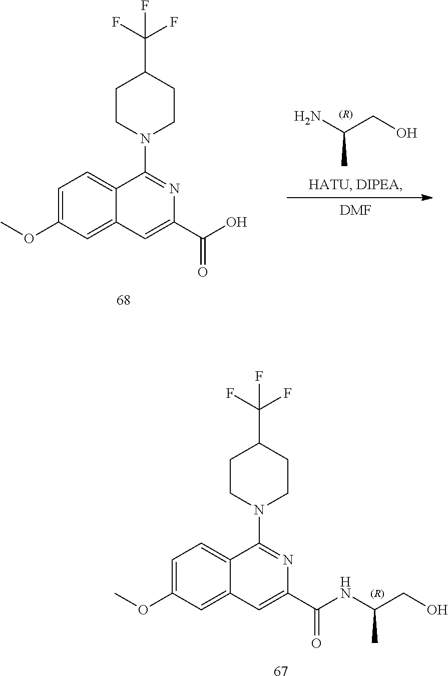

To a stirred solution of 6-methoxy-1-(4-(trifluoromethyl)piperidin-1-yl)isoquinoline-3-carboxylic acid (68) (0.300 g, 0.84 mmol) in DMF (1 mL) were added DIPEA (0.218 g, 1.69 mmol) and HATU (0.966 g, 2.54 mmol) at 0° C. under nitrogen. After 10 min of stirring at the same temperature, was added (R)-2-aminopropan-1-ol (0.127 g, 1.69 mmol) and stirring was continued for 1h at 0° C. under nitrogen. Reaction mixture was slowly poured into water (50 mL) and was extracted with ethyl acetate (50 mL×3). Combined organic extracts were dried over anhydrous Na₂SO₄ and concentrated under reduced pressure to get a crude product. The crude product was purified by reverse phase column chromatography on C-18 silica get using acetonitrile-water=0:1→4:6 as an eluent, to afford (R)-N-(1-hydroxypropan-2-yl)-6-methoxy-1-(4-(trifluoromethyl)piperidin-1-yl)isoquinoline-3-carboxamide (67) (0.150 g, 43%) as off-white. ¹H-NMR (400 MHz, DMSO-d₆) δ 8.23-8.21 (d, J=8.4 Hz, 1H), 8.04-8.03 (m, 2H), 7.52-7.51 (d, J=2.4 Hz, 1H), 7.29-7.27 (dd, J=2.4, 9.2 Hz, 1H), 4.94-4.93 (t, J=5.6 Hz, 1H), 4.04-4.01 (m, 1H), 3.91 (s, 3H), 3.89-3.86 (m, 2H), 3.50-3.45 (m, 2H), 3.02-2.96 (t, J=12.0 Hz, 2H), 2.62-2.59 (m, 1H), 1.99-1.96 (d, J=11.2 Hz, 2H), 1.86-1.80 (m, 2H), 1.20-1.18 (d, J=6.8 Hz, 3H). MS: [MH]⁺412.5.

### Example 1.30. Synthesis of (R)-3-ethoxy-N-(1-hydroxypropan-2-yl)-1-(4-(trifluoromethyl)piperidin-1-yl)isoquinoline-6-carboxamide (I-62)

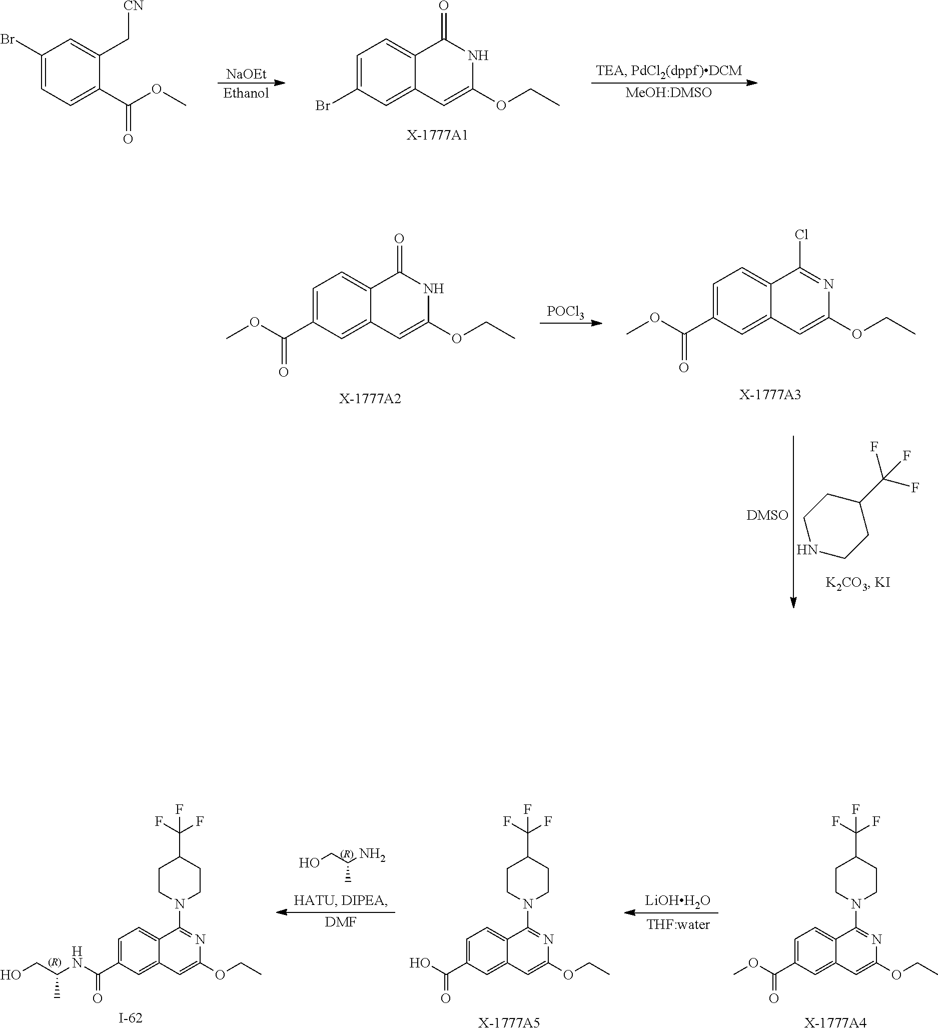

6-Bromo-3-ethoxyisoquinolin-1(2H)-one (X-1777A1). To a stirred solution of NaOEt (21% in Ethanol) (5.1 mL, 15.81 mmol) in ethanol (20 mL) was added methyl 4-bromo-2-(cyanomethyl)benzoate (2.0 g, 7.90 mmol) at room temperature and the resulting reaction mixture was stirred at 70° C. temperature for 2h. Reaction mixture was concentrated under reduced pressure, obtained crude was acidified (pH ˜7) with an aqueous solution of 1N HCl and the resulting precipitate was collected by filtration. Residue was washed with cold water and dried under high vacuum to get crude mass. [A second batch of reaction was performed in 3.0 g scale in parallel and mixed together prior to final trituration]. Combined crude was purified by trituration using diethyl ether to afford 6-bromo-3-ethoxyisoquinolin-1(2H)-one (X-1777A1) (3.40 g, 64%; crude) as a brown solid; along with starting material (˜22%), which was taken to next step without further purification. MS: [MH]⁺268.1/[MH+2]⁺270.2.

Methyl 3-ethoxy-1-oxo-1,2-dihydroisoquinoline-6-carboxylate (X-1777A2). To a stirred solution of 6-bromo-3-ethoxyisoquinolin-1(2H)-one (X-1777A1) [3.40 g (crude), 12.73 mmol] in a mixture of MeOH-DMSO (5:1: 600 mL) was added triethylamine (7.0 mL, 50.93 mmol) at room temperature under nitrogen. The reaction mixture was degassed (by purging nitrogen) for 30 min followed by the addition of PdCl₂(dppf).DCM (1.0 g, 1.27 mmol). The reaction mixture was purged with CO_{(g)} for 30 min and stirred at 70° C. under 70 psi in a Parr autoclave for 3h. Reaction mixture was cooled to room temperature, slowly poured into water (700 mL) and the resulting precipitate was collected by filtration. Isolated solid residue was washed with cold water and dried under high vacuum to get crude product, which was purified by trituration with DCM to afford methyl 3-ethoxy-1-oxo-1,2-dihydroisoquinoline-6-carboxylate (X-1777A2) (3.30 g, Quant.; crude) as a brown solid. MS: [MH]⁺248.3.

Methyl 1-chloro-3-ethoxyisoquinoline-6-carboxylate (X-1777A3). A solution of methyl 3-ethoxy-1-oxo-1,2-dihydroisoquinoline-6-carboxylate (X-1777A2) (3.40 g, 13.76 mmol) in POCl₃ (40.0 mL) was stirred at 90° C. for 1h. Reaction mixture was cooled to room temperature, diluted with ethyl acetate and slowly poured into ice water (1000 mL). Resulting solution was basified (pH ˜7) with slow addition of solid NaHCO₃ and was extracted with ethyl acetate (300.0 mL×3). Combined organic extracts were dried over anhydrous Na₂SO₄ and concentrated under reduced pressure to afford methyl 1-chloro-3-ethoxyisoquinoline-6-carboxylate (X-1777A3) (2.60 g, 71%; crude) as a brown solid. MS: [MH]⁺266.2/[MH+2]⁺268.2.

Methyl 3-ethoxy-1-(4-(trifluoromethyl)piperidin-1-yl)isoquinoline-6-carboxylate (X-1777A4). To a stirred solution of methyl 1-chloro-3-ethoxyisoquinoline-6-carboxylate (X-1777A3) (0.800 g, 3.01 mmol) in DMSO (8 mL) were added 4-(trifluoromethyl)piperidine (0.900 g, 6.037 mmol), KI (0.100 g, 0.603 mmol) and K₂CO₃ (1.45 g, 10.56 mmol) sequentially at room temperature and the resulting mixture was heated at 100° C. temperature for 1h. After cooling the reaction mixture to room temperature, reaction mixture was slowly poured into water (100 mL) and was extracted with ethyl acetate (100 mL×3). Combined organic extracts were dried over anhydrous Na₂SO₄ and concentrated under reduced pressure. The crude product was purified by column chromatography on silica gel using ethyl acetate-hexane=0:1→2:3 as eluent, to afford methyl 3-ethoxy-1-(4-(trifluoromethyl)piperidin-1-yl)isoquinoline-6-carboxylate (X-1777A4) (0.450 g, 39%) as an off-white solid. MS: [MH]⁺383.4.

3-Ethoxy-1-(4-(trifluoromethyl)piperidin-1-yl)isoquinoline-6-carboxylic acid (X-1777A5). To a stirred solution of methyl 3-ethoxy-1-(4-(trifluoromethyl)piperidin-1-yl)isoquinoline-6-carboxylate (X-1777A4) (0.450 g, 1.17 mmol) in a mixture of THF: water (3:1; 7 mL) was added lithium hydroxide monohydrate (0.140 g, 3.53 mmol) at room temperature and the resulting mixture was stirred for 2h at the same temperature. Reaction mixture was concentrated under reduced pressure, obtained crude was diluted with water (40 mL) and was extracted with ethyl acetate (40 mL×2) to remove unwanted organic impurities. Aqueous part was acidified (pH ˜5) with an aqueous solution of 1N HCl and the resulting precipitate was collected by filtration. Crude residue was washed with cold water until the pH of the filtrate became neutral (pH ˜6-7). Obtained solid was dried under high vacuum to afford 3-ethoxy-1-(4-(trifluoromethyl)piperidin-1-yl)isoquinoline-6-carboxylic acid (X-1777A5) (0.400 g, 92%; crude) as a yellow solid. MS: [MH]⁺369.3.

(R)-3-ethoxy-N-(1-hydroxypropan-2-yl)-1-(4-(trifluoromethyl)piperidin-1-yl)isoquinoline-6-carboxamide (I-62). To a stirred solution of 3-ethoxy-1-(4-(trifluoromethyl)piperidin-1-yl)isoquinoline-6-carboxylic acid (X-1777A5) (0.350 g, 0.951 mmol) in DMF (3 mL) were added DIPEA (0.49 mL, 2.853 mmol) and HATU (0.650 g, 1.711 mmol) sequentially at 0° C. After stirring at same temperature for 15 min, was added (R)-2-aminopropan-1-ol (0.14 g, 1.902 mmol) under nitrogen and stirred was continued at room temperature for another 1h. Reaction mixture was diluted with water (50 mL) and the resulting precipitate was collected by filtration. Obtained solid residue was washed with water (50×2 mL), dried under high vacuum to afford (R)-3-ethoxy-N-(1-hydroxypropan-2-yl)-1-(4-(trifluoromethyl)piperidin-1-yl)isoquinoline-6-carboxamide (I-62) (0.300 g, 74%) as a yellow solid.

¹H-NMR (400 MHz, DMSO-d₆) 8.30-8.28 (d, J=8.0 Hz, 1H), 8.17-816 (d, J=1.2 Hz, 1H) 7.98-7.96 (d, J=8.8 Hz, 1H), 7.70-7.67 (dd, J=8.8 Hz, 1.6 Hz, 1H), 6.72 (s, 1H), 4.77-4.74 (t, J=6.0 Hz, 1H), 4.32-4.27 (q, J=6.8 Hz, 2H), 4.08-4.01 (m, 1H), 3.91-3.88 (d, J=12.4 Hz, 2H), 3.51-3.46 (m, 1H), 3.39-3.35 (m, 1H), 3.03-2.97 (t, J=12.0 Hz, 2H), 2.67-2.58 (m, 1H), 1.96-1.93 (d, J=10.8 Hz, 2H), 1.84-1.75 (m, 2H), 1.38-1.34 (t, J=6.8 Hz, 3H), 1.16-1.14 (d, J=6.8 Hz, 3H). MS: [MH]⁺426.5.

The following compound was prepared in a manner analogous to the procedures described above for (R)-3-ethoxy-N-(1-hydroxypropan-2-yl)-1-(4-(trifluoromethyl)piperidin-1-yl)isoquinoline-6-carboxamide (I-62):

(R)-N-(1-hydroxypropan-2-yl)-1-(4-(trifluoromethyl)piperidin-1-yl)isoquinoline-6-carboxamide (I-63). (0.080 g, 34%) as an off-white solid. ¹H NMR (400 MHz, DMSO-d₆) δ 8.38-8.35 (m, 2H), 8.17-8.16 (d, J=5.6 Hz, 1H), 8.12-8.10 (d, J=8.8 Hz, 1H), 7.98-7.96 (d, J=8.4 Hz, 1H), 7.47-7.46 (d, J=5.6 Hz, 1H), 4.78-4.75 (t, J=5.6 Hz, 1H), 4.10-4.03 (m, 1H), 3.85-3.82 (d, J=12.8 Hz, 2H), 3.52-3.47 (m, 1H), 3.40-3.39 (m, 1H), 2.99-2.93 (t, J=12.0 Hz, 2H), 1.97-1.95 (d, J=11.2 Hz, 2H), 1.86-1.77 (m, 2H), 1.17-1.16 (d, J=6.8 Hz, 3H). [¹H merged in DMSO-d₆ peak]. MS: [MH]⁺382.5.

### Example 2. TEAD Compound Displacement and Proliferation Assays

Compound Displacement Assay.

A TEAD1 lipid pocket displacement assay was carried out according to the following protocol. Purified His-tagged TEAD1 protein (YAP Binding Domain) was pre-mixed with a Cy5-probe (Cy5-conjugated to a small molecule that binds in the TEAD1 lipid pocket) and Terbium-labeled anti-His antibody (Cisbio Cat 61HI2TLB). The binding of the Cy5-probe to anti-His-Tb/His-tag TEAD1 complex yielded a TR-FRET signal. Addition of compounds that are TEAD1 lipid pocket binders resulted in the displacement of the Cy5-probe from TEAD1 and a decrease in the TR-FRET signal. After 60 minutes incubation at room temperature of compounds with the His-TEAD1/anti-His-Tb/Cy5-probe complex, the plate was read on a plate reader (BMG ClarioStar Cat 430-1300) using TR-FRET mode with wavelengths of 665 nm/620 nm. The potency of compounds as TEAD1 lipid pocket binders was determined by IC50 value generated using a non-linear 4 parameter curve fit.

72H TEAD Proliferation Assay.

The effect of TEAD inhibition on cell proliferation was assayed using Cell Titer Glo (CTG) 2.0 to measure response in mesothelioma cell lines NCI-H226 (ATCC, #CRL-5826) and NCI-H28 (ATCC, #CRL-5820).

Description

The 72H TEAD Proliferation assay utilizes Cell Titer-Glo 2.0 (Promega, #G9243) to measure the proliferation of cells in the presence or absence of compound. Cell Titer-Glo 2.0 determines the amount of viable cells by quantifying ATP (an indication of metabolically active cells). It utilizes the conversion of Luciferin to Oxyluciferin and a luminescent signal with the use of ATP to report the quantity of viable cells in culture. Within cells that are continually growing, ATP is being synthesized to meet their metabolic demands, meanwhile the opposite is true for cells that are dying or slowing down their proliferation and either no longer using ATP or are using less, respectively. The NF2-deficient NCI-H226 has been genetically validated as a cell line that is sensitive to TEAD inhibition. The NF2-wild type NCI-H28 has been genetically validated as a cell line that is not sensitive to TEAD inhibition and grows independently of TEAD activity.

Application

Monitor for any effects on proliferation with compound treatment.

Compounds were screened against the responsive NCI-H226 cell line to assess the compounds' ability to inhibit TEAD and cell growth. Compounds were also screened against the non-responsive NCI-H28 cell line to ascertain whether the inhibition of cell growth was due to inhibition of the target TEAD or whether the inhibition was due to off-target cytotoxicity.

General Culture Conditions

Thaw Medium 1/Growth Medium 1: RPMI 1640 with GlutaMAX supplement medium (Gibco, #61870036) with 10% FBS (Gibco, #A3160402))

Assay Medium 1: RPMI 1640 medium with L-Glutamine, no phenol red (Gibco, #11835030) with 10% FBS (Gibco, #A3160402)

Both NCI-H226 and NCI-H28 cells were grown at 37° C. with 5% CO2 using Growth Medium 1.

To recover the cells, frozen stock was thawed quickly in a 37° C. water-bath after removal from liquid nitrogen, transferred to a tube containing 1 ml of pre-warmed Thaw Medium 1, spun down, resuspended with 1 ml of pre-warmed Growth Medium 1 and added into a T75 with 9 ml of Growth Medium 1. The cell culture was grown in an incubator at 37° C. with 5% CO₂. At first passage, cells were transferred into a T150 with 15 mL Growth Medium 1 to allow the cells to continue growing. Cells were split before they reached complete confluency and were not used past passage number 20.

The cells were passaged by first rinsing them with phosphate buffered saline (PBS), and then detaching them from the flask with TrypLE Express (1×) (Gibco, #12604013). Growth Medium 1 was added and the cell suspension was transferred to a tube. The cells were counted and the volume was reduced to get 1M cells was added to another tube. The cells were spun down and resuspended in 2 mL of fresh Growth Medium 1. 1 ml of the cell suspension was added into a new T150 with 14 mL of Growth Medium 1. Subcultivation ratio: 500,000 cells in a T150 weekly.

The cells were frozen by rinsing them with phosphate buffered saline (PBS), and detaching them from the flask with TrypLE Express (1×) (Gibco, #12604013). Growth Medium 1 was added and the cell suspension was transferred to a tube. The cells were spun down and resuspended in freezing medium (95% FBS+5% DMSO). The cells were then added to cryovials and stored at −80° C. overnight then transferred to liquid nitrogen the next day.

Functional Validation and Assay Performance

The following assays were designed for 384-well format. Performing the assay in different tissue culture formats will need the cell number and reagent volume to be scaled up appropriately.

Materials
Thaw Medium 1/Growth Medium 1 (Gibco, #61870036)+10% FBS (Gibco #A3160402)
   Assay Medium 1 (Gibco, #11835030) with 10% FBS (Gibco #A3160402)
   Phosphate Buffered Saline (Gibco, #10010023)
   TrypLE Express (Gibco, #12604013)
   Trypan Blue 0.4% (Invitrogen, #T10282)
   Countess II FL Automated Cell Counter (ThermoFisher Scientific, #AMQAF1000)
   Multidrop Combi Reagent Dispenser (ThermoFisher Scientific, #5840300)
   384-well Low Flange Black Flat Bottom Polystyrene TC-treated Microplates (Corning 3571)
   Echo (Beckman)
   CTG 2.0 (Promega, #G9243)
   Bravo Liquid Handler (Agilent)
   EnVision Multilabel Plate Reader (PerkinElmer)

Mycoplasma Testing of NCI-H226 and NCI-H28 cell lines

The 2 cell lines were tested for Mycoplasma by IDEXX BioAnalytics using PCR-based Mycoplasma detection and confirmed to be negative.

Anti-proliferative effect of compounds that inhibits TEAD activity measured by CTG

1) Assay ready plates (ARPs) were prepared by Echo acoustic liquid handler. For each compound, duplicate of 10-point half-log dilution series were dispensed in a 384-well microplate (Corning 3571).

2) Each well had 50nl of compound and had a final DMSO of 0.1% after cell plating.

3) Before use, ARPs were allowed to warm up to room temperature for 30 min.

4) ARPs were spun for 5 minutes at 1500RPM before removing the plate seals.

5) NCI-H226 or NCI-H28 cells were harvested from culture in Assay Medium 1 and the multidrop Combi reagent dispenser was used to seed cells at 500 cells per 50ul in each well of the ARPs and one Corning 3571 plate without compounds for Time 0 (TO) readout.

6) The TO plate was incubated for 2 hours at 37° C. with 5% CO₂ to allow cells to settle, then the CTG Assay was performed.

7) All ARPs were incubated for 72 hours at 37° C. with 5% CO₂, then the CTG Assay was performed for Time 72H (T72) readout.

8) CTG Assay: Bravo liquid handler (Agilent) was used to add 25ul of CTG 2.0 to all columns of the plate except for Column 24, which was used to subtract out the background. After CTG addition, plates were placed on shaker at 800RPM for 15 minutes at room temperature and kept in the dark. Luminescence was measured using EnVision multilabel plate reader with ultra-sensitive detection module.

9) Data Analysis: First background luminescence (no CTG wells) was subtracted from luminescence reading of all wells, then TO luminescence was subtracted from T72 luminescence. To compare anti-proliferative effect of compounds, GI₅₀ was obtained by fitting dose response curves with nonlinear regression curve fit.

Results are presented in Table 1. Compounds having an IC₅₀ less than or equal to 150 nM are represented as “A”; compounds having an ICS(greater than 150 nM but less than or equal to 300 nM are represented as “B”; compounds having an IC50 greater than 300 nM but less than or equal to 500 nM are represented as “C”; and compounds having an IC₅₀ greater than 500 nM are represented as “D”. Compounds having a GI₅₀ less than or equal to 500 nM are represented as “A”; compounds having a GI₅₀ greater than 500 nM but less than or equal to 1 μM are represented as “B”; compounds having a GI₅₀ greater than 1 μM but less than or equal to 5 μM are represented as “C”; and compounds having a GI₅₀ greater than 5 μM are represented as “D”.

TABLE 1

| | CDA | | | CDA | |
|---|---|---|---|---|---|
| Compound | IC₅₀ | GI₅₀ | Compound | IC₅₀ | GI₅₀ |
| I-1 | B | B | I-15 | A | A |
| I-2 | C | A | I-16 | B | C |
| I-3 | B | B | I-17 | A | A |
| I-4 | B | B | I-18 | B | C |
| I-5 | A | C | I-19 | B | D |
| I-6 | A | B | I-20 | A | A |
| I-7 | B | C | I-21 | A | A |
| I-8 | D | D | I-22 | B | A |
| I-9 | B | A | I-23 | C | C |
| I-10 | C | C | I-24 | D | D |
| I-11 | D | C | I-25 | D | C |
| I-12 | D | A | I-26 | C | B |
| I-13 | A | A | I-27 | C | C |
| I-14 | B | C | I-28 | B | B |
| I′-29 | A | C | I-49 | A | A |
| 30 | D | D | I-50 | A | B |
| 31 | D | C | I-51 | A | A |
| I′-32 | C | C | I-52 | A | A |
| I′-33 | B | B | I″-53 | A | A |
| I′-34 | C | D | I″-54 | A | A |
| I-35 | C | B | I″-55 | A | A |
| I-36 | B | B | I″-56 | D | D |
| I-37 | B | A | I-57 | B | D |
| I-38 | B | A | I-58 | A | A |
| I-39 | D | A | I-59 | B | A |
| I-40 | B | B | I-60 | B | B |
| I-41 | B | D | I-61 | B | B |
| I-42 | B | D | I-62 | A | A |
| I-43 | A | D | I-63 | C | C |
| I-44 | — | D | I-64 | A | A |
| I-45 | A | C | I-65 | A | A |
| I-46 | C | C | 66 | A | C |
| I-47 | D | D | 67 | D | D |
| I-48 | B | A | 68 | D | D |

### Example 3. TEAD Compound in Combination with EGFR Inhibitor

EGFR-mutant NSCLC PC-9 cells were plated in a 96 well tissue culture plate (Corning #3596). On the next day, PC-9 cells were pre-treated with Osimertinib (100 nM) for 24 h, then co-treated with Osimertinib (100 nM) and compound I-1 for 48 hours at various concentrations. Apoptosis was detected by CellEvent Caspase 3/7 Green ReadyProbes Reagent (ThermoFisher), a fluorogenic indicator of activated caspase-3/7. Cell death (apoptsis) and cell growth (phase confluence) over time were captured by IncuCyte live cell imaging system (Essen Bioscience) and quantified by IncuCyte S3 software (Essen Bioscience). Apoptotic Index was calculated by dividing apoptosis signal with phase confluence. Fold change was calculated by dividing apoptotic index of treatment by apoptotic index of DMSO sample, as shown in Table 2.

TABLE 2

| | Fold Change over |
|---|---|
| Sample | DMSO at 48 hrs |
| Osimertinib (100 nM) | 2.6 |
| I-1 (10 μM) | 1.0 |
| Osimertinib (100 nM) + | 4.1 |
| I-1 (0.37 μM) | |
| Osimertinib (100 nM) + | 5.4 |
| I-1 (1.1 μM) | |
| Osimertinib (100 nM) + | 7.0 |
| I-1 (10 μM) | |

### Exemplary Enumerated Embodiments

1. A compound of Formula I:

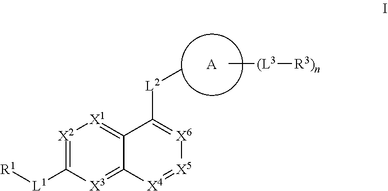

or a pharmaceutically acceptable salt thereof,
X¹ is C—R^{x1} or N;
X² is C—R^{x2} or N;
X³ is C—R^{x3} or N;
X⁴ is C—R^{x4} or N;
X⁵ is C—R^{x5} or N;
X⁶ is C—R^{x6} or N;
wherein no more than three of X¹, X², X³, X⁴, X⁵, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
   R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
L² is a covalent bond, —OCH₂—^{#}, or —N(R)CH₂—^{#}, wherein ^{#} represents the point of attachment to Ring A;
Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur; and
n is 0-5;
provided that when L² is a covalent bond and Ring A is phenyl, then at least one L³ is —O— or —NR—.
   2. The compound according to embodiment 1, wherein Ring A is phenyl.
   3. The compound according to embodiment 1, wherein Ring A is selected from the group consisting of a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur.
   4. The compound according to embodiment 1 or 3, wherein Ring A is selected from the group consisting of cyclohexyl, piperidinyl, piperazinyl, 6-azaspiro[2.5]octanyl, 7-azaspiro[3.5]nonanyl, or 8-azaspiro[4.5]decanyl.
   5. The compound according to any one of embodiments 1-4, wherein the compound is of Formulae II-a, II-a1, or II-a2:

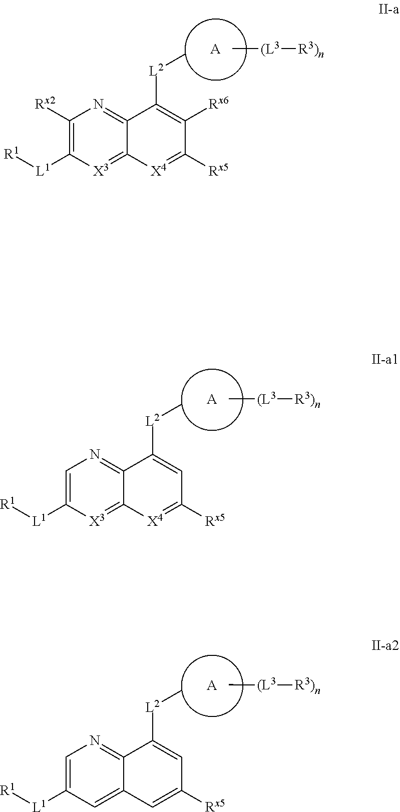

or a pharmaceutically acceptable salt thereof.
6. The compound according to any one of embodiments 1-4, wherein the compound is of Formulae III-a, III-a1, or III-a2:

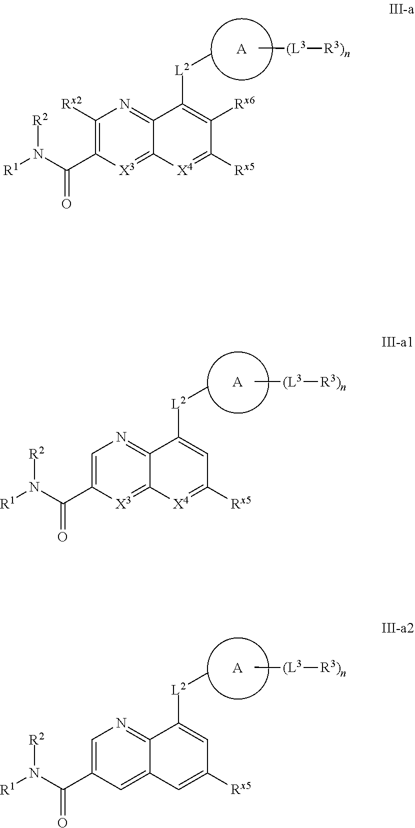

or a pharmaceutically acceptable salt thereof.
7. The compound according to embodiment 1 or 2, wherein the compound is of Formulae IV-a, IV-a1, or IV-a2:

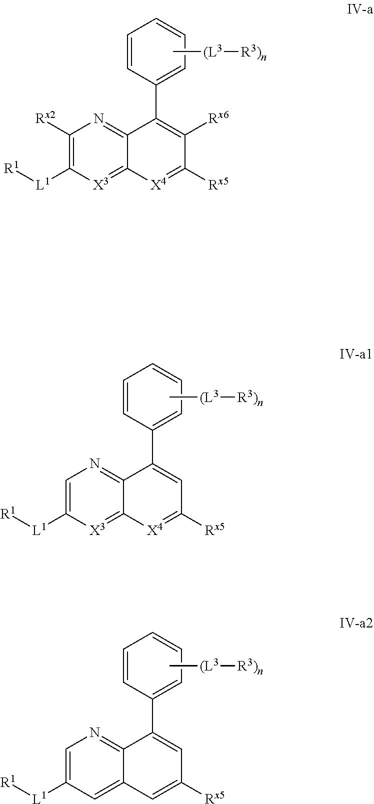

or a pharmaceutically acceptable salt thereof.
8. The compound according to embodiment 1 or 2, wherein the compound is of Formulae V-a, V-a1, or V-a2:

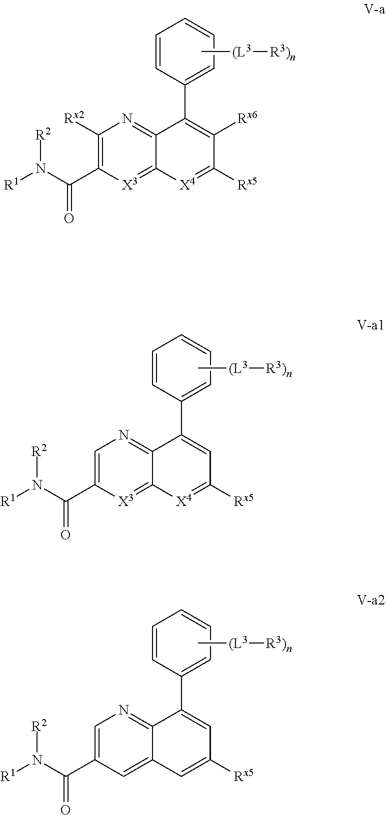

or a pharmaceutically acceptable salt thereof.
9. The compound according to embodiment 1 or 2, wherein the compound is of Formulae VI-a, VI-a1, or VI-a2:

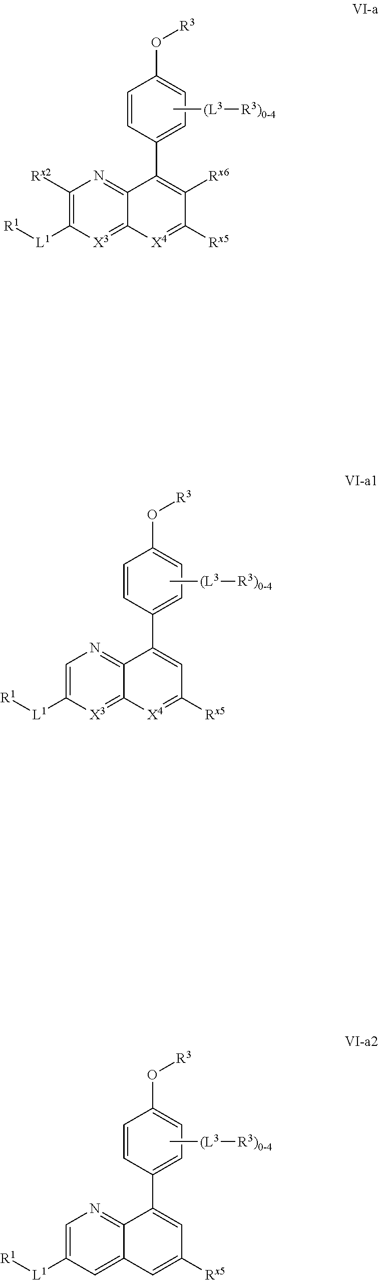

or a pharmaceutically acceptable salt thereof.
10. The compound according to embodiment 1 or 2, wherein the compound is of Formulae VII-a, VII-a1, or VII-a2:

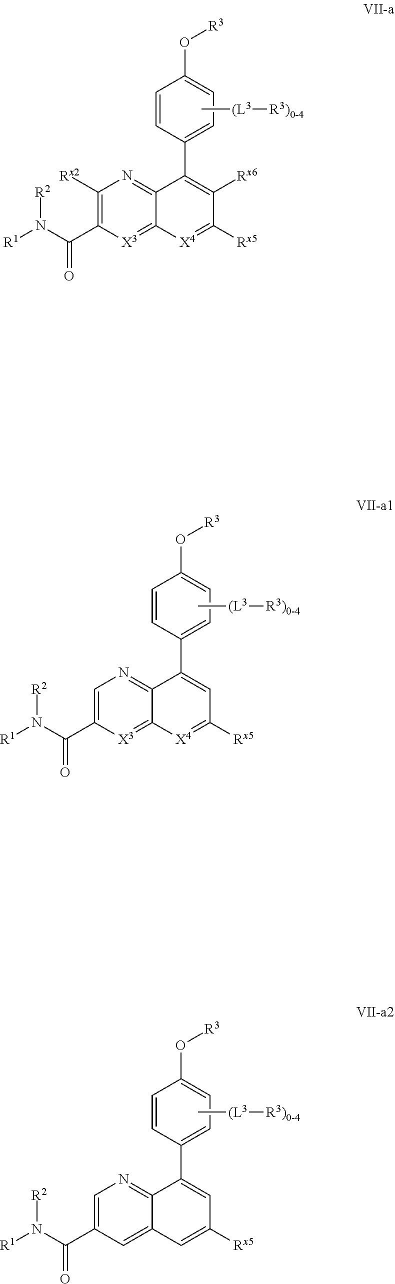

or a pharmaceutically acceptable salt thereof.
11. The compound according to any one of embodiments 1 or 3-4, wherein the compound is of Formulae VIII-a, VIII-a1, or VIII-a2:

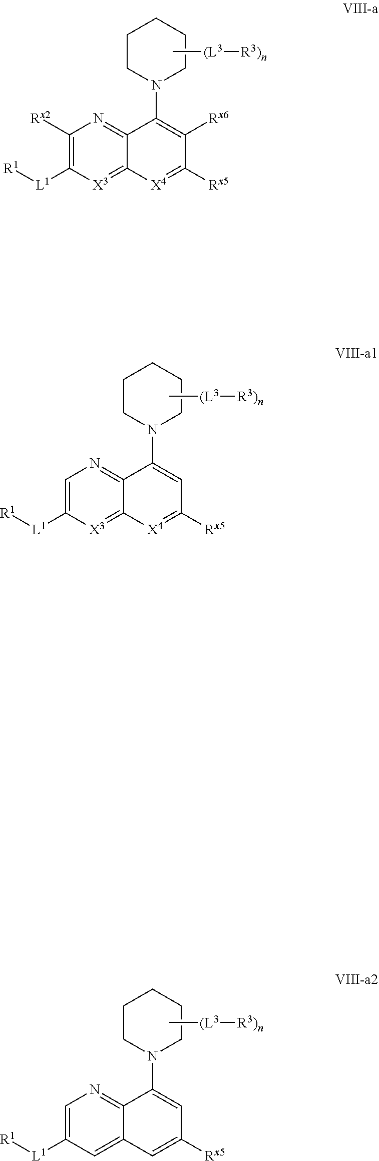

or a pharmaceutically acceptable salt thereof.
12. The compound according to any one of embodiments 1 or 3-4, wherein the compound is of Formulae IX-a, IX-a1, or IX-a2:

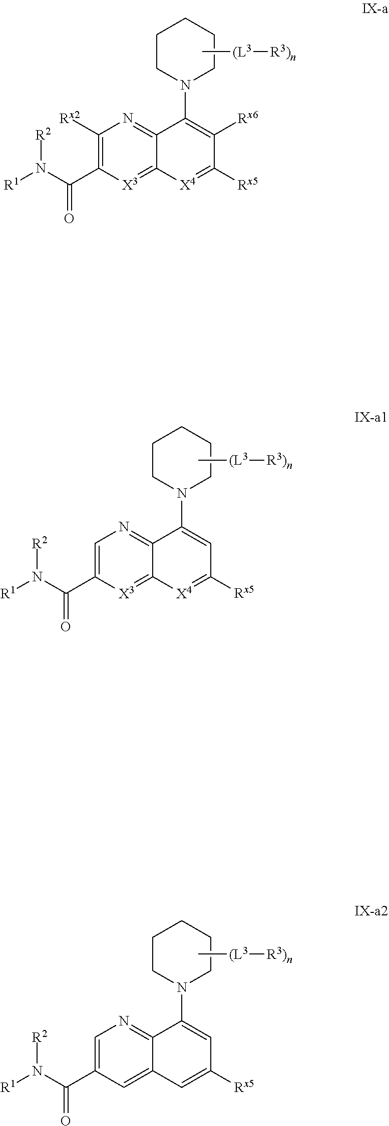

or a pharmaceutically acceptable salt thereof.
13. The compound according to any one of embodiments 1 or 3-4, wherein the compound is of Formulae X-a, X-a1, or X-a2:

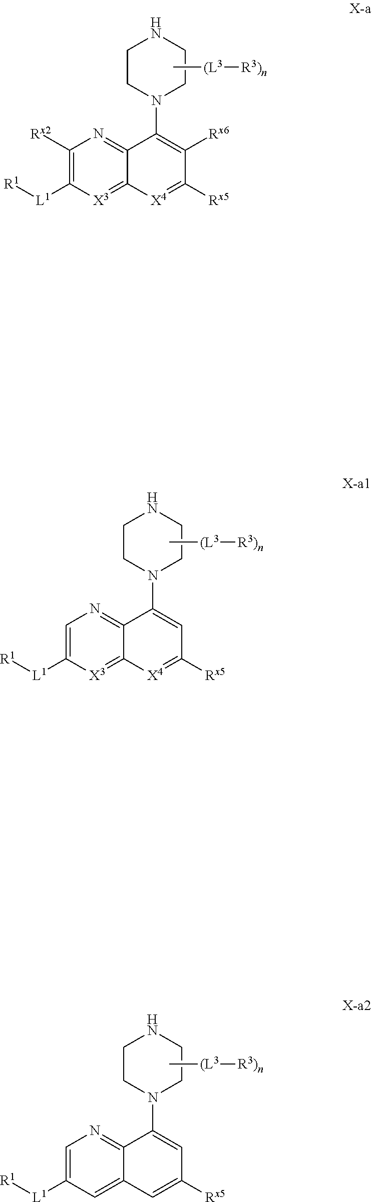

or a pharmaceutically acceptable salt thereof.
14. The compound according to any one of embodiments 1 or 3-4, wherein the compound is of Formulae XI-a, XI-a1, or XI-a2:

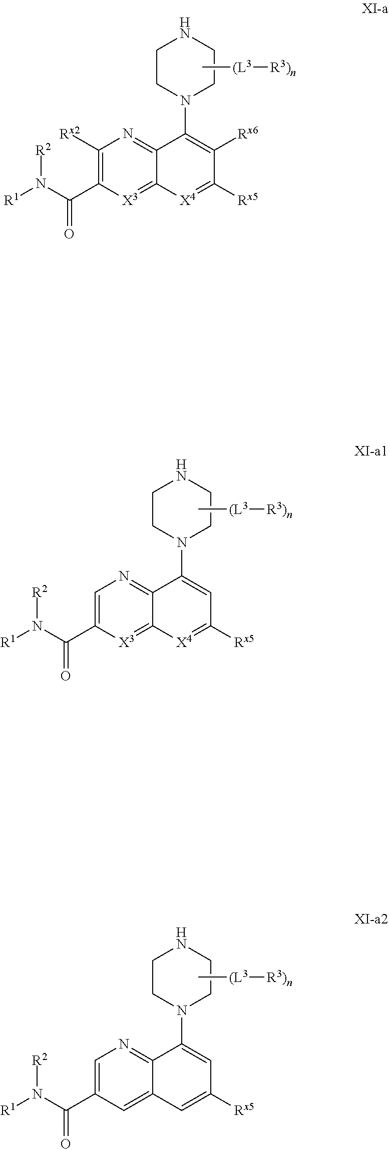

or a pharmaceutically acceptable salt thereof.
15. The compound according to any one of embodiments 1 or 3-4, wherein the compound is of Formulae XII-a, XII-a1, XII-a2, XIV-a, XIV-a1, XIV-a2, XVI-a, XVI-a1, or XVI-a2:

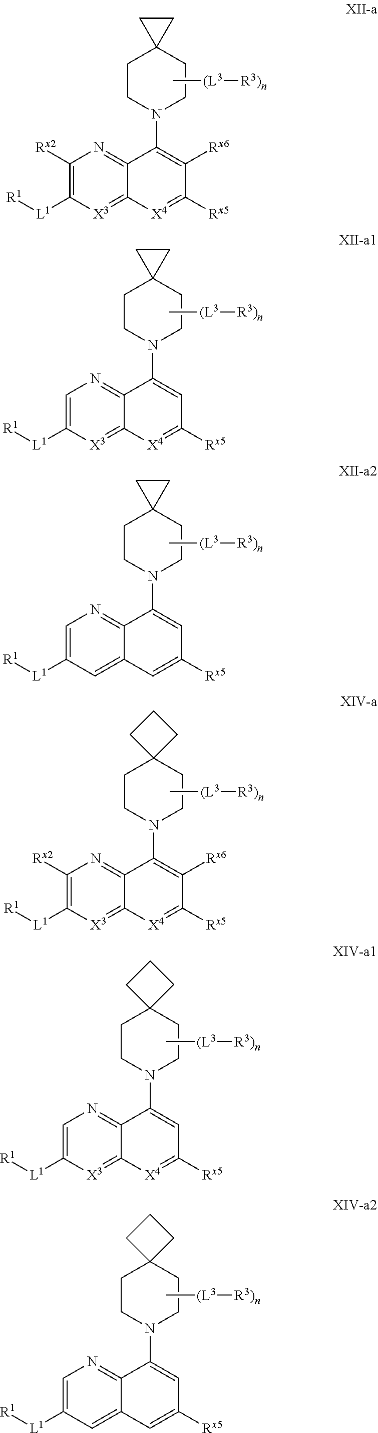

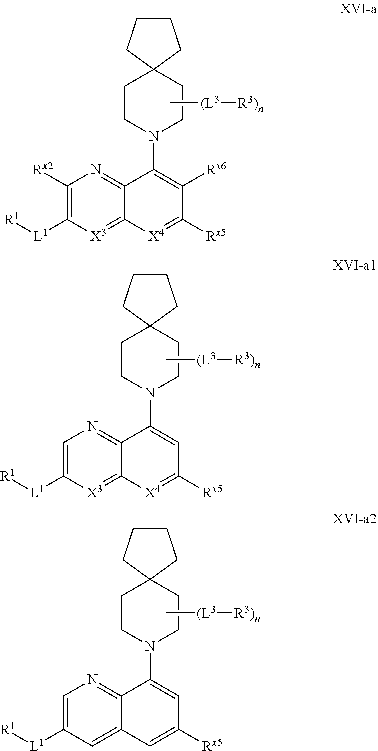

or a pharmaceutically acceptable salt thereof.
16. The compound according to any one of embodiments 1 or 3-4, wherein the compound is of Formulae XIII-a, XIII-a1, XIII-a2, XV-a, XV-a1, XV-a2, XVII-a, XVII-a1, or XVII-a2:

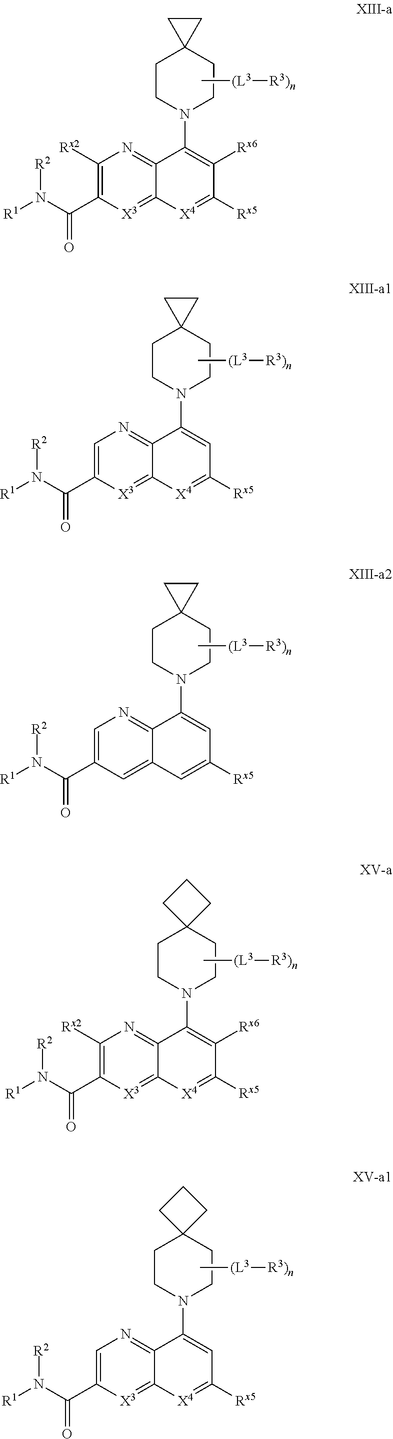

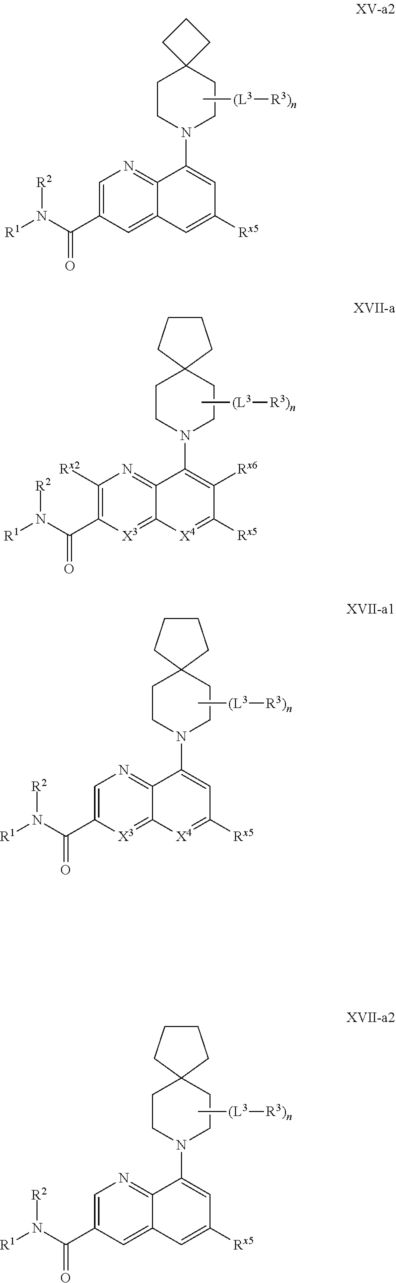

or a pharmaceutically acceptable salt thereof.
17. The compound according to any one of embodiments 1-5, 7, 9, 11, 13, or 15, wherein LI is —C(O)N(R²)—.
18. The compound according to any one of embodiments 1-6 or 17, wherein L² is a covalent bond.
19. The compound according to any one of embodiments 1-18, wherein X₃ is N.
20. The compound according to any one of embodiments 1-19, wherein X₄ is N.
21. The compound according to any one of embodiments 1-18, wherein X₃ is C—R^{x3}.
22. The compound according to any one of embodiments 1-18 or 21, wherein X⁴ is C-R^{x4}.
23. The compound according to embodiment 21 or 22, wherein R^{x3} is hydrogen.
24. The compound according to any one of embodiments 21-23, wherein R^{x4} is hydrogen.
25. The compound according to any one of embodiments 1-24, wherein R^{x2} is hydrogen.
26. The compound according to any one of embodiments 1-25, wherein R^{x6} is hydrogen.
27. The compound according to any one of embodiments 1-26, wherein R^{x5} is —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur.
28. The compound according to any one of embodiments 1-27, wherein R^{x5} is —CN, halogen, —OR, —N(R)₂, or an optionally substituted C₁₋₆ aliphatic.
29. The compound according to any one of embodiments 1-28, wherein R^{x5} hydrogen, —OCH₃, —OCF₂H, —OCF₃, or

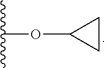 [SMILES: *OC1CC1]

30. The compound according to any one of embodiments 1-29, wherein R¹ is an optionally substituted C₁₋₆ aliphatic.
31. The compound according to any one of embodiments 1-30, wherein R¹ is selected from the group consisting of:

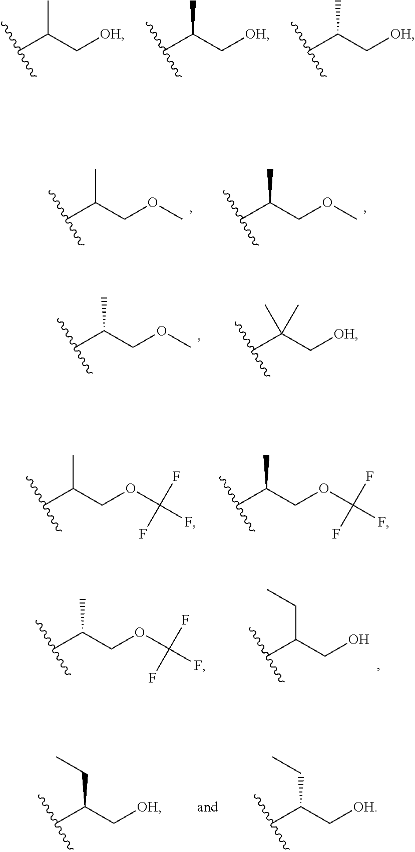

32. The compound according to any one of embodiments 1-31, wherein R² is hydrogen.
33. The compound according to any one of embodiments 1-29, wherein R¹ and R², together with their intervening atoms, form an optionally substituted 3- to 7-membered saturated or partially unsaturated heterocyclic ring having 1-3 heteroatoms atoms independently selected from nitrogen, oxygen, and sulfur.
34. The compound according to any one of embodiments 1-29 or 33, wherein R¹ and R², together with their intervening atoms, form an optionally substituted 3- to 7-membered saturated or partially unsaturated heterocyclic ring having 1 nitrogen heteroatom.
35. The compound according to any one of embodiments 1-34, wherein L³ is —O—.
36. The compound according to any one of embodiments 1-35, wherein R³ is an optionally substituted group selected from the group consisting of C₁₋₆ aliphatic, phenyl, or a 3- to 7-membered saturated or partially unsaturated carbocyclic ring.
37. The compound according to any one of embodiments 1-36, wherein R³ is t-butyl, —CHF₂, —CF₃, —CH₂CF₃, phenyl, or cyclopropyl.
38. The compound according to any one of embodiments 1-34, wherein L³ is a covalent bond.
39. The compound according to any one of embodiments 1-34 or 38, wherein R³ is halogen or optionally substituted C₁₋₆ aliphatic.
40. The compound according to any one of embodiments 1-34 or 38-39, wherein R³ is fluoro, t-butyl, —CHF₂, —CF₃, or —CH₂CF₃,
41. A pharmaceutical composition comprising a compound according to any one of embodiments 1-40, or a pharmaceutically acceptable salt thereof, and a pharmaceutically acceptable carrier, adjuvant, or vehicle.
42. A method of inhibiting activity of a TEAD transcription factor, or a mutant thereof, in a biological sample or in a patient, the method comprising a step of contacting the biological sample or administering to a patient a compound according to any one of embodiments 1-40, or a pharmaceutically acceptable salt thereof.
43. A method of treating a disease or disorder associated with TEAD, the method comprising a step of administering to a patient in need thereof a compound according to any one of embodiments 1-40, or a pharmaceutically acceptable salt thereof.
44. The method according to embodiment 43, wherein the disease or disorder associated with TEAD is a proliferative disease.
45. The method according to embodiment 44, wherein the proliferative disease is a cancer.
46. The method according to embodiment 45, wherein the cancer is selected from a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.
47. The method according to any one of embodiments 43-46, further comprising co-administration of at least one inhibitor of the RAS/MAPK pathway.
48. The method according to embodiment 47, wherein the RAS/MPAK pathway inhibitor is a KRAS inhibitor, RAF inhibitor, a MEK inhibitor, an ERK inhibitor, an EGFR inhibitor, or a MAPK inhibitor.

1. A compound of Formula I: 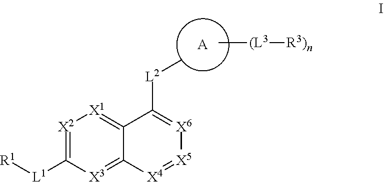
or a pharmaceutically acceptable salt thereof,
X¹ is C—R^{x1} or N;
X² is C—R^{x2} or N;
X³ is C—R^{x3} or N;
X⁴ is C—R^{x4} or N;
X⁵ is C—R^{x5} or N;
X⁶ is C—R^{x6} or N;
wherein no more than three of X¹, X², X³, X⁴, X⁵, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
L² is a covalent bond, —OCH₂—^{#}, or —N(R)CH₂—^{#}, wherein ^{#} represents the point of attachment to Ring A;
Ring A is selected from the group consisting of phenyl, a 5- to 6-membered heteroaryl ring having 1-3 heteroatoms independently selected from nitrogen, oxygen, and sulfur, a 3- to 7-membered saturated or partially unsaturated carbocyclic ring, a 3- to 7-membered saturated or partially unsaturated heterocyclic ring having 1-3 heteroatoms independently selected from nitrogen, oxygen, and sulfur, and a 8- to 11-membered spirofused saturated or partially unsaturated heterocyclic ring having 1-3 heteroatoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur; and
n is 0-5;
provided that when L² is a covalent bond and Ring A is phenyl, then at least one L³ is —O— or —NR—.

2-4. (canceled)

5. The compound according to claim 1, wherein the compound is of Formulae II-a, II-a1, II-a2, III-a, III-a1, or III-a2: 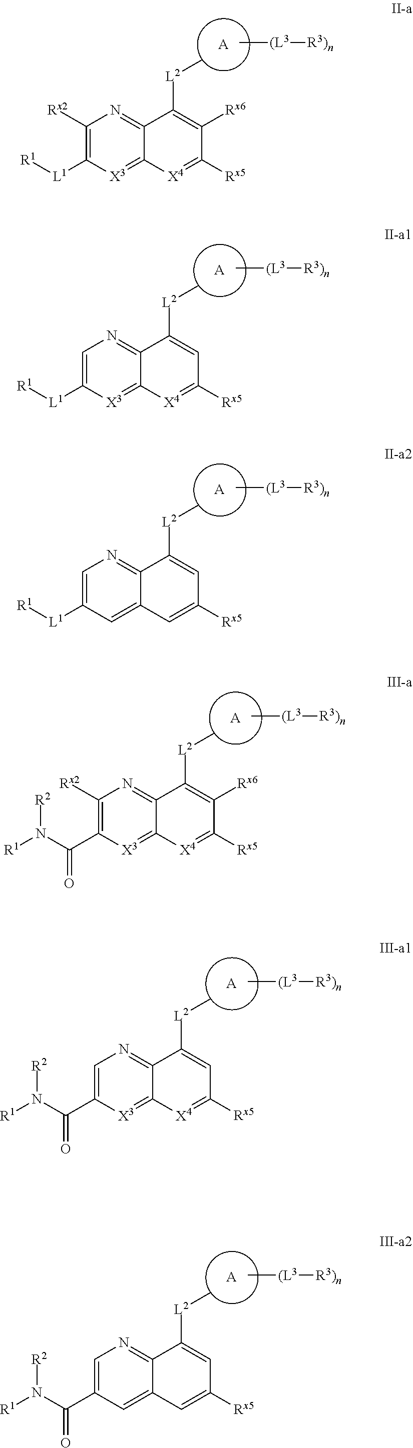
or a pharmaceutically acceptable salt thereof.

6. (canceled)

7. The compound according to claim 1, wherein the compound is of Formulae IV-a, IV-a1, IV-a2, V-a, V-a1, V-a2, VI-a, VI-a1, VI-a2, VII-a, VII-a1, or VII-a2: 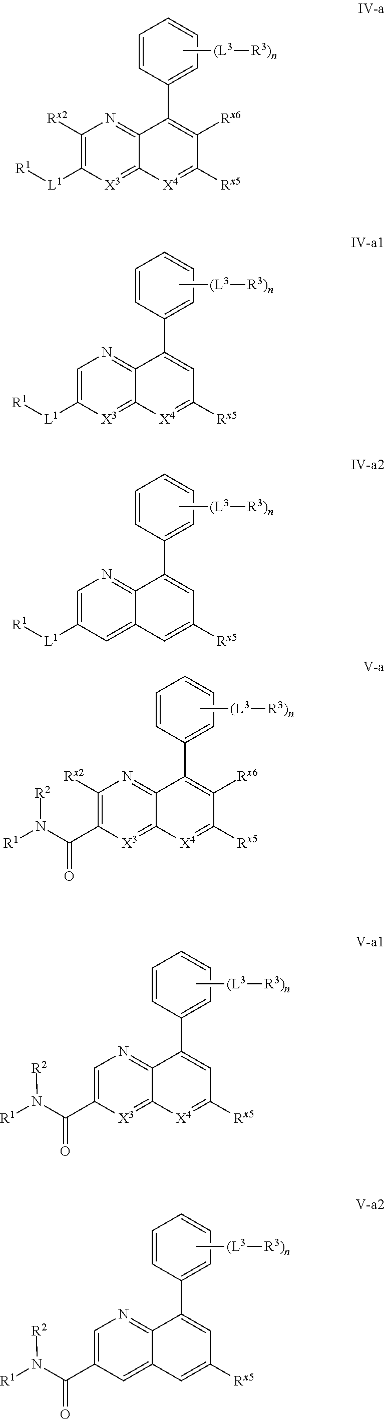 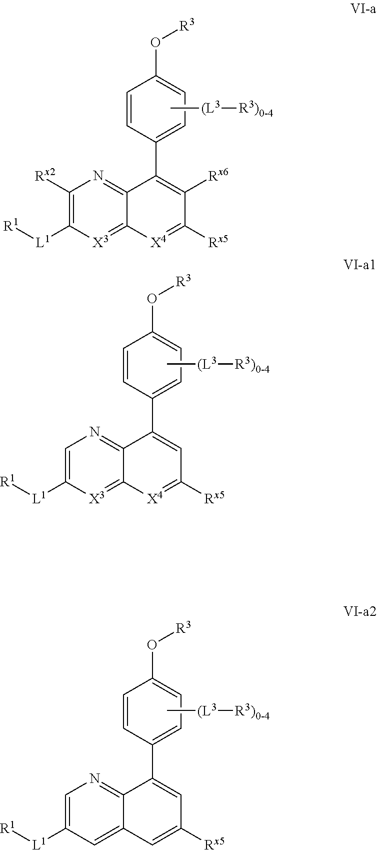 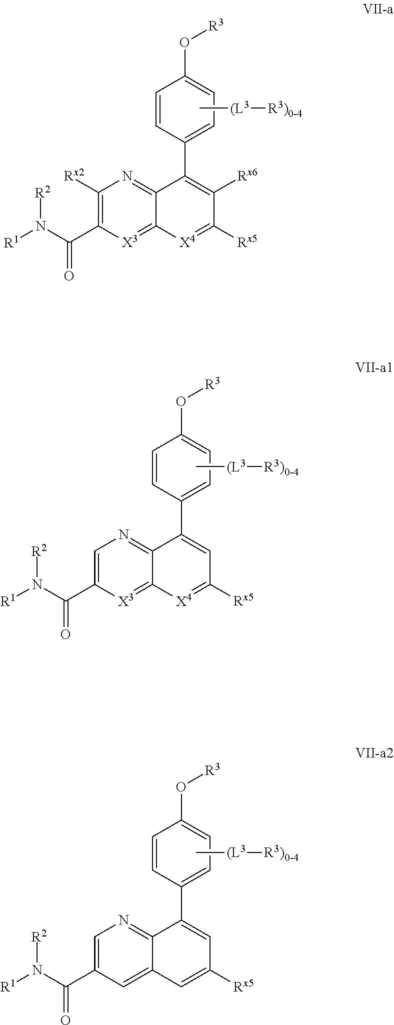
or a pharmaceutically acceptable salt thereof.

8-10. (canceled)

11. The compound according to claim 1, wherein the compound is of Formulae VIII-a, VIII-a1, VIII-a2, IX-a, IX-a1, or IX-a2: 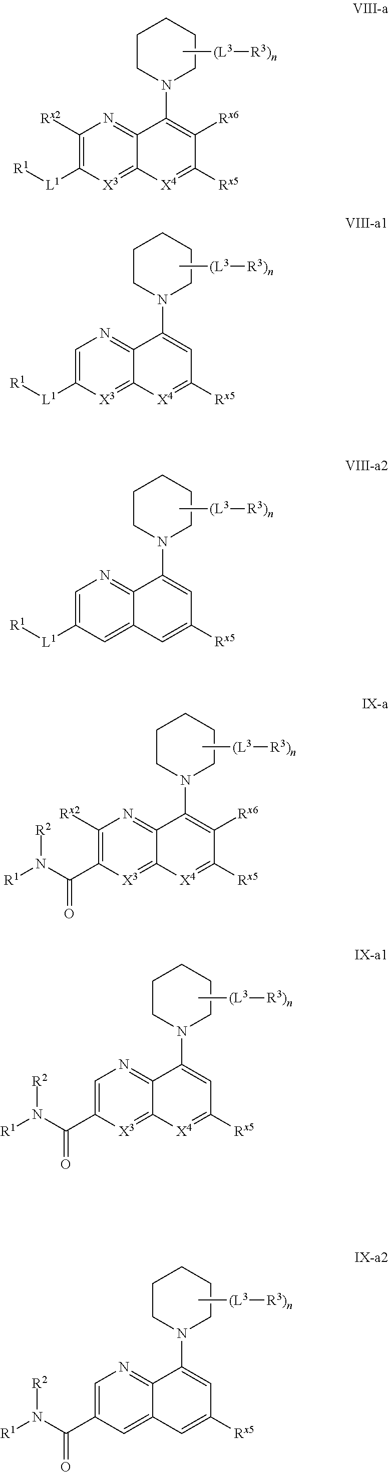
or a pharmaceutically acceptable salt thereof.

12. (canceled)

13. The compound according to claim 1, wherein the compound is of Formulae X-a, X-a1, X-a2, XI-a, XI-a1, or XI-a2: 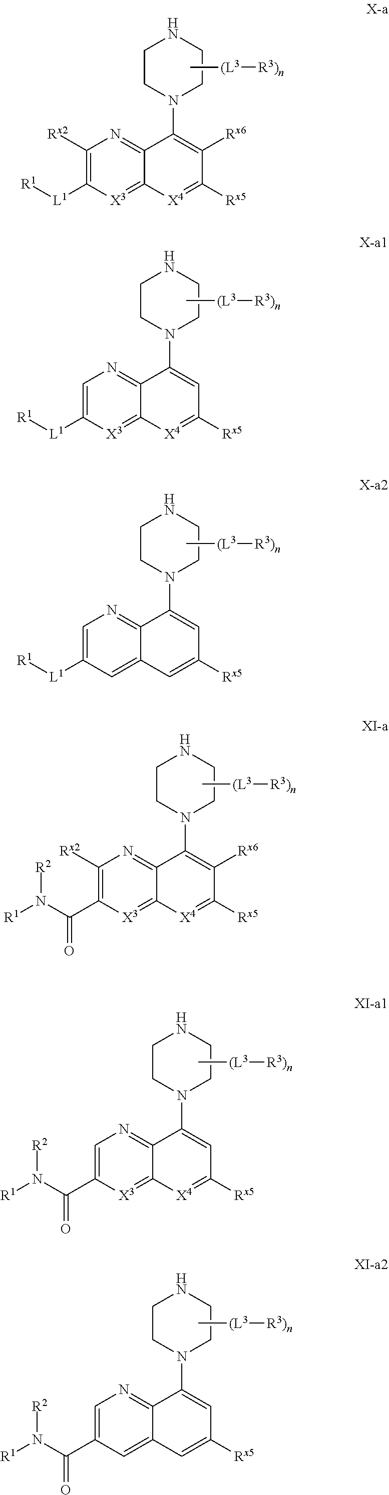
or a pharmaceutically acceptable salt thereof.

14. (canceled)

15. The compound according to claim 1, wherein the compound is of Formulae XII-a, XII-a1, XII-a2, XIV-a, XIV-a1, XIV-a2, XVI-a, XVI-a1, XVI-a2, XIII-a, XIII-a1, XIII-a2, XV-a, XV-a1, XV-a2, XVII-a, XVII-a1, or XVII-a2: 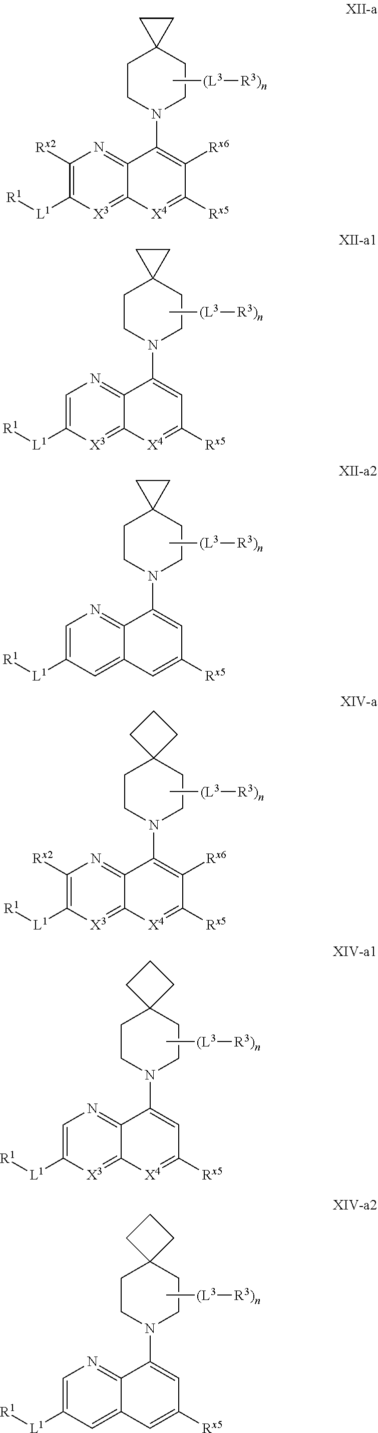 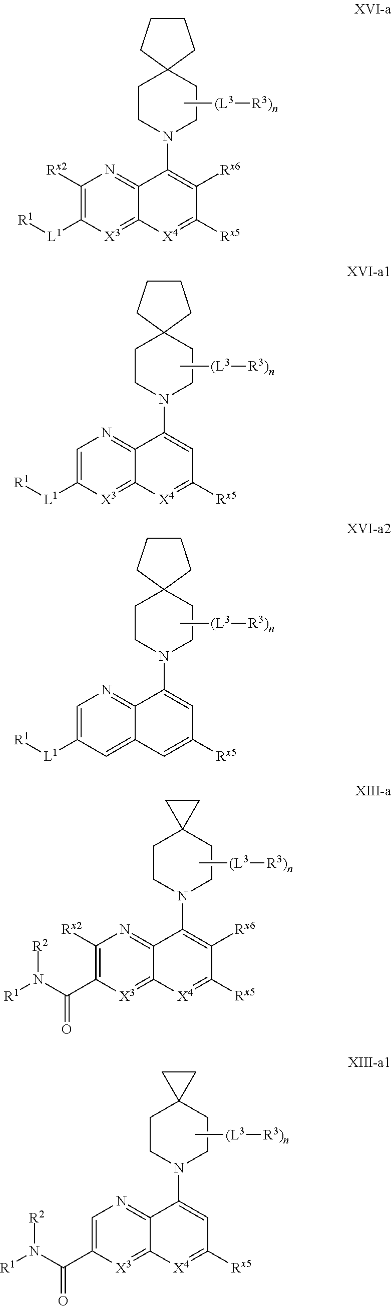 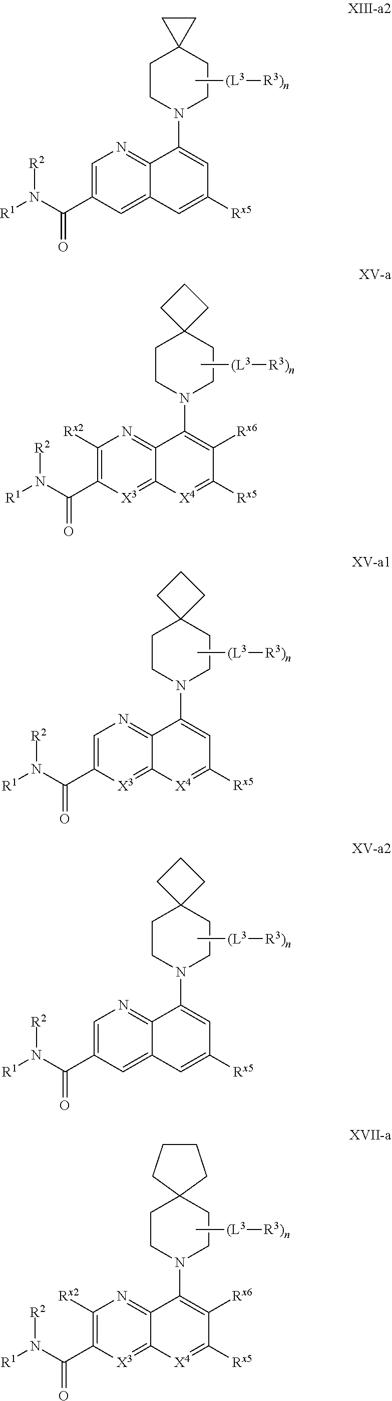 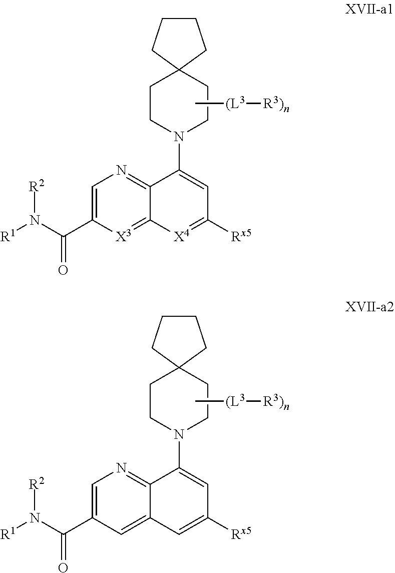
or a pharmaceutically acceptable salt thereof.

16. (canceled)

17. The compound according to claim 1, wherein the compound is of Formulae XVIII-d, XVIII-d1, XVIII-d1, XIX-d, XIX-d1, XIX-d2, XX-a, XX-a1, XX-a2, XX-d, XX-d1, XX-d2, XXI-a, XXI-a1, XXI-a2, XXI-d, XXI-d1, or XXI-d2: 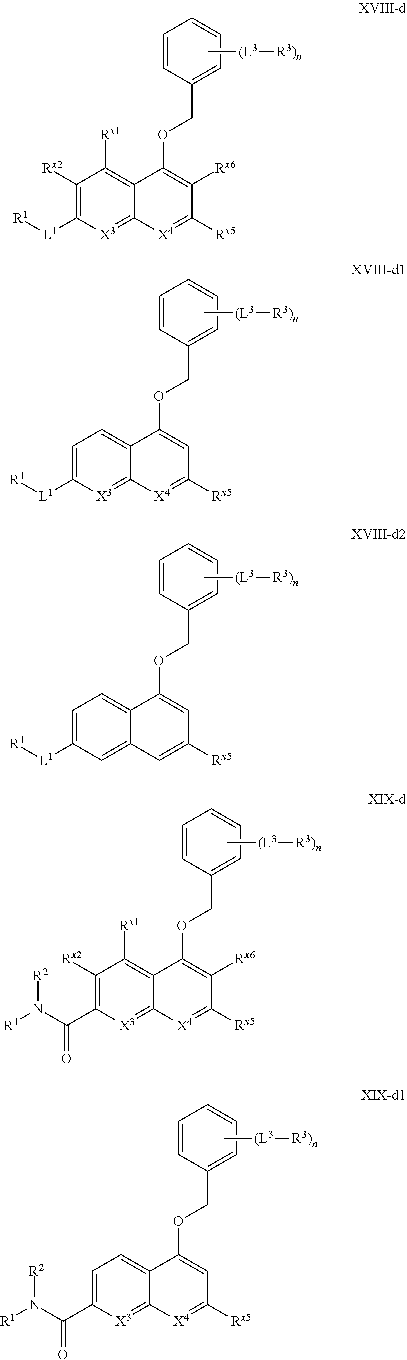 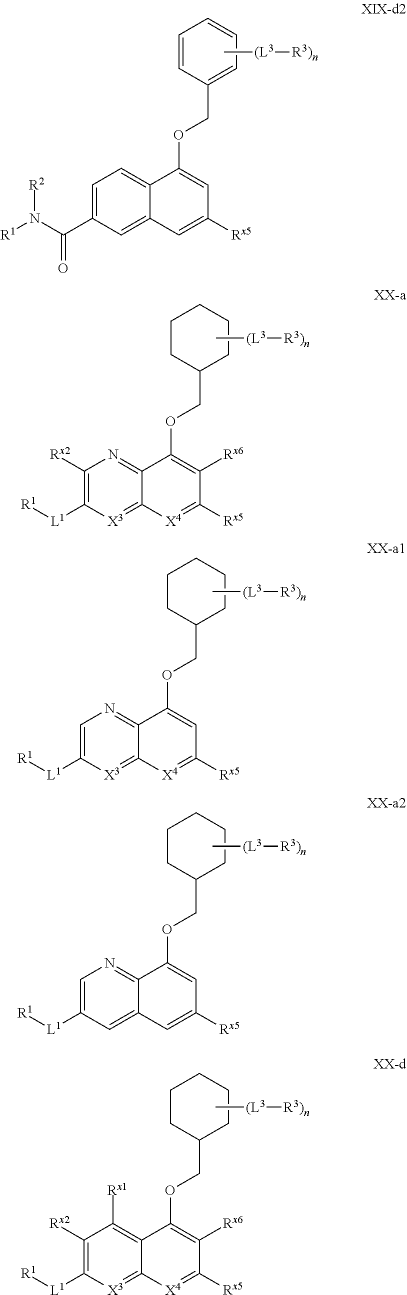 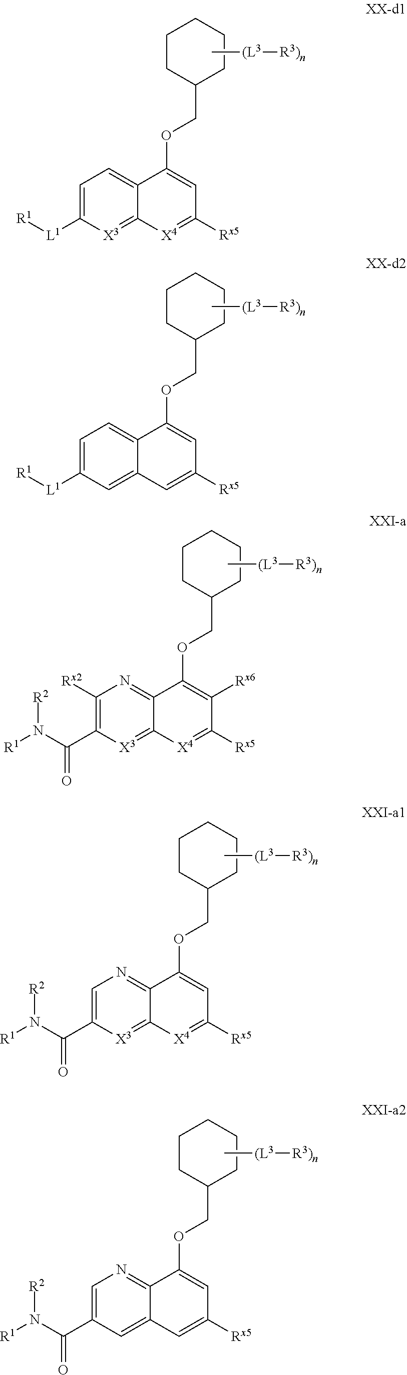 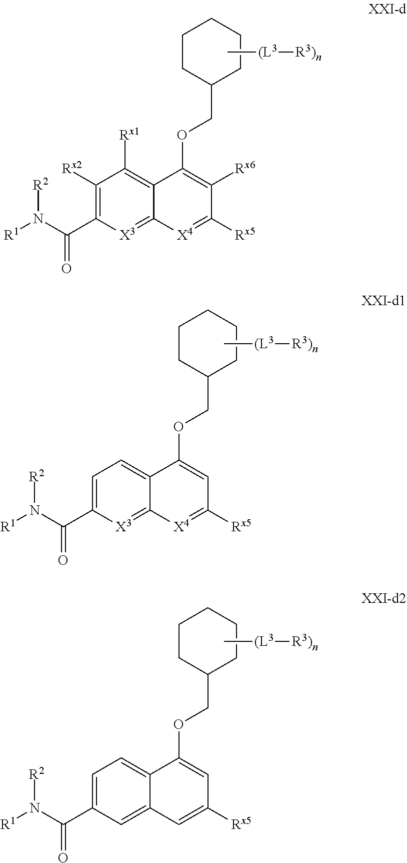
or a pharmaceutically acceptable salt thereof.

18-19. (canceled)

20. The compound according to claim 1, wherein L¹ is —C(O)N(R²)—.

21-23. (canceled)

24. The compound according to claim 1, wherein R^{x5} is hydrogen, —OCH₃, —OCF₂H, —OCF₃, 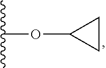 [SMILES: *OC1CC1]
—OCH₂CH₃, —OH, methyl, or —C₃.

25-26. (canceled)

27. The compound according to claim 1, wherein R¹ is selected from the group consisting of:

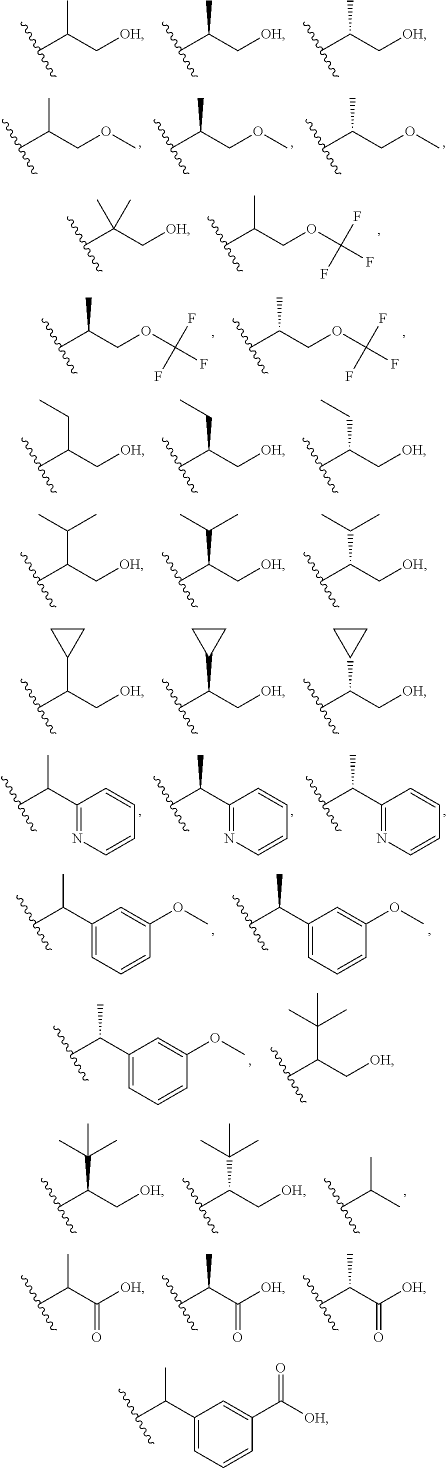

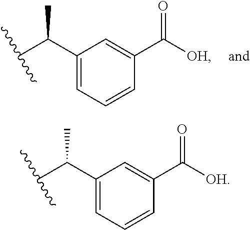

28-31. (canceled)

32. The compound according to claim 1, wherein L³ is —O—.

33. (canceled)

34. The compound according to claim 32, wherein R³ is t-butyl, —CHF₂, —CF₃, —CH₂CF₃, phenyl, or cyclopropyl.

35. The compound according to claim 1, wherein L³ is a covalent bond.

36. (canceled)

37. The compound according to claim 35, wherein R³ is fluoro, t-butyl, —CHF₂, —CF₃, or —CH₂CF₃.

38. A compound of Formula I′ 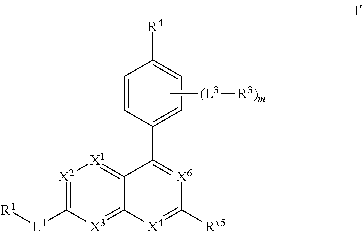
or a pharmaceutically acceptable salt thereof, wherein:
X¹ is C—R^{x1} or N;
X² is C—R^{x2} or N;
X³ is C—R^{x3} or N;
X⁴ is C—R^{x4} or N;
X⁶ is C—R^{x6} or N;
wherein no more than three of X¹, X², X³, X⁴, or X⁶ are N;
each R^{x1}, R^{x2}, R^{x3}, R^{x4}, R^{x5}, and R^{x6} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
each L³ is independently a covalent bond, —O—, or —NR—;
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R⁴ is —CN or C₁₋₆ aliphatic optionally substituted with —OR; and
m is 0-4.

39-44. (canceled)

45. A compound of Formula I″: 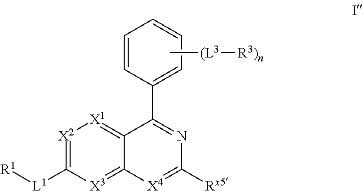
or a pharmaceutically acceptable salt thereof, wherein:
X¹ is C—R^{x1} or N;
X² is C—R^{x2} or N;
X³ is C—R^{x3} or N;
X⁴ is C—R^{x4} or N;
wherein no more than two of X¹, X², X³, or X⁴ are N;
each R^{x1}, R^{x2}, R^{x3}, and R^{x4} is independently selected from hydrogen, —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R^{x5′} is selected from —CN, halogen, —OR, —N(R)₂, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur
each R is independently hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
L¹ is —C(O)N(R²)—*, —S(O)₂—*, —S(O)₂N(R²)—*, or —C(O)O—*, wherein * represents the point of attachment to R¹;
R¹ is hydrogen or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur;
R² is hydrogen or an optionally substituted C₁₋₆ aliphatic; or
R¹ and R², together with their intervening atoms, may form an optionally substituted 3- to 7-membered saturated or partially unsaturated heterocyclic ring having 1-3 heteroatoms atoms independently selected from nitrogen, oxygen, and sulfur;
each L³ is independently a covalent bond, —O—, or —NR—; and
each R³ is independently selected from hydrogen, halogen, or an optionally substituted group selected from the group consisting of C₁₋₆ aliphatic, phenyl, a 5- to 6-membered heteroaryl ring having 1-3 heteroatoms independently selected from nitrogen, oxygen, and sulfur, a 3- to 7-membered saturated or partially unsaturated carbocyclic ring, and a 3- to 7-membered saturated or partially unsaturated heterocyclic ring having 1-3 heteroatoms independently selected from nitrogen, oxygen, and sulfur.

46-49. (canceled)

50. The compound of claim 1, wherein the compound is selected from the group consisting of: 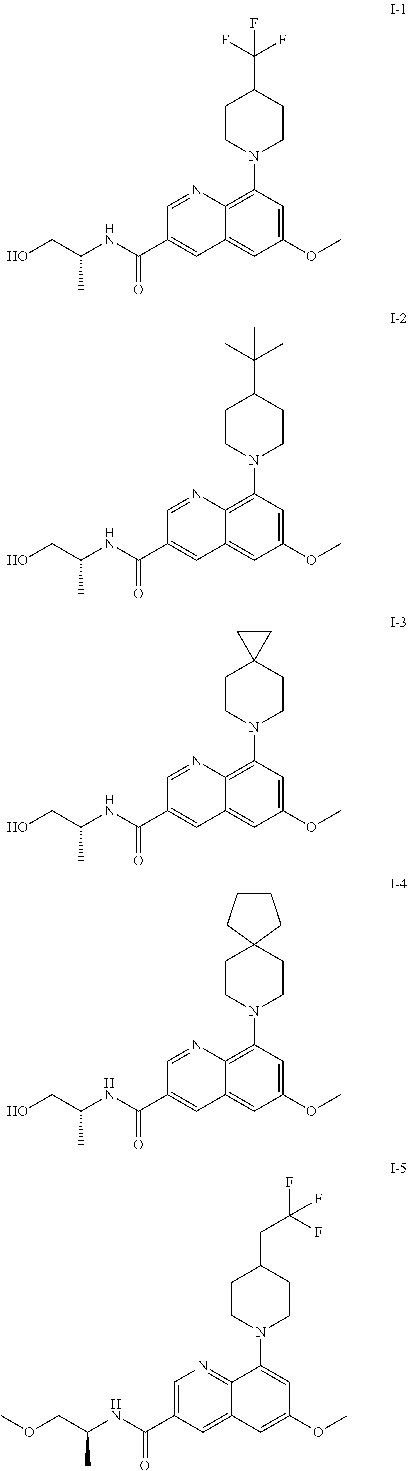 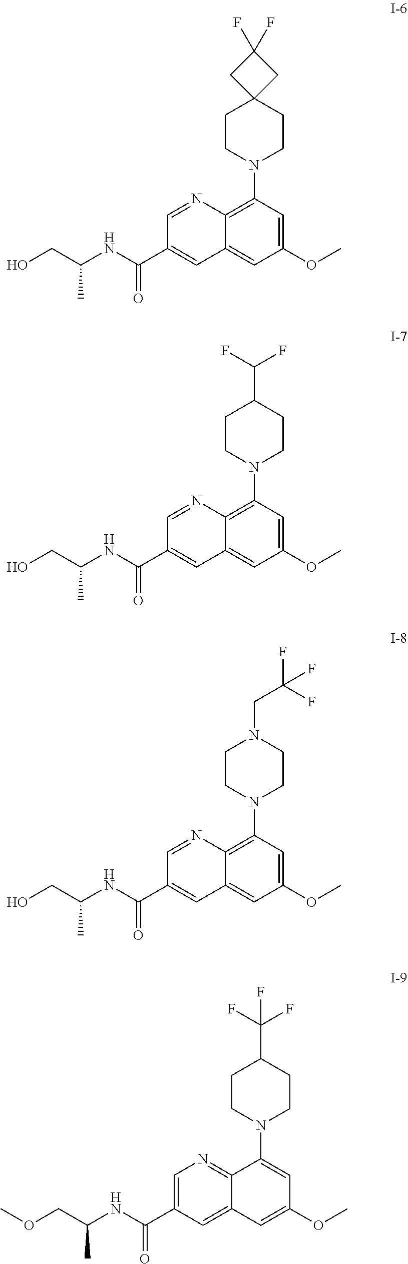 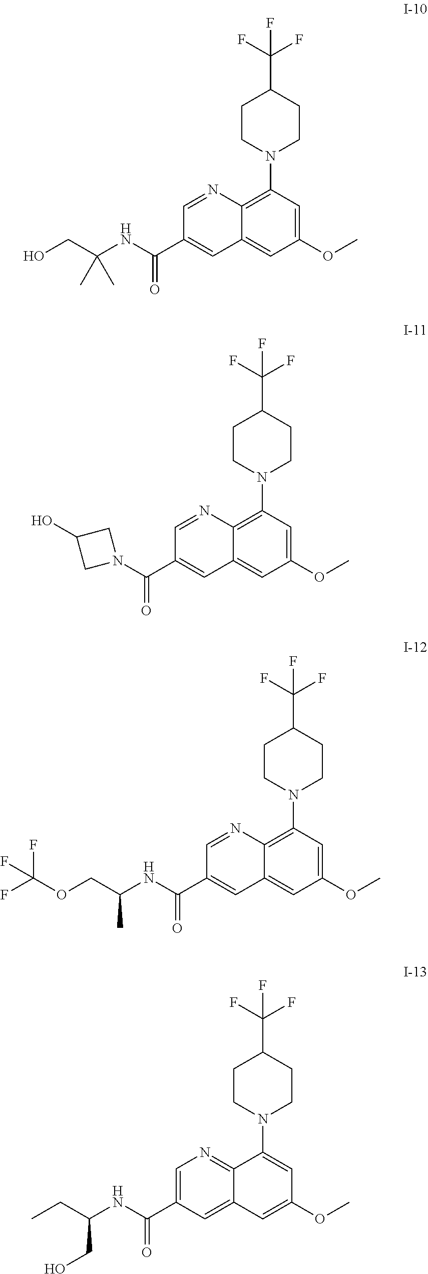 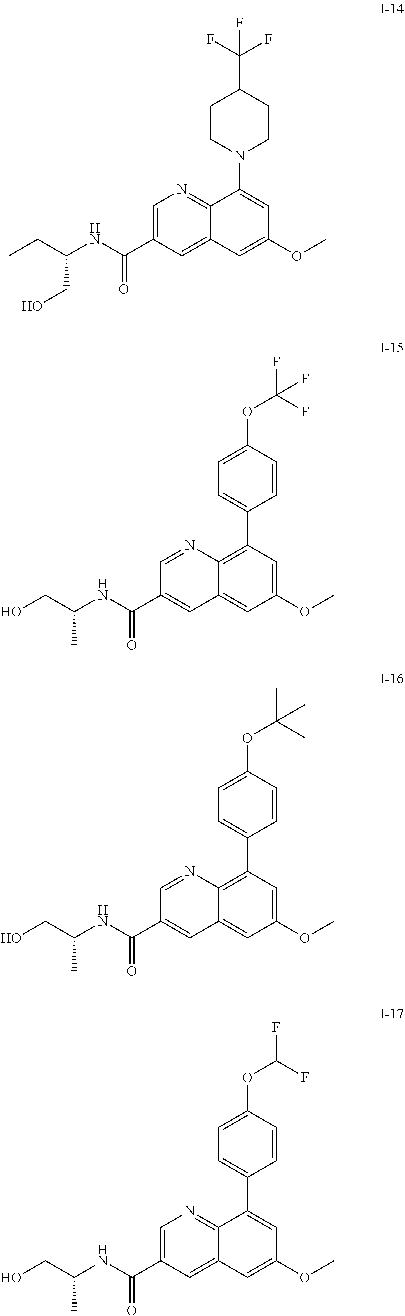 [SMILES: CC[C@@H](CO)NC(=O)c1cnc2c(N3CCC(C(F)(F)F)CC3)cc(OC)cc2c1 ; COc1cc(-c2ccc(OC(F)(F)F)cc2)c2ncc(C(=O)N[C@H](C)CO)cc2c1 ; COc1cc(-c2ccc(OC(C)(C)C)cc2)c2ncc(C(=O)N[C@H](C)CO)cc2c1 ; COc1cc(-c2ccc(OC(F)F)cc2)c2ncc(C(=O)N[C@H](C)CO)cc2c1] 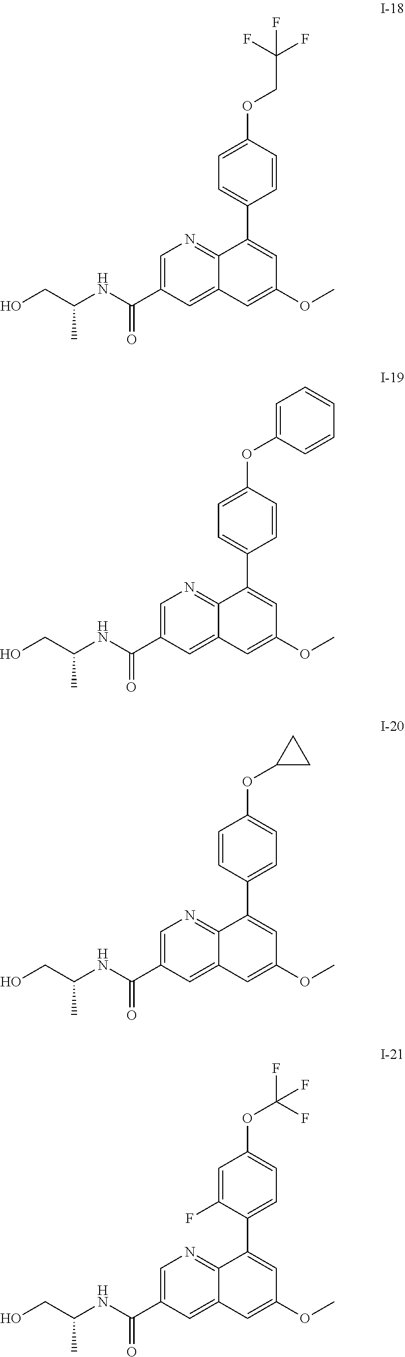 [SMILES: COc1cc(-c2ccc(OCC(F)(F)F)cc2)c2ncc(C(=O)N[C@H](C)CO)cc2c1 ; COc1cc(-c2ccc(Oc3ccccc3)cc2)c2ncc(C(=O)N[C@H](C)CO)cc2c1 ; COc1cc(-c2ccc(OC3CC3)cc2)c2ncc(C(=O)N[C@H](C)CO)cc2c1 ; COc1cc(-c2ccc(OC(F)(F)F)cc2F)c2ncc(C(=O)N[C@H](C)CO)cc2c1] 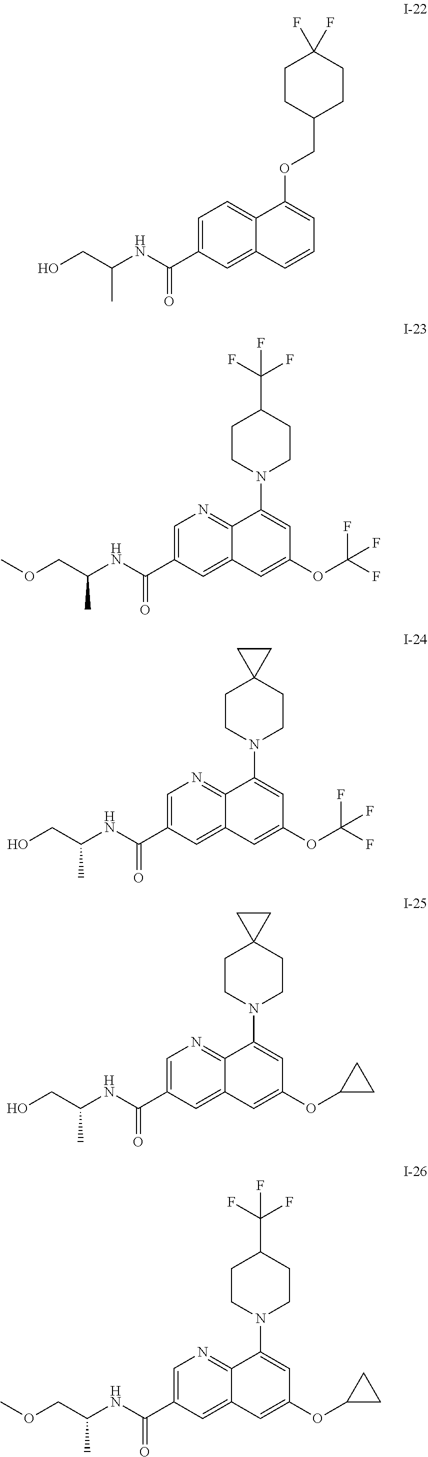 [SMILES: CC(CO)NC(=O)c1ccc2c(OCC3CCC(F)(F)CC3)cccc2c1 ; COC[C@H](C)NC(=O)c1cnc2c(N3CCC(C(F)(F)F)CC3)cc(OC(F)(F)F)cc2c1 ; C[C@H](CO)NC(=O)c1cnc2c(N3CCC4(CC3)CC4)cc(OC(F)(F)F)cc2c1 ; C[C@H](CO)NC(=O)c1cnc2c(N3CCC4(CC3)CC4)cc(OC3CC3)cc2c1 ; COC[C@@H](C)NC(=O)c1cnc2c(N3CCC(C(F)(F)F)CC3)cc(OC3CC3)cc2c1] 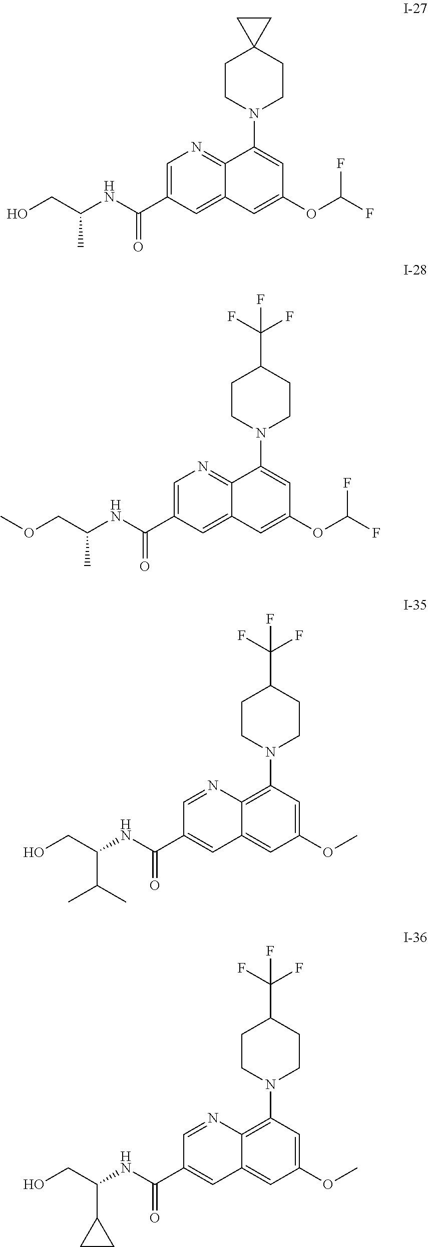 [SMILES: C[C@H](CO)NC(=O)c1cnc2c(N3CCC4(CC3)CC4)cc(OC(F)F)cc2c1 ; COC[C@@H](C)NC(=O)c1cnc2c(N3CCC(C(F)(F)F)CC3)cc(OC(F)F)cc2c1 ; COc1cc(N2CCC(C(F)(F)F)CC2)c2ncc(C(=O)N[C@@H](CO)C(C)C)cc2c1 ; COc1cc(N2CCC(C(F)(F)F)CC2)c2ncc(C(=O)N[C@@H](CO)C3CC3)cc2c1] 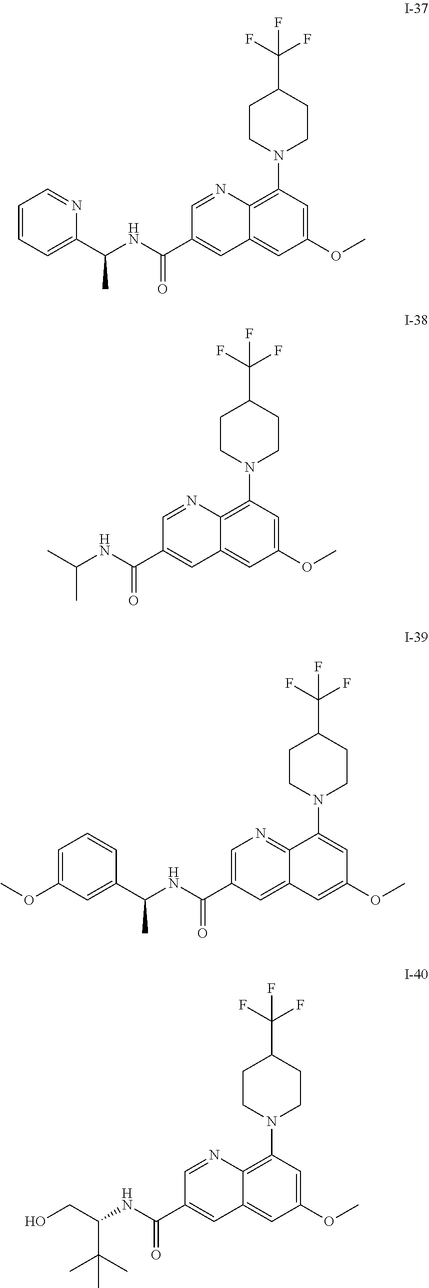 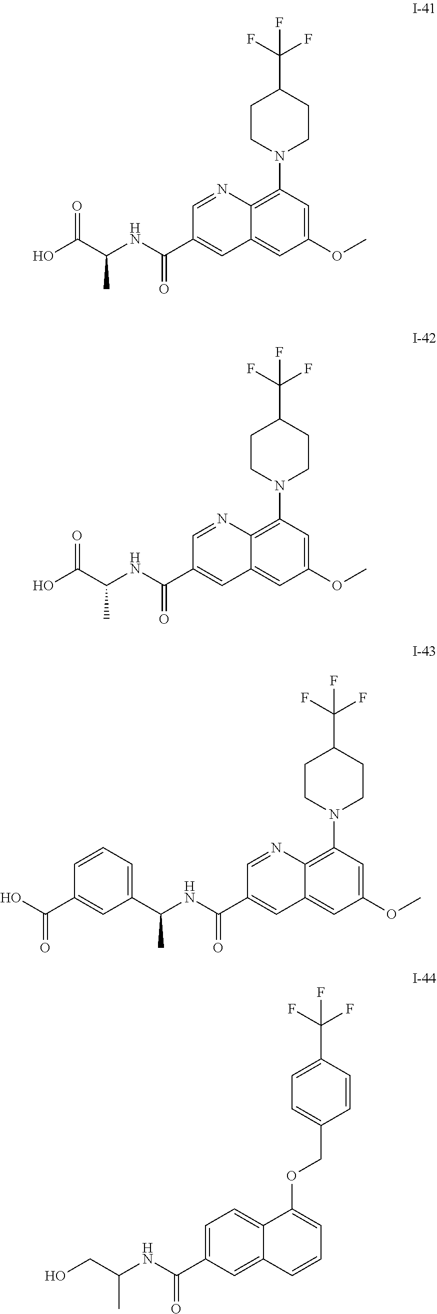 [SMILES: COc1cc(N2CCC(C(F)(F)F)CC2)c2ncc(C(=O)N[C@@H](C)C(=O)O)cc2c1 ; COc1cc(N2CCC(C(F)(F)F)CC2)c2ncc(C(=O)N[C@H](C)C(=O)O)cc2c1 ; COc1cc(N2CCC(C(F)(F)F)CC2)c2ncc(C(=O)N[C@@H](C)c3cccc(C(=O)O)c3)cc2c1 ; CC(CO)NC(=O)c1ccc2c(OCc3ccc(C(F)(F)F)cc3)cccc2c1] 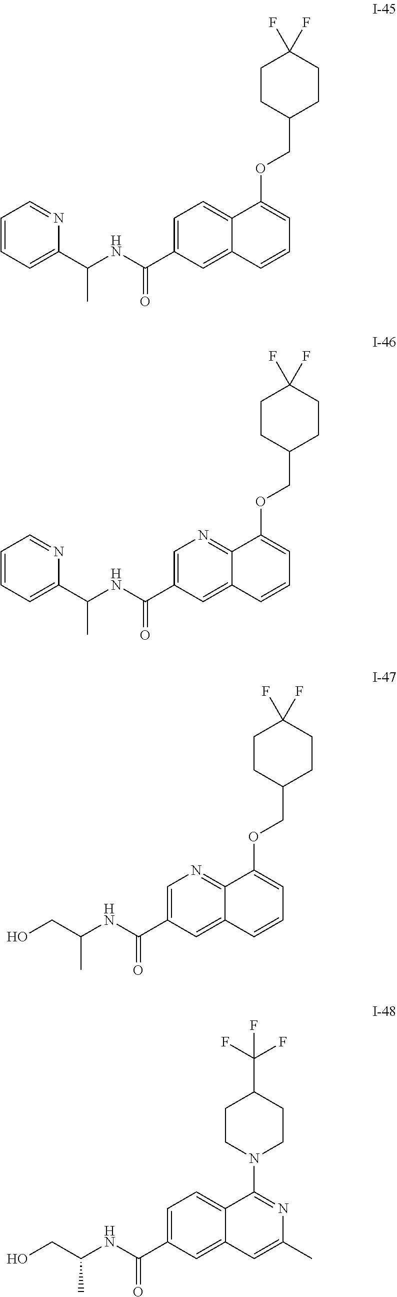 [SMILES: CC(NC(=O)c1ccc2c(OCC3CCC(F)(F)CC3)cccc2c1)c1ccccn1 ; CC(NC(=O)c1cnc2c(OCC3CCC(F)(F)CC3)cccc2c1)c1ccccn1 ; CC(CO)NC(=O)c1cnc2c(OCC3CCC(F)(F)CC3)cccc2c1 ; Cc1cc2cc(C(=O)N[C@H](C)CO)ccc2c(N2CCC(C(F)(F)F)CC2)n1] 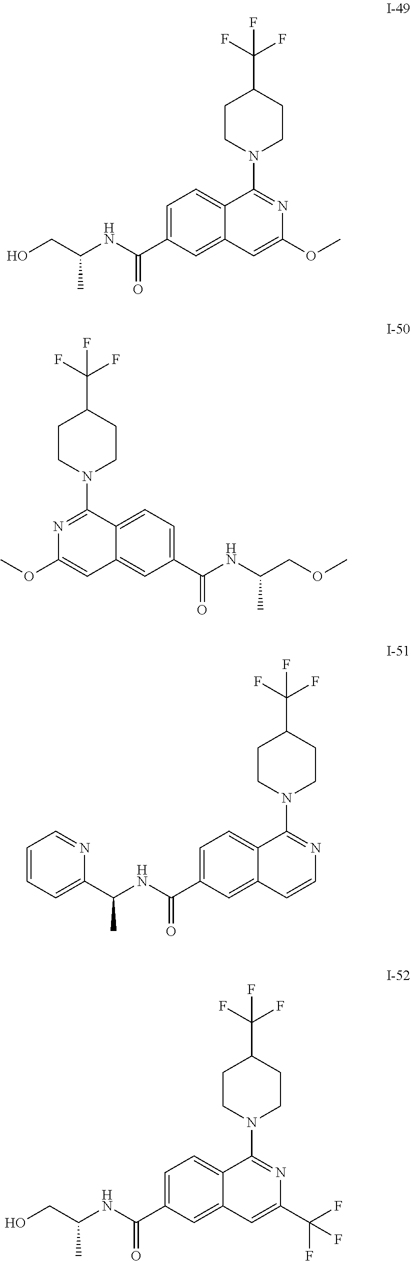 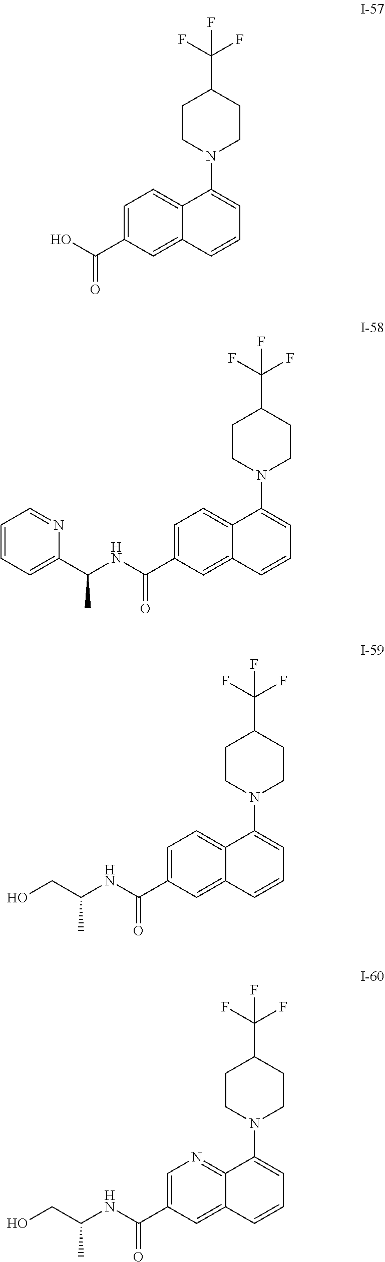 [SMILES: O=C(O)c1ccc2c(N3CCC(C(F)(F)F)CC3)cccc2c1 ; C[C@H](NC(=O)c1ccc2c(N3CCC(C(F)(F)F)CC3)cccc2c1)c1ccccn1 ; C[C@H](CO)NC(=O)c1ccc2c(N3CCC(C(F)(F)F)CC3)cccc2c1 ; C[C@H](CO)NC(=O)c1cnc2c(N3CCC(C(F)(F)F)CC3)cccc2c1] 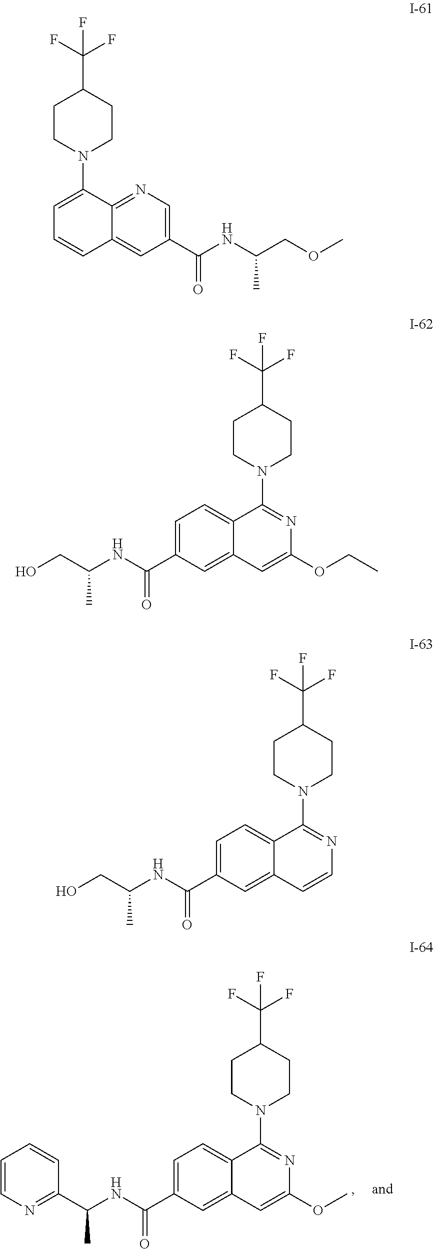 [SMILES: COC[C@H](C)NC(=O)c1cnc2c(N3CCC(C(F)(F)F)CC3)cccc2c1 ; CCOc1cc2cc(C(=O)N[C@H](C)CO)ccc2c(N2CCC(C(F)(F)F)CC2)n1 ; C[C@H](CO)NC(=O)c1ccc2c(N3CCC(C(F)(F)F)CC3)nccc2c1 ; COc1cc2cc(C(=O)N[C@@H](C)c3ccccn3)ccc2c(N2CCC(C(F)(F)F)CC2)n1] 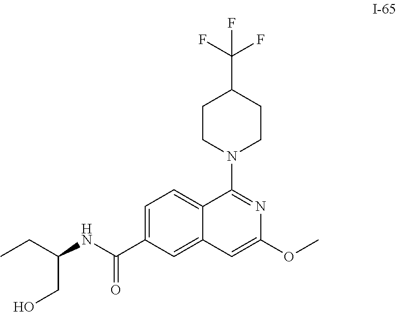 [SMILES: CC[C@H](CO)NC(=O)c1ccc2c(N3CCC(C(F)(F)F)CC3)nc(OC)cc2c1]
or a pharmaceutically acceptable salt thereof.

51. A pharmaceutical composition comprising a compound according to claim 1, or a pharmaceutically acceptable salt thereof, and a pharmaceutically acceptable carrier, adjuvant, or vehicle.

52. A method of inhibiting activity of a TEAD transcription factor, or a mutant thereof, in a biological sample or in a patient, the method comprising a step of contacting the biological sample or administering to a patient a compound according to claim 1, or a pharmaceutically acceptable salt thereof.

53. A method of treating a disease or disorder associated with TEAD, the method comprising a step of administering to a patient in need thereof a compound according to claim 1, or a pharmaceutically acceptable salt thereof.

54-58. (canceled)


SEQUENCE LISTING (5 sequences)

SEQ ID NO: 1 - protein, 426 aa, Homo sapiens
MEPSSWSGSESPAENMERMSDSADKPIDNDAEGVWSPDIEQSFQEALAIYPPCGRRKIILSDEGKMYGRNELIARYIKLRTGKTRTRKQVSSHIQVLARRKSRDFHSKLKDQTAKDKALQHMAAMSSAQIVSATAIHNKLGLPGIPRPTFPGAPGFWPGMIQTGQPGSSQDVKPFVQQAYPIQPAVTAPIPGFEPASAPAPSVPAWQGRSIGTTKLRLVEFSAFLEQQRDPDSYNKHLFVHIGHANHSYSDPLLESVDIRQIYDKFPEKKGGLKELFGKGPQNAFFLVKFWADLNCNIQDDAGAFYGVTSQYESSENMTVTCSTKVCSFGKQVVEKVETEYARFENGRFVYRINRSPMCEYMINFIHKLKHLPEKYMMNSVLENFTILLVVTNRDTQETLLCMACVFEVSNSEHGAQHHIYRLVKD

SEQ ID NO: 2 - protein, 447 aa, Homo sapiens
MGEPRAGAALDDGSGWTGSEEGSEEGTGGSEGAGGDGGPDAEGVWSPDIEQSFQEALAIYPPCGRRKIILSDEGKMYGRNELIARYIKLRTGKTRTRKQVSSHIQVLARRKSREIQSKLKDQVSKDKAFQTMATMSSAQLISAPSLQAKLGPTGPQASELFQFWSGGSGPPWNVPDVKPFSQTPFTLSLTPPSTDLPGYEPPQALSPLPPPTPSPPAWQARGLGTARLQLVEFSAFVEPPDAVDSYQRHLFVHISQHCPSPGAPPLESVDVRQIYDKFPEKKGGLRELYDRGPPHAFFLVKFWADLNWGPSGEEAGAGGSISSGGFYGVSSQYESLEHMTLTCSSKVCSFGKQVVEKVETERAQLEDGRFVYRLLRSPMCEYLVNFLHKLRQLPERYMMNSVLENFTILQVVTNRDTQELLLCTAYVFEVSTSERGAQHHIYRLVRD

SEQ ID NO: 3 - protein, 435 aa, Homo sapiens
MASNSWNASSSPGEAREDGPEGLDKGLDNDAEGVWSPDIEQSFQEALAIYPPCGRRKIILSDEGKMYGRNELIARYIKLRTGKTRTRKQVSSHIQVLARKKVREYQVGIKAMNLDQVSKDKALQSMASMSSAQIVSASVLQNKFSPPSPLPQAVFSTSSRFWSSPPLLGQQPGPSQDIKPFAQPAYPIQPPLPPTLSSYEPLAPLPSAAASVPVWQDRTIASSRLRLLEYSAFMEVQRDPDTYSKHLFVHIGQTNPAFSDPPLEAVDVRQIYDKFPEKKGGLKELYEKGPPNAFFLVKFWADLNSTIQEGPGAFYGVSSQYSSADSMTISVSTKVCSFGKQVVEKVETEYARLENGRFVYRIHRSPMCEYMINFIHKLKHLPEKYMMNSVLENFTILQVVTSRDSQETLLVIAFVFEVSTSEHGAQHHVYKLVKD

SEQ ID NO: 4 - protein, 434 aa, Homo sapiens
MEGTAGTITSNEWSSPTSPEGSTASGGSQALDKPIDNDAEGVWSPDIEQSFQEALAIYPPCGRRKIILSDEGKMYGRNELIARYIKLRTGKTRTRKQVSSHIQVLARRKAREIQAKLKDQAAKDKALQSMAAMSSAQIISATAFHSSMALARGPGRPAVSGFWQGALPGQAGTSHDVKPFSQQTYAVQPPLPLPGFESPAGPAPSPSAPPAPPWQGRSVASSKLWMLEFSAFLEQQQDPDTYNKHLFVHIGQSSPSYSDPYLEAVDIRQIYDKFPEKKGGLKDLFERGPSNAFFLVKFWADLNTNIEDEGSSFYGVSSQYESPENMIITCSTKVCSFGKQVVEKVETEYARYENGHYSYRIHRSPLCEYMINFIHKLKHLPEKYMMNSVLENFTILQVVTNRDTQETLLCIAYVFEVSASEHGAQHHIYRLVKE

SEQ ID NO: 5 - protein, 6 aa, Artificial Sequence
  Description of Artificial Sequence Synthetic peptide
  MOD_RES (2)..(2): Any amino acid
  MOD_RES (4)..(5): Any amino acid
HXRXXS